SEQUENCE LISTING (2 sequences)

SEQ ID NO: 1 - protein, 1130 aa, synthetic construct
  REGION 1..1130: note = Synthetic polypeptide
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 2 - protein, 23 aa, synthetic construct
  REGION 1..23: note = Synthetic polypeptide
MKSSHHHHHHHHHHENLYFQSNA

(19) United States
(12) Patent Application Publication
(10) Pub. No.: US 2025/0144224 A1
(43) Pub. Date: May 8, 2025

(54) ABL INHIBITORS AND USES THEREOF

(71) Applicant: The Regents of the University of California, Oakland, CA (US)

(72) Inventors: Kevan M. Shokat, San Francisco, CA (US); Kevin Lou, San Francisco, CA (US); Jack W. Stevenson, San Francisco, CA (US)

(21) Appl. No.: 18/718,781

(22) PCT Filed: Dec. 13, 2022
(86) PCT No.: PCT/US2022/081432

§ 371 Date: Jun. 11, 2024

Related U.S. Application Data
(60) Provisional application No. 63/288,945, filed on Dec. 13, 2021.

(51) Int. Cl. A61K 47/55; A61K 47/54; A61K 47/60

(52) U.S. Cl. CPC A61K 47/55; A61K 47/545; A61K 47/60

(57) ABSTRACT

Described herein, inter alia, are ABL inhibitors and uses thereof.

### CROSS-REFERENCES TO RELATED APPLICATIONS

This application claims the benefit of U.S. Provisional Application No. 63/288,945, filed Dec. 13, 2021, which is incorporated herein by reference in its entirety and for all purposes.

### REFERENCE TO AN ELECTRONIC SEQUENCE LISTING

The contents of the electronic sequence listing (048536-723001WO_Sequence_Listing_ST26.xml; Size: 4,681 bytes; and Date of Creation: Dec. 5, 2022) is hereby incorporated by reference in its entirety.

### BACKGROUND

The search for cell permeable drugs has conventionally been focused around low molecular weight, non-polar, and rigid chemical space. Emerging therapeutic strategies employing flexibly linked chemical entities composed of more than one ligand, however, necessitate exploration of new chemical space. The growing number of such “beyond rule of five” compounds in early drug discovery and the clinic suggests that mechanisms apart from passive diffusion may assist these molecules in navigating through cell membranes. Disclosed herein, inter alia, are solutions to these and other problems in the art.

### BRIEF SUMMARY

In an aspect is provided a compound, or a pharmaceutically acceptable salt thereof, including a monovalent ABL ATP binding site inhibitor covalently bound to a monovalent ABL myristoyl binding site inhibitor.

In an aspect is provided a pharmaceutical composition including a compound described herein, or a pharmaceutically acceptable salt thereof, and a pharmaceutically acceptable excipient.

In an aspect is provided a method of treating cancer in a subject in need thereof, the method including administering to the subject in need thereof a therapeutically effective amount of a compound described herein, or a pharmaceutically acceptable salt thereof.

In an aspect is provided a method of treating a neurodegenerative disease in a subject in need thereof, the method including administering to the subject in need thereof a therapeutically effective amount of a compound described herein, or a pharmaceutically acceptable salt thereof.

In an aspect is provided a method of treating an ABL-associated disease in a subject in need thereof, the method including administering to the subject in need thereof a therapeutically effective amount of a compound described herein, or a pharmaceutically acceptable salt thereof.

In an aspect is provided a method of reducing the level of activity of ABL in a cell, the method including contacting the cell with an effective amount of a compound described herein, or a pharmaceutically acceptable salt thereof.

### BRIEF DESCRIPTION OF THE DRAWINGS

FIGS. 1A-1C. IFITM proteins promote the inhibitory activity of a bitopic MTOR inhibitor. FIG. 1A: Chemical structures of MTOR inhibitors. FIG. 1B: Gene phenotypes from genome-scale CRISPRi and CRISPRa screens in K562 cells. Genes involved in MTOR complex 1 (MTOR and RPTOR), a requisite rapamycin inhibitory complex partner (FKBP12), and clade I IFITM proteins (IFITM1, IFITM2, and IFITM3) are highlighted. Data represent two biological replicates. FIG. 1C: Spearman correlation coefficients between RapaLink-1 sensitivity, as measured by dose-response data, and transcript abundance, as measured by RNA sequencing (see also FIGS. 8A-8C). Dose-response data are expressed as area under the curve (AUC) and RNA sequencing data are expressed as reads per kilobase of transcript, per million mapped reads (RPKM). Genes are highlighted as in FIG. 1B.

FIGS. 2A-2E. IFITM proteins promote the cellular uptake of linked chemotypes. FIG. 2A: Chemical structures of fluorescent RapaLink-1 analogs. FIGS. 2B-2C: Measurement of fluorescent molecule uptake in K562 CRISPRi (FIG. 2B) or CRISPRa (FIG. 2C) cells expressing sgRNAs (sgRNA+). Cells were incubated with TAMRA-N₃ (10 nM), TAMRA-PEG8-N₃ (1 μM), or RapaTAMRA (1 nM) for 24 h. Uptake modulation by sgRNAs was quantified by internal normalization to non-transduced cells (sgRNA−) present within the mixture (i.e., relative cellular uptake). Data representative of three biological replicates. FIG. 2D: Changes in uptake of fluorescent molecules by sgRNAs targeting IFITM1-3 as in (FIGS. 2B-2C). Relative cellular uptake <1 indicates decreased uptake and >1 indicates increased uptake. Data represent means of three biological replicates. FIG. 2E: Correlation between relative cellular uptake values for RapaTAMRA in (FIG. 2D) and sensitivity/resistance phenotypes from RapaLink-1 CRISPRi/a screens.

FIGS. 3A-3F. Design and characterization of an IFITM-dependent bitopic BCR-ABL1 inhibitor. FIG. 3A: Molecular model of ABL1 kinase domain (left) and chemical structures (right) of BCR-ABL1 inhibitors. The model was constructed by aligning two crystal structures: one bound to dasatinib (PDB, 2GQG) and one bound to asciminib (PDB, 5MO4). FIG. 3B: Viability of K562 CRISPRi (left) or CRISPRa (right) cells expressing sgRNAs treated with DasatiLink-1. Data represent means of three biological replicates; error bars denote SD. FIGS. 3C-3D: Immunoblots of K562 CRISPRi (FIG. 3C) or CRISPRa (FIG. 3D) cells expressing sgRNAs treated with DasatiLink-1 (10 nM) for the times indicated. FIG. 3E: ATP-site pulldown of ABL1 kinase domain in the presence of inhibitor with or without addition of 100-fold molar excess asciminib (Asc). Data represent two biological replicates. FIG. 3F: In-cell kinase occupancy profiling of DasatiLink-1 and an unlinked control (a 1:1 mixture of dasatinib and asciminib) at equimolar concentration (100 nM). Data represent three biological replicates.

FIGS. 4A-4C. IFITM proteins expand the chemical space of cell permeable molecules. FIG. 4A: Heavy atom skeletons of compounds assessed for IFITM dependency (see also FIGS. 10A-10D for chemical structures). Compounds were categorized as non-linked inhibitors (compounds 1-7), PROTACs (compounds 8-11), or bitopic inhibitors (compounds 12-13). FIG. 4B: Chemical-genetic interaction map of inhibitors in FIG. 4A with IFITM1-3. Potency, as measured by IC₅₀ in a cell viability assay, was normalized to that of non-sgRNA-expressing K562 CRISPRi or CRISPRa cells. Physicochemical properties, including molecular weight (MW) and number of rotatable bonds, with their respective traditional thresholds for drug-likeness are indicated (right). Data represent means of three biological replicates. FIG. 4C: Map of chemical space populated by 260 kinase inhibitors in clinical development (black), 2258 PROTACs reported in the literature (gray), and 2 bitopic inhibitors described herein. Boundaries represent traditional guidelines for drug-likeness.

FIGS. 5A-5H. CRISPRi/a screening in K562 cells identifies genes that determine cellular response to MTOR inhibitors. FIG. 5A: Population doublings of K562 CRISPRi cells over the course of functional genomics screens. Arms correspond to continuous inhibitor treatment with the indicated concentrations. Data represent means of two biological replicates; error bars denote SD. FIGS. 5B-5D: sgRNA phenotypes derived from growth selections in FIG. 5A. Targeting sgRNAs (black) and non-targeting sgRNAs (gray) are plotted for two biological replicates. FIG. 5E: As in FIG. 5A for K562 CRISPRa cells. FIGS. 5F-5H: As in FIGS. 5B-5D for K562 CRISPRa cells.

FIGS. 6A-6B. Established MTOR regulatory mechanisms modulate sensitivity/resistance to MTOR inhibitors. FIG. 6A: Pathway map of chemical-genetic interactions with a 1:1 mixture of sapanisertib and rapamycin in a genome-scale K562 CRISPRi screen. Color intensities portray phenotype strength and circle diameters represent −log₁₀ Mann-Whitney P values. Data represent two biological replicates. FIG. 6B: As in FIG. 6A for RapaLink-1.

FIGS. 7A-7E. IFITM protein expression synergizes specifically with RapaLink-1 inhibitory activity in K562 CRISPRi/a cells. FIG. 7A: Schematic of the human IFITM locus located within chromosome 11 annotated with positions targeted by sgRNAs described herein. FIG. 7B: Immunoblots of K562 CRISPRi cells stably expressing sgRNAs. Cells were collected for assessment 30 days following selection for sgRNA+ cells. Data representative of three biological replicates. FIG. 7C: as in FIG. 7B for K562 CRISPRa cells collected for assessment 15 days following selection for sgRNA+ cells. FIGS. 7D-7E: K562 CRISPRi (FIG. 7D) or CRISPRa (FIG. 7E) cells transduced with sgRNAs were grown in the presence or absence of continuous inhibitor treatment (1 nM) as in the corresponding genome-scale screens. Relative populations of transduced (sgRNA+) and non-transduced (sgRNA−) cells were determined by flow cytometry at the indicated times. Data represent means of three biological replicates; error bars denote SD.

FIGS. 8A-8C. Basal IFITM protein expression correlates specifically with RapaLink-1 inhibitory activity across diverse cancer cell lines. FIGS. 8A-8B: Spearman correlation coefficients between sapanisertib (FIG. 8A) or rapamycin (FIG. 8B) sensitivity, as measured by dose-response data, and transcript abundance, as measured by RNA sequencing (see also FIG. 1C). FIG. 8C: Data used to correlate IFITM1-3 transcript abundance and inhibitor sensitivity in (FIGS. 8A-8B and FIG. 1C). Points represent individual cell lines with Spearman correlation coefficients (p) indicated for each transcript. Pearson correlation coefficients (r) and linear regressions provided for visualization.

FIGS. 9A-9B. DasatiLink-1 engages ABL1 kinase domain through a bitopic mechanism. FIG. 9A: ¹H-¹⁵N heteronuclear single quantum coherence (HSQC) spectra of ABL1 kinase domain in the presence of dasatinib, asciminib, dasatinib+asciminib, and DasatiLink-1. FIG. 9B: Chemical shift differences for assigned residues in ABL1 kinase domain resulting from interactions with different inhibitors as in FIG. 9A. δ (ppm) >0.1 indicates a major chemical shift difference.

FIGS. 10A-10D. Chemical structures of inhibitors assessed for IFITM dependency.

FIG. 11. Computed physicochemical properties of compounds described herein.

FIG. 12. Artificial membrane permeability of bitopic inhibitors and their respective non-linked counterparts.

FIGS. 13A-13B. Biochemical inhibition of BCR-ABL (wild type) and BCR-ABL (T315I). FIG. 13A: Chemical structures of inhibitors tested. FIG. 13B: Top graphs: Inhibitors tested are dasatinib (circles), asciminib (squares), and combination of dasatinib and asciminib (triangles). Bottom graphs: Inhibitors tested are DasatiLink-1 (PEG12) (circles), DasatiLink-2 (PEG10) (squares), DasatiLink-3 (PEG8) (triangles tip up), and DasatiLink-4 (PEG6) (triangles tip down). Biochemical experiments performed by Thermo SelectScreen, under conditions which mask the effects of allosteric inhibition. Conditions contain 0.01% BRIJ-35, a common non-ionic detergent added to kinase assay buffers. Reference: Choi, Y. et al., J Biol Chem 284, 29005-29014 (2009).

FIG. 14. Reagents and conditions for synthesis of DasatiLink-1, DasatiLink-2, DasatiLink-3, and DasatiLink-4. (a) HATU, DIPEA, DMF, rt, 80%. (b) DIPEA, IPA, 140° C., 91%. (c) K₃PO₄, Pd(PPh₃)₄, toluene, 110° C., 40%. (d) LiOH·H₂O, H₂O, MeOH, 98%. (e) HATU, DIPEA, DMF, rt, 64-91%. (f) TFA, CH₂Cl₂, rt. (g) HATU, DIPEA, DMF, rt, 71-92% over two steps. (h) TFA, CH₂Cl₂, rt, 72-81%. Abbreviations: HATU, 1-[bis(dimethylamino)methylene]-1H-1,2,3-triazolo[4,5-b]pyridinium 3-oxide hexafluorophosphate; DIPEA, N,N-diisopropylethylamine; DMF, N,N-dimethylformamide; IPA, isopropyl alcohol; TFA, trifluoroacetic acid.

FIG. 15. Reagents and conditions for synthesis of PonatiLink-1. (a) TFA, CH₂Cl₂, rt. (b) HATU, DIPEA, DMF, rt, 94% over two steps. (c) TFA, CH₂Cl₂, rt. (d) HATU, DIPEA, DMF, rt, 69% over two steps. (e) TFA, CH₂Cl₂, rt, 46%. Abbreviations: HATU, 1-[bis(dimethylamino)methylene]-1H-1,2,3-triazolo[4,5-b]pyridinium 3-oxide hexafluorophosphate; DIPEA, N,N-diisopropylethylamine; DMF, N,N-dimethylformamide; TFA, trifluoroacetic acid.

FIGS. 16A-16C. FIG. 16A: Molecular model of ABL1 kinase domain with arrows indicating linkage vector used in DasatiLink series. The model was constructed by aligning two crystal structures: one bound to dasatinib (PDB, 2GQG) and one bound to asciminib (PDB, 5MO4). A representative structure of a DasatiLink series compound is shown. FIG. 16B: Molecular model of ABL1 kinase domain with arrows indicating linkage vector used in PonatiLink-1 series. The model was constructed by aligning two crystal structures: one bound to ponatinib (PDB, 3OXZ) and one bound to asciminib (PDB, 5MO4). A representative structure of a PonatiLink-1 series compound is shown. FIG. 16C: As in FIG. 16B, for the PonatiLink-2 series of compounds.

FIG. 17. Compounds were tested for ability to inhibit cell growth. K562 cells (wild-type or CRISPR base-edited Bcr-Abl T315I) were plated at 1000 cells/well in 96-well plates and treated with the indicated compounds at the indicated concentrations for three days in triplicate, then tested for cell viability by the CellTiter-Glo 2.0 assay (Promega). Compounds tested are combination of dasatinib and asciminib (circles); DasatiLink-1 (triangles); DasatiLink-2 (filled squares); DasatiLink-3 (plus symbols); and DasatiLink-4 (open squares).

FIG. 18. Compounds were tested for ability to inhibit wild-type and T315I Abl kinases in vitro at the indicated concentrations by the SelectScreen Z'LYTE assay (Thermo Scientific). Compounds tested are combination of ponatinib and asciminib (circles); PonatiLink-1-12 (triangles); PonatiLink-1-16 (filled squares); PonatiLink-1-20 (plus symbols); and PonatiLink-1-24 (open squares).

FIG. 19. Compounds were tested for ability to inhibit cell growth. K562 cells (wild-type or CRISPR base-edited Bcr-Abl T315I) were plated at 1000 cells/well in 96-well plates and treated with the indicated compounds at the indicated concentrations for three days in triplicate, then tested for cell viability by the CellTiter-Glo 2.0 assay (Promega). Compounds tested are combination of ponatinib and asciminib (circles); PonatiLink-1-12 (triangles); PonatiLink-1-16 (filled squares); PonatiLink-1-20 (plus symbols); PonatiLink-1-24 (open squares); and PonatiLink-1-28 (star symbols).

FIG. 20. Compounds were tested for ability to inhibit cell growth. K562 cells (wild-type or CRISPR base-edited Bcr-Abl T315I) were plated at 1000 cells/well in 96-well plates and treated with the indicated compounds at the indicated concentrations for three days in triplicate, then tested for cell viability by the CellTiter-Glo 2.0 assay (Promega). Compounds tested are combination of ponatinib and asciminib (circles); PonatiLink-2-7-4 (triangles); PonatiLink-2-7-6 (filled squares); PonatiLink-2-7-8 (plus symbols); and PonatiLink-2-7-10 (open squares).

FIG. 21. Comparison of potent compounds from the DasatiLink, PonatiLink-1, and PonatiLink-2 series. Compounds were tested for ability to inhibit cell growth. K562 cells (wild-type or CRISPR base-edited Bcr-Abl T315I) were plated at 1000 cells/well in 96-well plates and treated with the indicated compounds at the indicated concentrations for three days in triplicate, then tested for cell viability by the CellTiter-Glo 2.0 assay (Promega). Compounds tested are combination of ponatinib and asciminib (circles); combination of dasatinib and asciminib (triangles); DasatiLink-1 (filled squares); PonatiLink-1-24 (plus symbols); and PonatiLink-2-7-8 (open squares).

FIG. 22. Reagents and conditions for synthesis of PonatiLink-1 analogs. (i) HATU, DIPEA, DMF, rt, 68-80%. (j) TFA, DCM, rt. (k) HATU, DIPEA, DCM, rt, 27-68% over two steps. (l) TFA, DCM, rt, 6-54%. Abbreviations: HATU, 1-[bis(dimethylamino)methylene]-1H-1,2,3-triazolo[4,5-b]pyridinium 3-oxide hexafluorophosphate; DIPEA, N,N-diisopropylethylamine; DMF, N,N-dimethylformamide; IPA, isopropyl alcohol; DCM, dichloromethane; TFA, trifluoroacetic acid; rt, room temperature.

FIG. 23. (m) Cesium carbonate, DMF, 50° C. (n) TFA, DCM, rt, 49% over two steps. (o) HATU, DIPEA, DMF, rt. (p) TFA, DCM, rt, 63-74% over two steps. (q) HATU, DIPEA, DCM, rt. (r) TFA, DCM, rt, 22-47% over two steps. Abbreviations: HATU, 1-[bis(dimethylamino)methylene]-1H-1,2,3-triazolo[4,5-b]pyridinium 3-oxide hexafluorophosphate; DIPEA, N,N-diisopropylethylamine; DMF, N,N-dimethylformamide; IPA, isopropyl alcohol; DCM, dichloromethane; TFA, trifluoroacetic acid; rt, room temperature.

FIG. 24. Synthetic route for PonatiLink-2-7-8 (triazole).

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O—is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′—represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

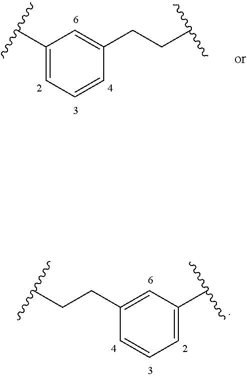 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, =NR′, =N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1} L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

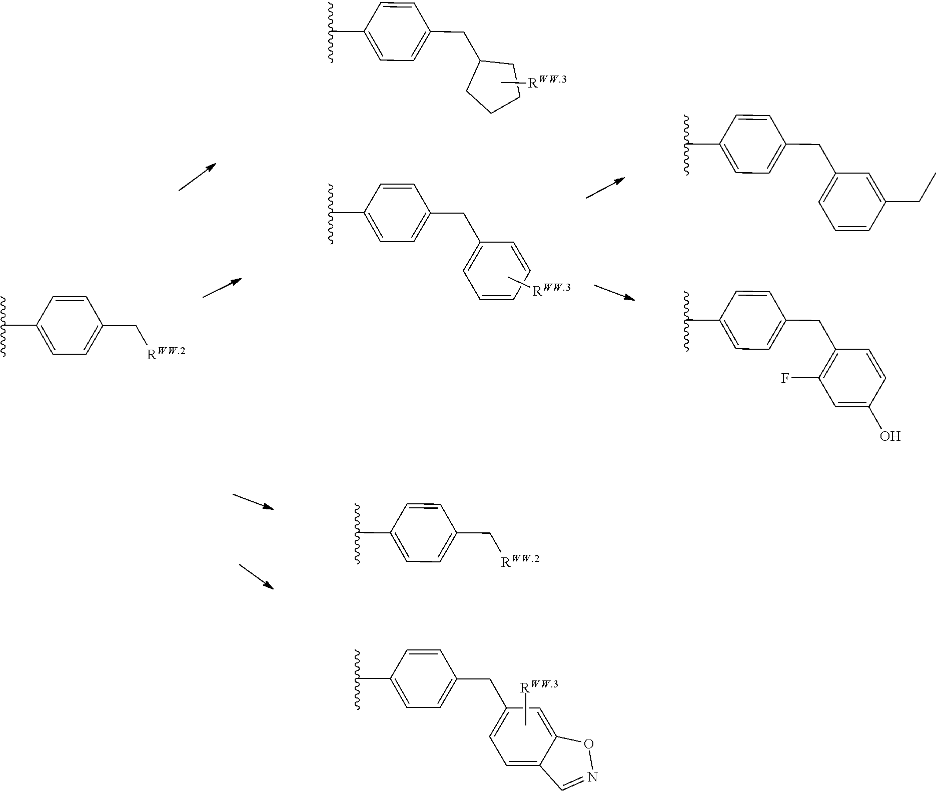

R^{WW.1} is independently oxo, halogen, —CX^{WW.1}^{₃}, —CHX^{WW.1}^{₂}, —CH₂X^{WW.1}, —OCX^{WW.1}^{₃}, —OCH₂X^{WW.1}, —OCHX^{WW.1}^{₂}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}^{₃}, —CHX^{WW.1}^{₂}, —CH₂X^{WW.1}, —OCX^{WW.1}^{₃}, —OCH₂X^{WW.1}, —OCHX^{WW.1}^{₂}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}^{₃}, —CHX^{WW.2}^{₂}, —CH₂X^{WW.2}, —OCX^{WW.2}^{₃}, —OCH₂X^{WW.2}, —OCHX^{WW.2}^{₂}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}^{₃}, —CHX^{WW.2}^{₂}, —CH₂X^{WW.2}, —OCX^{WW.2}^{₃}, —OCH₂X^{WW.2}, —OCHX^{WW.2}^{₂}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}^{₃}, —CHX^{WW.3}^{₂}, —CH₂X^{WW.3}, —OCX^{WW.3}^{₃}, —OCH₂X^{WW.3}, —OCHX^{WW.3}^{₂}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}^{₃}, —CHX^{LWW.1}^{₂}, —CH₂X^{LWW.1}, —OCXL^{LWW.1}^{₃}, —OCH₂X^{LWW.1}, —OCHX^{LWW.1}^{₂}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}_substituted or unsubstituted aryl (e.g., C₆-C₁₂, C₆-C₁₀, or phenyl), or R^{LWW.2}_substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}^{₃}, —CHXL^{LWW.1}^{₂}, —CH₂X^{LWW.1}, —OCX^{LWW.1}^{₃}, —OCH₂X^{LWW.1}, —OCHX^{LWW.1}^{₂}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}^{₃}, —CHX^{LWW.2}^{₂}, —CH₂X^{LWW.2}, —OCX^{LWW.2}^{₃}, —OCH₂X^{LWW.2}, —OCHX^{LWW.2}^{₂}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}^{₃}, —CHX^{LWW.2}^{₂}, —CH₂X^{LWW.2}, —OCX^{LWW.2}^{₃}, —OCH₂X^{LWW.2}, —OCHX^{LWW.2}^{₂}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}^{₃}, —CHX^{LWW.3}^{₂}, —CH₂X^{LWW.3}, —OCX^{LWW.3}^{₃}, —OCH₂X^{LWW.3}, —OCHX^{LWW.3}^{₂}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1} substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is cancer (e.g., chronic myeloid leukemia, acute lymphoblastic leukemia, acute myelogenous leukemia, or mixed-phenotype acute leukemia). In embodiments, the disease is a neurodegenerative disease (e.g., Parkinson's disease or Alzheimer's disease).

As used herein, the term “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifori carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” and “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refers to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., cancer or neurodegenerative disease) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., cancer or neurodegenerative disease) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to T315 of ABL1 when the selected residue occupies the same essential spatial or other structural relationship as T315 of ABL1. In some embodiments, where a selected protein is aligned for maximum homology with ABL1, the position in the aligned selected protein aligning with T315 is said to correspond to T315. Instead of a primary sequence alignment, a three dimensional structural alignment can also be used, e.g., where the structure of the selected protein is aligned for maximum correspondence with ABL and the overall structures compared. In this case, an amino acid that occupies the same essential position as T315 in the structural model is said to correspond to the T315 residue.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “tyrosine-protein kinase ABL1” or “ABL1” or “ABL” refers to a protein (including homologs, isoforms, and functional fragments thereof) encoded by the ABL1 gene involved in processes of cell differentiation, cell division, cell adhesion, and DNA repair. The term includes any recombinant or naturally-occurring form of ABL1 variants thereof that maintain ABL1 activity (e.g., within at least 30%, 40%, 50%, 60%, 70%, 80%, 90%, 95%, or 100% activity compared to wild type ABL1). In embodiments, the ABL1 protein encoded by the ABL1 gene has the amino acid sequence set forth in or corresponding to Entrez 25, UniProt P00519, RefSeq (protein) NP_005148.2, or RefSeq (protein) NP_009297.2. In embodiments, the ABL1 gene has the nucleic acid sequence set forth in RefSeq (mRNA) NM_005157.5 or RefSeq (mRNA) NM 007313.2. In embodiments, the amino acid sequence or nucleic acid sequence is the sequence known at the time of filing of the present application. In embodiments, the amino acid sequence is

(SEQ ID NO: 1)
MLEICLKLVGCKSKKGLSSSSSCYLEEALQRPVASDFEPQGLSEAA
RWNSKENLLAGPSENDPNLFVALYDFVASGDNTLSITKGEKLRVLG
YNHNGEWCEAQTKNGQGWVPSNYITPVNSLEKHSWYHGPVSRNAAE
YLLSSGINGSFLVRESESSPGQRSISLRYEGRVYHYRINTASDGKL
YVSSESRFNTLAELVHHHSTVADGLITTLHYPAPKRNKPTVYGVSP
NYDKWEMERTDITMKHKLGGGQYGEVYEGVWKKYSLTVAVKTLKED
TMEVEEFLKEAAVMKEIKHPNLVQLLGVCTREPPFYIITEFMTYGN
LLDYLRECNRQEVNAVVLLYMATQISSAMEYLEKKNFIHRDLAARN
CLVGENHLVKVADFGLSRLMTGDTYTAHAGAKFPIKWTAPESLAYN
KFSIKSDVWAFGVLLWEIATYGMSPYPGIDLSQVYELLEKDYRMER
PEGCPEKVYELMRACWQWNPSDRPSFAEIHQAFETMFQESSISDEV
EKELGKQGVRGAVSTLLQAPELPTKTRTSRRAAEHRDTTDVPEMPH
SKGQGESDPLDHEPAVSPLLPRKERGPPEGGLNEDERLLPKDKKTN
LFSALIKKKKKTAPTPPKRSSSFREMDGQPERRGAGEEEGRDISNG
ALAFTPLDTADPAKSPKPSNGAGVPNGALRESGGSGFRSPHLWKKS
STLTSSRLATGEEEGGGSSSKRFLRSCSASCVPHGAKDTEWRSVTL
PRDLQSTGRQFDSSTFGGHKSEKPALPRKRAGENRSDQVTRGTVTP
PPRLVKKNEEAADEVFKDIMESSPGSSPPNLTPKPLRRQVTVAPAS
GLPHKEEAGKGSALGTPAAAEPVTPTSKAGSGAPGGTSKGPAEESR
VRRHKHSSESPGRDKGKLSRLKPAPPPPPAASAGKAGGKPSQSPSQ
EAAGEAVLGAKTKATSLVDAVNSDAAKPSQPGEGLKKPVLPATPKP
QSAKPSGTPISPAPVPSTLPSASSALAGDQPSSTAFIPLISTRVSL
RKTRQPPERIASGAITKGVVLDSTEALCLAISRNSEQMASHSAVLE
AGKNLYTFCVSYVDSIQQMRNKFAFREAINKLENNLRELQICPATA
GSGPAATQDFSKLLSSVKEISDIVQR.

The term “ABL ATP binding site” is used in accordance with its plain ordinary meaning. The ABL ATP binding site is well-known and is described, for example, in Schindler, T. et al. Structural Mechanism for STI-571 Inhibition of Abelson Tyrosine Kinase. Science 289, 1938-1942 (2000). In embodiments, the ABL ATP binding site is a BCR-ABL ATP binding site.

The term “BCR-ABL” refers to a fusion protein (including homologs, isoforms, and functional fragments thereof) that is a constitutively active tyrosine kinase that drives uncontrolled cell proliferation.

The term “breakpoint cluster region protein” or “BCR” refers to a protein (including homologs, isoforms, and functional fragments thereof) encoded by the BCR gene. The term includes any recombinant or naturally-occurring form of BCR variants thereof that maintain BCR activity (e.g., within at least 30%, 40%, 50%, 60%, 70%, 80%, 90%, 95%, or 100% activity compared to wild type BCR). In embodiments, the BCR protein encoded by the BCR gene has the amino acid sequence set forth in or corresponding to Entrez 613, UniProt P11274, RefSeq (protein) NP 004318.3, or RefSeq (protein) NP 067585.2. In embodiments, the BCR gene has the nucleic acid sequence set forth in RefSeq (mRNA) NM_004327.3 or RefSeq (mRNA) NM_021574.2. In embodiments, the amino acid sequence or nucleic acid sequence is the sequence known at the time of filing of the present application.

The term “ABL ATP binding site inhibitor” refers to a compound that inhibits the activity of ABL (e.g., kinase activity) and binds to the ATP binding site of ABL1 (e.g., overlapping with the ATP binding site, blocking access by ATP to the ATP binding site of ABL1). Examples of ABL ATP binding site inhibitors include, but are not limited to, dasatinib (also known as BMS-354825; PDB ID: 4XEY), ponatinib (also known as AP24534; PDB ID: 31K3), imatinib (also known as ST1571; PDB ID: 2HYY), nilotinib (also known as AMN-107; PDB ID: 5MO4), bosutinib (also known as SKI-606; PDB ID: 3UE4), bafetinib (also known as INNO-406; PDB ID: 2E2B), olverembatinib (also known as GZD824 or HQP1351), tozasertib (also known as VX-680 or MK-0457; PDB ID: 2F4J), PF-114, rebastinib (also known as DCC-2036), danusertib (also known as PHA-739358; PDB ID: 2V7A), or HG-7-85-01 (PDB ID: 4AGW). In embodiments, the ABL ATP binding site inhibitor is a BCR-ABL ATP binding site inhibitor.

The term “ABL myristoyl binding site” is used in accordance with its plain ordinary meaning. The ABL myristoyl binding site is well-known and is described, for example, in Zhang, J. et al. Targeting Bcr-Abl by combining allosteric with ATP-binding-site inhibitors. Nature 463, 501-506 (2010). In embodiments, the ABL myristoyl binding site is a BCR-ABL myristoyl binding site.

The term “ABL myristoyl binding site inhibitor” refers to a compound that inhibits the activity of ABL (e.g., kinase activity) and binds to the myristoyl binding site, an allosteric binding site, of ABL1 (e.g., myristate binding site, overlapping with the myristoyl binding site, blocking access by a myristoyl group to the myristoyl binding site of ABL1). Examples of ABL myristoyl binding site inhibitors include, but are not limited to, asciminib (also known as ABL001; PDB ID: 5MO4) and GNF-2 (PDB ID: 3K5V). In embodiments, the ABL myristoyl binding site inhibitor is a BCR-ABL myristoyl binding site inhibitor.

The term “selective” or “selectivity” or the like in reference to a compound or agent refers to the compound's or agent's ability to cause an increase or decrease in activity of a particular molecular target (e.g., protein, enzyme, etc.) preferentially over one or more different molecular targets (e.g., a compound having selectivity toward ABL1 would preferentially inhibit ABL1 over other proteins). In embodiments, a “ABL1-selective compound” refers to a compound (e.g., compound described herein) having selectivity towards ABL1.

### II. Compounds

In an aspect is provided a compound, or a pharmaceutically acceptable salt thereof, including a monovalent ABL ATP binding site inhibitor covalently bound to a monovalent ABL myristoyl binding site inhibitor. In embodiments, the compound includes a monovalent BCR-ABL ATP binding site inhibitor covalently bound to a monovalent BCR-ABL myristoyl binding site inhibitor.

In embodiments, a divalent linker binds the monovalent ABL (e.g., BCR-ABL) ATP binding site inhibitor to the monovalent ABL (e.g., BCR-ABL) myristoyl binding site inhibitor. In embodiments, the divalent linker is at least about or about 5 Å in length (e.g., at least about or about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Å in length). In embodiments, the divalent linker is at least about or about the length of 9 methylene groups (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 methylene groups). In embodiments, the divalent linker is at least about or about the length of 12 methylene groups (e.g., at least about or about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 methylene groups). In embodiments, the divalent linker is at least about or about the length of 36 methylene groups (e.g., 36, 37, 38, 39, 40, 41, 42, 43, 44, 45, 46, 47, 48, 49, or 50 methylene groups). In embodiments, the divalent linker is from about 10 to about 80 Å in length. In embodiments, the divalent linker is from about 20 to about 60 Å in length. In embodiments, the divalent linker is from about 30 to about 50 Å in length. In embodiments, the divalent linker is at least about or about 30 Å in length (e.g., at least about or about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Å in length).

The specified length of a linker is the through space distance between the ends of the linker (i.e., the ends or termini that are connected to the two parts of the molecule connected by the linker) wherein the length of the linker is measured when the linker is fully extended and wherein the linker termini are the furthest apart they may naturally exist in solution (i.e., the longest distance between the ends of the linker wherein the linker adopts allowable conformations, bond lengths, and bond angles following the principles of chemistry), e.g., without adopting non-natural bond lengths, non-allowed or non-preferred bond angles, or high energy non-preferred or non-natural interactions of different components of the linker. In embodiments, the linker length is measured when included in a compound as described herein (e.g., aspect, embodiment, example, figures, table, claim). It will be understood that a linker may adopt a through space distance (e.g., in solution, when bound to ABL (e.g., BCR-ABL1)) that is less than the fully extended conformation used to define the linker length.

In embodiments, the linker is a hydrolysable linker (e.g., in solution). In embodiments, the linker is a non-hydrolysable linker (e.g., in solution). In embodiments, the linker may be cleaved by an enzyme (e.g., hydrolase, protease, cytochrome). In embodiments, the linker is not cleavable by an enzyme (e.g., under normal cellular conditions). In embodiments, the linker is a polyethylene glycol linker. In embodiments, the linker is hydrophilic. In embodiments, the linker is hydrophobic. In embodiments, the linker includes a disulfide bond. In embodiments, the linker includes a hydrazone bond. In embodiments, the linker includes an ester. In embodiments, the linker includes a sulfonyl. In embodiments, the linker includes a thioether. In embodiments, the linker includes a phosphinate. In embodiments, the linker includes an alkyloxime bond. In embodiments, the linker includes one or more amino acids. In embodiments, the linker consists of amino acids. In embodiments, the linker includes an amino acid analog. In embodiments, the linker includes an amino acid mimetic. In embodiments, the linker is a linker known in the art for use in linking antibodies to agents (e.g., antibody drug conjugates). In embodiments, the linker is a linker as described in Bioconjugate Techniques (Second Edition) by Greg T. Hermanson (2008), which is herein incorporated by reference in its entirety for all purposes. In embodiments, the linker is a linker as described in Flygare J A, Pillow T H, Aristoff P., Antibody-drug conjugates for the treatment of cancer. Chemical Biology and Drug Design. 2013 January; 81(1):113-21, which is herein incorporated by reference in its entirety for all purposes. In embodiments, the linker is a linker as described in Drachman J G, Senter P D., Antibody-drug conjugates: the chemistry behind empowering antibodies to fight cancer. Hematology Am Soc Hematol Educ Program. 2013; 2013:306-10, which is herein incorporated by reference in its entirety for all purposes.

In embodiments, the compound has the formula: A-L¹-B.

A is the monovalent ABL ATP binding site inhibitor.

B is the monovalent ABL myristoyl binding site inhibitor.

L¹ is the divalent linker.

In embodiments, ABL is BCR-ABL. In embodiments, BCR-ABL is BCR-ABL1.

In embodiments, BCR-ABL1 is BCR-ABL1 wild type. In embodiments, the BCR-ABL1 is a mutant BCR-ABL1. In embodiments, the BCR-ABL1 is a T315I BCR-ABL1 mutant. In embodiments, the BCR-ABL1 is a Y253 BCR-ABL1 mutant. In embodiments, the BCR-ABL1 is a E255 BCR-ABL1 mutant. In embodiments, the BCR-ABL1 is a T315 BCR-ABL1 mutant. In embodiments, the BCR-ABL1 is a M244 BCR-ABL1 mutant. In embodiments, the BCR-ABL1 is a L248 BCR-ABL1 mutant. In embodiments, the BCR-ABL1 is a G250 BCR-ABL1 mutant. In embodiments, the BCR-ABL1 is a Q252 BCR-ABL1 mutant. In embodiments, the BCR-ABL1 is a F317 BCR-ABL1 mutant. In embodiments, the BCR-ABL1 is a M351 BCR-ABL1 mutant. In embodiments, the BCR-ABL1 is a M355 BCR-ABL1 mutant. In embodiments, the BCR-ABL1 is a F359 BCR-ABL1 mutant. In embodiments, the BCR-ABL1 is a H396 BCR-ABL1 mutant. In embodiments, the BCR-ABL1 is a V299 BCR-ABL1 mutant. In embodiments, the BCR-ABL1 is a A337 BCR-ABL1 mutant. In embodiments, the BCR-ABL1 is a W464 BCR-ABL1 mutant. In embodiments, the BCR-ABL1 is a P465 BCR-ABL1 mutant. In embodiments, the BCR-ABL1 is a V468 BCR-ABL1 mutant. In embodiments, the BCR-ABL1 is a 1502 BCR-ABL1 mutant.

In embodiments, the divalent linker includes at least 9 linear atoms between the covalent bond connecting L¹ and A and the covalent bond connecting L¹ and B. In embodiments, the divalent linker includes at least 18 linear atoms between the covalent bond connecting L¹ and A and the covalent bond connecting L¹ and B.

In embodiments, the divalent linker includes from 20 to 45 linear atoms between the covalent bond connecting L¹ and A and the covalent bond connecting L¹ and B. In embodiments, the divalent linker includes from 20 to 30 linear atoms between the covalent bond connecting L¹ and A and the covalent bond connecting L¹ and B. In embodiments, the divalent linker includes from 25 to 35 linear atoms between the covalent bond connecting L¹ and A and the covalent bond connecting L¹ and B. In embodiments, the divalent linker includes from 35 to 45 linear atoms between the covalent bond connecting L¹ and A and the covalent bond connecting L¹ and B. In embodiments, the divalent linker includes from 35 to 60 linear atoms between the covalent bond connecting L¹ and A and the covalent bond connecting L¹ and B. In embodiments, the divalent linker includes from 40 to 50 linear atoms between the covalent bond connecting L¹ and A and the covalent bond connecting L¹ and B. In embodiments, the divalent linker includes from 50 to 60 linear atoms between the covalent bond connecting L¹ and A and the covalent bond connecting L¹ and B. In embodiments, the divalent linker includes from 65 to 90 linear atoms between the covalent bond connecting L¹ and A and the covalent bond connecting L¹ and B. In embodiments, the divalent linker includes from 65 to 75 linear atoms between the covalent bond connecting L¹ and A and the covalent bond connecting L¹ and B. In embodiments, the divalent linker includes from 75 to 85 linear atoms between the covalent bond connecting L¹ and A and the covalent bond connecting L¹ and B. In embodiments, the divalent linker includes from 80 to 90 linear atoms between the covalent bond connecting L¹ and A and the covalent bond connecting L¹ and B.

In embodiments, L¹ is -L¹⁰¹-L¹⁰²-L¹⁰³-L¹⁰⁴-L¹⁰⁵-.

L¹⁰¹ is connected directly to said monovalent ABL (e.g., BCR-ABL) ATP binding site inhibitor.

L¹⁰¹ is a bond, —C(O)—, —C(O)O—, —OC(O)—, —O—, —S—, —NR¹⁰¹—, —C(O)NR¹⁰¹—, —NR¹⁰¹C(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰² is a bond, —C(O)—, —C(O)O—, —OC(O)—, —O—, —S—, —NR¹⁰²—, —C(O)NR¹⁰²—, —NR¹⁰²C(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰³ is a bond, —C(O)—, —C(O)O—, —OC(O)—, —O—, —S—, —NR¹⁰³—, —C(O)NR¹⁰³—, —NR¹⁰³C(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L¹⁰⁵ is a bond, —C(O)—, —C(O)O—, —OC(O)—, —O—, —S—, —NR¹⁰⁵—, —C(O)NR¹⁰⁵, —NR¹⁰⁵C(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R¹⁰¹, R¹⁰², R¹⁰³, R¹⁰⁴, and R¹⁰⁵ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted L¹⁰¹ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹⁰¹ is substituted, it is substituted with at least one size-limited substituent group. In embodiments, when L¹⁰¹ is substituted, it is substituted with at least one lower substituent group.

In embodiments, L¹⁰¹ is a bond. In embodiments, L¹⁰¹ is —C(O)—. In embodiments, L¹⁰¹ is —C(O)O—. In embodiments, L¹⁰¹ is —OC(O)—. In embodiments, L¹⁰¹ is —O—. In embodiments, L¹⁰¹ is —S—. In embodiments, L¹⁰¹ is —NR⁰¹—. In embodiments, L¹⁰¹ is —NH—. In embodiments, L¹⁰¹ is —C(O)NR¹⁰¹—. In embodiments, L¹⁰¹ is —C(O)NH—. In embodiments, L¹⁰¹ is —NR¹⁰¹C(O)—. In embodiments, L¹⁰¹ is —NHC(O)—. In embodiments, L¹⁰¹ is substituted or unsubstituted C₁-C₆ alkylene. In embodiments, L¹⁰¹ is substituted C₁-C₆ alkylene. In embodiments, L¹⁰¹ is substituted oxo-substituted C₁-C₆ alkylene. In embodiments, L¹⁰¹ is substituted (e.g., oxo-substituted) methylene. In embodiments, L¹⁰¹ is substituted (e.g., oxo-substituted) ethylene. In embodiments, L¹⁰¹ is substituted (e.g., oxo-substituted) propylene. In embodiments, L¹⁰¹ is substituted (e.g., oxo-substituted) n-propylene. In embodiments, L¹⁰ is substituted (e.g., oxo-substituted) isopropylene. In embodiments, L¹⁰¹ is substituted (e.g., oxo-substituted) butylene. In embodiments, L¹⁰¹ is substituted (e.g., oxo-substituted) n-butylene. In embodiments, L¹⁰¹ is substituted (e.g., oxo-substituted) isobutylene. In embodiments, L¹⁰¹ is substituted (e.g., oxo-substituted) tert-butylene. In embodiments, L¹⁰ is substituted (e.g., oxo-substituted) pentylene. In embodiments, L¹⁰¹ is substituted (e.g., oxo-substituted) hexylene. In embodiments, L¹⁰¹ is unsubstituted C₁-C₆ alkylene. In embodiments, L¹⁰¹ is unsubstituted methylene. In embodiments, L¹⁰¹ is unsubstituted ethylene. In embodiments, L¹⁰¹ is unsubstituted propylene. In embodiments, L¹⁰¹ is unsubstituted n-propylene. In embodiments, L¹⁰¹ is unsubstituted isopropylene. In embodiments, L¹⁰¹ is unsubstituted butylene. In embodiments, L¹⁰¹ is unsubstituted n-butylene. In embodiments, L¹⁰¹ is unsubstituted isobutylene. In embodiments, L¹⁰¹ is unsubstituted tert-butylene. In embodiments, L¹⁰¹ is unsubstituted pentylene. In embodiments, L¹⁰¹ is unsubstituted hexylene.

In embodiments, a substituted R¹⁰¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰¹ is substituted with a plurality of groups selected from substituent groups, size-limited substituent groups, and lower substituent groups; each substituent group, size-limited substituent group, and/or lower substituent group may optionally be different. In embodiments, when R¹⁰¹ is substituted, it is substituted with at least one substituent group. In embodiments, when R¹⁰¹ is substituted, it is substituted with at least one size-limited substituent group. In embodiments, when R¹⁰¹ is substituted, it is substituted with at least one lower substituent group.

In embodiments, R¹⁰¹ is hydrogen or unsubstituted C₁-C₄ alkyl. In embodiments, R¹⁰¹ is hydrogen. In embodiments, R¹⁰¹ is unsubstituted C₁-C₄ alkyl. In embodiments, R¹⁰¹ is unsubstituted methyl. In embodiments, R¹⁰¹ is unsubstituted ethyl. In embodiments, R¹⁰¹ is unsubstituted propyl. In embodiments, R¹⁰¹ is unsubstituted n-propyl. In embodiments, R¹⁰¹ is unsubstituted isopropyl. In embodiments, R¹⁰¹ is unsubstituted butyl. In embodiments, R¹⁰¹ is unsubstituted n-butyl. In embodiments, R¹⁰¹ is unsubstituted isobutyl. In embodiments, R¹⁰¹ is unsubstituted tert-butyl.

In embodiments, a substituted L¹⁰² (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰² is substituted with a plurality of groups selected from substituent groups, size-limited substituent groups, and lower substituent groups; each substituent group, size-limited substituent group, and/or lower substituent group may optionally be different. In embodiments, when L¹⁰² is substituted, it is substituted with at least one substituent group. In embodiments, when L¹⁰² is substituted, it is substituted with at least one size-limited substituent group. In embodiments, when L¹⁰² is substituted, it is substituted with at least one lower substituent group.

In embodiments, L¹⁰² is a bond. In embodiments, L¹⁰² is —C(O)—. In embodiments, L¹⁰² is —C(O)O—. In embodiments, L¹⁰² is —OC(O)—. In embodiments, L¹⁰² is —O—. In embodiments, L¹⁰² is —S—. In embodiments, L¹⁰² is —NR¹⁰²—. In embodiments, L¹⁰² is —NH—. In embodiments, L¹⁰² is —C(O)NR¹⁰²—. In embodiments, L¹⁰² is —C(O)NH—. In embodiments, L¹⁰² is —NR¹⁰²C(O)—. In embodiments, L¹⁰² is —NHC(O)—. In embodiments, L¹⁰² is a bond or unsubstituted 2 to 40 membered heteroalkylene. In embodiments, L¹⁰² is unsubstituted 2 to membered heteroalkylene. In embodiments, L¹⁰² is a divalent polyethylene glycol chain ranging in size from about 3 to about 50 ethylene glycol units. In embodiments, L¹⁰² is a divalent polyethylene glycol chain ranging in size from about 3 to about 20 ethylene glycol units. In embodiments, L¹⁰² is a divalent polyethylene glycol chain ranging in size from about 6 to about 18 ethylene glycol units. In embodiments, L¹⁰² is —(OCH₂CH₂)ₙ—; and n is an integer from 3 to 50. In embodiments, n is an integer from 6 to 20.

In embodiments, a substituted R¹⁰² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰² is substituted with a plurality of groups selected from substituent groups, size-limited substituent groups, and lower substituent groups; each substituent group, size-limited substituent group, and/or lower substituent group may optionally be different. In embodiments, when R¹⁰² is substituted, it is substituted with at least one substituent group. In embodiments, when R¹⁰² is substituted, it is substituted with at least one size-limited substituent group. In embodiments, when R¹⁰² is substituted, it is substituted with at least one lower substituent group.

In embodiments, R¹⁰² is hydrogen or unsubstituted C₁-C₄ alkyl. In embodiments, R¹⁰² is hydrogen. In embodiments, R¹⁰² is unsubstituted C₁-C₄ alkyl. In embodiments, R¹⁰² is unsubstituted methyl. In embodiments, R¹⁰² is unsubstituted ethyl. In embodiments, R¹⁰² is unsubstituted propyl. In embodiments, R¹⁰² is unsubstituted n-propyl. In embodiments, R¹⁰² is unsubstituted isopropyl. In embodiments, R¹⁰² is unsubstituted butyl. In embodiments, R¹⁰² is unsubstituted n-butyl. In embodiments, R¹⁰² is unsubstituted isobutyl. In embodiments, R¹⁰² is unsubstituted tert-butyl.

In embodiments, a substituted L¹⁰³ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰³ is substituted with a plurality of groups selected from substituent groups, size-limited substituent groups, and lower substituent groups; each substituent group, size-limited substituent group, and/or lower substituent group may optionally be different. In embodiments, when L¹⁰³ is substituted, it is substituted with at least one substituent group. In embodiments, when L¹⁰³ is substituted, it is substituted with at least one size-limited substituent group. In embodiments, when L¹⁰³ is substituted, it is substituted with at least one lower substituent group.

In embodiments, L¹⁰³ is a bond. In embodiments, L¹⁰³ is —C(O)—. In embodiments, L¹⁰³ is —C(O)O—. In embodiments, L¹⁰³ is —OC(O)—. In embodiments, L¹⁰³ is —O—. In embodiments, L¹⁰³ is —S—. In embodiments, L¹⁰³ is —NR¹⁰³—. In embodiments, L¹⁰³ is —NH—. In embodiments, L¹⁰³ is —C(O)NR¹⁰³—. In embodiments, L¹⁰³ is —C(O)NH—. In embodiments, L¹⁰³ is —NR¹⁰³C(O)—. In embodiments, L¹⁰³ is —NHC(O)—. In embodiments, L¹⁰³ is a bond or unsubstituted 2 to 40 membered heteroalkylene. In embodiments, L¹⁰³ is unsubstituted 2 to membered heteroalkylene. In embodiments, L¹⁰³ is a divalent polyethylene glycol chain ranging in size from about 3 to about 50 ethylene glycol units. In embodiments, L¹⁰³ is a divalent polyethylene glycol chain ranging in size from about 3 to about 20 ethylene glycol units. In embodiments, L¹⁰³ is a divalent polyethylene glycol chain ranging in size from about 6 to about 18 ethylene glycol units. In embodiments, L¹⁰³ is —(OCH₂CH₂)ₙ—; and n is an integer from 3 to 50. In embodiments, n is an integer from 6 to 20.

In embodiments, a substituted R¹⁰³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰³ is substituted with a plurality of groups selected from substituent groups, size-limited substituent groups, and lower substituent groups; each substituent group, size-limited substituent group, and/or lower substituent group may optionally be different. In embodiments, when R¹⁰³ is substituted, it is substituted with at least one substituent group. In embodiments, when R¹⁰³ is substituted, it is substituted with at least one size-limited substituent group. In embodiments, when R¹⁰³ is substituted, it is substituted with at least one lower substituent group.

In embodiments, R¹⁰³ is hydrogen or unsubstituted C₁-C₄ alkyl. In embodiments, R¹⁰³ is hydrogen. In embodiments, R¹⁰³ is unsubstituted C₁-C₄ alkyl. In embodiments, R¹⁰³ is unsubstituted methyl. In embodiments, R¹⁰³ is unsubstituted ethyl. In embodiments, R¹⁰³ is unsubstituted propyl. In embodiments, R¹⁰³ is unsubstituted n-propyl. In embodiments, R¹⁰³ is unsubstituted isopropyl. In embodiments, R¹⁰³ is unsubstituted butyl. In embodiments, R¹⁰³ is unsubstituted n-butyl. In embodiments, R¹⁰³ is unsubstituted isobutyl. In embodiments, R¹⁰³ is unsubstituted tert-butyl.

In embodiments, a substituted L¹⁰⁴ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰⁴ is substituted with a plurality of groups selected from substituent groups, size-limited substituent groups, and lower substituent groups; each substituent group, size-limited substituent group, and/or lower substituent group may optionally be different. In embodiments, when L¹⁰⁴ is substituted, it is substituted with at least one substituent group. In embodiments, when L¹⁰⁴ is substituted, it is substituted with at least one size-limited substituent group. In embodiments, when L¹⁰⁴ is substituted, it is substituted with at least one lower substituent group.

In embodiments, L¹⁰⁴ is a bond. In embodiments, L¹⁰⁴ is —C(O)—. In embodiments, L¹⁰⁴ is —C(O)O—. In embodiments, L¹⁰⁴ is —OC(O)—. In embodiments, L¹⁰⁴ is —O—. In embodiments, L¹⁰⁴ is —S—. In embodiments, L¹⁰⁴ is —NR¹⁰⁴—. In embodiments, L¹⁰⁴ is —NH—. In embodiments, L¹⁰⁴ is —C(O)NR¹⁰⁴—. In embodiments, L¹⁰⁴ is —C(O)NH—. In embodiments, L¹⁰⁴ is —NR¹⁰⁴C(O)—. In embodiments, L¹⁰⁴ is —NHC(O)—.

In embodiments, a substituted R¹⁰⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰⁴ is substituted with a plurality of groups selected from substituent groups, size-limited substituent groups, and lower substituent groups; each substituent group, size-limited substituent group, and/or lower substituent group may optionally be different. In embodiments, when R¹⁰⁴ is substituted, it is substituted with at least one substituent group. In embodiments, when R¹⁰⁴ is substituted, it is substituted with at least one size-limited substituent group. In embodiments, when R¹⁰⁴ is substituted, it is substituted with at least one lower substituent group.

In embodiments, R¹⁰⁴ is hydrogen or unsubstituted C₁-C₄ alkyl. In embodiments, R¹⁰⁴ is hydrogen. In embodiments, R¹⁰⁴ is unsubstituted C₁-C₄ alkyl. In embodiments, R¹⁰⁴ is unsubstituted methyl. In embodiments, R¹⁰⁴ is unsubstituted ethyl. In embodiments, R¹⁰⁴ is unsubstituted propyl. In embodiments, R¹⁰⁴ is unsubstituted n-propyl. In embodiments, R¹⁰⁴ is unsubstituted isopropyl. In embodiments, R¹⁰⁴ is unsubstituted butyl. In embodiments, R¹⁰⁴ is unsubstituted n-butyl. In embodiments, R¹⁰⁴ is unsubstituted isobutyl. In embodiments, R¹⁰⁴ is unsubstituted tert-butyl.

In embodiments, a substituted L¹⁰⁵ (e.g., substituted alkylene, substituted heteroalkylene, substituted cycloalkylene, substituted heterocycloalkylene, substituted arylene, and/or substituted heterarylene) is substituted with at least one substituent group, size-limited substituent group, or lower substituent group; wherein if the substituted L¹⁰⁵ is substituted with a plurality of groups selected from substituent groups, size-limited substituent groups, and lower substituent groups; each substituent group, size-limited substituent group, and/or lower substituent group may optionally be different. In embodiments, when L¹⁰⁵ is substituted, it is substituted with at least one substituent group. In embodiments, when L¹⁰⁵ is substituted, it is substituted with at least one size-limited substituent group. In embodiments, when L¹⁰⁵ is substituted, it is substituted with at least one lower substituent group.

In embodiments, L¹⁰⁵ is a bond. In embodiments, L¹⁰⁵ is —C(O)—. In embodiments, L¹⁰⁵ is —C(O)O—. In embodiments, L¹⁰⁵ is —OC(O)—. In embodiments, L¹⁰⁵ is —O—. In embodiments, L¹⁰⁵ is —S—. In embodiments, L¹⁰⁵ is —NR¹⁰⁵—. In embodiments, L¹⁰⁵ is —NH—. In embodiments, L¹⁰⁵ is —C(O)NR¹⁰⁵—. In embodiments, L¹⁰⁵ is —C(O)NH—. In embodiments, L¹⁰⁵ is —NR¹⁰⁵C(O)—. In embodiments, L¹⁰⁵ is —NHC(O)—. In embodiments, L¹⁰⁵ is substituted or unsubstituted 3 to 8 membered heterocycloalkylene. In embodiments, L¹⁰⁵ is unsubstituted 3 to 8 membered heterocycloalkylene. In embodiments, L¹⁰⁵ is unsubstituted piperidinylene. In embodiments, L¹⁰⁵ is

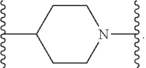 [SMILES: *C1CCN(*)CC1]

In embodiments, a substituted R¹⁰⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰⁵ is substituted with a plurality of groups selected from substituent groups, size-limited substituent groups, and lower substituent groups; each substituent group, size-limited substituent group, and/or lower substituent group may optionally be different. In embodiments, when R¹⁰⁵ is substituted, it is substituted with at least one substituent group. In embodiments, when R¹⁰⁵ is substituted, it is substituted with at least one size-limited substituent group. In embodiments, when R¹⁰⁵ is substituted, it is substituted with at least one lower substituent group.

In embodiments, R¹⁰⁵ is hydrogen or unsubstituted C₁-C₄ alkyl. In embodiments, R¹⁰⁵ is hydrogen. In embodiments, R¹⁰⁵ is unsubstituted C₁-C₄ alkyl. In embodiments, R¹⁰⁵ is unsubstituted methyl. In embodiments, R¹⁰⁵ is unsubstituted ethyl. In embodiments, R¹⁰⁵ is unsubstituted propyl. In embodiments, R¹⁰⁵ is unsubstituted n-propyl. In embodiments, R¹⁰⁵ is unsubstituted isopropyl. In embodiments, R¹⁰⁵ is unsubstituted butyl. In embodiments, R¹⁰⁵ is unsubstituted n-butyl. In embodiments, R¹⁰⁵ is unsubstituted isobutyl. In embodiments, R¹⁰⁵ is unsubstituted tert-butyl.

In embodiments, L¹⁰¹ is substituted C₁-C₆ alkylene; L¹⁰² is unsubstituted 2 to 40 membered heteroalkylene; L¹⁰³ is unsubstituted 2 to 40 membered heteroalkylene; L¹⁰⁴ is —NHC(O)—; and L¹⁰⁵ is unsubstituted 3 to 8 membered heterocycloalkylene.

In embodiments, L¹ is -L¹⁰¹-(OCH₂CH₂)ₙ-L¹⁰⁴-L¹⁰⁵-; and n is an integer from 3 to 50. In embodiments, L¹ is -L¹⁰¹-(OCH₂CH₂)ₙ-L¹⁰⁴-L¹⁰⁵-; L¹⁰¹ is substituted oxo-substituted C₁-C₆ alkyl; L¹⁰⁴ is —NHC(O)—; L¹⁰⁵ is unsubstituted piperidinylene; and n is an integer from 3 to 50. In embodiments, n is an integer from 6 to 20. In embodiments, n is 20. In embodiments, n is 18. In embodiments, n is 16. In embodiments, n is 14. In embodiments, n is 12. In embodiments, n is 10. In embodiments, n is 8. In embodiments, n is 6.

In embodiments, L¹ is -L¹⁰¹-(OCH₂CH₂)ₙ-L¹⁰⁴-L¹⁰⁵-; and n is an integer from 3 to 50. In embodiments, L¹ is -L¹⁰¹-(OCH₂CH₂)ₙ-L¹⁰⁴-L¹⁰⁵-; L¹⁰¹ is oxo-substituted C₁-C₆ alkylene; L¹⁰⁴ is —NHC(O)—; L¹⁰⁵ is unsubstituted piperidinylene; and n is an integer from 3 to 50. In embodiments, n is an integer from 6 to 20. In embodiments, n is 20. In embodiments, n is 18. In embodiments, n is 16. In embodiments, n is 14. In embodiments, n is 12. In embodiments, n is 10. In embodiments, n is 8. In embodiments, n is 6.

In embodiments, L¹ is

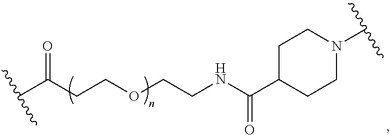

wherein n is an integer from 3 to 50. In embodiments, n is an integer from 6 to 12. In embodiments, n is an integer from 12 to 28. In embodiments, n is an integer from 20 to 28. In embodiments, n is 20. In embodiments, n is 18. In embodiments, n is 16. In embodiments, n is 14. In embodiments, n is 12. In embodiments, n is 10. In embodiments, n is 8. In embodiments, n is 6.

In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20. In embodiments, n is 21. In embodiments, n is 22. In embodiments, n is 23. In embodiments, n is 24. In embodiments, n is 25. In embodiments, n is 26. In embodiments, n is 27. In embodiments, n is 28. In embodiments, n is 29. In embodiments, n is 30. In embodiments, n is 31. In embodiments, n is 32. In embodiments, n is 33. In embodiments, n is 34. In embodiments, n is 35. In embodiments, n is 36. In embodiments, n is 37. In embodiments, n is 38. In embodiments, n is 39. In embodiments, n is 40. In embodiments, n is 41. In embodiments, n is 42. In embodiments, n is 43. In embodiments, n is 44. In embodiments, n is 45. In embodiments, n is 46. In embodiments, n is 47. In embodiments, n is 48. In embodiments, n is 49. In embodiments, n is 50.

In embodiments, L¹ is —(OCH₂CH₂)ₘ-L¹⁰²-(OCH₂CH₂)ₚ-L¹⁰⁴-L¹⁰⁵-; and m and p are independently an integer from 3 to 50. In embodiments, L¹ is —(OCH₂CH₂)ₘ-L¹⁰²-(OCH₂CH₂)ₚ-L¹⁰⁴-L¹⁰⁵-. L¹⁰² is oxo-substituted 2 to 6 membered heteroalkylene; L¹⁰⁴ is —NHC(O)—; L¹⁰⁵ is unsubstituted piperidinylene; and m and p are independently an integer from 3 to 50. In embodiments, m is an integer from 6 to 8. In embodiments, m is 7. In embodiments, p is an integer from 4 to 10. In embodiments, p is 4. In embodiments, p is 6. In embodiments, p is 8. In embodiments, p is 10.

In embodiments, L¹ is -L¹⁰¹-(OCH₂CH₂)ₘ—NHC(O)CH₂CH₂—(OCH₂CH₂)ₚ-L¹⁰⁴-L¹⁰⁵-; and m and p are independently an integer from 3 to 50. In embodiments, L¹ is -L¹¹¹-(OCH₂CH₂)ₘ—NHC(O)CH₂CH₂—(OCH₂CH₂)ₚ-L¹⁰⁴-L¹⁰⁵-. L¹⁰⁴ is —NHC(O)—; L¹⁰⁵ is unsubstituted piperidinylene; and m and p are independently an integer from 3 to 50. In embodiments, m is an integer from 6 to 8. In embodiments, m is 7. In embodiments, p is an integer from 4 to 10. In embodiments, p is 4. In embodiments, p is 6. In embodiments, p is 8. In embodiments, p is 10.

In embodiments, L¹ is -L¹⁰¹-(OCH₂CH₂)ₘ—C(O)NHCH₂CH₂—(OCH₂CH₂)ₚ-L¹⁰⁴-L¹⁰⁵-; and m and p are independently an integer from 3 to 50. In embodiments, L¹ is -L¹⁰¹-(OCH₂CH₂)ₘ—C(O)NHCH₂CH₂—(OCH₂CH₂)ₚ-L¹⁰⁴-L¹⁰⁵-; L¹⁰⁴ is —NHC(O)—; L¹⁰⁵ is unsubstituted piperidinylene; and m and p are independently an integer from 3 to 50. In embodiments, m is an integer from 6 to 8. In embodiments, m is 7. In embodiments, p is an integer from 4 to 10. In embodiments, p is 4. In embodiments, p is 6. In embodiments, p is 8. In embodiments, p is 10.

In embodiments, L¹ is

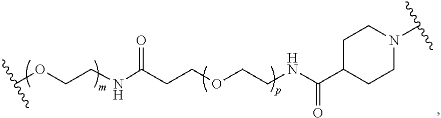

wherein m and p are independently an integer from 3 to 50. In embodiments, m is an integer from 6 to 8. In embodiments, m is 7. In embodiments, p is an integer from 4 to 10. In embodiments, p is 4. In embodiments, p is 6. In embodiments, p is 8. In embodiments, p is 10.

In embodiments, L¹ is

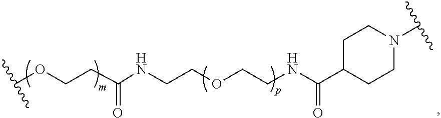

wherein m and p are independently an integer from 3 to 50. In embodiments, m is an integer from 6 to 8. In embodiments, m is 7. In embodiments, p is an integer from 4 to 10. In embodiments, p is 4. In embodiments, p is 6. In embodiments, p is 8. In embodiments, p is 10.

In embodiments, L¹ is -L¹⁰¹-L¹⁰²-L¹⁰³-L¹⁰⁴-L¹⁰⁵-; wherein L¹⁰¹ is substituted or unsubstituted heteroalkylene and includes —(OCH₂CH₂)ₘ—; L¹⁰² is substituted or unsubstituted heteroarylene; L¹⁰³ is substituted or unsubstituted heteroalkylene and includes —(OCH₂CH₂)ₚ—; L¹⁰⁴ is —NHC(O)—; L¹⁰⁵ is unsubstituted piperidinylene; and m and p are independently an integer from 3 to 50. In embodiments, L¹ is —(OCH₂CH₂)ₘ-L¹⁰²-CH₂—(OCH₂CH₂)ₚ-L¹⁰⁴-L¹⁰⁵-; wherein L¹⁰² is substituted or unsubstituted heteroarylene; L¹⁰⁴ is —NHC(O)—; L¹⁰⁵ is unsubstituted piperidinylene; and m and p are independently an integer from 3 to 50. In embodiments, L¹ is —(OCH₂CH₂)ₘ-L¹⁰²-L¹⁰³-L¹⁰⁴-L¹⁰⁵-; wherein L¹⁰² is substituted or unsubstituted heteroarylene; L¹⁰³ is substituted or unsubstituted alkylene or substituted or unsubstituted heteroalkylene; L¹⁰⁴ is substituted or unsubstituted heteroalkylene; L¹⁰⁵ is unsubstituted piperidinylene; and m and p are independently an integer from 3 to 50. In embodiments, L¹ is —(OCH₂CH₂)ₘ-L¹⁰²-L¹⁰³-(OCH₂CH₂)ₚ—NHC(O)-L¹⁰⁵-; wherein L¹⁰² is substituted or unsubstituted heteroarylene; L¹⁰³ is substituted or unsubstituted alkylene or substituted or unsubstituted heteroalkylene; L¹⁰⁵ is unsubstituted piperidinylene; and m and p are independently an integer from 3 to 50. In embodiments, L¹⁰² is unsubstituted triazolylene. In embodiments, L¹⁰³ is unsubstituted C₁-C₄ alkylene. In embodiments, L¹⁰³ is unsubstituted methylene. In embodiments, m is an integer from 6 to 8. In embodiments, m is 7. In embodiments, p is an integer from 4 to 10. In embodiments, p is 4. In embodiments, p is 6. In embodiments, p is 8. In embodiments, p is 10.

In embodiments, m is 3. In embodiments, m is 4. In embodiments, m is 5. In embodiments, m is 6. In embodiments, m is 7. In embodiments, m is 8. In embodiments, m is 9. In embodiments, m is 10. In embodiments, m is 11. In embodiments, m is 12. In embodiments, m is 13. In embodiments, m is 14. In embodiments, m is 15. In embodiments, m is 16. In embodiments, m is 17. In embodiments, m is 18. In embodiments, m is 19. In embodiments, m is 20. In embodiments, m is 21. In embodiments, m is 22. In embodiments, m is 23. In embodiments, m is 24. In embodiments, m is 25. In embodiments, m is 26. In embodiments, m is 27. In embodiments, m is 28. In embodiments, m is 29. In embodiments, m is 30. In embodiments, m is 31. In embodiments, m is 32. In embodiments, m is 33. In embodiments, m is 34. In embodiments, m is 35. In embodiments, m is 36. In embodiments, m is 37. In embodiments, m is 38. In embodiments, m is 39. In embodiments, m is 40. In embodiments, m is 41. In embodiments, m is 42. In embodiments, m is 43. In embodiments, m is 44. In embodiments, m is 45. In embodiments, m is 46. In embodiments, m is 47. In embodiments, m is 48. In embodiments, m is 49. In embodiments, m is 50.

In embodiments, p is 3. In embodiments, p is 4. In embodiments, p is 5. In embodiments, p is 6. In embodiments, p is 7. In embodiments, p is 8. In embodiments, p is 9. In embodiments, p is 10. In embodiments, p is 11. In embodiments, p is 12. In embodiments, p is 13. In embodiments, p is 14. In embodiments, p is 15. In embodiments, p is 16. In embodiments, p is 17. In embodiments, p is 18. In embodiments, p is 19. In embodiments, p is 20. In embodiments, p is 21. In embodiments, p is 22. In embodiments, p is 23. In embodiments, p is 24. In embodiments, p is 25. In embodiments, p is 26. In embodiments, p is 27. In embodiments, p is 28. In embodiments, p is 29. In embodiments, p is 30. In embodiments, p is 31. In embodiments, p is 32. In embodiments, p is 33. In embodiments, p is 34. In embodiments, p is 35. In embodiments, p is 36. In embodiments, p is 37. In embodiments, p is 38. In embodiments, p is 39. In embodiments, p is 40. In embodiments, p is 41. In embodiments, p is 42. In embodiments, p is 43. In embodiments, p is 44. In embodiments, p is 45. In embodiments, p is 46. In embodiments, p is 47. In embodiments, p is 48. In embodiments, p is 49. In embodiments, p is 50.

In embodiments, A is a monovalent form of dasatinib (e.g., BMS-354825), a monovalent form of ponatinib (e.g., AP24534), a monovalent form of imatinib (e.g., ST1571), a monovalent form of nilotinib (e.g., AMN-107), a monovalent form of bosutinib (e.g., SKI-606), a monovalent form of bafetinib (e.g., INNO-406), a monovalent form of olverembatinib (e.g., GZD824 or HQP1351), a monovalent form of tozasertib (e.g., VX-680 or MK-0457), a monovalent form of PF-114, a monovalent form of rebastinib (e.g., DCC-2036), a monovalent form of danusertib (e.g., PHA-739358), or a monovalent form of HG-7-85-01. In embodiments, A is a monovalent form of dasatinib (e.g., BMS-354825). In embodiments, A is a monovalent form of ponatinib (e.g., AP24534). In embodiments, A is a monovalent form of imatinib (e.g., ST1571). In embodiments, A is a monovalent form of nilotinib (e.g., AMN-107). In embodiments, A is a monovalent form of bosutinib (e.g., SKI-606). In embodiments, A is a monovalent form of bafetinib (e.g., INNO-406). In embodiments, A is a monovalent form of olverembatinib (e.g., GZD824 or HQP1351). In embodiments, A is a monovalent form of tozasertib (e.g., VX-680 or MK-0457). In embodiments, A is a monovalent form of PF-114. In embodiments, A is a monovalent form of rebastinib (e.g., DCC-2036). In embodiments, A is a monovalent form of danusertib (e.g., PHA-739358). In embodiments, A is a monovalent form of HG-7-85-01.

In embodiments, A is a monovalent form of dasatinib (e.g., BMS-354825). In embodiments, A is a monovalent form of a compound as described in U.S. Pat. No. 7,491,725, which is herein incorporated by reference in its entirety for all purposes. In embodiments, A is a monovalent form of

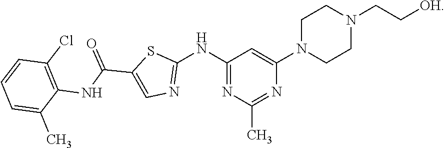 [SMILES: Cc1nc(Nc2ncc(C(=O)Nc3c(C)cccc3Cl)s2)cc(N2CCN(CCO)CC2)n1]

In embodiments, A is

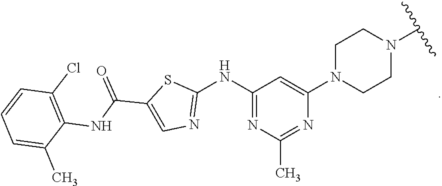 [SMILES: *N1CCN(c2cc(Nc3ncc(C(=O)Nc4c(C)cccc4Cl)s3)nc(C)n2)CC1]

In embodiments, A is

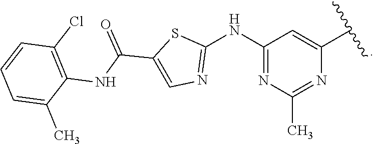 [SMILES: *c1cc(Nc2ncc(C(=O)Nc3c(C)cccc3Cl)s2)nc(C)n1]

In embodiments, A is

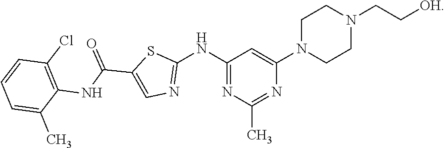 [SMILES: Cc1nc(Nc2ncc(C(=O)Nc3c(C)cccc3Cl)s2)cc(N2CCN(CCO)CC2)n1]

In embodiments, A is a monovalent form of ponatinib (e.g., AP24534). In embodiments, A is a monovalent form of a compound as described in U.S. Pat. No. 8,114,874, which is herein incorporated by reference in its entirety for all purposes. In embodiments, A is a monovalent form of

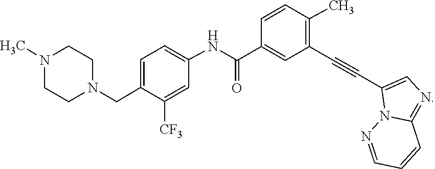 [SMILES: Cc1ccc(C(=O)Nc2ccc(CN3CCN(C)CC3)c(C(F)(F)F)c2)cc1C#Cc1cnc2cccnn12]

In embodiments, A is

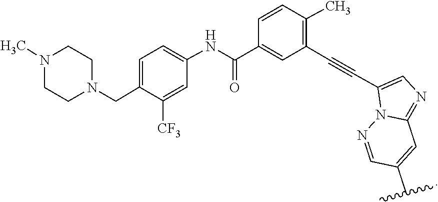 [SMILES: *c1cnn2c(C#Cc3cc(C(=O)Nc4ccc(CN5CCN(C)CC5)c(C(F)(F)F)c4)ccc3C)cnc2c1]

In embodiments, A is

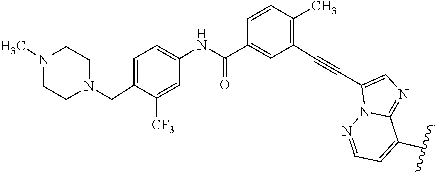 [SMILES: *c1ccnn2c(C#Cc3cc(C(=O)Nc4ccc(CN5CCN(C)CC5)c(C(F)(F)F)c4)ccc3C)cnc12]

In embodiments, A is

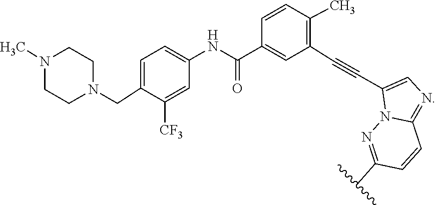 [SMILES: *c1ccc2ncc(C#Cc3cc(C(=O)Nc4ccc(CN5CCN(C)CC5)c(C(F)(F)F)c4)ccc3C)n2n1]

In embodiments, A is

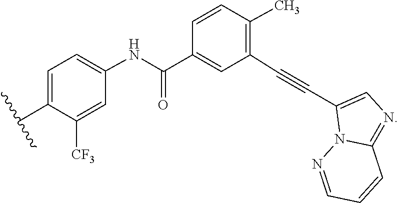 [SMILES: *c1ccc(NC(=O)c2ccc(C)c(C#Cc3cnc4cccnn34)c2)cc1C(F)(F)F]

In embodiments, A is

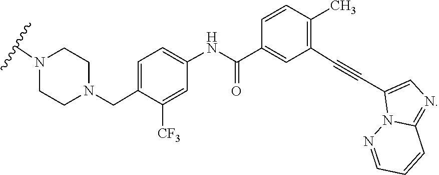 [SMILES: *N1CCN(Cc2ccc(NC(=O)c3ccc(C)c(C#Cc4cnc5cccnn45)c3)cc2C(F)(F)F)CC1]

In embodiments, A is

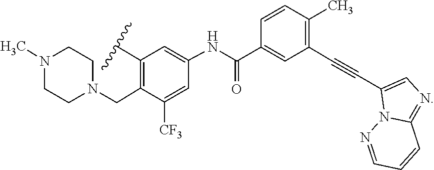 [SMILES: *c1cc(NC(=O)c2ccc(C)c(C#Cc3cnc4cccnn34)c2)cc(C(F)(F)F)c1CN1CCN(C)CC1]

In embodiments, A is a monovalent form of a compound as described in Huang, et al., J Med. Chem., 53, 4701 (2010), which is herein incorporated by reference in its entirety for all purposes. In embodiments, A is a monovalent form of

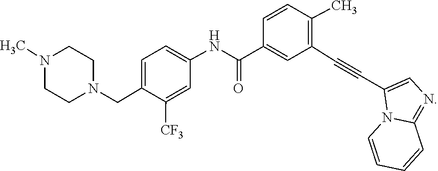 [SMILES: Cc1ccc(C(=O)Nc2ccc(CN3CCN(C)CC3)c(C(F)(F)F)c2)cc1C#Cc1cnc2ccccn12]

In embodiments, A is

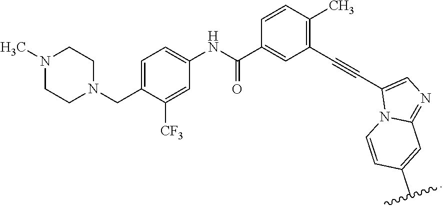 [SMILES: *c1ccn2c(C#Cc3cc(C(=O)Nc4ccc(CN5CCN(C)CC5)c(C(F)(F)F)c4)ccc3C)cnc2c1]

In embodiments, A is

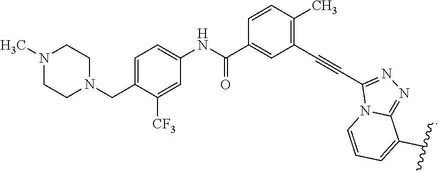 [SMILES: *c1cccn2c(C#Cc3cc(C(=O)Nc4ccc(CN5CCN(C)CC5)c(C(F)(F)F)c4)ccc3C)nnc12]

In embodiments, A is

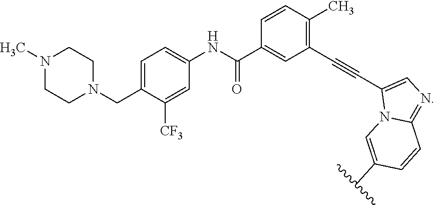 [SMILES: *c1ccc2ncc(C#Cc3cc(C(=O)Nc4ccc(CN5CCN(C)CC5)c(C(F)(F)F)c4)ccc3C)n2c1]

In embodiments, A is

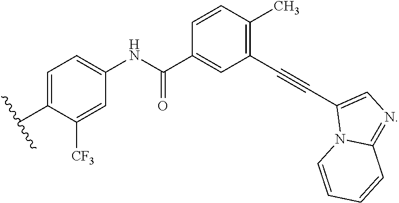 [SMILES: *c1ccc(NC(=O)c2ccc(C)c(C#Cc3cnc4ccccn34)c2)cc1C(F)(F)F]

In embodiments, A is

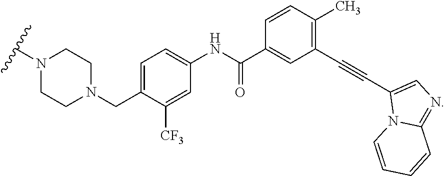 [SMILES: *N1CCN(Cc2ccc(NC(=O)c3ccc(C)c(C#Cc4cnc5ccccn45)c3)cc2C(F)(F)F)CC1]

In embodiments, A is

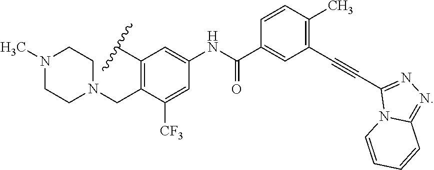 [SMILES: *c1cc(NC(=O)c2ccc(C)c(C#Cc3nnc4ccccn34)c2)cc(C(F)(F)F)c1CN1CCN(C)CC1]

In embodiments, A is a monovalent form of imatinib (e.g., ST1571). In embodiments, A is a monovalent form of a compound as described in U.S. Pat. No. 6,958,335, which is herein incorporated by reference in its entirety for all purposes. In embodiments, A is a monovalent form of

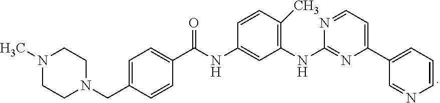 [SMILES: Cc1ccc(NC(=O)c2ccc(CN3CCN(C)CC3)cc2)cc1Nc1nccc(-c2cccnc2)n1]

In embodiments, A is

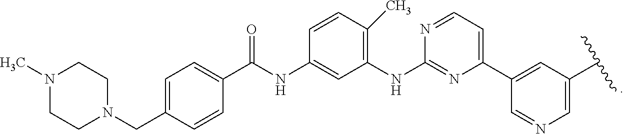 [SMILES: *c1cncc(-c2ccnc(Nc3cc(NC(=O)c4ccc(CN5CCN(C)CC5)cc4)ccc3C)n2)c1]

In embodiments, A is

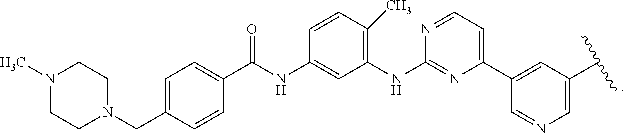 [SMILES: *c1cncc(-c2ccnc(Nc3cc(NC(=O)c4ccc(CN5CCN(C)CC5)cc4)ccc3C)n2)c1]

In embodiments, A is

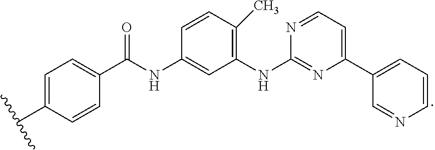 [SMILES: *c1ccc(C(=O)Nc2ccc(C)c(Nc3nccc(-c4cccnc4)n3)c2)cc1]

In embodiments, A is

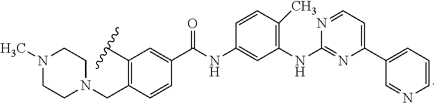 [SMILES: *c1cc(C(=O)Nc2ccc(C)c(Nc3nccc(-c4cccnc4)n3)c2)ccc1CN1CCN(C)CC1]

In embodiments, A is a monovalent form of nilotinib (e.g., AMN-107). In embodiments, A is a monovalent form of a compound as described in U.S. Pat. No. 7,169,791, which is herein incorporated by reference in its entirety for all purposes. In embodiments, A is a monovalent form of

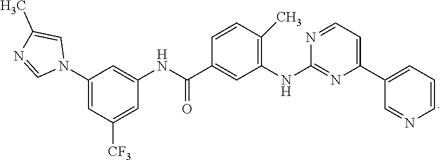 [SMILES: Cc1cn(-c2cc(NC(=O)c3ccc(C)c(Nc4nccc(-c5cccnc5)n4)c3)cc(C(F)(F)F)c2)cn1]

In embodiments, A is

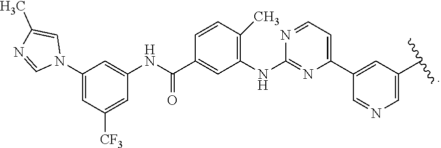 [SMILES: *c1cncc(-c2ccnc(Nc3cc(C(=O)Nc4cc(-n5cnc(C)c5)cc(C(F)(F)F)c4)ccc3C)n2)c1]

In embodiments, A is

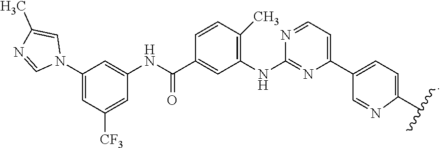 [SMILES: *c1ccc(-c2ccnc(Nc3cc(C(=O)Nc4cc(-n5cnc(C)c5)cc(C(F)(F)F)c4)ccc3C)n2)cn1]

In embodiments, A is

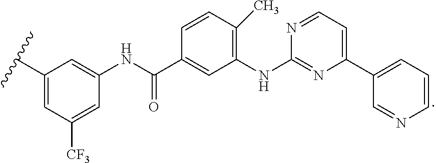 [SMILES: *c1cc(NC(=O)c2ccc(C)c(Nc3nccc(-c4cccnc4)n3)c2)cc(C(F)(F)F)c1]

In embodiments, A is

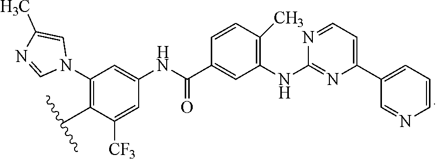 [SMILES: *c1c(-n2cnc(C)c2)cc(NC(=O)c2ccc(C)c(Nc3nccc(-c4cccnc4)n3)c2)cc1C(F)(F)F]

In embodiments, A is a monovalent form of bosutinib (e.g., SKI-606). In embodiments, A is a monovalent form of a compound as described in U.S. Pat. No. 7,417,148, which is herein incorporated by reference in its entirety for all purposes. In embodiments, A is a monovalent form of

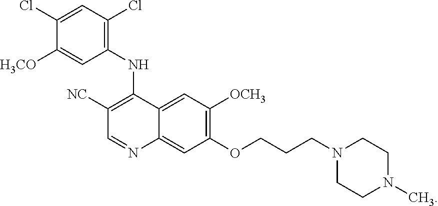 [SMILES: COc1cc(Nc2c(C#N)cnc3cc(OCCCN4CCN(C)CC4)c(OC)cc23)c(Cl)cc1Cl]

In embodiments, A is

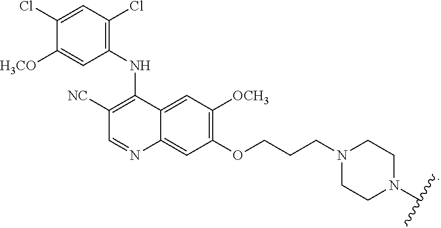 [SMILES: *N1CCN(CCCOc2cc3ncc(C#N)c(Nc4cc(OC)c(Cl)cc4Cl)c3cc2OC)CC1]

In embodiments, A is

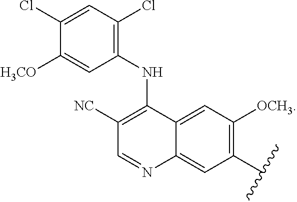 [SMILES: *c1cc2ncc(C#N)c(Nc3cc(OC)c(Cl)cc3Cl)c2cc1OC]

In embodiments, A is

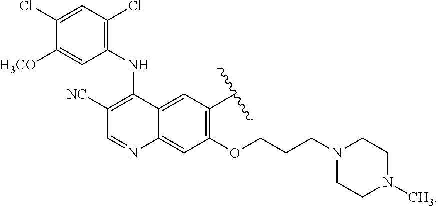 [SMILES: *c1cc2c(Nc3cc(OC)c(Cl)cc3Cl)c(C#N)cnc2cc1OCCCN1CCN(C)CC1]

In embodiments, A is a monovalent form of bafetinib (e.g., INNO-406). In embodiments, A is a monovalent form of a compound as described in U.S. Pat. No. 7,728,131, which is herein incorporated by reference in its entirety for all purposes. In embodiments, A is a monovalent form of

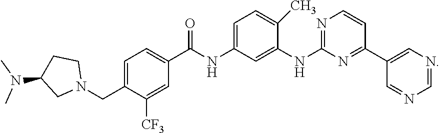 [SMILES: Cc1ccc(NC(=O)c2ccc(CN3CC[C@H](N(C)C)C3)c(C(F)(F)F)c2)cc1Nc1nccc(-c2cncnc2)n1]

In embodiments, A is

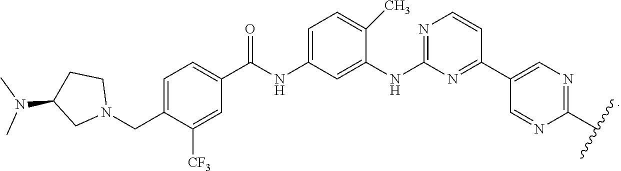 [SMILES: *c1ncc(-c2ccnc(Nc3cc(NC(=O)c4ccc(CN5CC[C@H](N(C)C)C5)c(C(F)(F)F)c4)ccc3C)n2)cn1]

In embodiments, A is

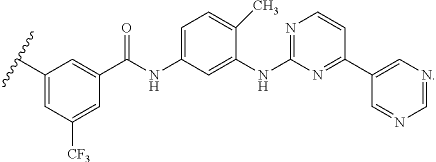 [SMILES: *c1cc(C(=O)Nc2ccc(C)c(Nc3nccc(-c4cncnc4)n3)c2)cc(C(F)(F)F)c1]

In embodiments, A is

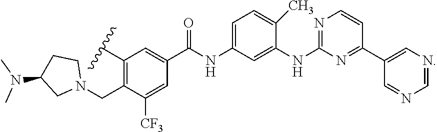 [SMILES: *c1cc(C(=O)Nc2ccc(C)c(Nc3nccc(-c4cncnc4)n3)c2)cc(C(F)(F)F)c1CN1CC[C@H](N(C)C)C1]

In embodiments, A is a monovalent form of olverembatinib (e.g., GZD824 or HQP1351). In embodiments, A is a monovalent form of a compound as described in WO 2012/000304, which is herein incorporated by reference in its entirety for all purposes. In embodiments, A is a monovalent form of

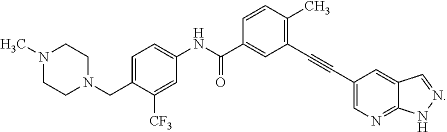 [SMILES: Cc1ccc(C(=O)Nc2ccc(CN3CCN(C)CC3)c(C(F)(F)F)c2)cc1C#Cc1cnc2[nH]ncc2c1]

In embodiments, A is

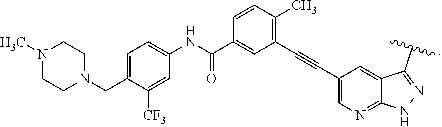 [SMILES: *c1n[nH]c2ncc(C#Cc3cc(C(=O)Nc4ccc(CN5CCN(C)CC5)c(C(F)(F)F)c4)ccc3C)cc12]

In embodiments, A is

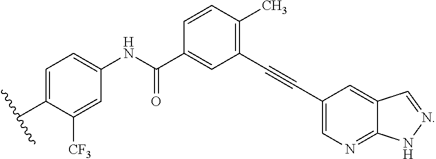 [SMILES: *c1ccc(NC(=O)c2ccc(C)c(C#Cc3cnc4[nH]ncc4c3)c2)cc1C(F)(F)F]

In embodiments, A is

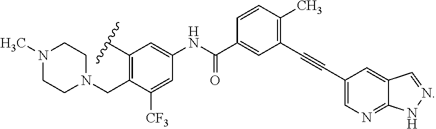 [SMILES: *c1cc(NC(=O)c2ccc(C)c(C#Cc3cnc4[nH]ncc4c3)c2)cc(C(F)(F)F)c1CN1CCN(C)CC1]

In embodiments, A is a monovalent form of tozasertib (e.g., VX-680 or MK-0457). In embodiments, A is a monovalent form of a compound as described in WO 2004/000833, which is herein incorporated by reference in its entirety for all purposes. In embodiments, A is a monovalent form of

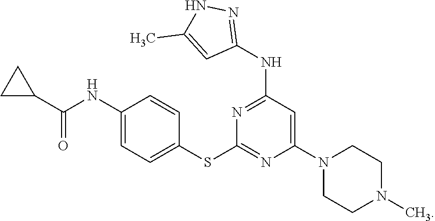 [SMILES: Cc1cc(Nc2cc(N3CCN(C)CC3)nc(Sc3ccc(NC(=O)C4CC4)cc3)n2)n[nH]1]

In embodiments, A is

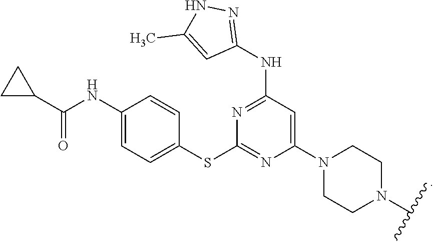 [SMILES: *N1CCN(c2cc(Nc3cc(C)[nH]n3)nc(Sc3ccc(NC(=O)C4CC4)cc3)n2)CC1]

In embodiments, A is

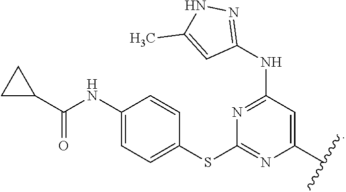 [SMILES: *c1cc(Nc2cc(C)[nH]n2)nc(Sc2ccc(NC(=O)C3CC3)cc2)n1]

In embodiments, A is a monovalent form of PF-114. In embodiments, A is a monovalent form of a compound as described in WO 2012/173521, which is herein incorporated by reference in its entirety for all purposes. In embodiments, A is a monovalent form of

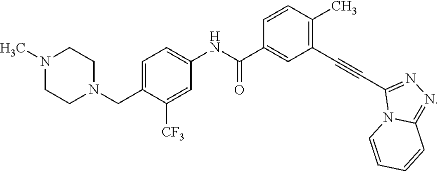 [SMILES: Cc1ccc(C(=O)Nc2ccc(CN3CCN(C)CC3)c(C(F)(F)F)c2)cc1C#Cc1nnc2ccccn12]

In embodiments, A is

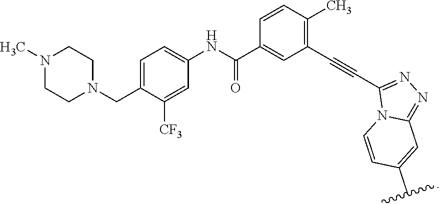 [SMILES: *c1ccn2c(C#Cc3cc(C(=O)Nc4ccc(CN5CCN(C)CC5)c(C(F)(F)F)c4)ccc3C)nnc2c1]

In embodiments, A is

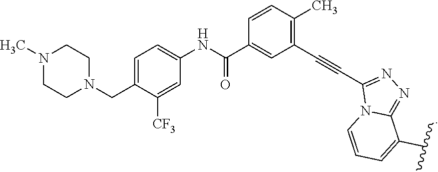 [SMILES: *c1cccn2c(C#Cc3cc(C(=O)Nc4ccc(CN5CCN(C)CC5)c(C(F)(F)F)c4)ccc3C)nnc12]

In embodiments, A is

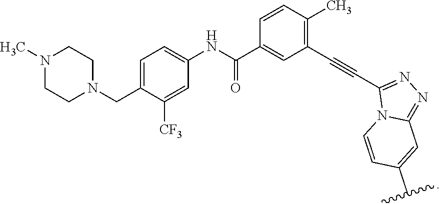 [SMILES: *c1ccn2c(C#Cc3cc(C(=O)Nc4ccc(CN5CCN(C)CC5)c(C(F)(F)F)c4)ccc3C)nnc2c1]

In embodiments, A is

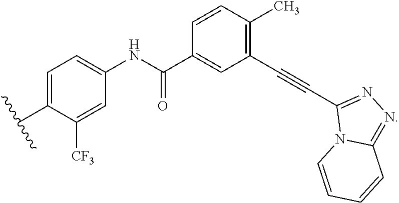 [SMILES: *c1ccc(NC(=O)c2ccc(C)c(C#Cc3nnc4ccccn34)c2)cc1C(F)(F)F]

In embodiments, A is

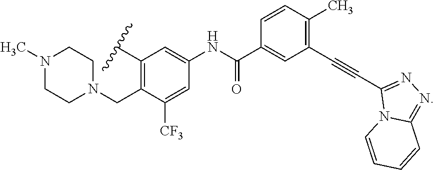 [SMILES: *c1cc(NC(=O)c2ccc(C)c(C#Cc3nnc4ccccn34)c2)cc(C(F)(F)F)c1CN1CCN(C)CC1]

In embodiments, A is a monovalent form of rebastinib (e.g., DCC-2036). In embodiments, A is a monovalent form of a compound as described in WO 2008/046003, which is herein incorporated by reference in its entirety for all purposes. In embodiments, A is a monovalent form of

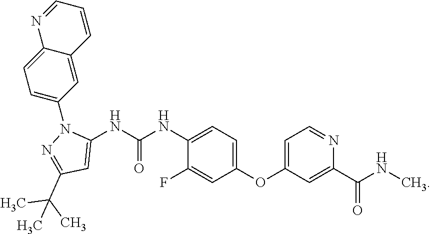 [SMILES: CNC(=O)c1cc(Oc2ccc(NC(=O)Nc3cc(C(C)(C)C)nn3-c3ccc4ncccc4c3)c(F)c2)ccn1]

In embodiments, A is

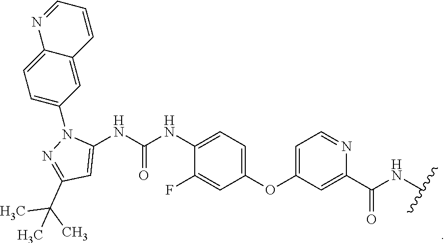 [SMILES: *NC(=O)c1cc(Oc2ccc(NC(=O)Nc3cc(C(C)(C)C)nn3-c3ccc4ncccc4c3)c(F)c2)ccn1]

In embodiments, A is

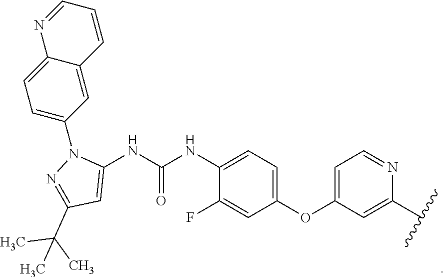 [SMILES: *c1cc(Oc2ccc(NC(=O)Nc3cc(C(C)(C)C)nn3-c3ccc4ncccc4c3)c(F)c2)ccn1]

In embodiments, A is

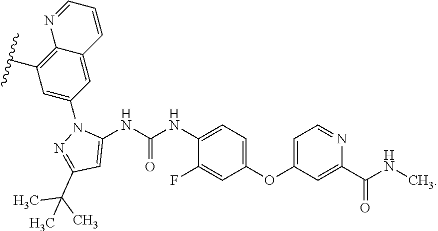 [SMILES: *c1cc(-n2nc(C(C)(C)C)cc2NC(=O)Nc2ccc(Oc3ccnc(C(=O)NC)c3)cc2F)cc2cccnc12]

In embodiments, A is a monovalent form of danusertib (e.g., PHA-739358). In embodiments, A is a monovalent form of a compound as described in WO 2005/005427, which is herein incorporated by reference in its entirety for all purposes. In embodiments, A is a monovalent form of

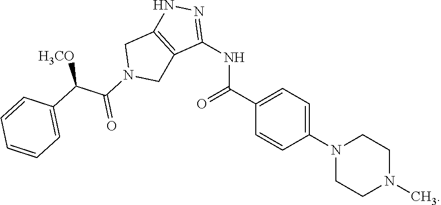 [SMILES: CO[C@@H](C(=O)N1Cc2[nH]nc(NC(=O)c3ccc(N4CCN(C)CC4)cc3)c2C1)c1ccccc1]

In embodiments, A is

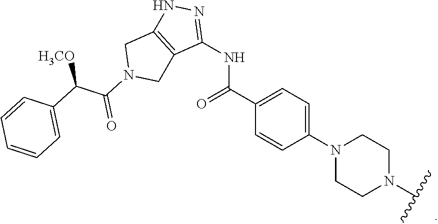 [SMILES: *N1CCN(c2ccc(C(=O)Nc3n[nH]c4c3CN(C(=O)[C@H](OC)c3ccccc3)C4)cc2)CC1]

In embodiments, A is

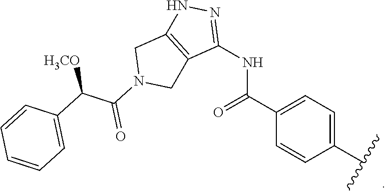 [SMILES: *c1ccc(C(=O)Nc2n[nH]c3c2CN(C(=O)[C@H](OC)c2ccccc2)C3)cc1]

In embodiments, A is

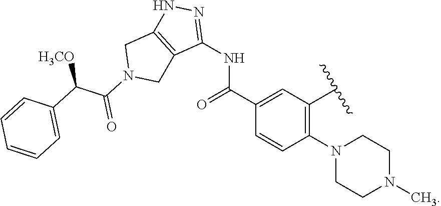 [SMILES: *c1cc(C(=O)Nc2n[nH]c3c2CN(C(=O)[C@H](OC)c2ccccc2)C3)ccc1N1CCN(C)CC1]

In embodiments, A is a monovalent form of HG-7-85-01. In embodiments, A is a monovalent form of a compound as described in WO 2010/144909, which is herein incorporated by reference in its entirety for all purposes. In embodiments, A is a monovalent form of

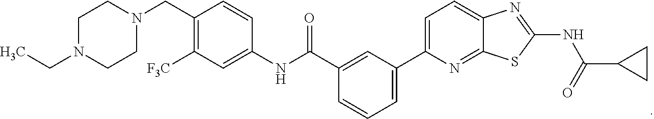 [SMILES: CCN1CCN(Cc2ccc(NC(=O)c3cccc(-c4ccc5nc(NC(=O)C6CC6)sc5n4)c3)cc2C(F)(F)F)CC1]

In embodiments, A is

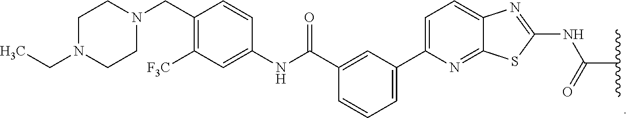 [SMILES: *C(=O)Nc1nc2ccc(-c3cccc(C(=O)Nc4ccc(CN5CCN(CC)CC5)c(C(F)(F)F)c4)c3)nc2s1]

In embodiments, A is

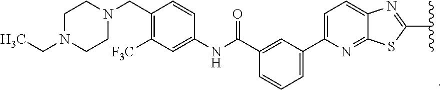 [SMILES: *c1nc2ccc(-c3cccc(C(=O)Nc4ccc(CN5CCN(CC)CC5)c(C(F)(F)F)c4)c3)nc2s1]

In embodiments, A is

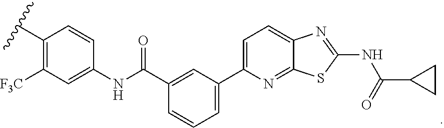 [SMILES: *c1ccc(NC(=O)c2cccc(-c3ccc4nc(NC(=O)C5CC5)sc4n3)c2)cc1C(F)(F)F]

In embodiments, A is

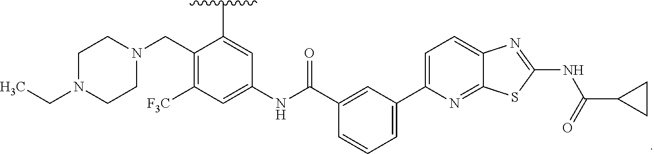 [SMILES: *c1cc(NC(=O)c2cccc(-c3ccc4nc(NC(=O)C5CC5)sc4n3)c2)cc(C(F)(F)F)c1CN1CCN(CC)CC1]

In embodiments, A is a monovalent form of a compound as described in Rossari, et al., J. Hematol. Oncol. 11, 84 (2018), which is herein incorporated by reference in its entirety for all purposes.

In embodiments, B is a monovalent form of asciminib (e.g., ABL001) or a monovalent form of GNF-2. In embodiments, B is a monovalent form of asciminib (e.g., ABL001). In embodiments, B is a monovalent form of GNF-2.

In embodiments, B is a monovalent form of asciminib (e.g., ABL001). In embodiments, B is a monovalent form of a compound as described in WO 2013/171639, which is herein incorporated by reference in its entirety for all purposes. In embodiments, B is a monovalent form of

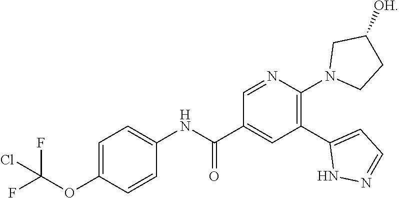 [SMILES: O=C(Nc1ccc(OC(F)(F)Cl)cc1)c1cnc(N2CC[C@@H](O)C2)c(-c2ccn[nH]2)c1]

In embodiments, B is

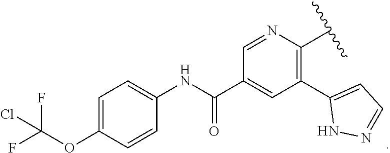 [SMILES: *c1ncc(C(=O)Nc2ccc(OC(F)(F)Cl)cc2)cc1-c1ccn[nH]1]

In embodiments, B is

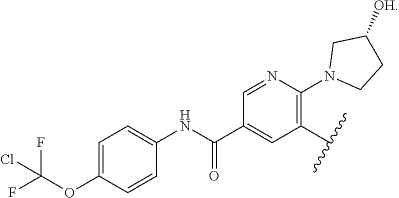 [SMILES: *c1cc(C(=O)Nc2ccc(OC(F)(F)Cl)cc2)cnc1N1CC[C@@H](O)C1]

In embodiments, B is

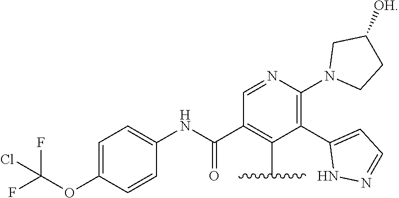 [SMILES: *c1c(C(=O)Nc2ccc(OC(F)(F)Cl)cc2)cnc(N2CC[C@@H](O)C2)c1-c1ccn[nH]1]

In embodiments, B is a monovalent form of GNF-2. In embodiments, B is a monovalent form of a compound as described in WO 2004/089286, which is herein incorporated by reference in its entirety for all purposes. In embodiments, B is a monovalent form of

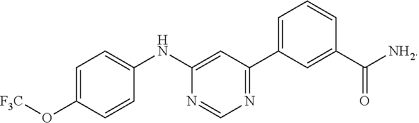 [SMILES: NC(=O)c1cccc(-c2cc(Nc3ccc(OC(F)(F)F)cc3)ncn2)c1]

In embodiments, B is

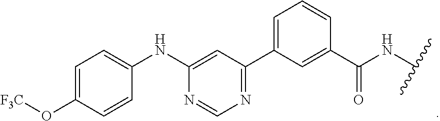 [SMILES: *NC(=O)c1cccc(-c2cc(Nc3ccc(OC(F)(F)F)cc3)ncn2)c1]

In embodiments, B is

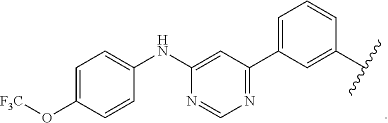 [SMILES: *c1cccc(-c2cc(Nc3ccc(OC(F)(F)F)cc3)ncn2)c1]

In embodiments, B is

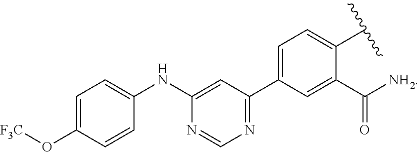 [SMILES: *c1ccc(-c2cc(Nc3ccc(OC(F)(F)F)cc3)ncn2)cc1C(N)=O]

In embodiments, B is

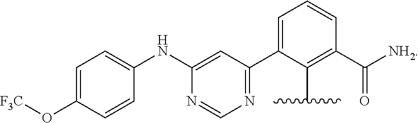 [SMILES: *c1c(C(N)=O)cccc1-c1cc(Nc2ccc(OC(F)(F)F)cc2)ncn1]

In embodiments, when R¹⁰¹ is substituted, R¹⁰¹ is substituted with one or more first substituent groups denoted by R^{101.1} as explained in the definitions section above in the description of “first substituent group(s)”. In embodiments, when an R^{101.1} substituent group is substituted, the R^{101.1} substituent group is substituted with one or more second substituent groups denoted by R^{101.2} as explained in the definitions section above in the description of “first substituent group(s)”. In embodiments, when an R^{101.2} substituent group is substituted, the R^{101.2} substituent group is substituted with one or more third substituent groups denoted by R^{101.3} as explained in the definitions section above in the description of “first substituent group(s)”. In the above embodiments, R¹⁰¹, R^{101.1}, R^{101.2}, and R^{101.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰¹, R^{101.1}, R^{101.2}, and R^{101.3}, respectively.

In embodiments, when R¹⁰² is substituted, R¹⁰² is substituted with one or more first substituent groups denoted by R^{102.1} as explained in the definitions section above in the description of “first substituent group(s)”. In embodiments, when an R^{102.1} substituent group is substituted, the R^{102.1} substituent group is substituted with one or more second substituent groups denoted by R^{102.2} as explained in the definitions section above in the description of “first substituent group(s)”. In embodiments, when an R^{102.2} substituent group is substituted, the R^{102.2} substituent group is substituted with one or more third substituent groups denoted by R^{102.3} as explained in the definitions section above in the description of “first substituent group(s)”. In the above embodiments, R¹⁰², R^{102.1}, R^{102.2}, and R^{102.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰², R^{102.1}, R^{102.2}, and R^{102.3}, respectively.

In embodiments, when R¹⁰³ is substituted, R¹⁰³ is substituted with one or more first substituent groups denoted by R^{103.1} as explained in the definitions section above in the description of “first substituent group(s)”. In embodiments, when an R^{103.1} substituent group is substituted, the R^{103.1} substituent group is substituted with one or more second substituent groups denoted by R^{103.2} as explained in the definitions section above in the description of “first substituent group(s)”. In embodiments, when an R^{103.2} substituent group is substituted, the R^{103.2} substituent group is substituted with one or more third substituent groups denoted by R^{103.3} as explained in the definitions section above in the description of “first substituent group(s)”. In the above embodiments, R¹⁰³, R^{103.1}, R^{103.2}, and R^{103.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰³, R^{103.1}, R^{103.2}, and R^{103.3}, respectively.

In embodiments, when R¹⁰⁴ is substituted, R¹⁰⁴ is substituted with one or more first substituent groups denoted by R^{104.1} as explained in the definitions section above in the description of “first substituent group(s)”. In embodiments, when an R^{104.1} substituent group is substituted, the R^{104.1} substituent group is substituted with one or more second substituent groups denoted by R^{104.2} as explained in the definitions section above in the description of “first substituent group(s)”. In embodiments, when an R^{104.2} substituent group is substituted, the R^{104.2} substituent group is substituted with one or more third substituent groups denoted by R^{104.3} as explained in the definitions section above in the description of “first substituent group(s)”. In the above embodiments, R¹⁰⁴, R^{104.1}, R^{104.2}, and R^{104.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰⁴, R^{104.1}, R^{104.2}, and R^{104.3}, respectively.

In embodiments, when R¹⁰⁵ is substituted, R¹⁰⁵ is substituted with one or more first substituent groups denoted by R^{105.1} as explained in the definitions section above in the description of “first substituent group(s)”. In embodiments, when an R^{105.1} substituent group is substituted, the R^{105.1} substituent group is substituted with one or more second substituent groups denoted by R^{105.2} as explained in the definitions section above in the description of “first substituent group(s)”. In embodiments, when an R^{105.2} substituent group is substituted, the R^{105.2} substituent group is substituted with one or more third substituent groups denoted by R^{105.3} as explained in the definitions section above in the description of “first substituent group(s)”. In the above embodiments, R¹⁰⁵, R^{105.1}, R^{105.2}, and R^{105.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰⁵, R^{105.1}, R^{105.2}, and R^{105.3}, respectively.

In embodiments, when L¹⁰¹ is substituted, L¹⁰¹ is substituted with one or more first substituent groups denoted by R^{L101.1} as explained in the definitions section above in the description of “first substituent group(s)”. In embodiments, when an R^{L101.1} substituent group is substituted, the R^{L101.1} substituent group is substituted with one or more second substituent groups denoted by R^{L101.2} as explained in the definitions section above in the description of “first substituent group(s)”. In embodiments, when an R^{L101.2} substituent group is substituted, the R^{L101.2} substituent group is substituted with one or more third substituent groups denoted by R^{L101.3} as explained in the definitions section above in the description of “first substituent group(s)”. In the above embodiments, L¹⁰¹, R^{L101.1}, R^{L101.2}, and R^{L101.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰¹, R^{L101.1}, R^{L101.2}, and R^{L101.3}, respectively.

In embodiments, when L¹⁰² is substituted, L¹⁰² is substituted with one or more first substituent groups denoted by R^{L102.1} as explained in the definitions section above in the description of “first substituent group(s)”. In embodiments, when an R^{L102.1} substituent group is substituted, the R^{L102.1} substituent group is substituted with one or more second substituent groups denoted by R^{L102.2} as explained in the definitions section above in the description of “first substituent group(s)”. In embodiments, when an R^{L102.2} substituent group is substituted, the R^{L102.2} substituent group is substituted with one or more third substituent groups denoted by R^{L102.3} as explained in the definitions section above in the description of “first substituent group(s)”. In the above embodiments, L¹⁰², R^{L102.1}, R^{L102.2} and R^{L102.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰², R^{L102.1}, R^{L102.2}, and R^{L102.3}, respectively.

In embodiments, when L¹⁰³ is substituted, L¹⁰³ is substituted with one or more first substituent groups denoted by R^{L103.1} as explained in the definitions section above in the description of “first substituent group(s)”. In embodiments, when an R^{L103.1} substituent group is substituted, the R^{L103.1} substituent group is substituted with one or more second substituent groups denoted by R^{L103.2} as explained in the definitions section above in the description of “first substituent group(s)”. In embodiments, when an R^{L103.2} substituent group is substituted, the R^{L103.2} substituent group is substituted with one or more third substituent groups denoted by R^{L103.3} as explained in the definitions section above in the description of “first substituent group(s)”. In the above embodiments, L¹⁰³, R^{L103.1}, R^{L103.2} and R^{L103.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰³, R^{L103.1}, R^{L103.2}, and R^{L103.3}, respectively.

In embodiments, when L¹⁰⁴ is substituted, L¹⁰⁴ is substituted with one or more first substituent groups denoted by R^{L104.1} as explained in the definitions section above in the description of “first substituent group(s)”. In embodiments, when an R^{L104.1} substituent group is substituted, the R^{L104.1} substituent group is substituted with one or more second substituent groups denoted by R^{L104.2} as explained in the definitions section above in the description of “first substituent group(s)”. In embodiments, when an R^{L104.2} substituent group is substituted, the R^{L104.2} substituent group is substituted with one or more third substituent groups denoted by R^{L104.3} as explained in the definitions section above in the description of “first substituent group(s)”. In the above embodiments, L¹⁰⁴, R^{L104.1}, R^{L104.2} and R^{L104.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰⁴, R^{L104.1}, R^{L104.2}, and R^{L104.3}, respectively.

In embodiments, when L¹⁰⁵ is substituted, L¹⁰⁵ is substituted with one or more first substituent groups denoted by R^{L105.1} as explained in the definitions section above in the description of “first substituent group(s)”. In embodiments, when an R^{L105.1} substituent group is substituted, the R^{L105.1} substituent group is substituted with one or more second substituent groups denoted by R^{L105.2} as explained in the definitions section above in the description of “first substituent group(s)”. In embodiments, when an R^{L105.2} substituent group is substituted, the R^{L105.2} substituent group is substituted with one or more third substituent groups denoted by R^{L105.3} as explained in the definitions section above in the description of “first substituent group(s)”. In the above embodiments, L¹⁰⁵, R^{L105.1}, R^{L105.2} and R^{L105.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰⁵, R^{L105.1}, R^{L105.2}, and R^{L105.3}, respectively.

In embodiments, the compound has the formula:

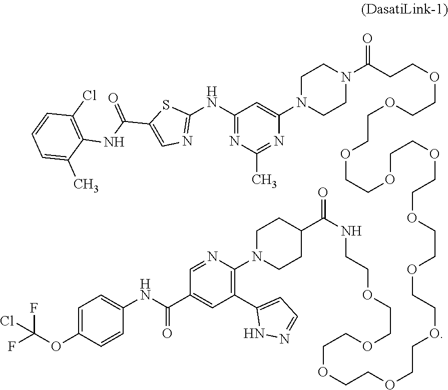 [SMILES: Cc1nc(Nc2ncc(C(=O)Nc3c(C)cccc3Cl)s2)cc(N2CCN(C(=O)CCOCCOCCOCCOCCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

In embodiments, the compound has the formula:

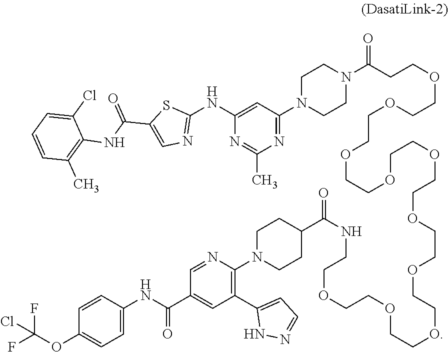 [SMILES: Cc1nc(Nc2ncc(C(=O)Nc3c(C)cccc3Cl)s2)cc(N2CCN(C(=O)CCOCCOCCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

In embodiments, the compound has the formula:

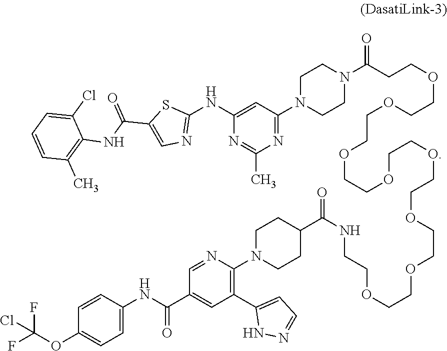 [SMILES: Cc1nc(Nc2ncc(C(=O)Nc3c(C)cccc3Cl)s2)cc(N2CCN(C(=O)CCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

In embodiments, the compound has the formula:

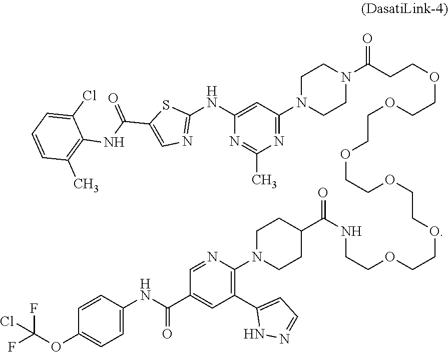 [SMILES: Cc1nc(Nc2ncc(C(=O)Nc3c(C)cccc3Cl)s2)cc(N2CCN(C(=O)CCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

In embodiments, the compound has the formula:

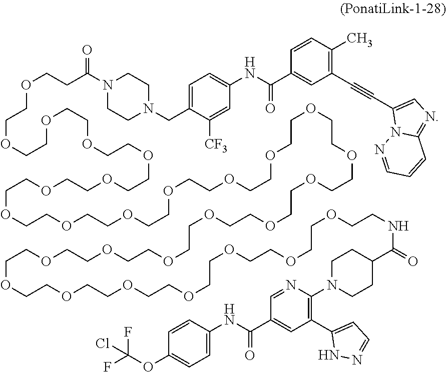 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

In embodiments, the compound has the formula:

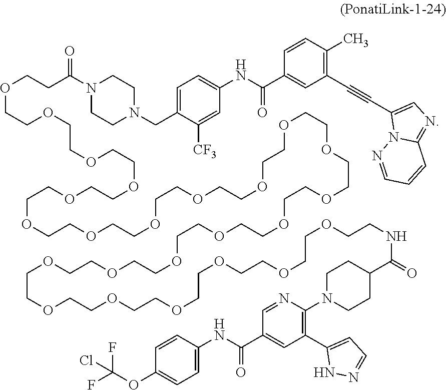 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

In embodiments, the compound has the formula:

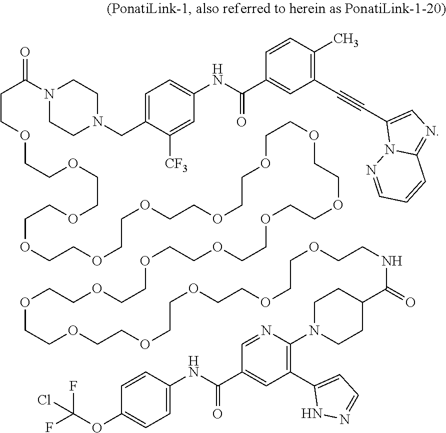 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

In embodiments, the compound has the formula:

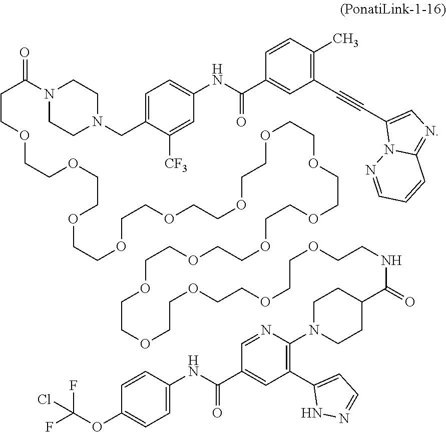 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

In embodiments, the compound has the formula:

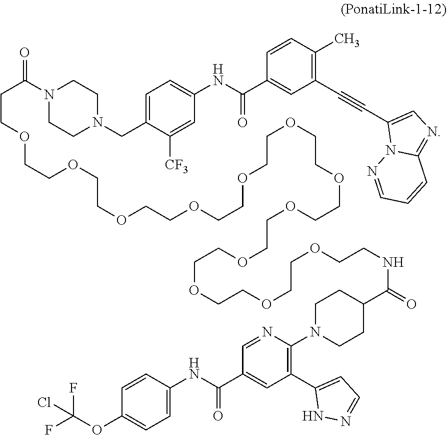 [SMILES: Cc1ccc(C(=O)Nc2ccc(CN3CCN(C(=O)CCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

In embodiments, the compound has the formula:

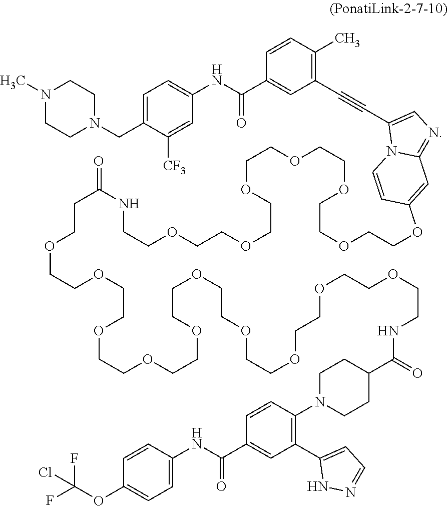 [SMILES: Cc1ccc(C(=O)Nc2ccc(CN3CCN(C)CC3)c(C(F)(F)F)c2)cc1C#Cc1cnc2cc(OCCOCCOCCOCCOCCOCCOCCNC(=O)CCOCCOCCOCCOCCOCCOCCOCCOCCOCCOCCNC(=O)C3CCN(c4ccc(C(=O)Nc5ccc(OC(F)(F)Cl)cc5)cc4-c4ccn[nH]4)CC3)ccn12]

In embodiments, the compound has the formula:

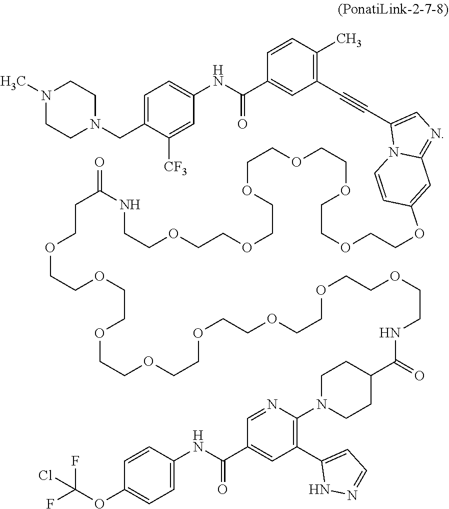 [SMILES: Cc1ccc(C(=O)Nc2ccc(CN3CCN(C)CC3)c(C(F)(F)F)c2)cc1C#Cc1cnc2cc(OCCOCCOCCOCCOCCOCCOCCNC(=O)CCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)ccn12]

In embodiments, the compound has the formula:

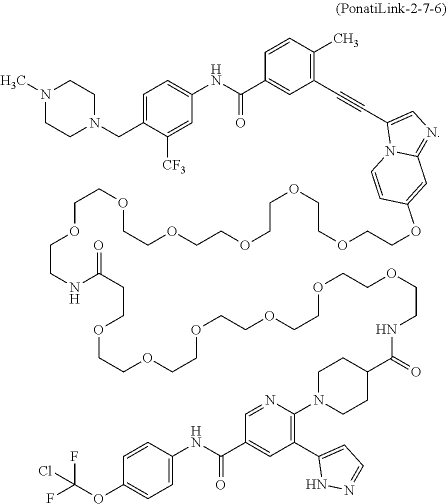 [SMILES: Cc1ccc(C(=O)Nc2ccc(CN3CCN(C)CC3)c(C(F)(F)F)c2)cc1C#Cc1cnc2cc(OCCOCCOCCOCCOCCOCCOCCNC(=O)CCOCCOCCOCCOCCOCCOCCNC(=O)C3CCN(c4ncc(C(=O)Nc5ccc(OC(F)(F)Cl)cc5)cc4-c4ccn[nH]4)CC3)ccn12]

In embodiments, the compound has the formula:

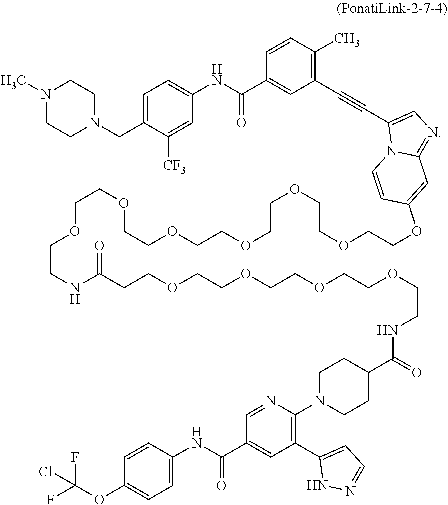 [SMILES: Cc1ccc(C(=O)Nc2ccc(CN3CCN(C)CC3)c(C(F)(F)F)c2)cc1C#Cc1cnc2cc(OCCOCCOCCOCCOCCOCCOCCNC(=O)CCOCCOCCOCCOCCNC(=O)C3CCN(c4ncc(C(=O)Nc5ccc(OC(F)(F)Cl)cc5)cc4-c4ccn[nH]4)CC3)ccn12]

In embodiments, the compound has the formula:

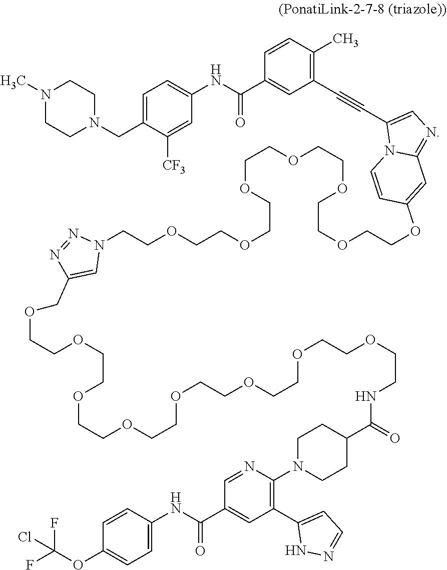 [SMILES: Cc1ccc(C(=O)Nc2ccc(CN3CCN(C)CC3)c(C(F)(F)F)c2)cc1C#Cc1cnc2cc(OCCOCCOCCOCCOCCOCCOCCn3cc(COCCOCCOCCOCCOCCOCCOCCOCCNC(=O)C4CCN(c5ncc(C(=O)Nc6ccc(OC(F)(F)Cl)cc6)cc5-c5ccn[nH]5)CC4)nn3)ccn12]

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound.

### IV. Methods of Use

In an aspect is provided a method of treating cancer in a subject in need thereof, the method including administering to the subject in need thereof a therapeutically effective amount of a compound described herein, or a pharmaceutically acceptable salt thereof.

In embodiments, the cancer is leukemia. In embodiments, the cancer is chronic myeloid leukemia, acute lymphoblastic leukemia, acute myelogenous leukemia, or mixed-phenotype acute leukemia. In embodiments, the cancer is chronic myeloid leukemia. In embodiments, the cancer is acute lymphoblastic leukemia. In embodiments, the cancer is acute myelogenous leukemia. In embodiments, the cancer is mixed-phenotype acute leukemia.

In an aspect is provided a method of treating a neurodegenerative disease in a subject in need thereof, the method including administering to the subject in need thereof a therapeutically effective amount of a compound described herein, or a pharmaceutically acceptable salt thereof.

In embodiments, the neurodegenerative disease is Parkinson's disease or Alzheimer's disease. In embodiments, the neurodegenerative disease is Parkinson's disease. In embodiments, the neurodegenerative disease is Alzheimer's disease.

In an aspect is provided a method of treating an ABL-associated disease in a subject in need thereof, the method including administering to the subject in need thereof a therapeutically effective amount of a compound described herein, or a pharmaceutically acceptable salt thereof.

In embodiments, the ABL-associated disease is cancer or a neurodegenerative disease.

In embodiments, ABL is BCR-ABL. In embodiments, BCR-ABL is BCR-ABL1.

In embodiments, the BCR-ABL1 is BCR-ABL1 wild type. In embodiments, the BCR-ABL1 is a mutant BCR-ABL1. In embodiments, the BCR-ABL1 is a T315I BCR-ABL1 mutant. In embodiments, the BCR-ABL1 is a Y253 BCR-ABL1 mutant. In embodiments, the BCR-ABL1 is a E255 BCR-ABL1 mutant. In embodiments, the BCR-ABL1 is a T315 BCR-ABL1 mutant. In embodiments, the BCR-ABL1 is a M244 BCR-ABL1 mutant. In embodiments, the BCR-ABL1 is a L248 BCR-ABL1 mutant. In embodiments, the BCR-ABL1 is a G250 BCR-ABL1 mutant. In embodiments, the BCR-ABL1 is a Q252 BCR-ABL1 mutant. In embodiments, the BCR-ABL1 is a F317 BCR-ABL1 mutant. In embodiments, the BCR-ABL1 is a M351 BCR-ABL1 mutant. In embodiments, the BCR-ABL1 is a M355 BCR-ABL1 mutant. In embodiments, the BCR-ABL1 is a F359 BCR-ABL1 mutant. In embodiments, the BCR-ABL1 is a H396 BCR-ABL1 mutant. In embodiments, the BCR-ABL1 is a V299 BCR-ABL1 mutant. In embodiments, the BCR-ABL1 is a A337 BCR-ABL1 mutant. In embodiments, the BCR-ABL1 is a W464 BCR-ABL1 mutant. In embodiments, the BCR-ABL1 is a P465 BCR-ABL1 mutant. In embodiments, the BCR-ABL1 is a V468 BCR-ABL1 mutant. In embodiments, the BCR-ABL1 is a 1502 BCR-ABL1 mutant.

In an aspect is provided a method of reducing the level of activity of ABL (e.g., BCR-ABL) in a cell, the method including contacting the cell with an effective amount of a compound described herein, or a pharmaceutically acceptable salt thereof.

In embodiments, the level of activity of ABL (e.g., BCR-ABL) is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of ABL (e.g., BCR-ABL) is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

### V. Embodiments

Embodiment P1. A compound comprising a monovalent ABL ATP binding site inhibitor covalently bound to a monovalent ABL myristoyl binding site inhibitor.

Embodiment P2. The compound of embodiment P1, having the formula: A-L¹-B; or a pharmaceutically salt thereof, wherein A is said monovalent ABL ATP binding site inhibitor; B is said monovalent ABL myristoyl binding site inhibitor; and L¹ is a divalent linker.

Embodiment P3. The compound of embodiment P2, wherein said divalent linker comprises at least 9 linear atoms between the covalent bond connecting L¹ and A and the covalent bond connecting L¹ and B.

Embodiment P4. The compound of embodiment P2, wherein said divalent linker comprises at least 18 linear atoms between the covalent bond connecting L¹ and A and the covalent bond connecting L¹ and B.

Embodiment P5. The compound of one of embodiments P2 to P4, wherein
L¹ is -L¹⁰¹-L¹⁰²-L¹⁰³-L¹⁰⁴-L¹⁰⁵-;
L¹⁰¹ is connected directly to said monovalent ABL ATP binding site inhibitor;
L¹⁰¹ is a bond, —C(O)—, —C(O)O—, —OC(O)—, —O—, —S—, —NR¹⁰¹—, —C(O)NR¹⁰¹—, —NR¹⁰¹C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁰¹, R¹⁰², R¹⁰³, R¹⁰⁴, and R¹⁰⁵ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P6. The compound of embodiment P5, wherein
L¹⁰¹ is substituted C₁-C₆ alkylene;
L¹⁰² is unsubstituted 2 to 40 membered heteroalkylene;
L¹⁰³ is unsubstituted 2 to 40 membered heteroalkylene;
L¹⁰⁴ is —NHC(O)—; and
L¹⁰⁵ is unsubstituted 3 to 8 membered heterocycloalkylene.

Embodiment P7. The compound of embodiment P5, wherein L¹ is -L¹⁰¹-(OCH₂CH₂)-L¹⁰⁴-L¹⁰⁵-; and n is an integer from 3 to 50.

Embodiment P8. The compound of embodiment P9, wherein n is an integer from 6 to 20.

Embodiment P9. The compound of embodiment P5, wherein L¹ is -L¹⁰¹-(OCH₂CH₂)ₙ-L¹⁰⁴-L¹⁰⁵-.
L¹⁰¹ is substituted oxo-substituted C₁-C₆ alkylene;
L¹⁰⁴ is —NHC(O)—;
L¹⁰⁵ is unsubstituted piperidinylene; and
n is an integer from 3 to 50.

Embodiment P10. The compound of embodiment P9, wherein n is 12.

Embodiment P11. The compound of one of embodiments P2 to P10, wherein A is a monovalent form of dasatinib, a monovalent form of ponatinib, a monovalent form of imatinib, a monovalent form of nilotinib, a monovalent form of bosutinib, a monovalent form of bafetinib, a monovalent form of olverembatinib, a monovalent form of tozasertib, a monovalent form of PF-114, a monovalent form of rebastinib, a monovalent form of danusertib, or a monovalent form of HG-7-85-01.

Embodiment P12. The compound of one of embodiments P2 to P10, wherein A is a monovalent form of

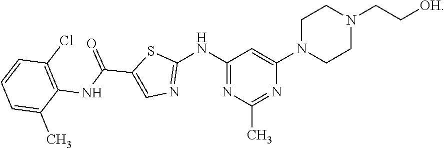 [SMILES: Cc1nc(Nc2ncc(C(=O)Nc3c(C)cccc3Cl)s2)cc(N2CCN(CCO)CC2)n1]

Embodiment P13. The compound of embodiment P12, wherein A is

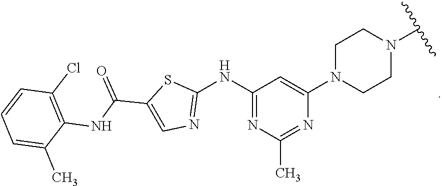 [SMILES: *N1CCN(c2cc(Nc3ncc(C(=O)Nc4c(C)cccc4Cl)s3)nc(C)n2)CC1]

Embodiment P14. The compound of one of embodiments P2 to P10, wherein A is a monovalent form of

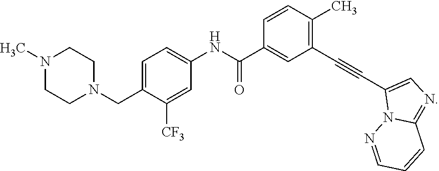 [SMILES: Cc1ccc(C(=O)Nc2ccc(CN3CCN(C)CC3)c(C(F)(F)F)c2)cc1C#Cc1cnc2cccnn12]

Embodiment P15. The compound of embodiment P14, wherein A is

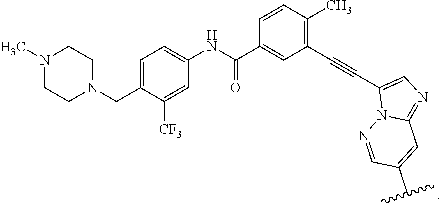 [SMILES: *c1cnn2c(C#Cc3cc(C(=O)Nc4ccc(CN5CCN(C)CC5)c(C(F)(F)F)c4)ccc3C)cnc2c1]

Embodiment P16. The compound of one of embodiments P2 to P10, wherein A is a monovalent form of

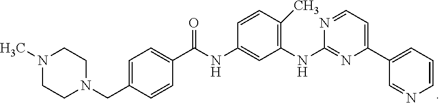 [SMILES: Cc1ccc(NC(=O)c2ccc(CN3CCN(C)CC3)cc2)cc1Nc1nccc(-c2cccnc2)n1]

Embodiment P17. The compound of embodiment P16, wherein A is

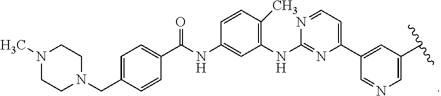 [SMILES: *c1cncc(-c2ccnc(Nc3cc(NC(=O)c4ccc(CN5CCN(C)CC5)cc4)ccc3C)n2)c1]

Embodiment P18. The compound of one of embodiments P2 to P10, wherein A is a monovalent form of

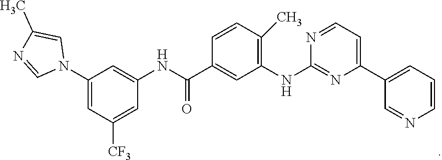 [SMILES: Cc1cn(-c2cc(NC(=O)c3ccc(C)c(Nc4nccc(-c5cccnc5)n4)c3)cc(C(F)(F)F)c2)cn1]

Embodiment P19. The compound of embodiment P18, wherein A is

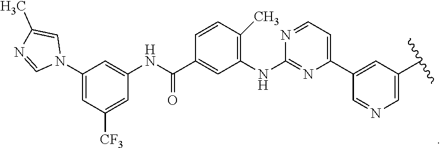 [SMILES: *c1cncc(-c2ccnc(Nc3cc(C(=O)Nc4cc(-n5cnc(C)c5)cc(C(F)(F)F)c4)ccc3C)n2)c1]

Embodiment P20. The compound of one of embodiments P2 to P10, wherein A is a monovalent form of

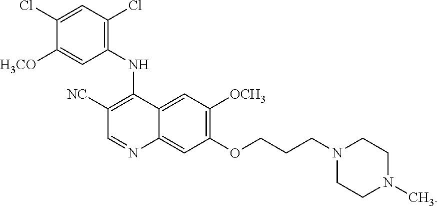 [SMILES: COc1cc(Nc2c(C#N)cnc3cc(OCCCN4CCN(C)CC4)c(OC)cc23)c(Cl)cc1Cl]

Embodiment P21. The compound of embodiment P20, wherein A is

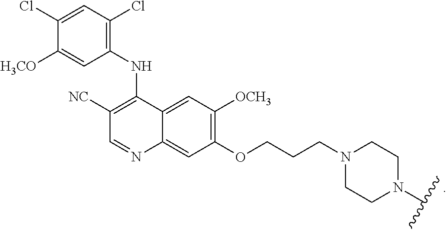 [SMILES: *N1CCN(CCCOc2cc3ncc(C#N)c(Nc4cc(OC)c(Cl)cc4Cl)c3cc2OC)CC1]

Embodiment P22. The compound of one of embodiments P2 to P10, wherein A is a monovalent form of

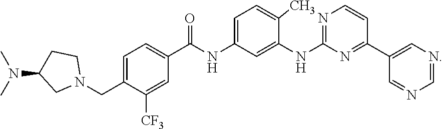 [SMILES: Cc1ccc(NC(=O)c2ccc(CN3CC[C@H](N(C)C)C3)c(C(F)(F)F)c2)cc1Nc1nccc(-c2cncnc2)n1]

Embodiment P23. The compound of embodiment P22, wherein A is

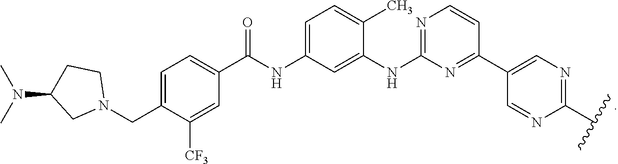 [SMILES: *c1ncc(-c2ccnc(Nc3cc(NC(=O)c4ccc(CN5CC[C@H](N(C)C)C5)c(C(F)(F)F)c4)ccc3C)n2)cn1]

Embodiment P24. The compound of one of embodiments P2 to P10, wherein A is a monovalent form of

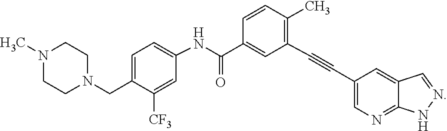 [SMILES: Cc1ccc(C(=O)Nc2ccc(CN3CCN(C)CC3)c(C(F)(F)F)c2)cc1C#Cc1cnc2[nH]ncc2c1]

Embodiment P25. The compound of embodiment P24, wherein A is

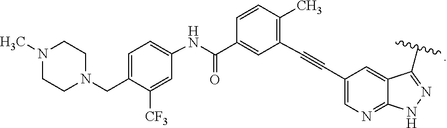 [SMILES: *c1n[nH]c2ncc(C#Cc3cc(C(=O)Nc4ccc(CN5CCN(C)CC5)c(C(F)(F)F)c4)ccc3C)cc12]

Embodiment P26. The compound of one of embodiments P2 to P10, wherein A is a monovalent form of

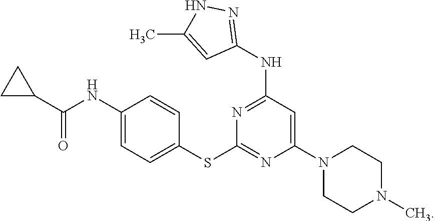 [SMILES: Cc1cc(Nc2cc(N3CCN(C)CC3)nc(Sc3ccc(NC(=O)C4CC4)cc3)n2)n[nH]1]

Embodiment P27. The compound of embodiment P26, wherein A is

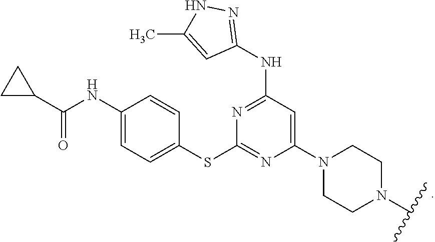 [SMILES: *N1CCN(c2cc(Nc3cc(C)[nH]n3)nc(Sc3ccc(NC(=O)C4CC4)cc3)n2)CC1]

Embodiment P28. The compound of one of embodiments P2 to P10, wherein A is a monovalent form of

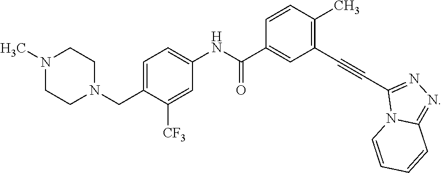 [SMILES: Cc1ccc(C(=O)Nc2ccc(CN3CCN(C)CC3)c(C(F)(F)F)c2)cc1C#Cc1nnc2ccccn12]

Embodiment P29. The compound of embodiment P28, wherein A is

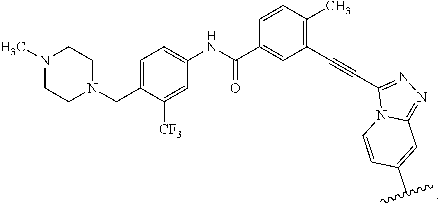 [SMILES: *c1ccn2c(C#Cc3cc(C(=O)Nc4ccc(CN5CCN(C)CC5)c(C(F)(F)F)c4)ccc3C)nnc2c1]

Embodiment P30. The compound of one of embodiments P2 to P10, wherein A is a monovalent form of

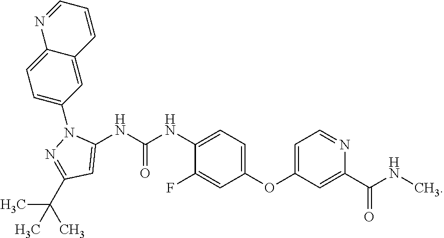 [SMILES: CNC(=O)c1cc(Oc2ccc(NC(=O)Nc3cc(C(C)(C)C)nn3-c3ccc4ncccc4c3)c(F)c2)ccn1]

Embodiment P31. The compound of embodiment P30, wherein A is

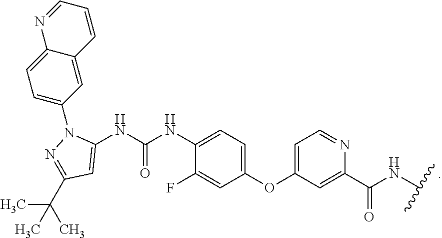 [SMILES: *NC(=O)c1cc(Oc2ccc(NC(=O)Nc3cc(C(C)(C)C)nn3-c3ccc4ncccc4c3)c(F)c2)ccn1]

Embodiment P32. The compound of one of embodiments P2 to P10, wherein A is a monovalent form of

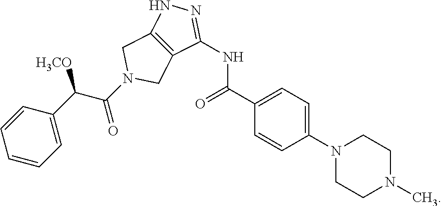 [SMILES: CO[C@@H](C(=O)N1Cc2[nH]nc(NC(=O)c3ccc(N4CCN(C)CC4)cc3)c2C1)c1ccccc1]

Embodiment P33. The compound of embodiment P32, wherein A is

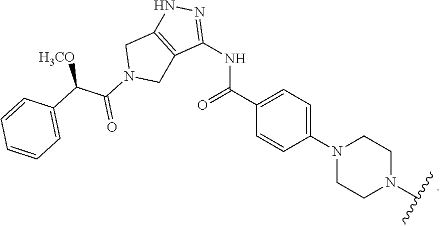 [SMILES: *N1CCN(c2ccc(C(=O)Nc3n[nH]c4c3CN(C(=O)[C@H](OC)c3ccccc3)C4)cc2)CC1]

Embodiment P34. The compound of one of embodiments P2 to P10, wherein A is a monovalent form of

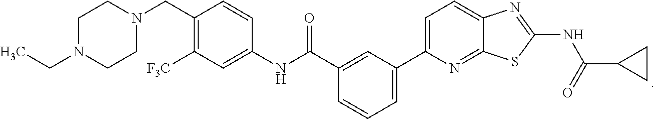 [SMILES: CCN1CCN(Cc2ccc(NC(=O)c3cccc(-c4ccc5nc(NC(=O)C6CC6)sc5n4)c3)cc2C(F)(F)F)CC1]

Embodiment P35. The compound of embodiment P34, wherein A is

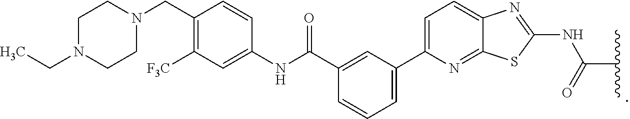 [SMILES: *C(=O)Nc1nc2ccc(-c3cccc(C(=O)Nc4ccc(CN5CCN(CC)CC5)c(C(F)(F)F)c4)c3)nc2s1]

Embodiment P36. The compound of one of embodiments P2 to P35, wherein B is a monovalent form of asciminib or a monovalent form of GNF-2.

Embodiment P37. The compound of one of embodiments P2 to P35, wherein B is a monovalent form of

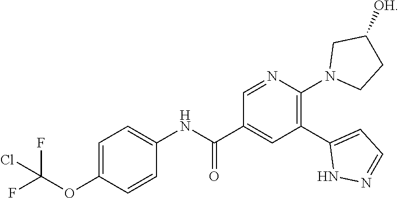 [SMILES: O=C(Nc1ccc(OC(F)(F)Cl)cc1)c1cnc(N2CC[C@@H](O)C2)c(-c2ccn[nH]2)c1]

Embodiment P38. The compound of one of embodiments P2 to P35, wherein B is

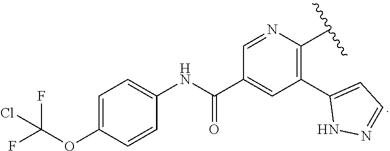 [SMILES: *c1ncc(C(=O)Nc2ccc(OC(F)(F)Cl)cc2)cc1-c1ccn[nH]1]

Embodiment P39. The compound of one of embodiments P2 to P35, wherein B is a monovalent form of

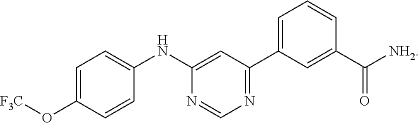 [SMILES: NC(=O)c1cccc(-c2cc(Nc3ccc(OC(F)(F)F)cc3)ncn2)c1]

Embodiment P40. The compound of embodiment P39, wherein B is

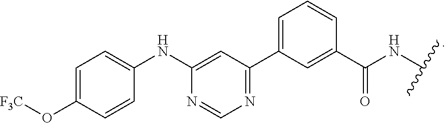 [SMILES: *NC(=O)c1cccc(-c2cc(Nc3ccc(OC(F)(F)F)cc3)ncn2)c1]

Embodiment P41. The compound of embodiment P1, having the formula:

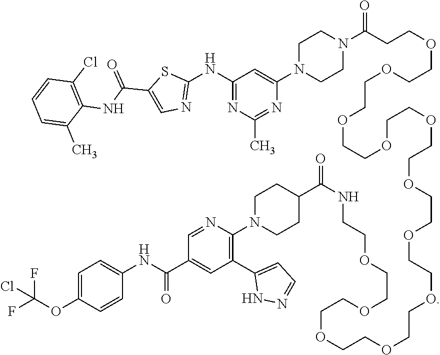 [SMILES: Cc1nc(Nc2ncc(C(=O)Nc3c(C)cccc3Cl)s2)cc(N2CCN(C(=O)CCOCCOCCOCCOCCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

Embodiment P42. A pharmaceutical composition comprising a pharmaceutically acceptable excipient and a compound of one of embodiments P1 to P41, or a pharmaceutically acceptable salt thereof.

Embodiment P43. A method of treating cancer in a subject in need thereof, said method comprising administering to the subject in need thereof a therapeutically effective amount of a compound of one of embodiments P1 to P41, or a pharmaceutically acceptable salt thereof.

Embodiment P44. The method of embodiment P43, wherein the cancer is leukemia.

Embodiment P45. The method of embodiment P43, wherein the cancer is chronic myeloid leukemia, acute lymphoblastic leukemia, acute myelogenous leukemia, or mixed-phenotype acute leukemia.

Embodiment P46. A method of treating a neurodegenerative disease in a subject in need thereof, said method comprising administering to the subject in need thereof a therapeutically effective amount of a compound of one of embodiments P1 to P41, or a pharmaceutically acceptable salt thereof.

Embodiment P47. The method of embodiment P46, wherein the neurodegenerative disease is Parkinson's disease or Alzheimer's disease.

Embodiment P48. A method of treating an ABL-associated disease in a subject in need thereof, said method comprising administering to the subject in need thereof a therapeutically effective amount of a compound of one of embodiments P1 to P41, or a pharmaceutically acceptable salt thereof.

Embodiment P49. The method of embodiment P48, wherein said ABL-associated disease is cancer or a neurodegenerative disease.

Embodiment P50. The method of embodiment P48, wherein ABL is BCR-ABL.

Embodiment P51. The method of embodiment P50, wherein BCR-ABL is BCR-ABL1.

Embodiment P52. The method of embodiment P51, wherein the BCR-ABL1 is BCR-ABL1 wild type.

Embodiment P53. The method of embodiment P51, wherein the BCR-ABL1 is a T315I BCR-ABL1 mutant.

Embodiment P54. A method of reducing the level of activity of ABL in a cell, said method comprising contacting the cell with an effective amount of a compound of one of embodiments P1 to P41, or a pharmaceutically acceptable salt thereof.

Embodiment P55. The method of embodiment P54, wherein ABL is BCR-ABL.

Embodiment P56. The method of embodiment P55, wherein the BCR-ABL is BCR-ABL1.

Embodiment P57. The method of embodiment P56, wherein the BCR-ABL1 is BCR-ABL1 wild type.

Embodiment P58. The method of embodiment P56, wherein the BCR-ABL1 is a T315I BCR-ABL1 mutant.

### VI. Additional Embodiments

Embodiment 1. A compound comprising a monovalent ABL ATP binding site inhibitor covalently bound to a monovalent ABL myristoyl binding site inhibitor.

Embodiment 2. The compound of embodiment 1, having the formula:
A-L¹-B; or a pharmaceutically salt thereof, wherein A is said monovalent ABL ATP binding site inhibitor; B is said monovalent ABL myristoyl binding site inhibitor; and L¹ is a divalent linker.

Embodiment 3. The compound of embodiment 2, wherein said divalent linker comprises at least 9 linear atoms between the covalent bond connecting L¹ and A and the covalent bond connecting L¹ and B.

Embodiment 4. The compound of embodiment 2, wherein said divalent linker comprises at least 18 linear atoms between the covalent bond connecting L¹ and A and the covalent bond connecting L¹ and B.

Embodiment 5. The compound of one of embodiments 2 to 4, wherein
L¹ is -L¹⁰¹-L¹⁰²-L¹⁰³-L¹⁰⁴-L¹⁰⁵-.
L¹⁰¹ is connected directly to said monovalent ABL ATP binding site inhibitor;
L¹⁰¹ is a bond, —C(O)—, —C(O)O—, —OC(O)—, —O—, —S—, —NR¹⁰¹-, —C(O)NR¹⁰¹—, —NR¹⁰¹C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁰¹, R¹⁰², R¹⁰³, R¹⁰⁴, and R¹⁰⁵ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 6. The compound of embodiment 5, wherein
L¹⁰¹ is substituted C₁-C₆ alkylene;
L¹⁰² is unsubstituted 2 to 40 membered heteroalkylene;
L¹⁰³ is unsubstituted 2 to 40 membered heteroalkylene;
L¹⁰⁴ is —NHC(O)—; and
L¹⁰⁵ is unsubstituted 3 to 8 membered heterocycloalkylene.

Embodiment 7. The compound of embodiment 5, wherein L¹ is -L¹⁰¹-(OCH₂CH₂)-L¹⁰⁴-L¹⁰⁵-; and n is an integer from 3 to 50.
Embodiment 8. The compound of embodiment 7, wherein n is an integer from 6 to 20.

Embodiment 9. The compound of embodiment 5, wherein L¹ is -L¹⁰¹-(OCH₂CH₂)ₙ-L¹⁰⁴-L¹⁰⁵-.
L¹⁰¹ is substituted oxo-substituted C₁-C₆ alkylene;
L¹⁰⁴ is —NHC(O)—;
L¹⁰⁵ is unsubstituted piperidinylene; and
n is an integer from 3 to 50.

Embodiment 10. The compound of embodiment 9, wherein n is 12.

Embodiment 11. The compound of one of embodiments 2 to 10, wherein A is a monovalent form of dasatinib, a monovalent form of ponatinib, a monovalent form of imatinib, a monovalent form of nilotinib, a monovalent form of bosutinib, a monovalent form of bafetinib, a monovalent form of olverembatinib, a monovalent form of tozasertib, a monovalent form of PF-114, a monovalent form of rebastinib, a monovalent form of danusertib, or a monovalent form of HG-7-85-01.

Embodiment 12. The compound of one of embodiments 2 to 10, wherein A is a monovalent form of

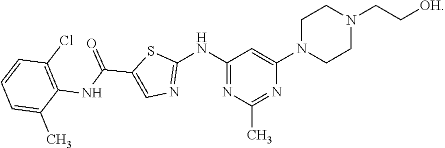 [SMILES: Cc1nc(Nc2ncc(C(=O)Nc3c(C)cccc3Cl)s2)cc(N2CCN(CCO)CC2)n1]

Embodiment 13. The compound of embodiment 12, wherein A is

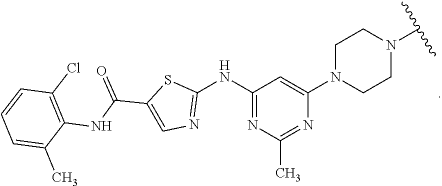 [SMILES: *N1CCN(c2cc(Nc3ncc(C(=O)Nc4c(C)cccc4Cl)s3)nc(C)n2)CC1]

Embodiment 14. The compound of one of embodiments 12 to 13, wherein the divalent linker comprises from 20 to 45 linear atoms between the covalent bond connecting L¹ and A and the covalent bond connecting L¹ and B.

Embodiment 15. The compound of one of embodiments 12 to 13, wherein L¹ is

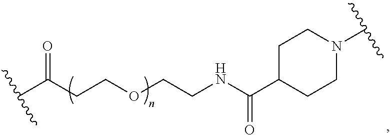

wherein n is an integer from 3 to 50.

Embodiment 16. The compound of embodiment 15, wherein n is an integer from 6 to 12.

Embodiment 17. The compound of one of embodiments 2 to 10, wherein A is a monovalent form of

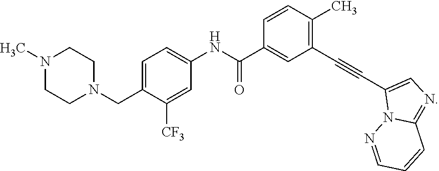 [SMILES: Cc1ccc(C(=O)Nc2ccc(CN3CCN(C)CC3)c(C(F)(F)F)c2)cc1C#Cc1cnc2cccnn12]

Embodiment 18. The compound of embodiment 17, wherein A is

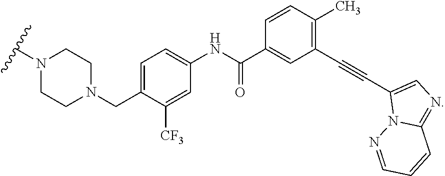 [SMILES: *N1CCN(Cc2ccc(NC(=O)c3ccc(C)c(C#Cc4cnc5cccnn45)c3)cc2C(F)(F)F)CC1]

Embodiment 19. The compound of one of embodiments 17 to 18, wherein the divalent linker comprises from 65 to 90 linear atoms between the covalent bond connecting L¹ and A and the covalent bond connecting L¹ and B.

Embodiment 20. The compound of one of embodiments 17 to 18, wherein L¹ is

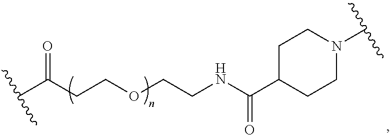

wherein n is an integer from 3 to 50.

Embodiment 21. The compound of embodiment 20, wherein n is an integer from 12 to 28.

Embodiment 22. The compound of embodiment 20, wherein n is an integer from to 28.

Embodiment 23. The compound of embodiment 17, wherein A is

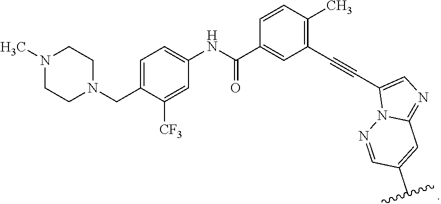 [SMILES: *c1cnn2c(C#Cc3cc(C(=O)Nc4ccc(CN5CCN(C)CC5)c(C(F)(F)F)c4)ccc3C)cnc2c1]

Embodiment 24. The compound of one of embodiments 2 to 10, wherein A is a monovalent form of

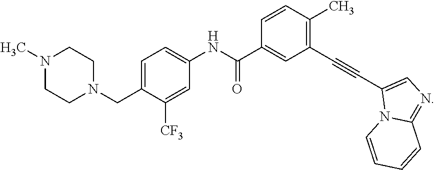 [SMILES: Cc1ccc(C(=O)Nc2ccc(CN3CCN(C)CC3)c(C(F)(F)F)c2)cc1C#Cc1cnc2ccccn12]

Embodiment 25. The compound of embodiment 24, wherein A is

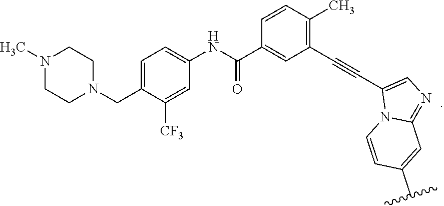 [SMILES: *c1ccn2c(C#Cc3cc(C(=O)Nc4ccc(CN5CCN(C)CC5)c(C(F)(F)F)c4)ccc3C)cnc2c1]

Embodiment 26. The compound of one of embodiments 24 to 25, wherein the divalent linker comprises from 35 to 60 linear atoms between the covalent bond connecting L¹ and A and the covalent bond connecting L¹ and B.

Embodiment 27. The compound of one of embodiments 24 to 25, wherein L¹ is

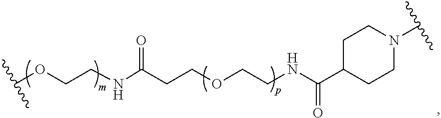

wherein m and p are independently an integer from 3 to 50.

Embodiment 28. The compound of embodiment 27, wherein m is an integer from 6 to 8.

Embodiment 29. The compound of embodiment 27, wherein m is 7.

Embodiment 30. The compound of one of embodiments 27 to 29, wherein p is an integer from 4 to 10.

Embodiment 31. The compound of one of embodiments 2 to 10, wherein A is a monovalent form of

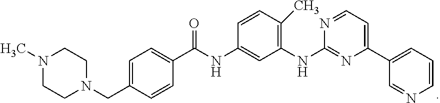 [SMILES: Cc1ccc(NC(=O)c2ccc(CN3CCN(C)CC3)cc2)cc1Nc1nccc(-c2cccnc2)n1]

Embodiment 32. The compound of embodiment 31, wherein A is

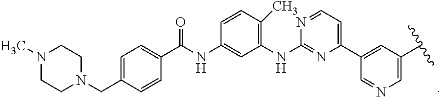 [SMILES: *c1cncc(-c2ccnc(Nc3cc(NC(=O)c4ccc(CN5CCN(C)CC5)cc4)ccc3C)n2)c1]

Embodiment 33. The compound of one of embodiments 2 to 10, wherein A is a monovalent form of

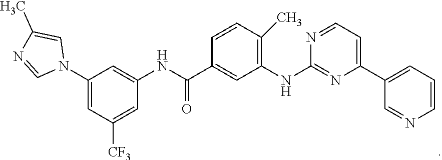 [SMILES: Cc1cn(-c2cc(NC(=O)c3ccc(C)c(Nc4nccc(-c5cccnc5)n4)c3)cc(C(F)(F)F)c2)cn1]

Embodiment 34. The compound of embodiment 33, wherein A is

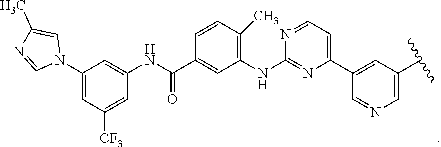 [SMILES: *c1cncc(-c2ccnc(Nc3cc(C(=O)Nc4cc(-n5cnc(C)c5)cc(C(F)(F)F)c4)ccc3C)n2)c1]

Embodiment 35. The compound of one of embodiments 2 to 10, wherein A is a monovalent form of

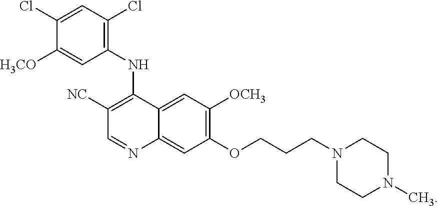 [SMILES: COc1cc(Nc2c(C#N)cnc3cc(OCCCN4CCN(C)CC4)c(OC)cc23)c(Cl)cc1Cl]

Embodiment 36. The compound of embodiment 35, wherein A is

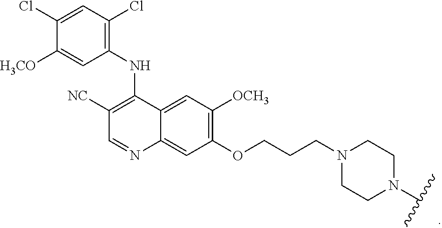 [SMILES: *N1CCN(CCCOc2cc3ncc(C#N)c(Nc4cc(OC)c(Cl)cc4Cl)c3cc2OC)CC1]

Embodiment 37. The compound of one of embodiments 2 to 10, wherein A is a monovalent form of

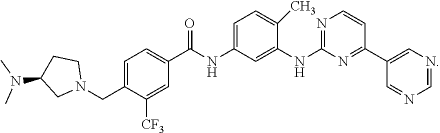 [SMILES: Cc1ccc(NC(=O)c2ccc(CN3CC[C@H](N(C)C)C3)c(C(F)(F)F)c2)cc1Nc1nccc(-c2cncnc2)n1]

Embodiment 38. The compound of embodiment 37, wherein A is

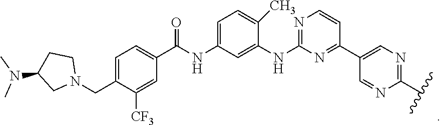 [SMILES: *c1ncc(-c2ccnc(Nc3cc(NC(=O)c4ccc(CN5CC[C@H](N(C)C)C5)c(C(F)(F)F)c4)ccc3C)n2)cn1]

Embodiment 39. The compound of one of embodiments 2 to 10, wherein A is a monovalent form of

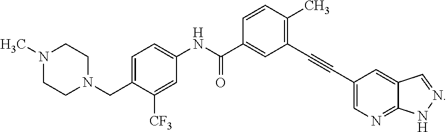 [SMILES: Cc1ccc(C(=O)Nc2ccc(CN3CCN(C)CC3)c(C(F)(F)F)c2)cc1C#Cc1cnc2[nH]ncc2c1]

Embodiment 40. The compound of embodiment 39, wherein A is

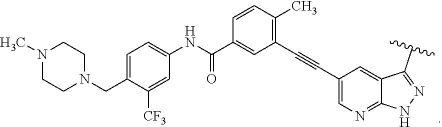 [SMILES: *c1n[nH]c2ncc(C#Cc3cc(C(=O)Nc4ccc(CN5CCN(C)CC5)c(C(F)(F)F)c4)ccc3C)cc12]

Embodiment 41. The compound of one of embodiments 2 to 10, wherein A is a monovalent form of

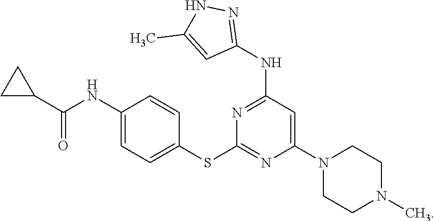 [SMILES: Cc1cc(Nc2cc(N3CCN(C)CC3)nc(Sc3ccc(NC(=O)C4CC4)cc3)n2)n[nH]1]

Embodiment 42. The compound of embodiment 41, wherein A is monovalent form of

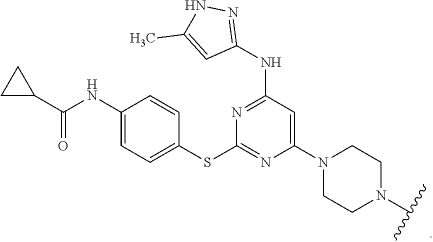 [SMILES: *N1CCN(c2cc(Nc3cc(C)[nH]n3)nc(Sc3ccc(NC(=O)C4CC4)cc3)n2)CC1]

Embodiment 43. The compound of one of embodiments 2 to 10, wherein A is a monovalent form of

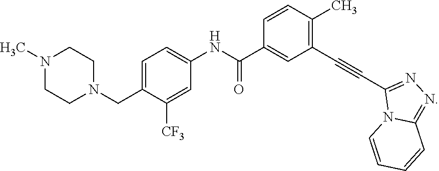 [SMILES: Cc1ccc(C(=O)Nc2ccc(CN3CCN(C)CC3)c(C(F)(F)F)c2)cc1C#Cc1nnc2ccccn12]

Embodiment 44. The compound of embodiment 43, wherein A is

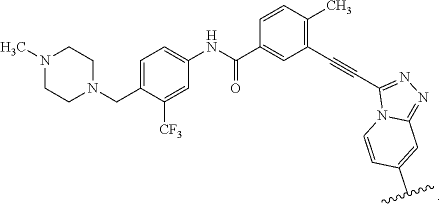 [SMILES: *c1ccn2c(C#Cc3cc(C(=O)Nc4ccc(CN5CCN(C)CC5)c(C(F)(F)F)c4)ccc3C)nnc2c1]

Embodiment 45. The compound of one of embodiments 2 to 10, wherein A is a monovalent form of

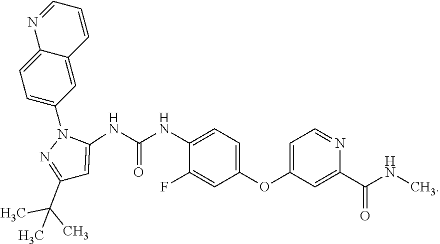 [SMILES: CNC(=O)c1cc(Oc2ccc(NC(=O)Nc3cc(C(C)(C)C)nn3-c3ccc4ncccc4c3)c(F)c2)ccn1]

Embodiment 46. The compound of embodiment 45, wherein A is

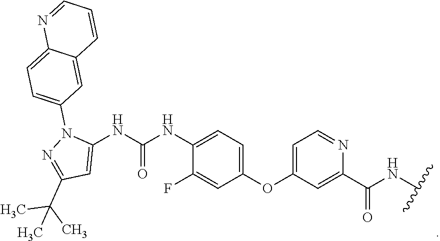 [SMILES: *NC(=O)c1cc(Oc2ccc(NC(=O)Nc3cc(C(C)(C)C)nn3-c3ccc4ncccc4c3)c(F)c2)ccn1]

Embodiment 47. The compound of one of embodiments 2 to 10, wherein A is a monovalent form of

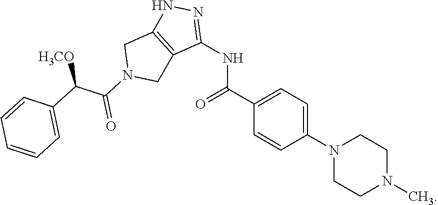 [SMILES: CO[C@@H](C(=O)N1Cc2[nH]nc(NC(=O)c3ccc(N4CCN(C)CC4)cc3)c2C1)c1ccccc1]

Embodiment 48. The compound of embodiment 47, wherein A is

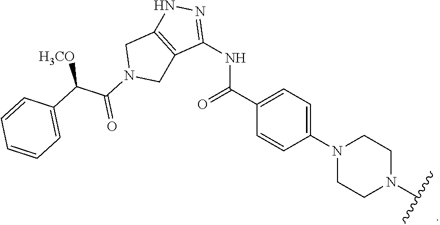 [SMILES: *N1CCN(c2ccc(C(=O)Nc3n[nH]c4c3CN(C(=O)[C@H](OC)c3ccccc3)C4)cc2)CC1]

Embodiment 49. The compound of one of embodiments 2 to 10, wherein A is a monovalent form of

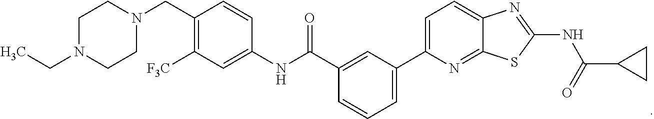 [SMILES: CCN1CCN(Cc2ccc(NC(=O)c3cccc(-c4ccc5nc(NC(=O)C6CC6)sc5n4)c3)cc2C(F)(F)F)CC1]

Embodiment 50. The compound of embodiment 49, wherein A is

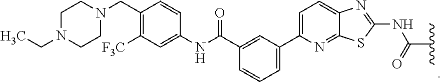 [SMILES: *C(=O)Nc1nc2ccc(-c3cccc(C(=O)Nc4ccc(CN5CCN(CC)CC5)c(C(F)(F)F)c4)c3)nc2s1]

Embodiment 51. The compound of one of embodiments 2 to 50, wherein B is a monovalent form of asciminib or a monovalent form of GNF-2.

Embodiment 52. The compound of one of embodiments 2 to 50, wherein B is a monovalent form of

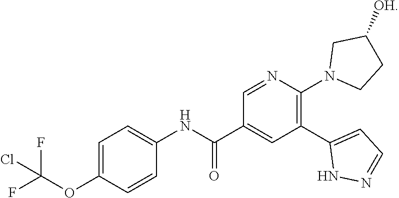 [SMILES: O=C(Nc1ccc(OC(F)(F)Cl)cc1)c1cnc(N2CC[C@@H](O)C2)c(-c2ccn[nH]2)c1]

Embodiment 53. The compound of one of embodiments 2 to 50, wherein B is

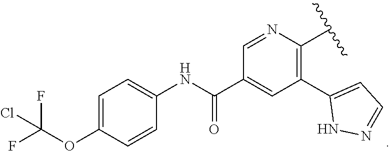 [SMILES: *c1ncc(C(=O)Nc2ccc(OC(F)(F)Cl)cc2)cc1-c1ccn[nH]1]

Embodiment 54. The compound of one of embodiments 2 to 50, wherein B is a monovalent form of

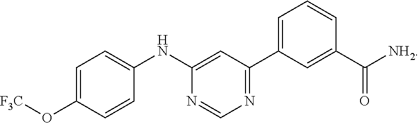 [SMILES: NC(=O)c1cccc(-c2cc(Nc3ccc(OC(F)(F)F)cc3)ncn2)c1]

Embodiment 55. The compound of embodiment 54, wherein B is

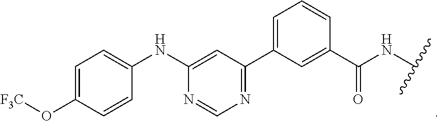 [SMILES: *NC(=O)c1cccc(-c2cc(Nc3ccc(OC(F)(F)F)cc3)ncn2)c1]

Embodiment 56. The compound of embodiment 1, having the formula:

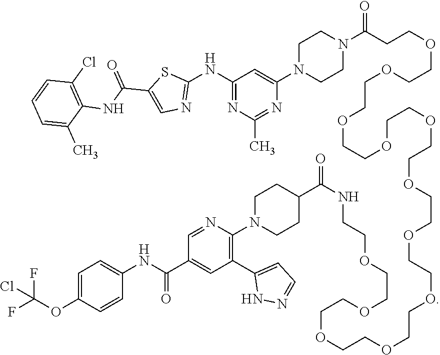 [SMILES: Cc1nc(Nc2ncc(C(=O)Nc3c(C)cccc3Cl)s2)cc(N2CCN(C(=O)CCOCCOCCOCCOCCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

Embodiment 57. The compound of embodiment 1, having the formula:

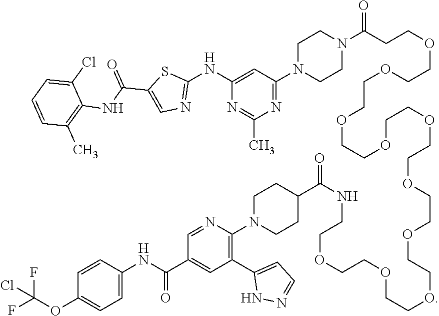 [SMILES: Cc1nc(Nc2ncc(C(=O)Nc3c(C)cccc3Cl)s2)cc(N2CCN(C(=O)CCOCCOCCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

Embodiment 58. The compound of embodiment 1, having the formula:

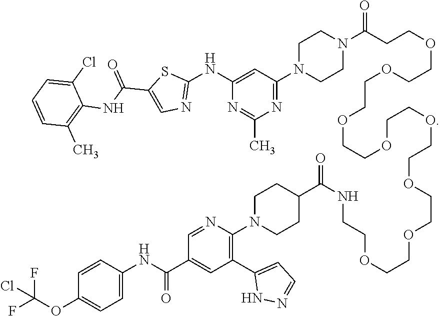 [SMILES: Cc1nc(Nc2ncc(C(=O)Nc3c(C)cccc3Cl)s2)cc(N2CCN(C(=O)CCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

Embodiment 59. The compound of embodiment 1, having the formula:

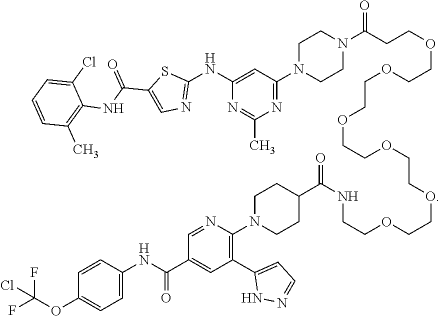 [SMILES: Cc1nc(Nc2ncc(C(=O)Nc3c(C)cccc3Cl)s2)cc(N2CCN(C(=O)CCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

Embodiment 60. The compound of embodiment 1, having the formula:

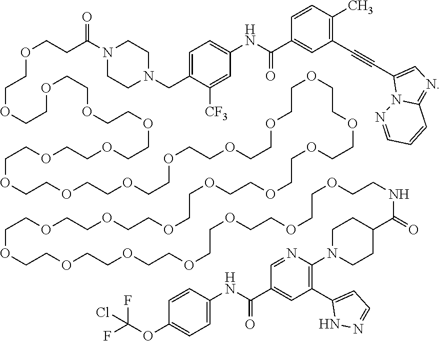 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

Embodiment 61. The compound of embodiment 1, having the formula:

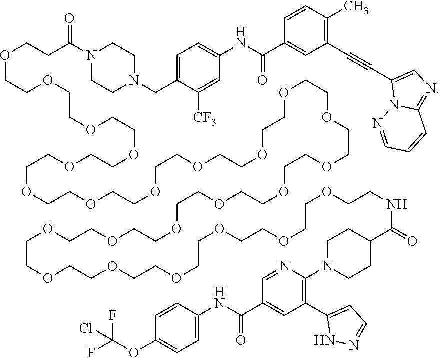 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

Embodiment 62. The compound of embodiment 1, having the formula:

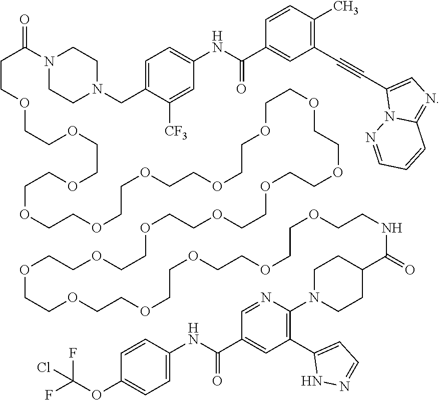 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

Embodiment 63. The compound of embodiment 1, having the formula:

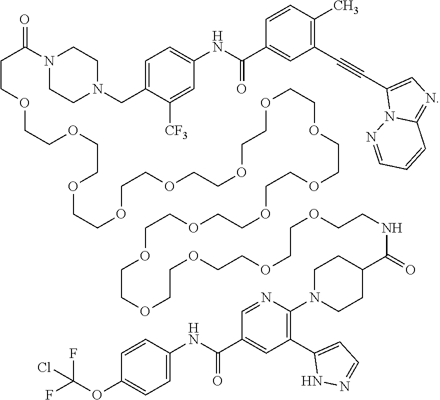

Embodiment 64. The compound of embodiment 1, having the formula:

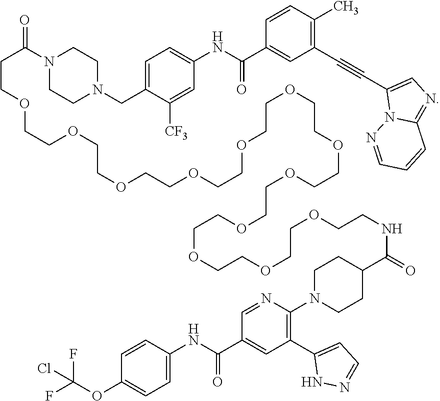

Embodiment 65. The compound of embodiment 1, having the formula:

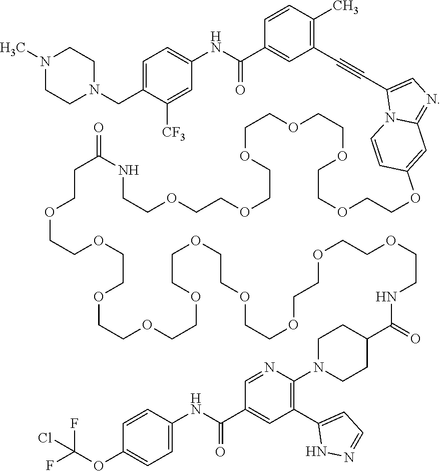

Embodiment 66. The compound of embodiment 1, having the formula:

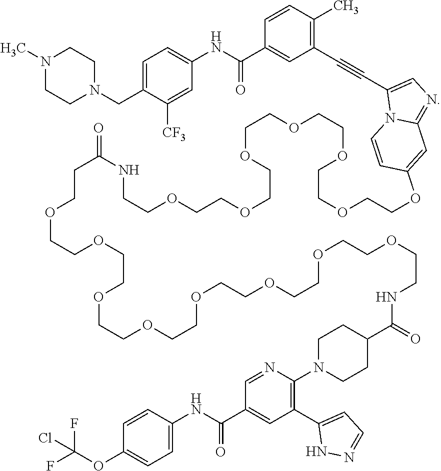

Embodiment 67. The compound of embodiment 1, having the formula:

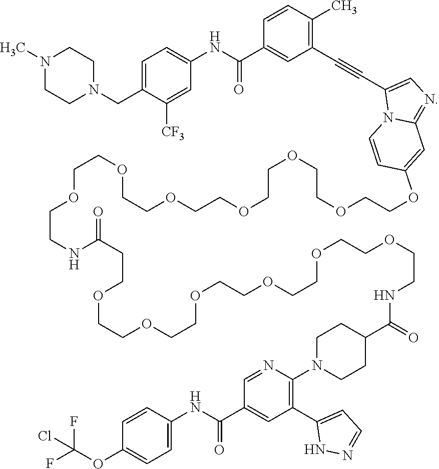

Embodiment 68. The compound of embodiment 1, having the formula:

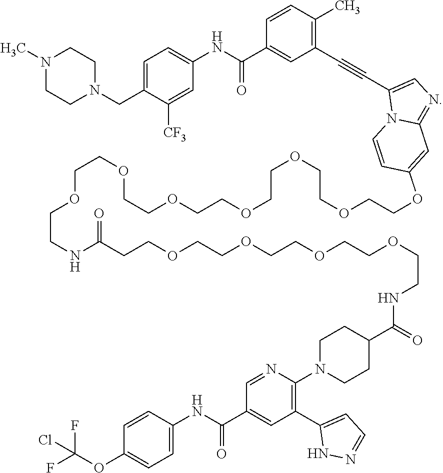 [SMILES: Cc1ccc(C(=O)Nc2ccc(CN3CCN(C)CC3)c(C(F)(F)F)c2)cc1C#Cc1cnc2cc(OCCOCCOCCOCCOCCOCCOCCNC(=O)CCOCCOCCOCCOCCNC(=O)C3CCN(c4ncc(C(=O)Nc5ccc(OC(F)(F)Cl)cc5)cc4-c4ccn[nH]4)CC3)ccn12]

Embodiment 69. A pharmaceutical composition comprising a pharmaceutically acceptable excipient and a compound of one of embodiments 1 to 68, or a pharmaceutically acceptable salt thereof.

Embodiment 70. A method of treating cancer in a subject in need thereof, said method comprising administering to the subject in need thereof a therapeutically effective amount of a compound of one of embodiments 1 to 68, or a pharmaceutically acceptable salt thereof.

Embodiment 71. The method of embodiment 70, wherein the cancer is leukemia.

Embodiment 72. The method of embodiment 70, wherein the cancer is chronic myeloid leukemia, acute lymphoblastic leukemia, acute myelogenous leukemia, or mixed-phenotype acute leukemia.

Embodiment 73. A method of treating a neurodegenerative disease in a subject in need thereof, said method comprising administering to the subject in need thereof a therapeutically effective amount of a compound of one of embodiments 1 to 68, or a pharmaceutically acceptable salt thereof.

Embodiment 74. The method of embodiment 73, wherein the neurodegenerative disease is Parkinson's disease or Alzheimer's disease.

Embodiment 75. A method of treating an ABL-associated disease in a subject in need thereof, said method comprising administering to the subject in need thereof a therapeutically effective amount of a compound of one of embodiments 1 to 68, or a pharmaceutically acceptable salt thereof.

Embodiment 76. The method of embodiment 75, wherein said ABL-associated disease is cancer or a neurodegenerative disease.

Embodiment 77. The method of embodiment 75, wherein ABL is BCR-ABL.

Embodiment 78. The method of embodiment 77, wherein BCR-ABL is BCR-ABL1.

Embodiment 79. The method of embodiment 78, wherein the BCR-ABL1 is BCR-ABL1 wild type.

Embodiment 80. The method of embodiment 78, wherein the BCR-ABL1 is a T315I BCR-ABL1 mutant.

Embodiment 81. A method of reducing the level of activity of ABL in a cell, said method comprising contacting the cell with an effective amount of a compound of one of embodiments 1 to 68, or a pharmaceutically acceptable salt thereof.

Embodiment 82. The method of embodiment 81, wherein ABL is BCR-ABL.

Embodiment 83. The method of embodiment 82, wherein the BCR-ABL is BCR-ABL1.

Embodiment 84. The method of embodiment 83, wherein the BCR-ABL1 is BCR-ABL1 wild type.

Embodiment 85. The method of embodiment 83, wherein the BCR-ABL1 is a T315I BCR-ABL1 mutant.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: An Expanded Chemical Space for Cell Permeable Molecules

Using complementary genome-scale chemical-genetic approaches, we identify, inter alia, an endogenous chemical uptake pathway involving interferon-induced transmembrane (IFITM) proteins that modulates the cell permeability of diverse linked chemotypes, exemplified by a prototype bitopic inhibitor of MTOR (molecular weight: 1784 g/mol). We harness this pathway in the design of a highly selective bitopic inhibitor of oncogenic BCR-ABL1 and validate the IFITM dependency of other linked inhibitors in preclinical development. This uptake pathway should provide a general mechanism by which large, flexibly linked chimeric molecules can gain access to the cytoplasm, including compounds with novel mechanisms of action not currently explored in drug discovery.

Any therapeutic that binds to an intracellular target must first navigate through the cell membrane. Retrospective analyses of compound libraries and their biological activities have yielded empirical guidelines (e.g., Lipinski's rule of five) that define modern drug-like chemical space (1-3), enriching for lead-like scaffolds with high passive permeability. While these principles have been useful for streamlining the search for novel therapeutics, many important intracellular drug targets are currently refractory to inhibition by these compact, hydrophobic, and rigid molecules. An emerging design framework that seeks to address these challenges involves increasing pharmacological complexity by linking multiple ligands in a single chemical entity. Doing so can imbue compounds with desirable properties such as enhanced potency (4), greater selectivity (4-6), and the capacity to induce the association of more than one target (7-10). These advances exemplify how high molecular weight, amphiphilicity, and rotational flexibility can enable rapid, modular access to useful chemical probes and therapeutic leads, as long as they remain cell permeable.

Mechanisms to understand and predict the cell permeability of linked chemotypes in the context of conventional drug design principles, however, remain limited. These typically nonionic molecules can be distinguished from cell penetrating proteins and peptides, which commonly require appendage of highly charged moieties to enable productive electrostatic interactions with the plasma membrane and subsequent internalization (11-13). Studies involving the most rapidly expanding linked chemotype in the literature, proteolysis targeting chimeras (PROTACs) (14), provide varying insights into the determinants of cell permeability (15-20), with one report finding no correlation between cell permeability and artificial membrane permeability (18). Despite their atypical properties, PROTACs and additional large molecules such as the dimeric immunophilin ligand rimiducid have entered clinical trials (21) (NCT03888612 and NCT04072952). Given this discrepancy with traditional concepts of passive permeability, we hypothesized that linked chemotypes might hijack cellular processes to assist their passage through the cell membrane. We selected as an example a bitopic inhibitor of MTOR, RapaLink-1 (4), whose molecular weight (1784 g/mol) falls well beyond common guidelines (≤500 g/mol) (1) and even typical PROTACs (800-1200 g/mol) (18). RapaLink-1 is highly active in vivo (4, 5, 22), penetrates the blood-brain barrier (5, 22), and serves as a prototype for the clinical candidate RMC-5552 (NCT04774952), establishing itself as a drug-like compound that defies most traditional notions of drug-likeness.

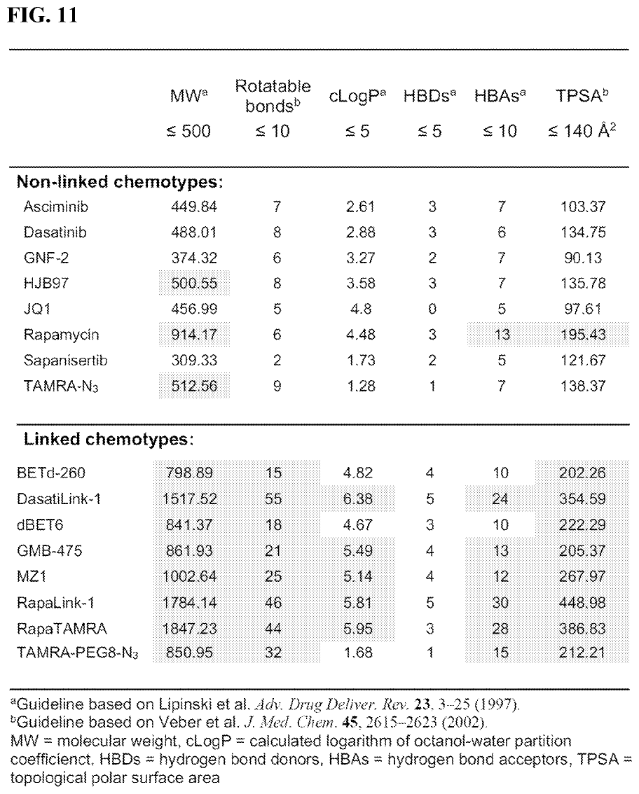

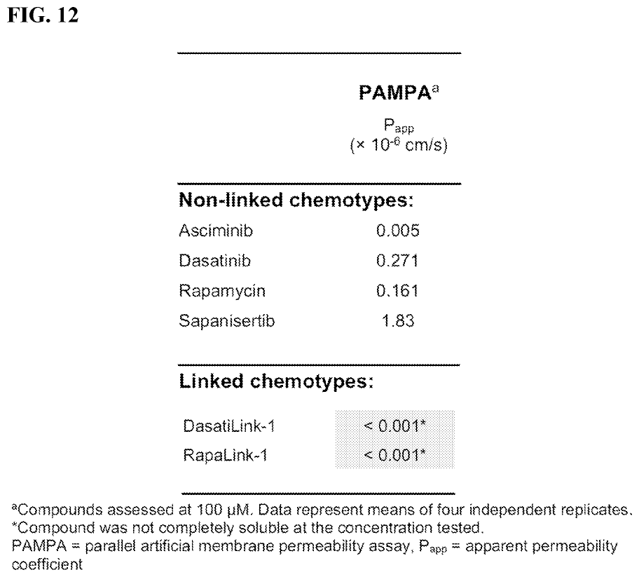

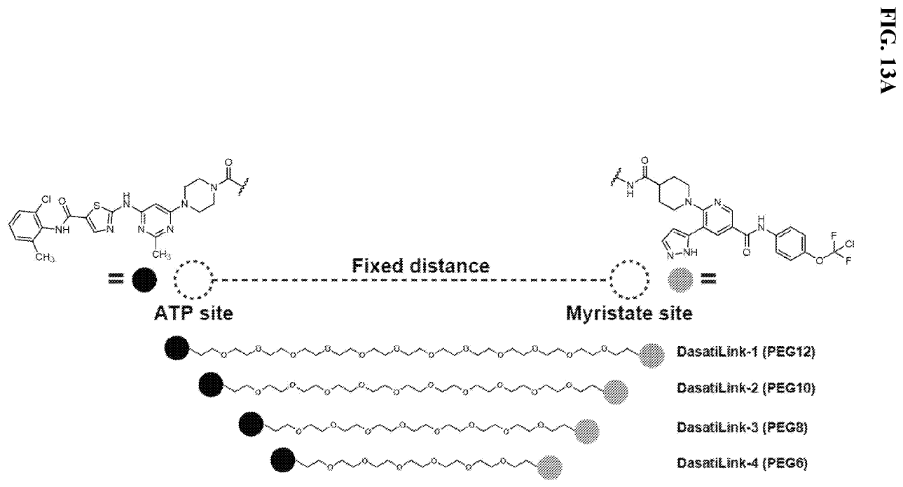

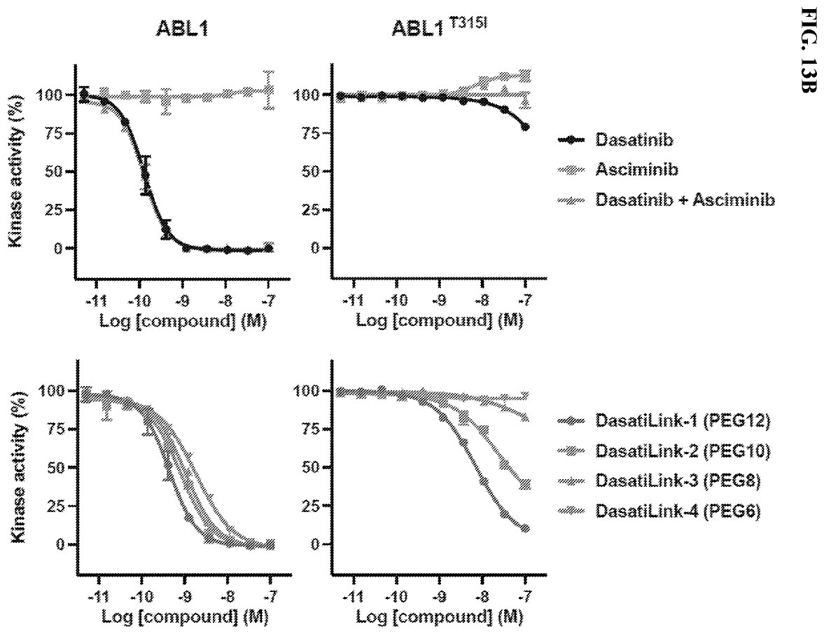

We first assessed the intrinsic permeability of RapaLink-1 outside the context of a living system. Considering the linked chemotype's anomalous physicochemical properties (FIG. 11), RapaLink-1 would be predicted to face difficulty crossing the crowded, hydrophobic interior of a lipid membrane (23). Indeed, RapaLink-1 displayed no measurable permeability by parallel artificial membrane assay (PAMPA), while its individual chemical modules (orthosteric inhibitor sapanisertib and allosteric inhibitor rapamycin) were readily permeable (FIG. 12). This observation contrasts with RapaLink-1's robust ability to partition into living cells (4, 5, 22, 24), which we reasoned may result from proteins and processes not present in an abiotic system such as PAMPA. We hypothesized that, by systematically perturbing these processes, we could identify cellular mechanisms that interacted with RapaLink-1 to permit its cytoplasmic entry.

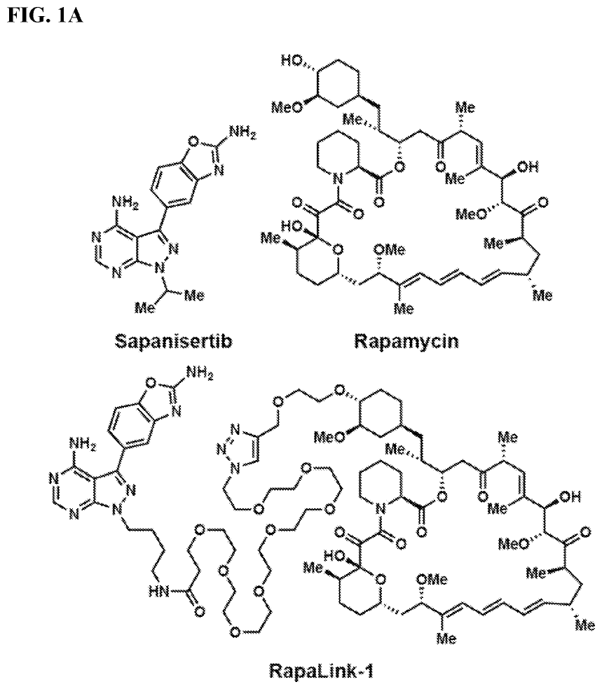

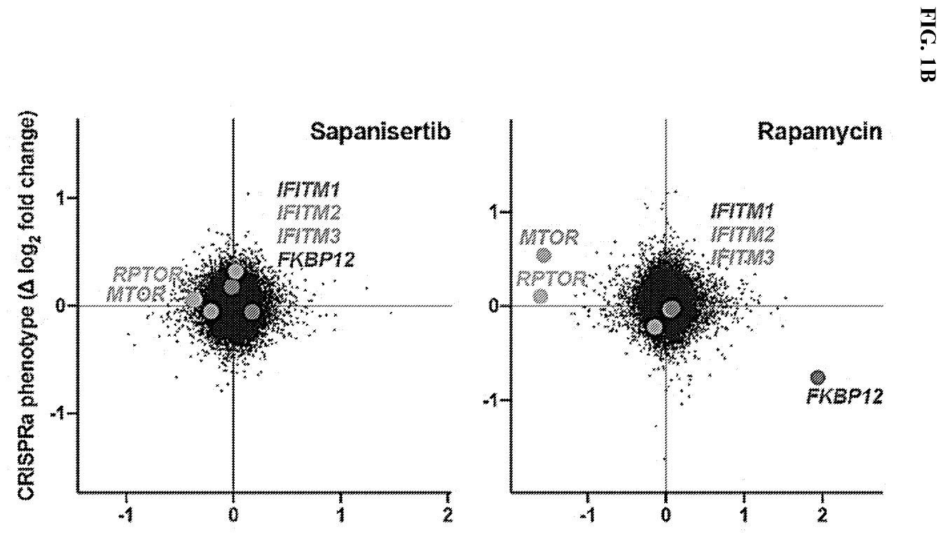

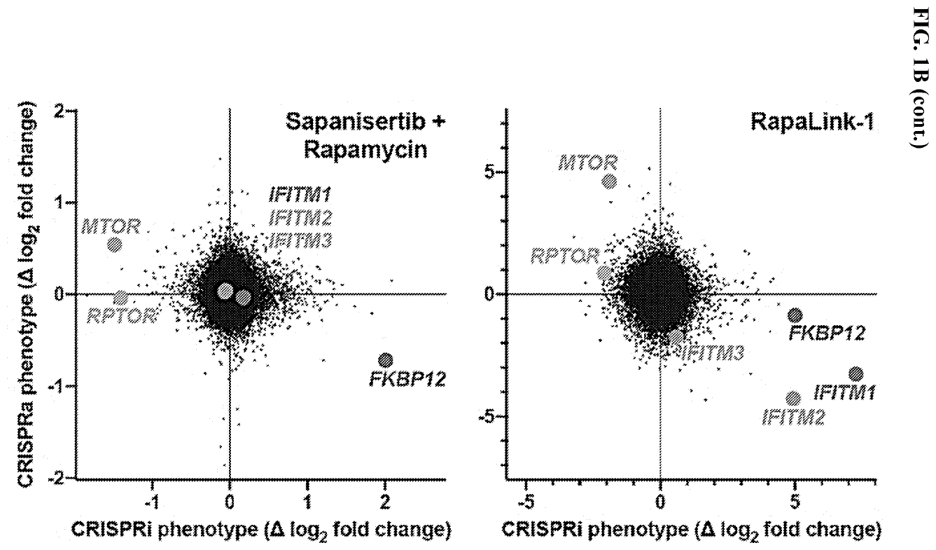

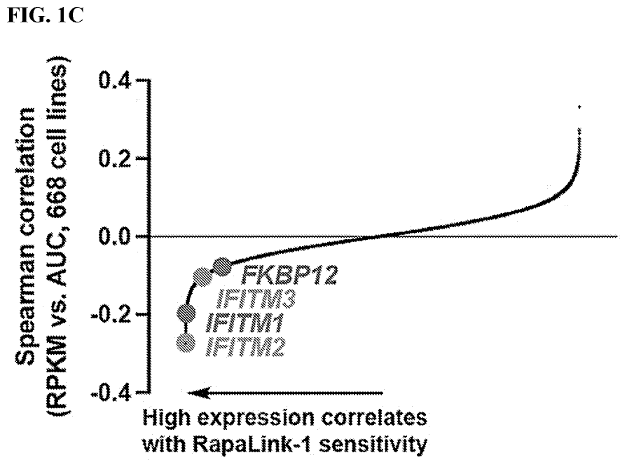

We probed canonical protein coding genes for cellular factors that determine RapaLink-1 uptake and sensitivity using a dCas9-based CRISPRi/a functional genomics platform (25, 26). Gene expression inhibition and activation, through CRISPRi and CRISPRa respectively, act as complementary approaches to map chemical-genetic interactions at genome-scale. In particular, genes displaying strong mirrored (i.e., resistance upon knockdown and sensitivity upon overexpression) phenotypes are likely to be directly involved in a small molecule's mechanism of action (27). In addition to the bitopic inhibitor, we included assessment of sapaniserib, rapamycin, and an unlinked control (a 1:1 mixture of sapanisertib and rapamycin) to distinguish chemical-genetic interactions specific to the linked chemotype (FIG. 1A).

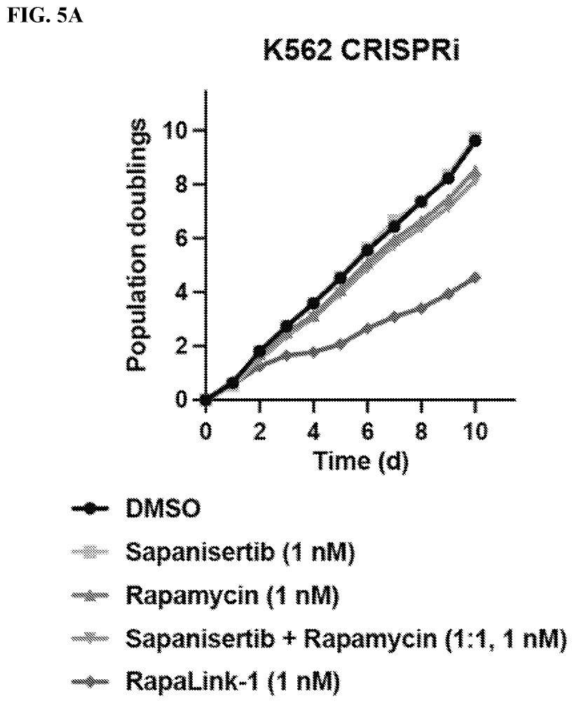

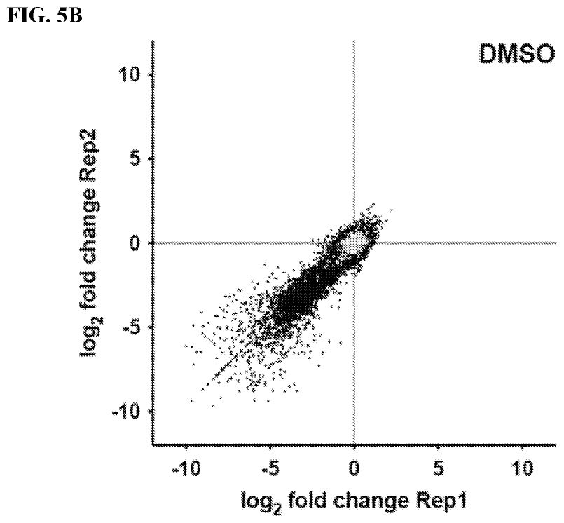

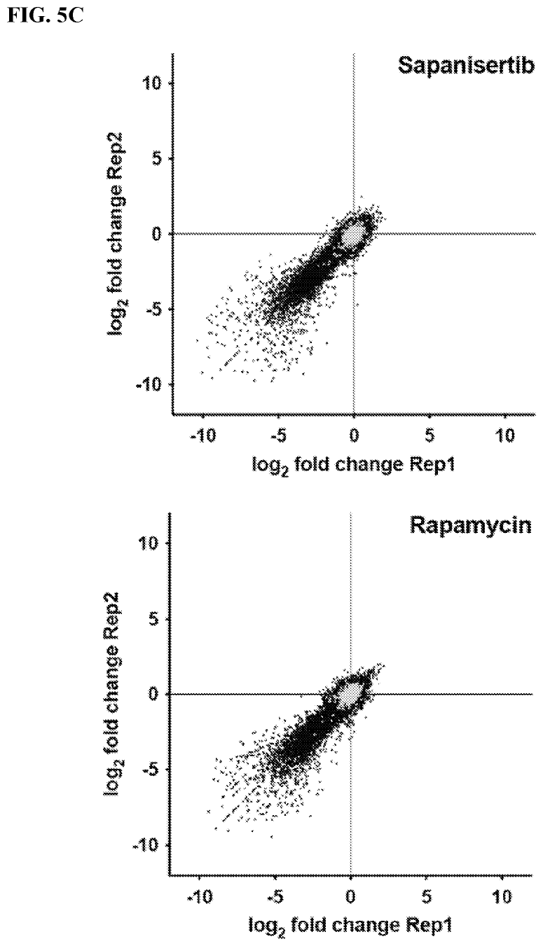

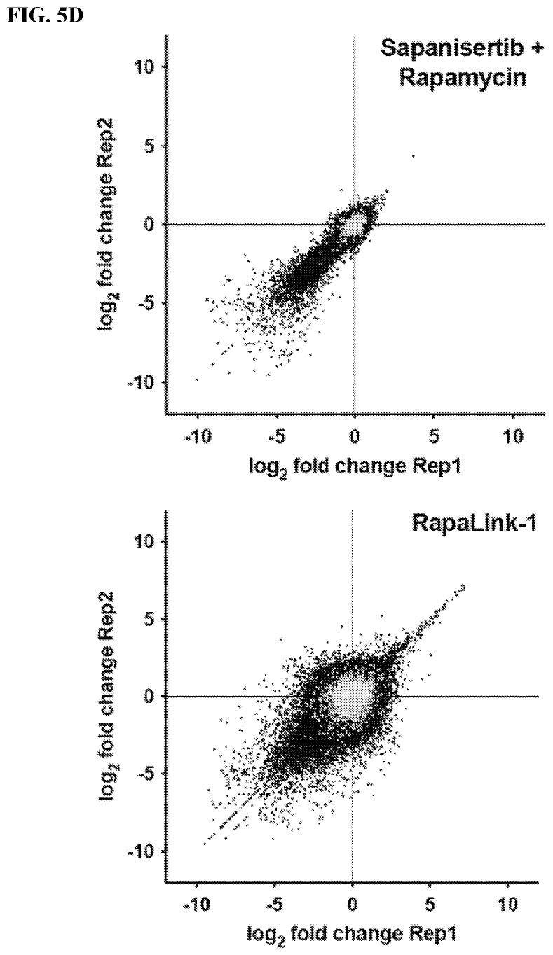

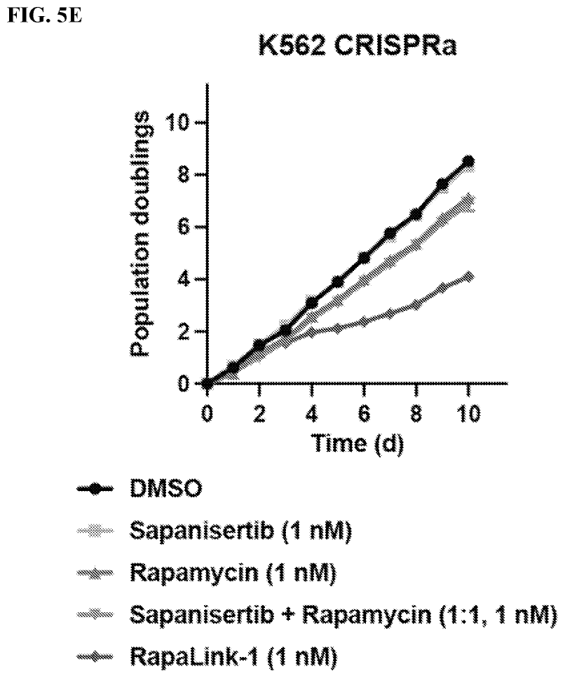

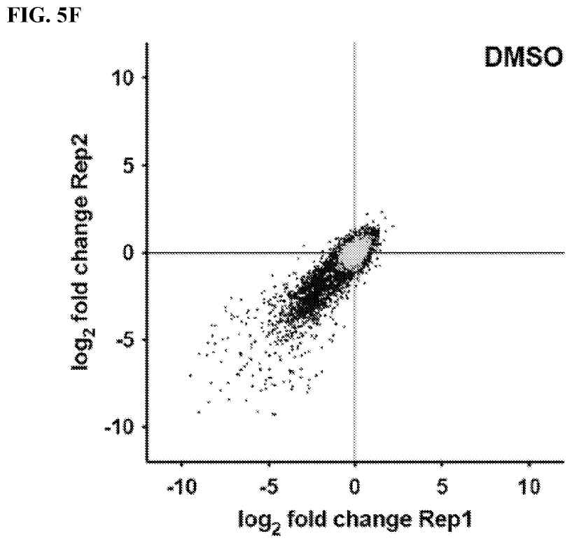

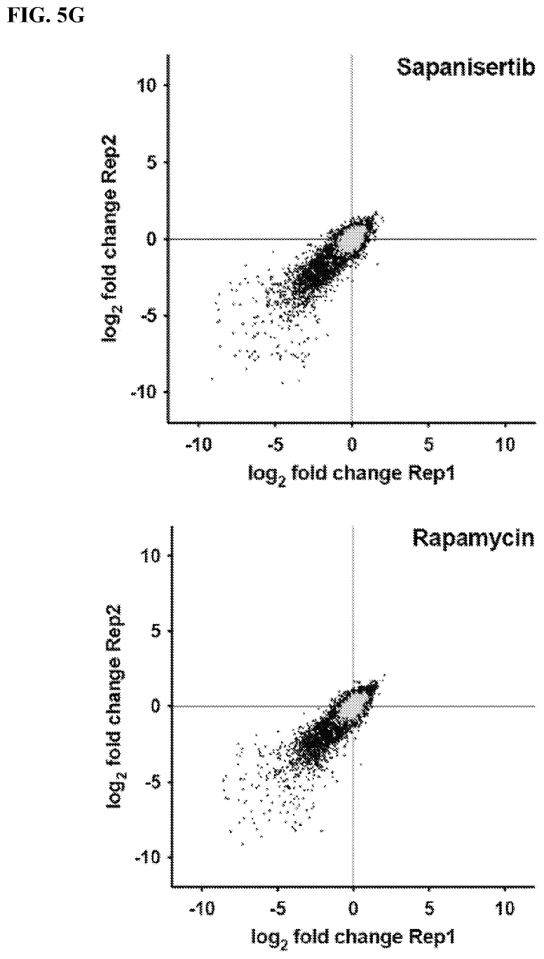

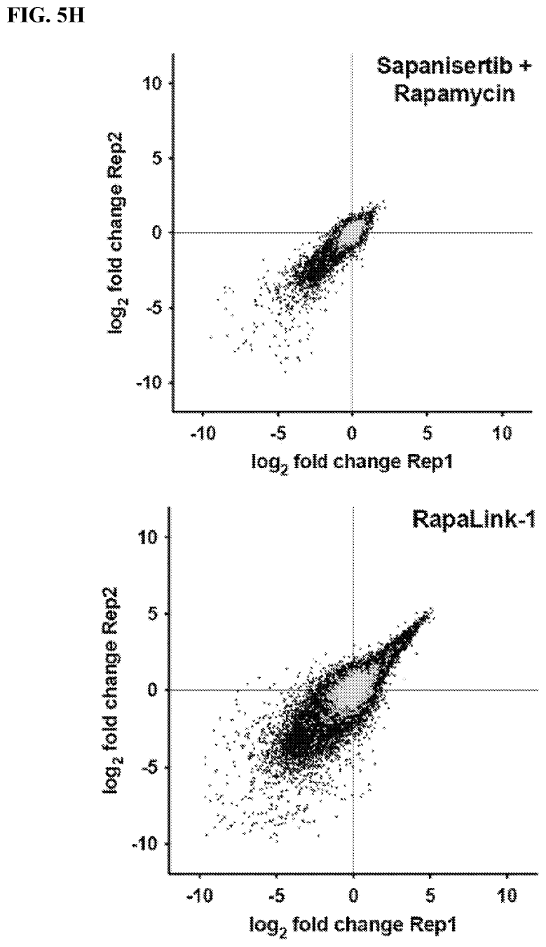

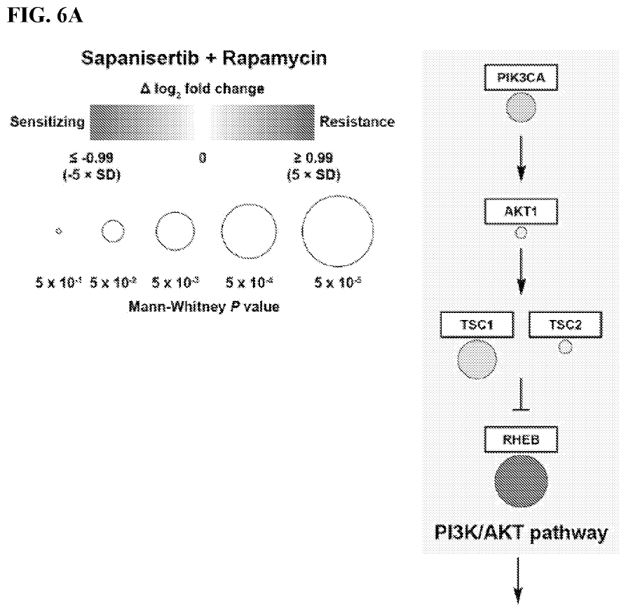

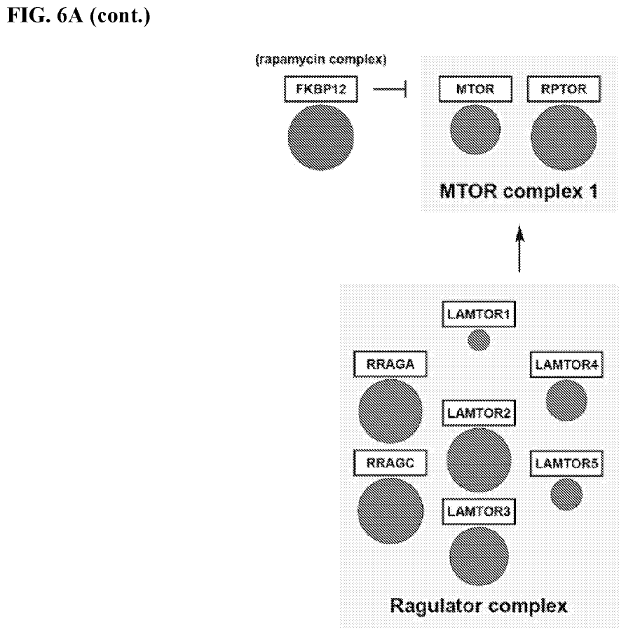

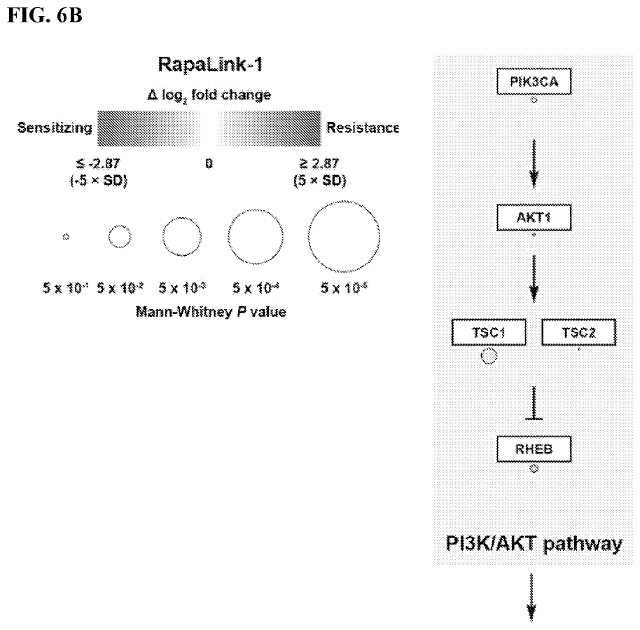

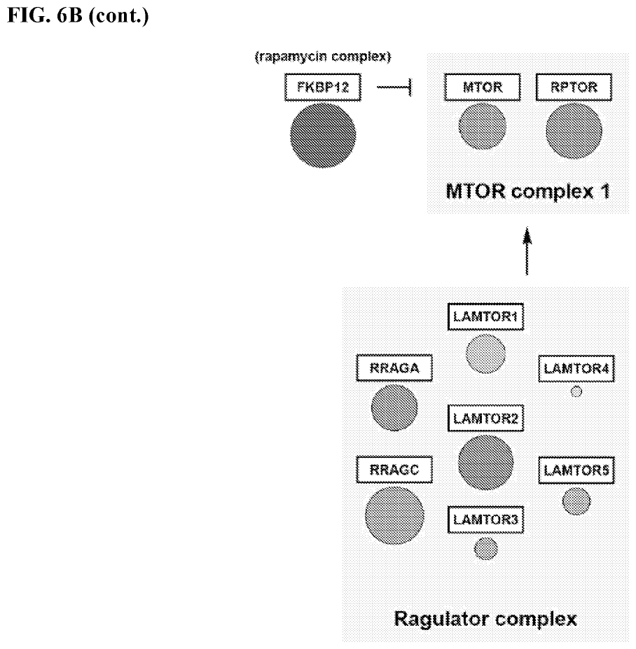

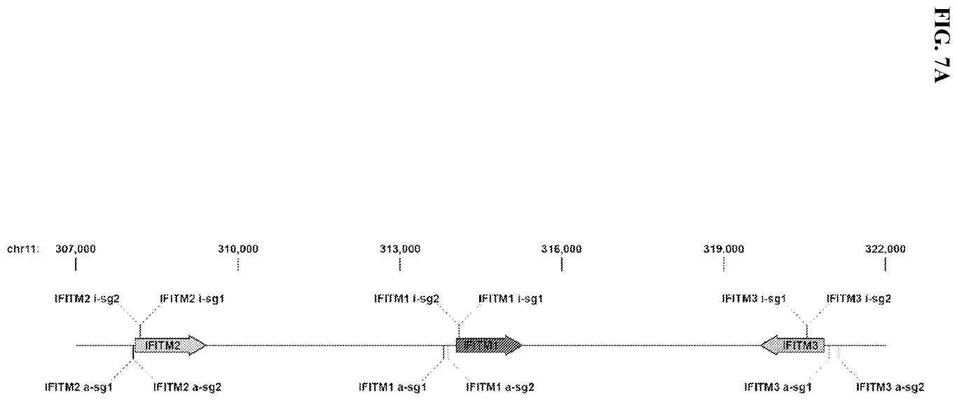

Patient-derived chronic myeloid leukemia (CML) cells, K562, preinstalled with CRISPRi or CRISPRa machinery, were transduced with their respective genome-scale sgRNA libraries, selected with puromycin to remove non-transduced cells, and divided amongst the various chemical perturbations (DMSO, sapanisertib, rapamycin, sapanisertib+rapamycin, or RapaLink-1). The experiments were conducted with high replicate reproducibility (FIGS. 5A-5H), and data from the genome-scale CRISPRi and CRISPRa screens were juxtaposed to highlight genes that displayed mirrored phenotypes (FIG. 1B). This arrangement distributes genes which functionally synergize with the inhibitor in the lower right (e.g., requisite inhibitory complex partner of rapamycin FKBP12) and those which antagonize the inhibitor in the upper left (e.g., direct target MTOR) (27). Chemical-genetic interactions with MTOR signaling components, particularly the Ragulator complex (RRAGA, RRAGC, and LAMTOR1-5) and nodes downstream of PI3K/AKT (TSC1, TSC2, and RHEB), were observed across multiple inhibitor conditions (FIGS. 6A-6B), corroborating known pathway relationships (28) and prior functional genomics studies (29, 30).

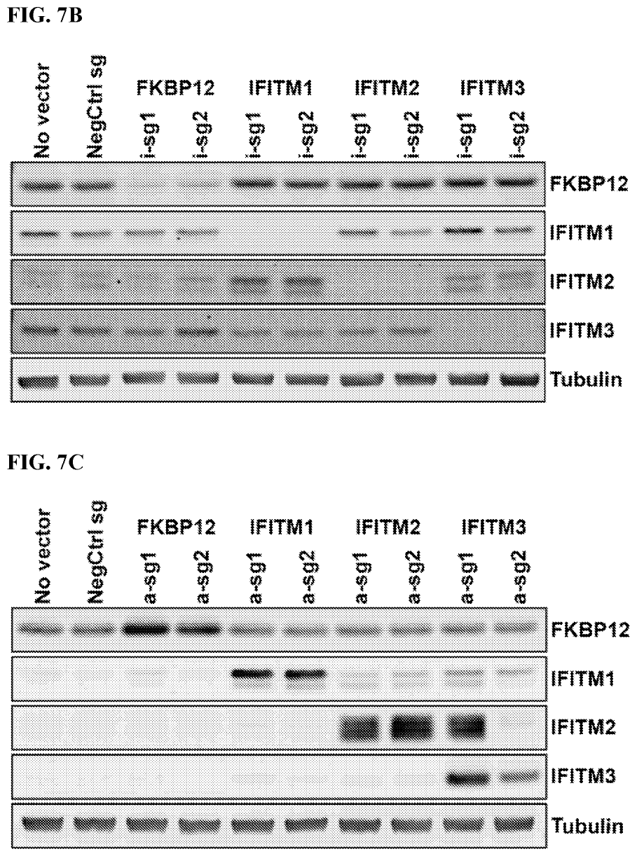

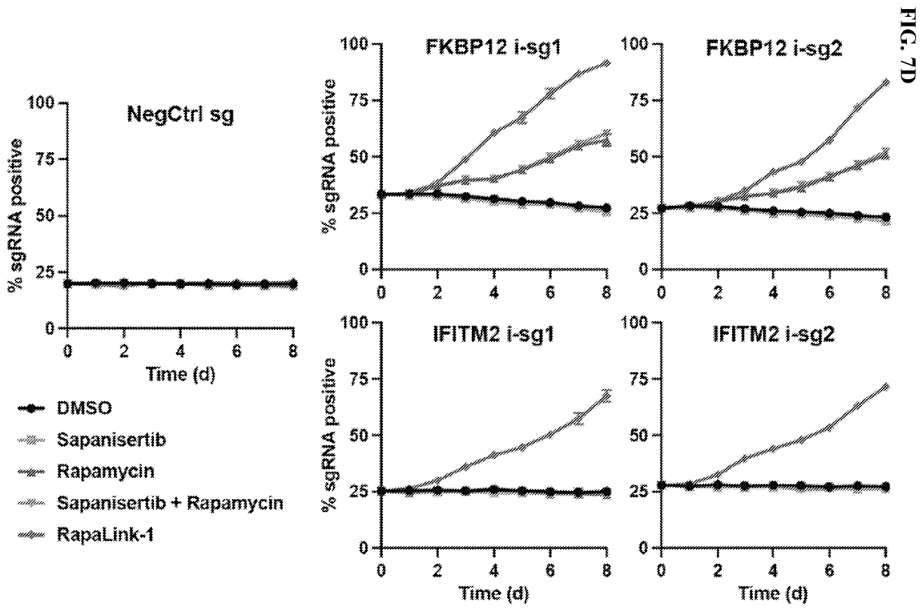

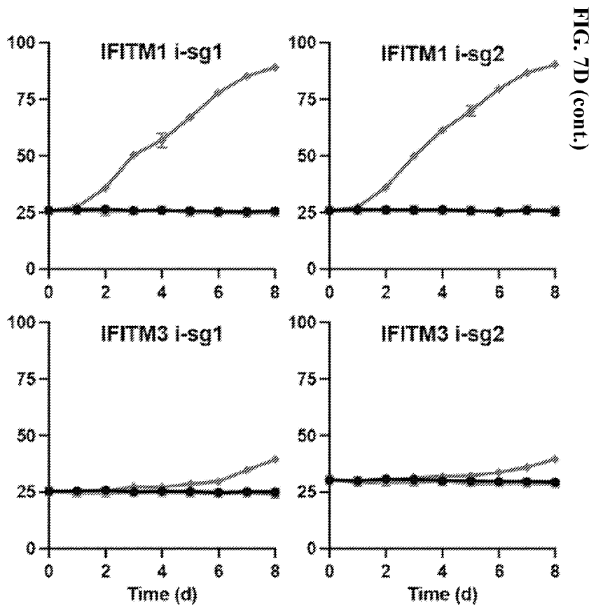

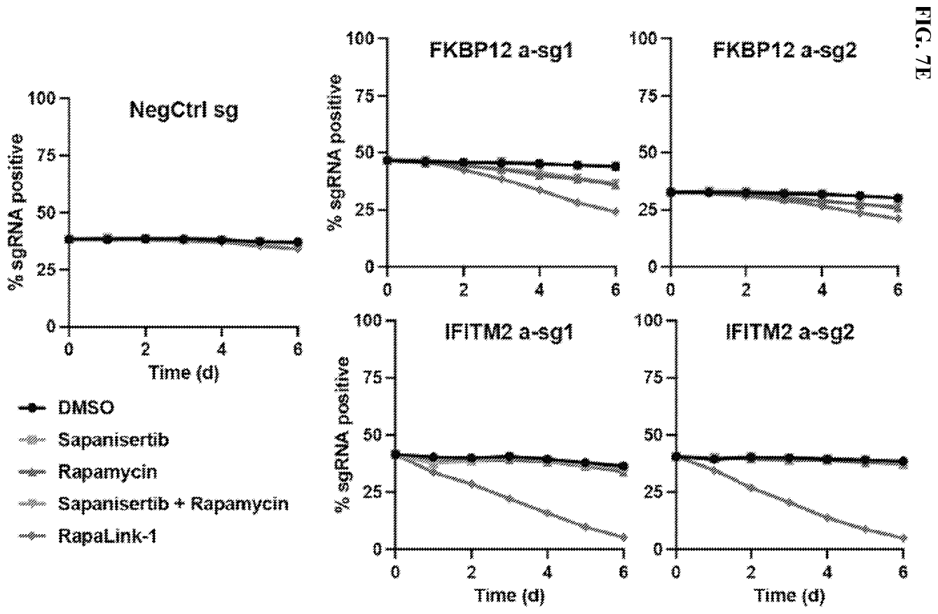

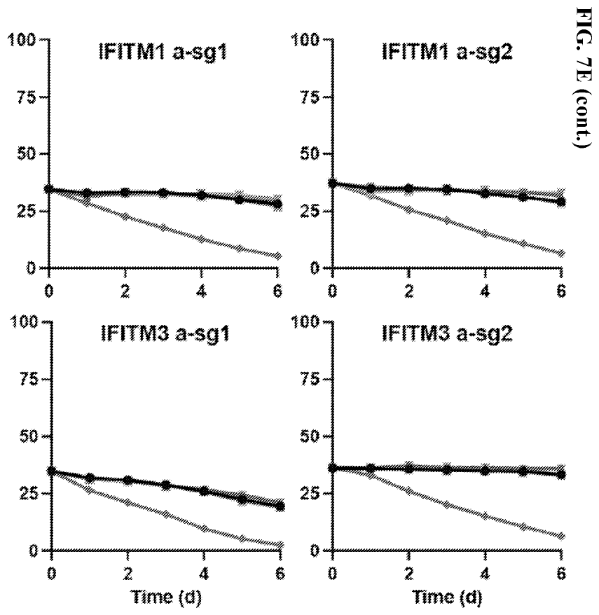

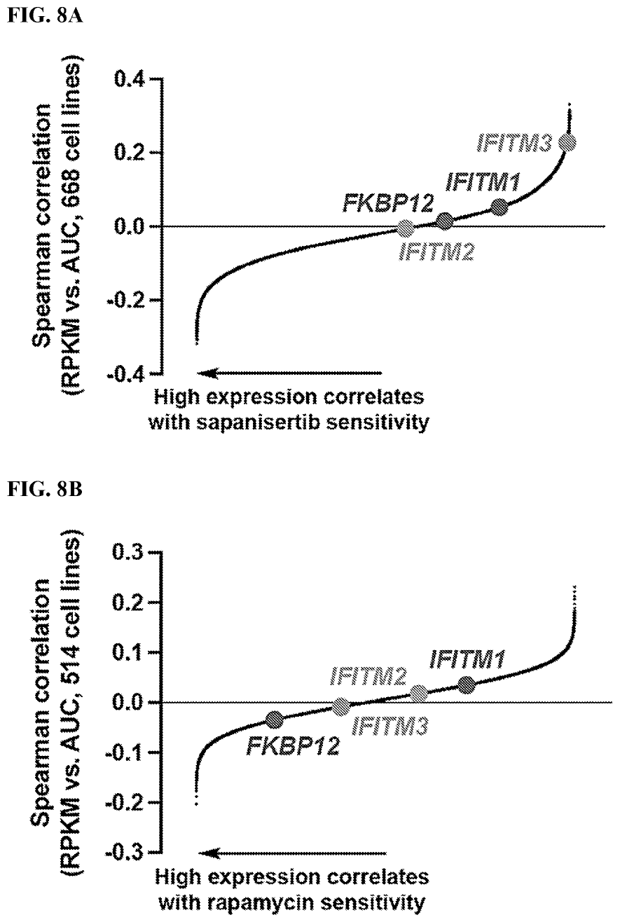

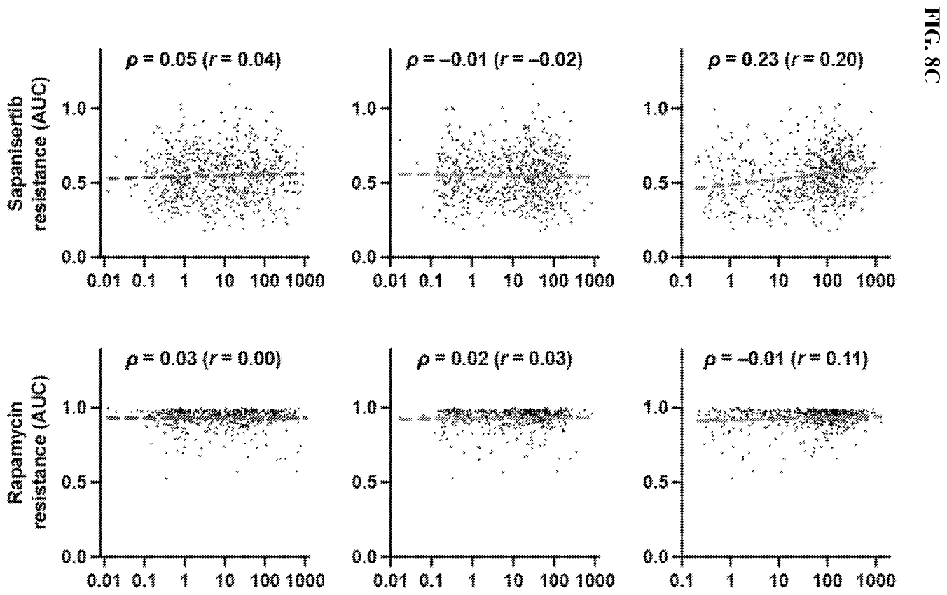

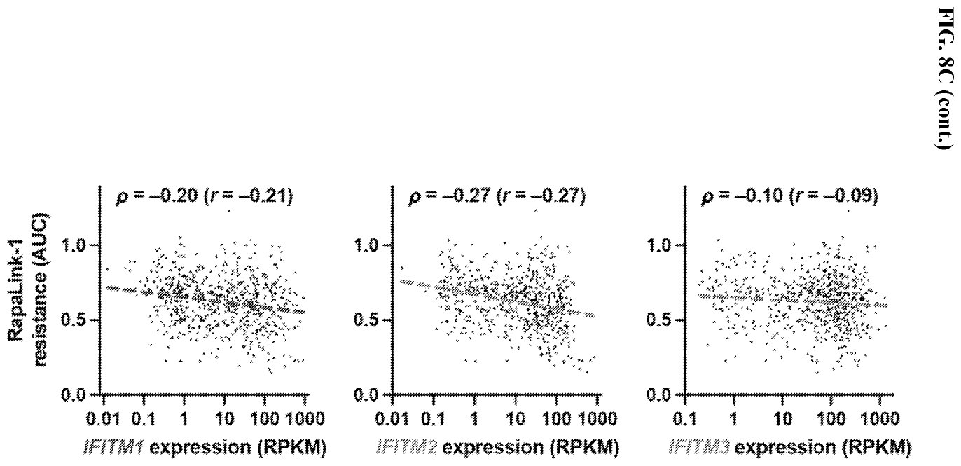

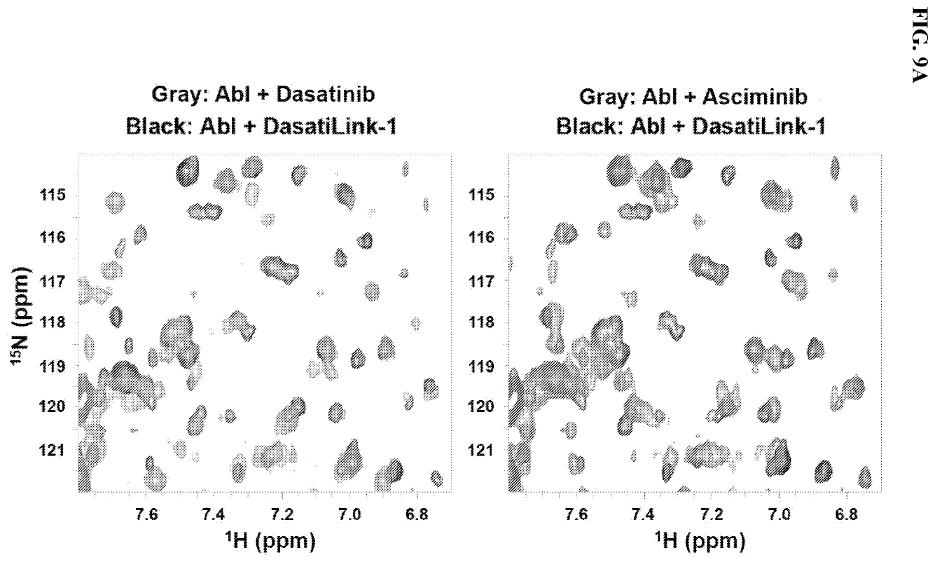

Notably, a distinct set of chemical-genetic interactions were identified as top genetic hits with RapaLink-1, suggesting the involvement of a biological pathway that uniquely promotes the activity of the linked chemotype. The expression of members of a highly homologous gene family, interferon-induced transmembrane (IFITM) proteins 1, 2 and 3 (31), synergized with the activity of RapaLink-1 and not its closely related non-linked counterparts, sapanisertib and rapamycin (FIG. 1B). To validate this finding, we tested individual sgRNAs targeting various IFITM family members for transcriptional repression or activation (FIG. 7A). CRISPRi-mediated knockdown of IFITM1-3 was potent and selective as assessed by immunoblotting (FIG. 7B). CRISPRa-mediated overexpression was also potent although we observed variable cross activation between family members (FIG. 7C), possibly related to the proposed concerted manner in which the three neighboring genes are transcriptionally regulated upstream of IFITM3 on chromosome 11 (FIG. 7A) (32). Cells transduced with these sgRNAs in a competitive growth assay were incubated in the presence of MTOR inhibitor (sapanisertib, rapamycin, sapanisertib+rapamycin, or RapaLink-1) to confirm the specificity of the RapaLink-1-IFITM1-3 chemical-genetic interactions as well as select positive control hits (FKBP12) observed in the screens (FIGS. 7D-7E). Seeking to generalize these observations beyond a single cell type, we also employed an independent chemical-genetic approach correlating MTOR inhibitor sensitivity data with basal gene expression in diverse in vitro models (33-35). High IFITM1-3 expression by RNA sequencing was strongly associated with enhanced RapaLink-1 sensitivity across 668 cell lines (FIG. 1C). This correlation was absent for sapanisertib and rapamycin (FIGS. 8A-8C), recapitulating the CRISPRi/a screens. Together, our combined analysis of the CRISPRi/a screens and large-scale chemogenomic cell line profiling experiments suggested a general role of IFITM proteins in promoting the activity of the linked chemotype.

As neither of the non-linked inhibitors demonstrated chemical-genetic interactions with IFITM1-3, we reasoned that IFITM proteins did not directly modulate MTOR signaling, but instead cooperated with the unique physicochemical nature of RapaLink-1. Clade I IFITM family members, IFITM1-3, are closely related broad spectrum viral restriction factors (31). They enact their antiviral function, in part, by rendering local membrane biophysics at the viral-endosomal juncture unfavorable for viral entry (36-38). In addition to their established immunologic function, clade I IFITM proteins are also reported to modulate an oncogenic phenotype (39, 40), affect placenta formation (41), and contribute to endosomal homeostasis (42). Considering that IFITM proteins function as molecular gatekeepers at the interface of the extracellular and intracellular space, we hypothesized that the chemotype-specific chemical-genetic interactions detailed above derived from a cellular uptake mechanism specifically engaged by the linked chemotype.

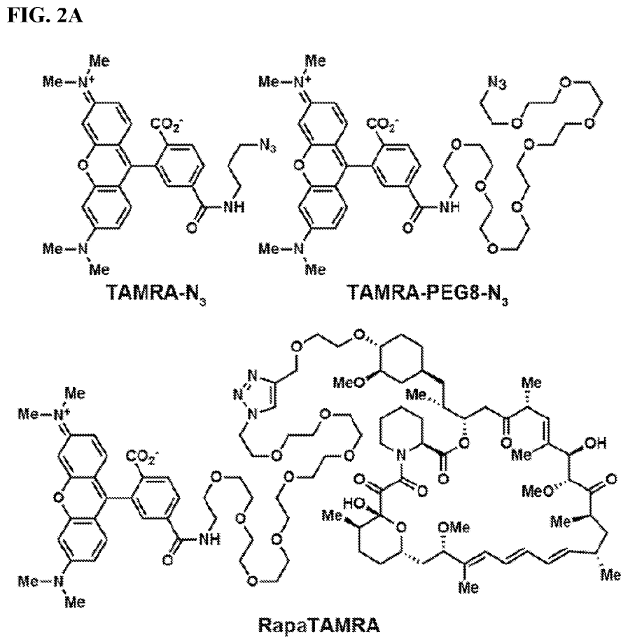

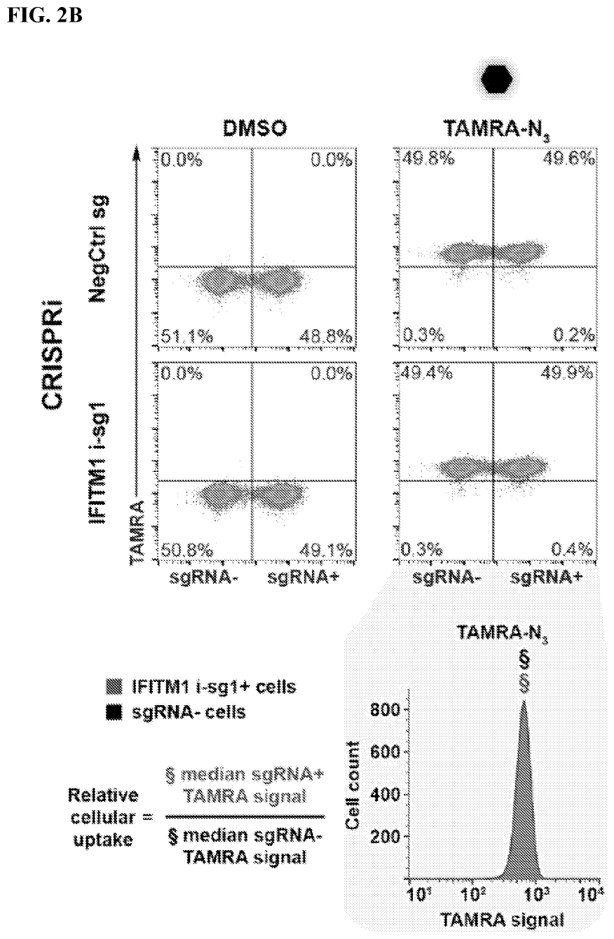

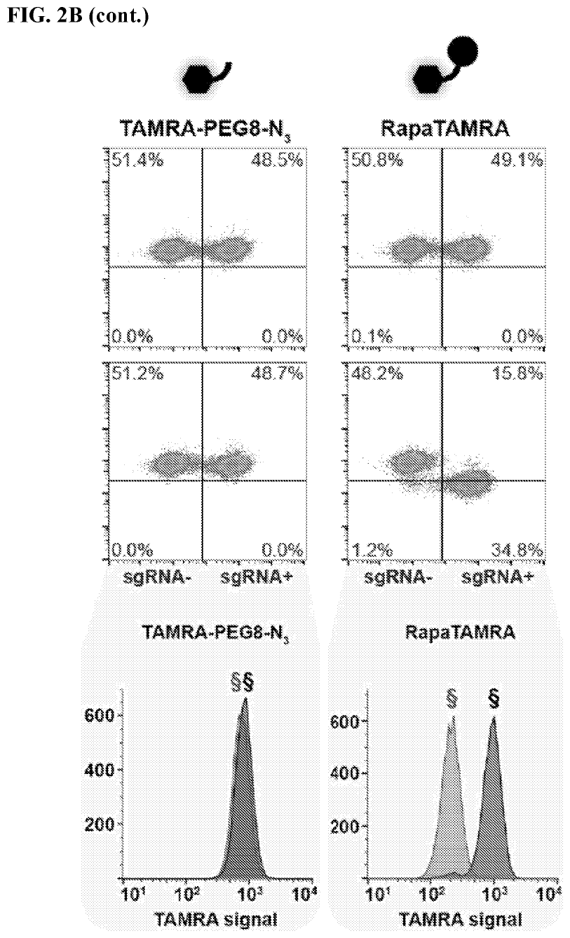

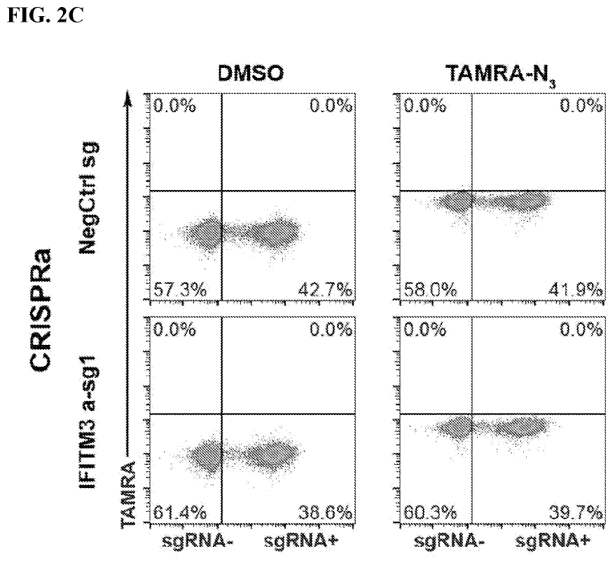

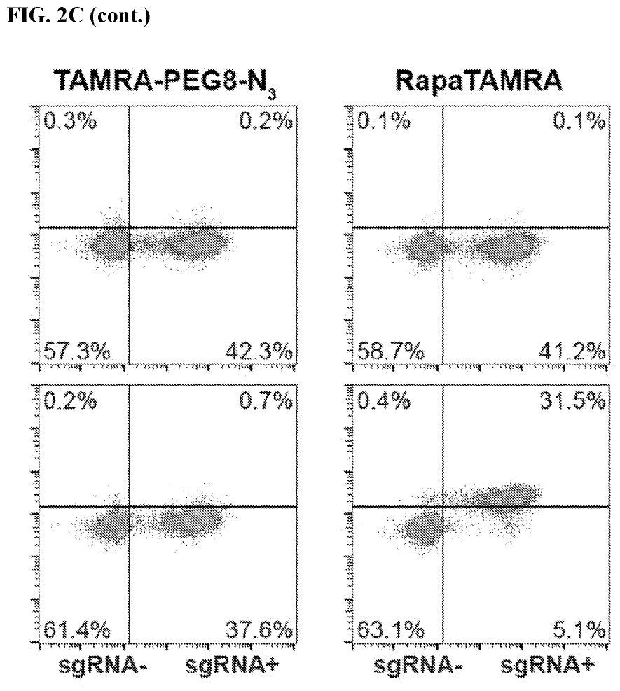

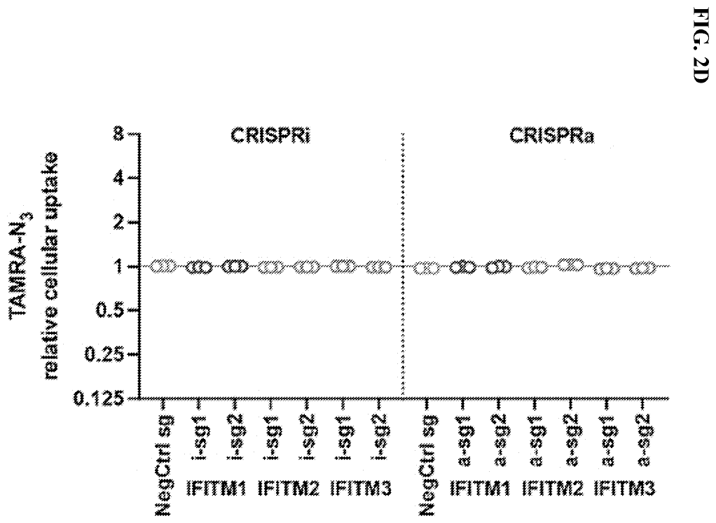

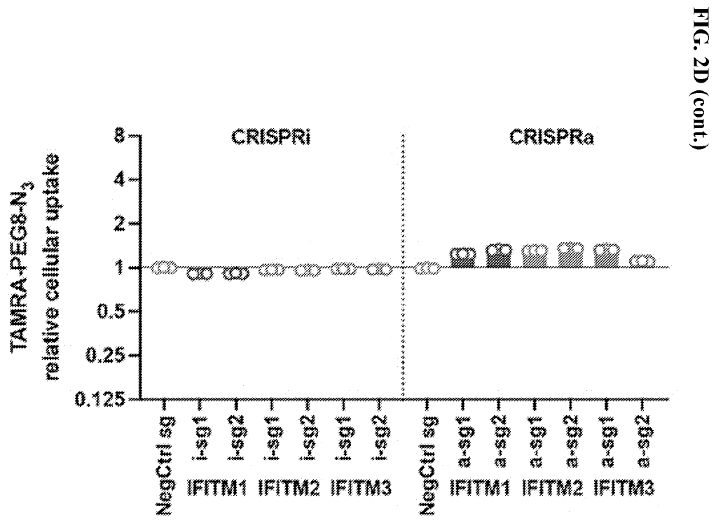

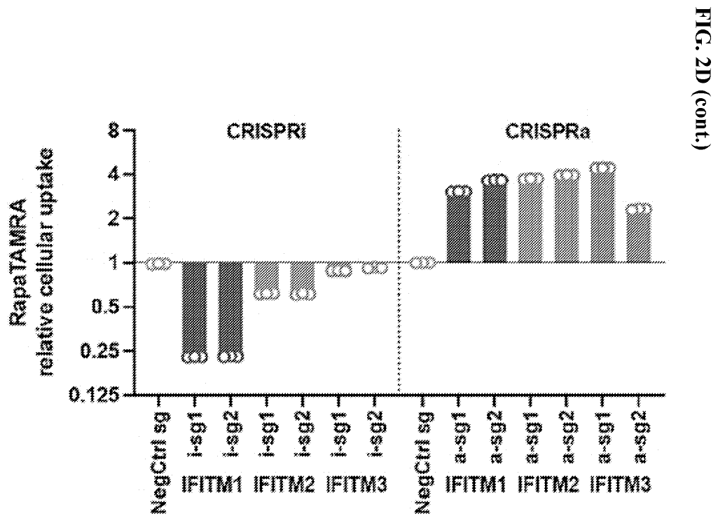

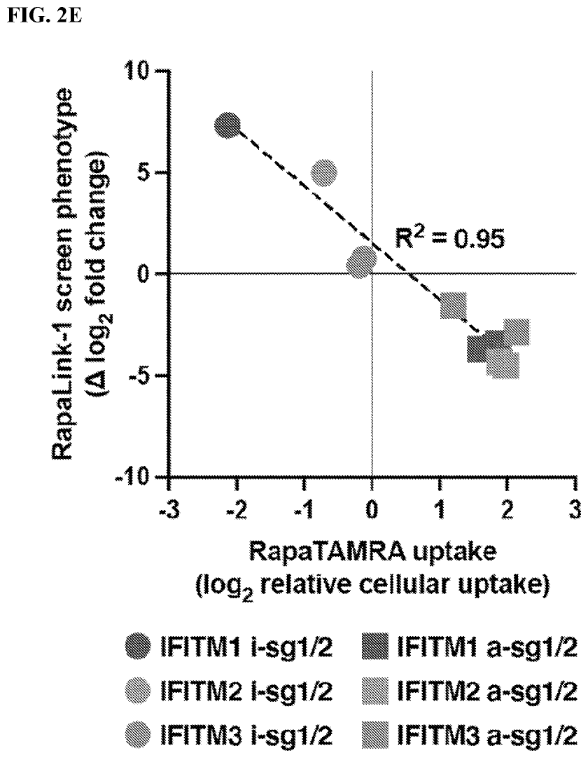

We applied a fluorescent analog of RapaLink-1 to directly observe the effect of IFITM protein expression on uptake of the linked chemotype in live cells. This fluorescent molecule, RapaTAMRA, was designed by replacing the adenosine triphosphate (ATP)-site binding element in RapaLink-1 with tetramethylrhodamine (TAMRA), resulting in a traceable derivative that closely mimicked the physicochemical properties of the original molecule (FIG. 2A and FIG. 11) (22). Analogs representing partial components of RapaTAMRA, TAMRA-N₃ and TAMRA-PEG8-N₃, were additionally included to assess whether the uptake pathway extended to generic compact-hydrophobic or linked-amphiphilic chemotypes respectively (FIG. 2A).

We tested these molecules using a quantitative live cell fluorescence uptake assay in which a mixture of transduced (e.g., with a targeting sgRNA) and non-transduced cells were equally exposed to compound within the same well. The amount of fluorescent molecule present in the cells was quantified using flow cytometry, and the two cell populations could be resolved for internal normalization (FIGS. 2B-2C). Changes in cellular uptake resulting from CRISPRi/a expression modulation by sgRNAs (FIGS. 7A-7E) revealed again an IFITM dependency pattern that was chemotype-specific (FIGS. 2B-2D). Both linked chemotypes, TAMRA-PEG8-N₃ and RapaTAMRA, demonstrated decreased uptake upon knockdown of IFITM1-3 and increased uptake upon overexpression (FIGS. 2B-2D). The linker-less chemotype, TAMRA-N₃, in contrast exhibited no such chemical-genetic interactions (FIGS. 2B-2D). Notably, CRISPRi/a-induced uptake differences observed for RapaTAMRA correlated strongly with resistance and sensitivity phenotypes for RapaLink-1 (FIG. 2E), drawing a direct association between measured uptake and functional target inhibition. The observation that a generic linked chemotype not specifically bound by any cellular protein, TAMRA-PEG8-N₃, was also affected suggested a broader utilization of this uptake mechanism by other linked molecules.

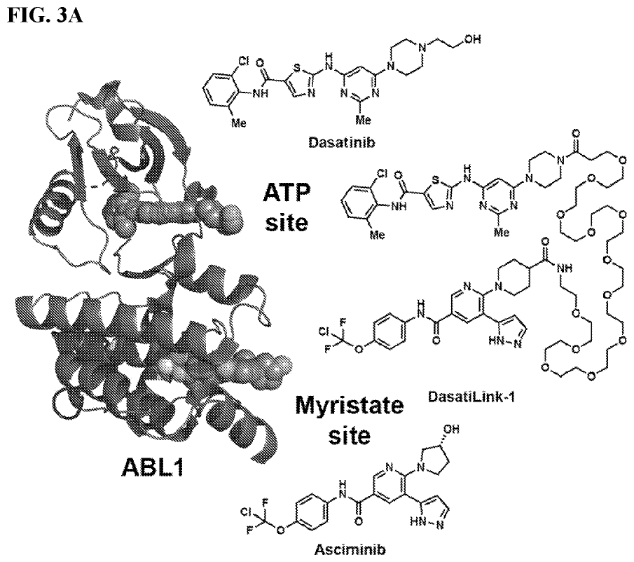

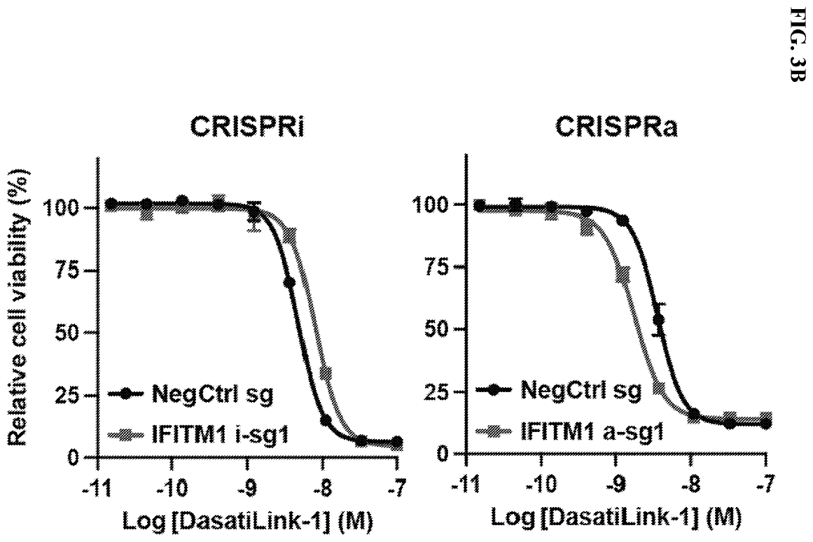

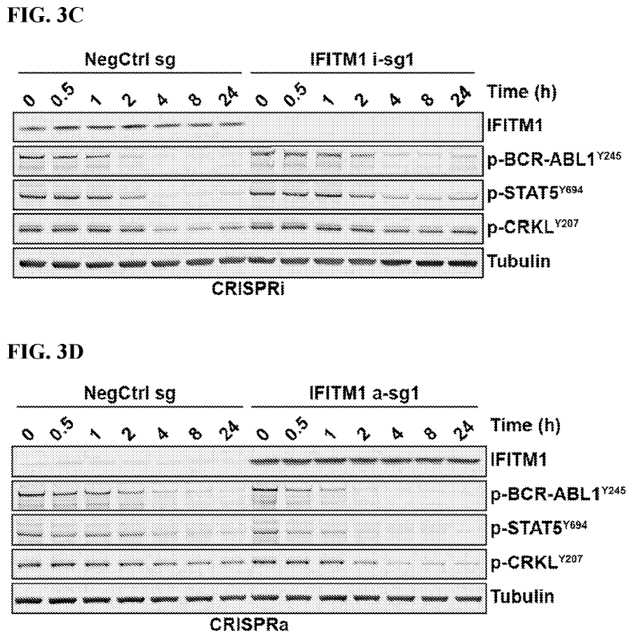

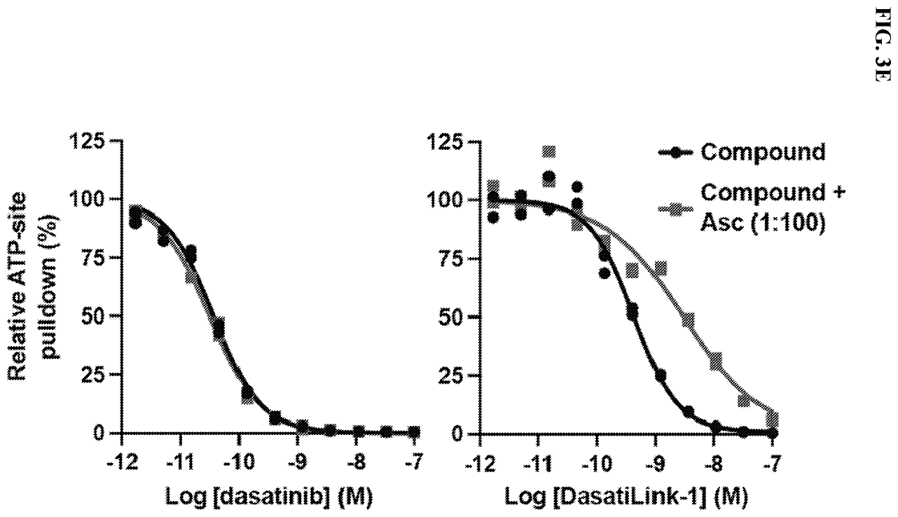

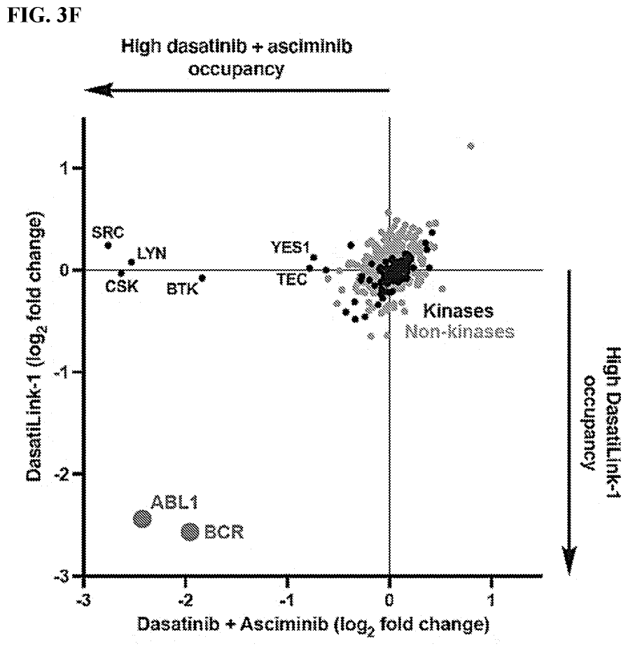

To further establish the generalizability of this IFITM-promoted cellular uptake mechanism, we designed, synthesized, and characterized a bitopic inhibitor that was, aside from being linked, compositionally unrelated to RapaLink-1. This inhibitor targeted a different intracellular protein, BCR-ABL1, a fusion oncoprotein pathognomonic of CML and other leukemias (43). BCR-ABL1 harbors two well-defined small molecule binding sites within its kinase domain (FIG. 3A): the ATP pocket (44) targeted by five clinical compounds (e.g., dasatinib) and the myristoyl pocket (45, 46) targeted by the recently clinically approved first-in-class inhibitor asciminib (47). These sites can also be bound by the two classes of inhibitors simultaneously when used in concert (45, 46, 48). Considering that the two pockets span a similar distance as those engaged by RapaLink-1 in MTOR (4), we reasoned that a similar linkage strategy could also apply to BCR-ABL1. We devised a bitopic inhibitor of BCR-ABL1, DasatiLink-1, based on the merging of dasatinib and asciminib by a flexible tether whose length (41 heavy atoms) emulated that of RapaLink-1 (39 heavy atoms) (FIG. 3A).

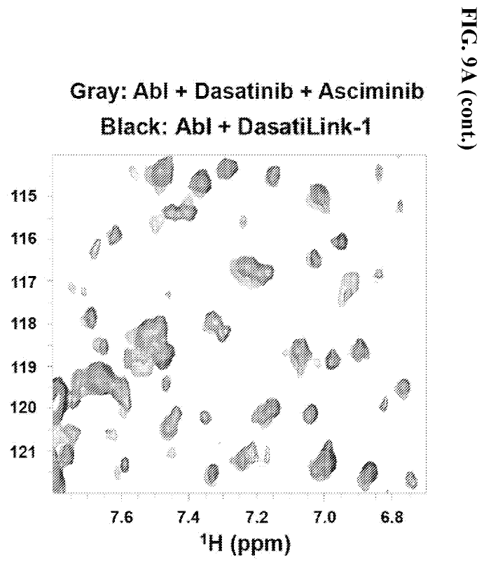

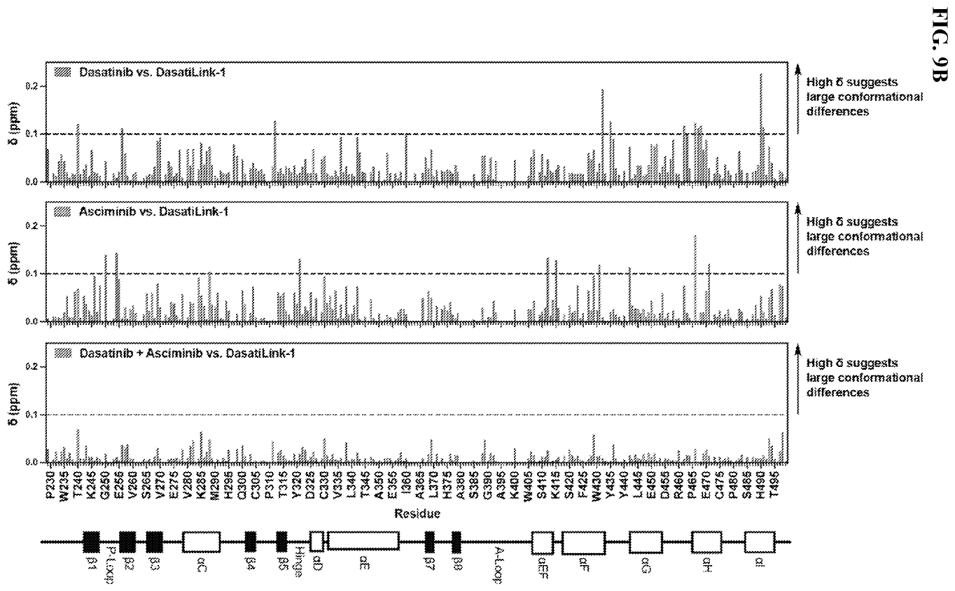

To assess whether the bitopic inhibitor functioned as designed, we characterized the interaction between DasatiLink-1 and its target by solution nuclear magnetic resonance (NMR) spectroscopy. In comparison to the bitopic inhibitor, treatment of BCR-ABL1 kinase domain with dasatinib or asciminib resulted in marked (>0.1 ppm) NMR chemical shift differences in residues involved in binding to the monomeric inhibitors (FIGS. 9A-9B), consistent with previous reports (45, 46, 49). However, the NMR spectrum observed with the two inhibitor mixture closely matched that of DasatiLink-1 (FIGS. 9A-9B), suggesting the linked inhibitor's simultaneous binding to both sites in a conformation devoid of structural impingements imposed by the flexible tether. These data corroborated a bitopic mechanism of action in which DasatiLink-1 occupies both ATP and myristoyl pockets within the kinase domain of BCR-ABL1.

Anticipating that DasatiLink-1 might face similar challenges traversing lipid bilayers as RapaLink-1 due to its comparable physicochemical properties (FIG. 11), we also evaluated the bitopic BCR-ABL1 inhibitor's artificial membrane permeability. DasatiLink-1 was, like RapaLink-1, impermeable by PAMPA although its individual components (dasatinib and asciminib) were readily permeable (FIG. 12). While the presence of a linker seemed to preclude the bitopic compounds from passively diffusing through an abiotic membrane, we postulated that DasatiLink-1 might, in a live cell context, harness the same IFITM-dependent mechanisms as RapaLink-1 to license access to intracellular BCR-ABL1.

Returning to our BCR-ABL1-mutant CRISPRi/a cell models, we validated the IFITM dependency of DasatiLink-1 (FIG. 3B). In addition to observing the molecule's potent action on cell viability, we probed DasatiLink-1's capacity to engage intracellular BCR-ABL1 by measuring pharmacodynamic markers of inhibition (FIGS. 3C-3D). DasatiLink-1's ability to inhibit its target was slowed or hastened as a result of IFITM1 expression modulation, consistent with an IFITM-dependent uptake mechanism. The inhibition kinetics we observed, requiring multiple hours for maximal inhibition at nanomolar concentrations (FIGS. 3C-3D), were also exhibited by RapaLink-1 (4, 5). This contrasts with the typical finding that passively diffusing small molecules reach their intracellular targets in the immediate timescale (50), again suggesting that linked chemotypes employ a distinct uptake mechanism from traditional drug-like molecules.

In addition to exhibiting discrete permeability properties, DasatiLink-1, akin to RapaLink-1, was anticipated to be uniquely selective for its target on the basis of its multivalent binding mechanism. We tested the assumption that DasatiLink-1 required an allosteric foothold to achieve high occupancy of BCR-ABL1 kinase domain using a pulldown assay for ATP-site availability (51). We validated that the assay recapitulated a biochemical IC₅₀ of <1 nM for dasatinib (52), which was unaffected by inclusion of 100-fold excess allosteric inhibitor (FIG. 3E). Conversely, addition of excess asciminib impaired the ability of DasatiLink-1 to occupy the ATP-site, likely resulting from a loss of avidity following steric occlusion of the allosteric pocket (FIG. 3E). This posits an AND logic between the orthosteric and allosteric sites for bitopic binding, supporting the enhanced selectively often observed in bitopic inhibitors (4-6, 24, 53). We then evaluated the kinome-wide selectivity of DasatiLink-1 in live cells using a promiscuous kinase occupancy probe, XO44 (54), by which kinase occupancy can be determined through competitive activity-based protein profiling (55). In contrast to an unlinked control (a 1:1 mixture of dasatinib and asciminib) at equimolar concentration, which competed with XO44 for labeling of numerous known dasatinib targets (54), pretreatment with DasatiLink-1 resulted in observable intracellular occupancy of only a single kinase (FIG. 3F). These data suggested an exquisite target selectivity conferred by two-site binding, analogous to RapaLink-1's heightened selectivity for MTOR complex 1 over MTOR complex 2 (4, 5, 24, 56). This transposition of emergent properties from RapaLink-1 to DasatiLink-1 establishes a generalizable strategy for the design of highly selective, cell permeable bitopic inhibitors.

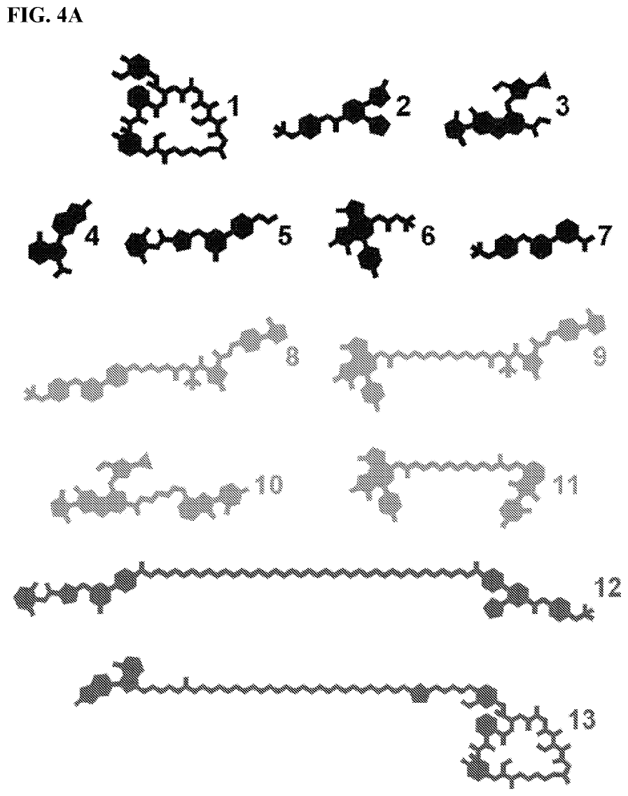

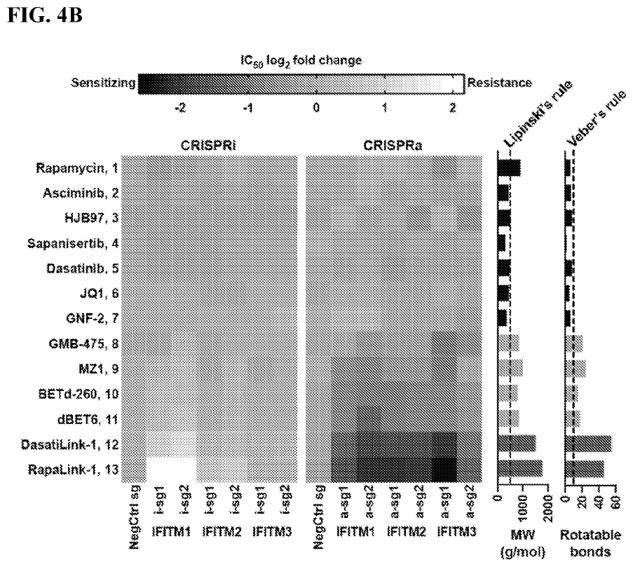

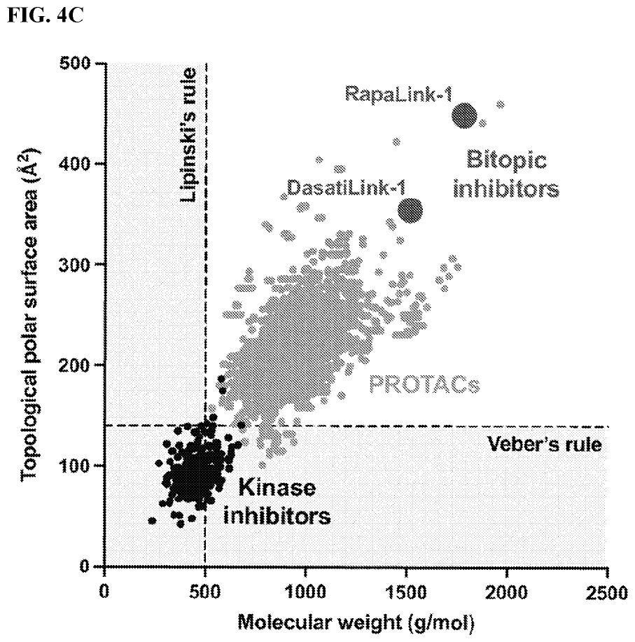

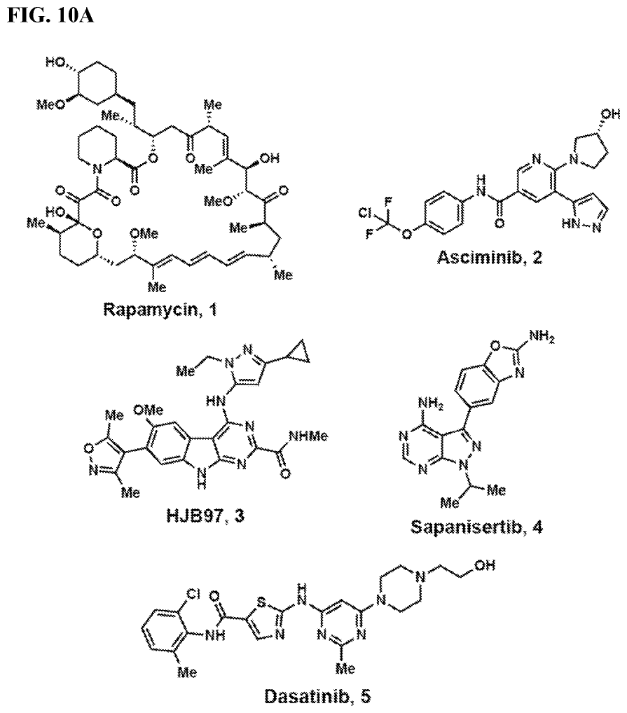

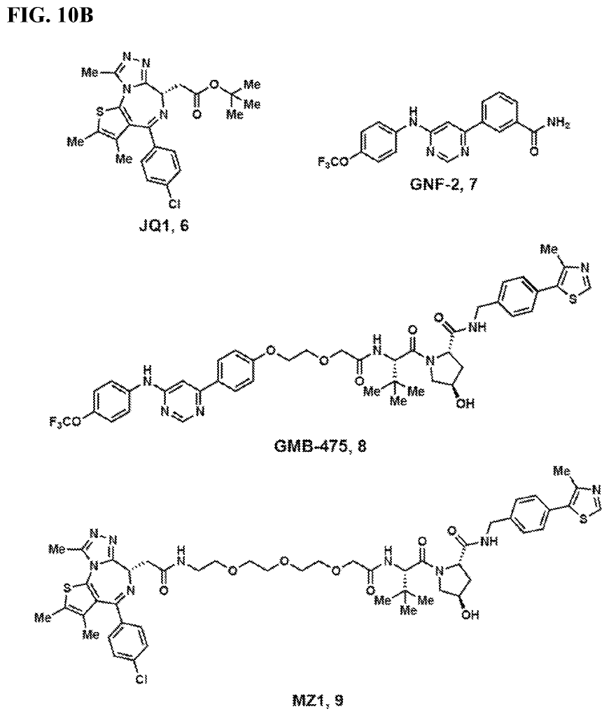

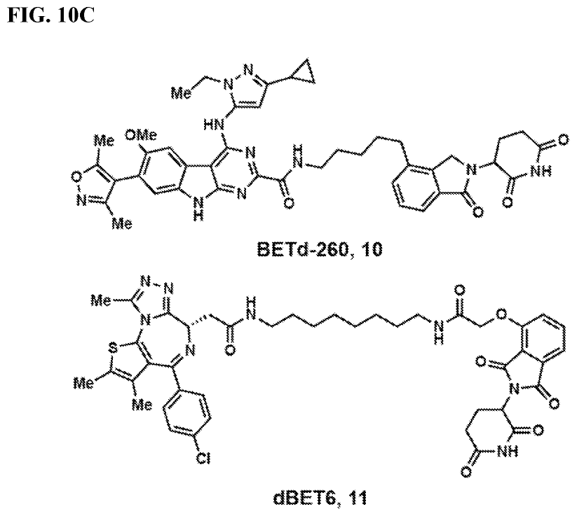

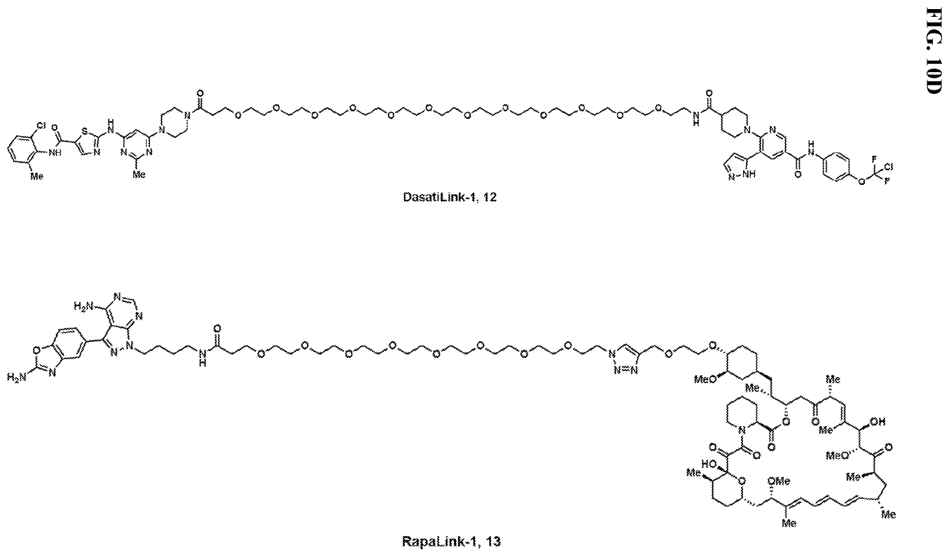

Given the ubiquitous presence of IFITM proteins in cells, we hypothesized that other linked inhibitors in the literature may have already been utilizing an IFITM-dependent uptake mechanism incidentally. While not as large as the bitopic inhibitors described above, PROTACs are also composed of two chemical entities covalently attached by a flexible tether (14), and several examples display comparably diminished passive diffusion in a non-cellular context (16, 17). Thus, we included several PROTACs and their non-linked targeting elements in a survey of known inhibitors for chemical-genetic interactions with IFITM proteins (FIG. 4A, FIGS. 10A-10D, and FIG. 11). We treated our K562 CRISPRi and CRISPRa models with these inhibitors and evaluated differences in potency resulting from IFITM protein expression modulation, as measured by half-maximal inhibitory concentration (IC₅₀) shift in a cell viability assay. Using RapaLink-1 and DasatiLink-1 as chemical benchmarks, we observed that IFITM1-3 overexpression broadly sensitized cells to linked chemotypes (FIG. 4B; compounds 8-13). The inverse finding, resistance to linked chemotypes, resulted from gene knockdown (FIG. 4B). The magnitudes of these chemical-genetic interactions correlated with inhibitor size (molecular weight) and flexibility (number of rotatable bonds) as the bitopic molecules were more dependent than the PROTACs tested, and non-linked chemotypes (FIG. 4B; compounds 1-7) appeared non-dependent (FIG. 4B). Despite their cellular activities, the physicochemical properties of these linked chemotypes largely violate Lipinski's (1) and Veber's (2) classic guidelines (FIG. 4B and FIG. 11), raising the need for a revised drug design framework that considers IFITM-mediated uptake and other cell assisted import processes. While a full characterization of the rules governing IFITM dependency will require further study, we propose that this uptake pathway can serve as a general entry mechanism for diverse large, flexible molecules of suitable amphiphilicity.

Through a combination of functional genomics and chemical methods, we uncovered an endogenous chemical uptake pathway involving IFITM proteins harnessed by diverse linked chemotypes. With the clinical advancement of a dimeric immunophilin ligand (21), PROTACs (NCT03888612 and NCT04072952), and a RapaLink-1 derivative (NCT04774952), the notion of ‘drug-like’ is continually being revised. As evidence, the chemical space (57, 58) populated by an ever-expanding set of linked preclinical compounds in the literature ventures beyond that occupied by lead inhibitors developed under traditional guidelines (FIG. 4C) (1-3). The bitopic inhibitors RapaLink-1 and DasatiLink-1 reach even further past these boundaries (FIG. 4C), and the absolute limits to molecular size, polarity, and flexibility among cell permeable compounds have likely not yet been fully realized.

In order to examine the chemical determinants of bitopic BCR-ABL1 inhibitors, we varied the composition of the ATP-site binding element (dasatinib or ponatinib), varied the length of the linker between the ATP-site binding element and allosteric binding element (asciminib), and varied the point of linkage from the ATP-site binding element. We found the above variables to have a strong influence on inhibitor potency both in biochemical assays and in cells—discussed further below. In summary, our data demonstrate that optimal choice of the above variables is necessary to achieve high potency for bitopic BCR-ABL1 inhibitors, particularly against common resistance mutants such at T315I.

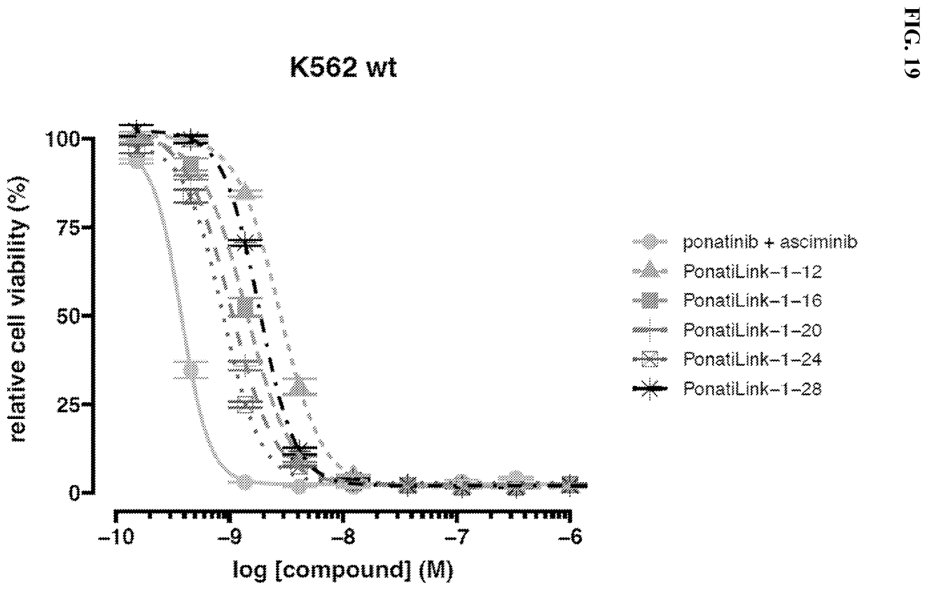

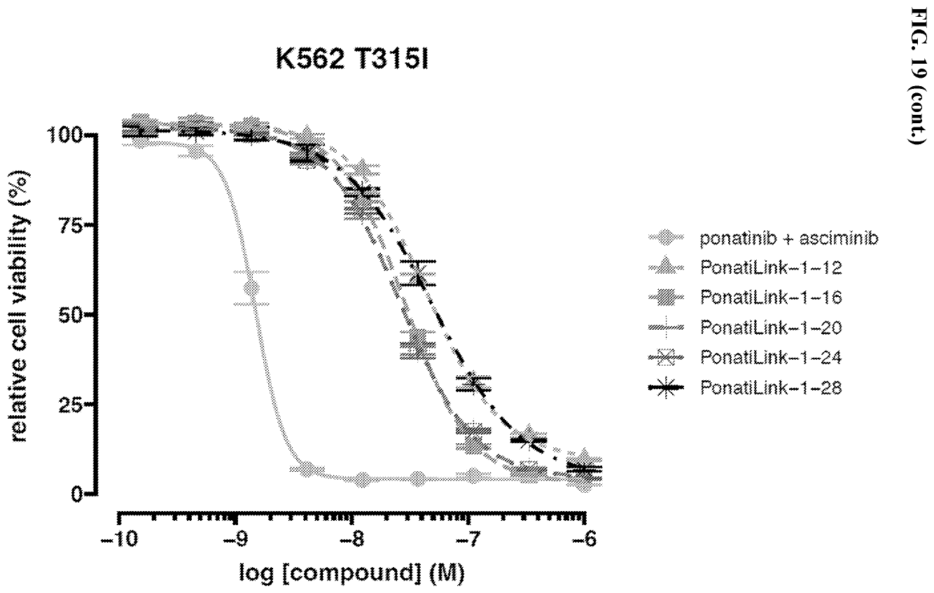

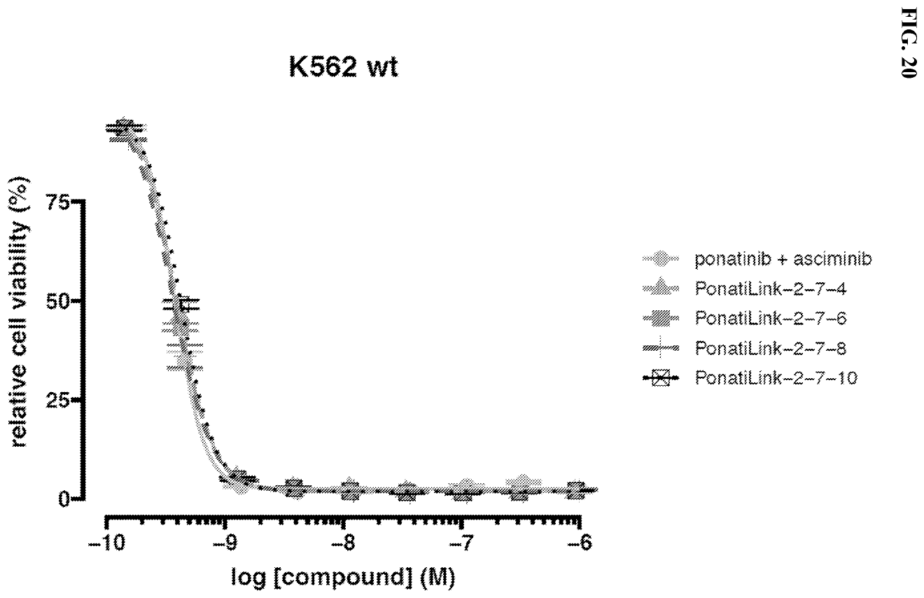

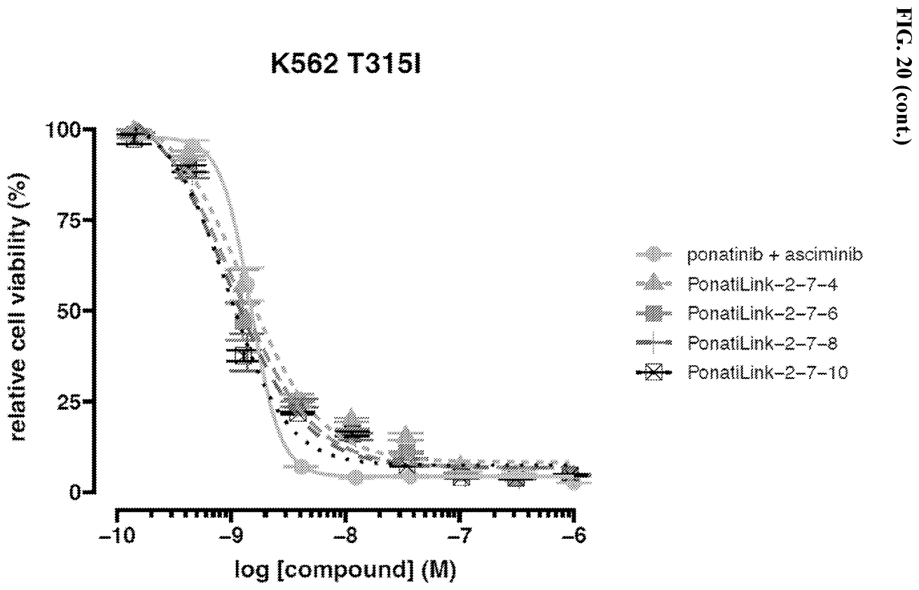

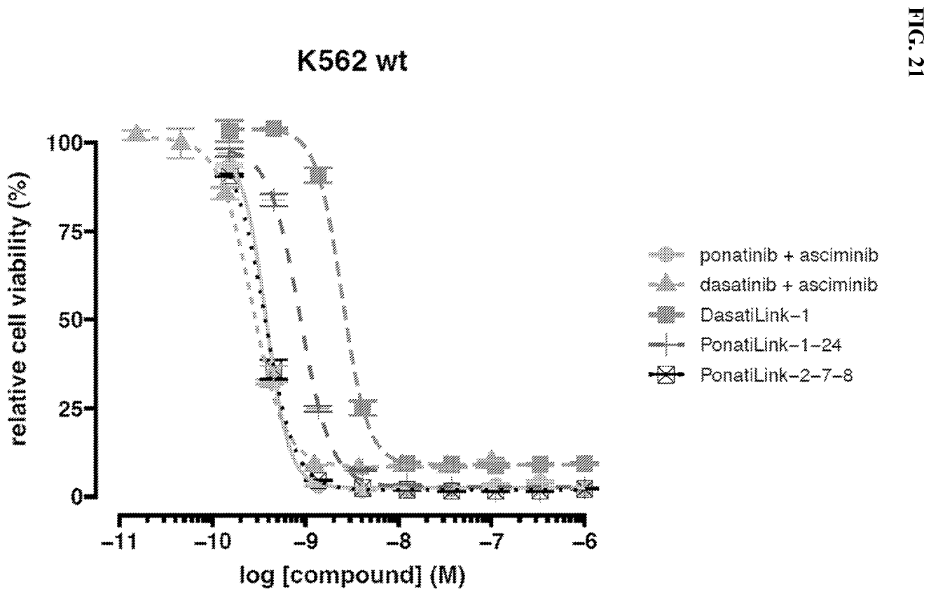

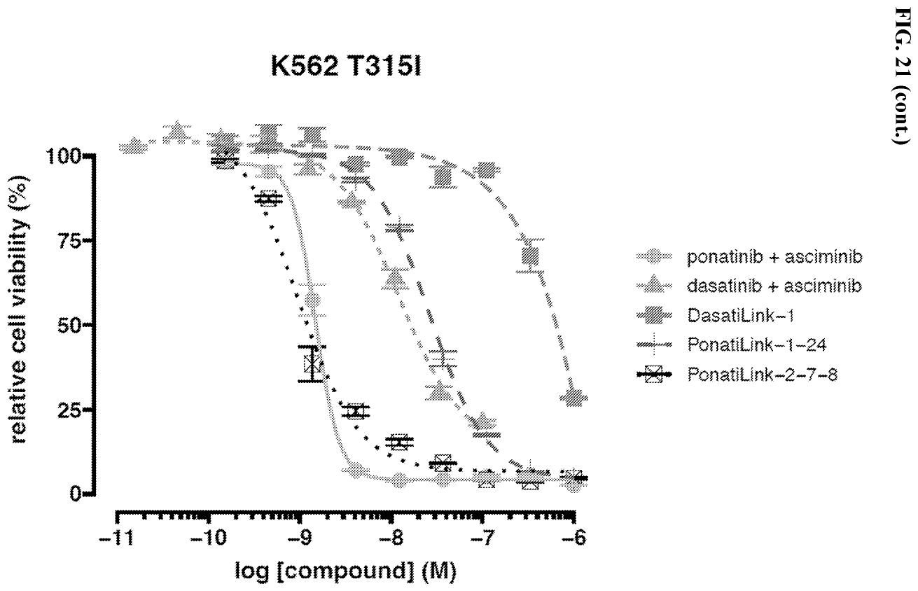

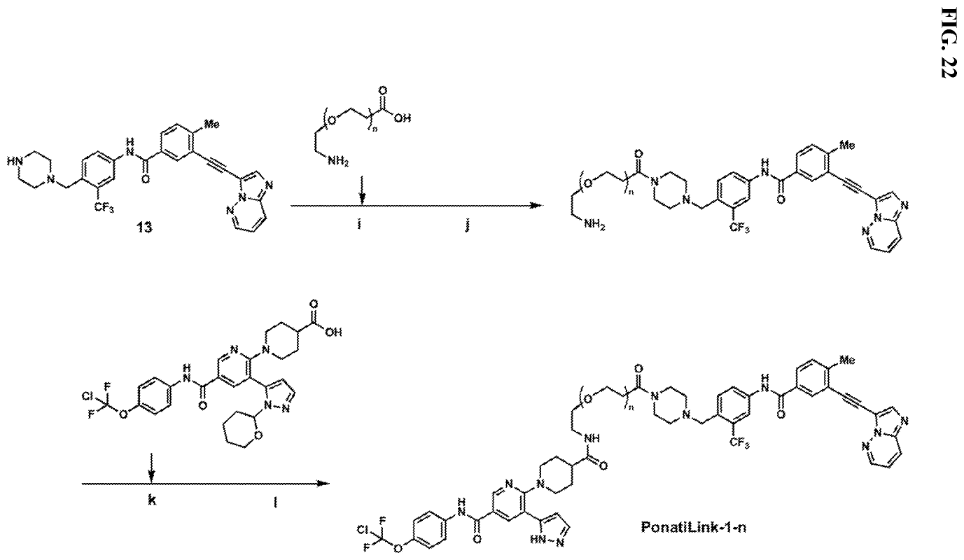

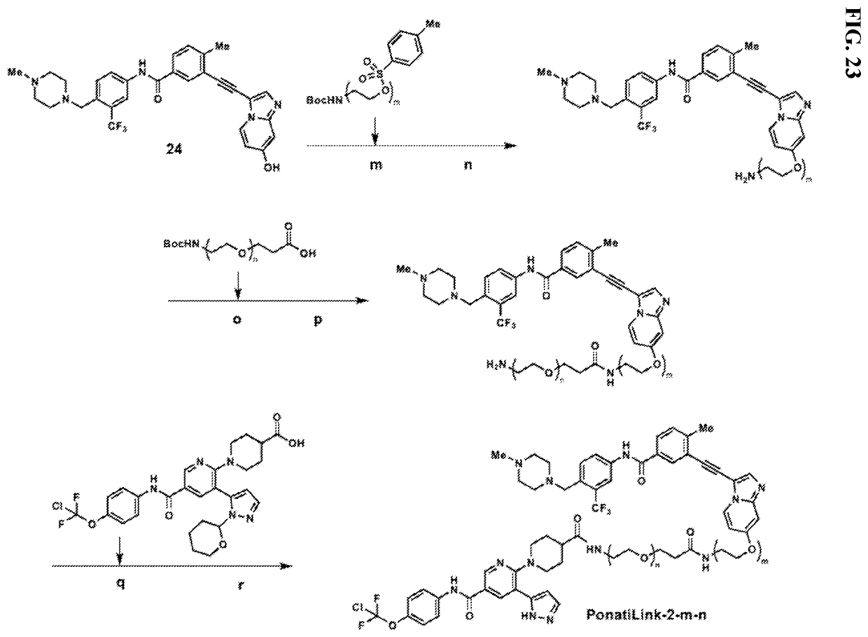

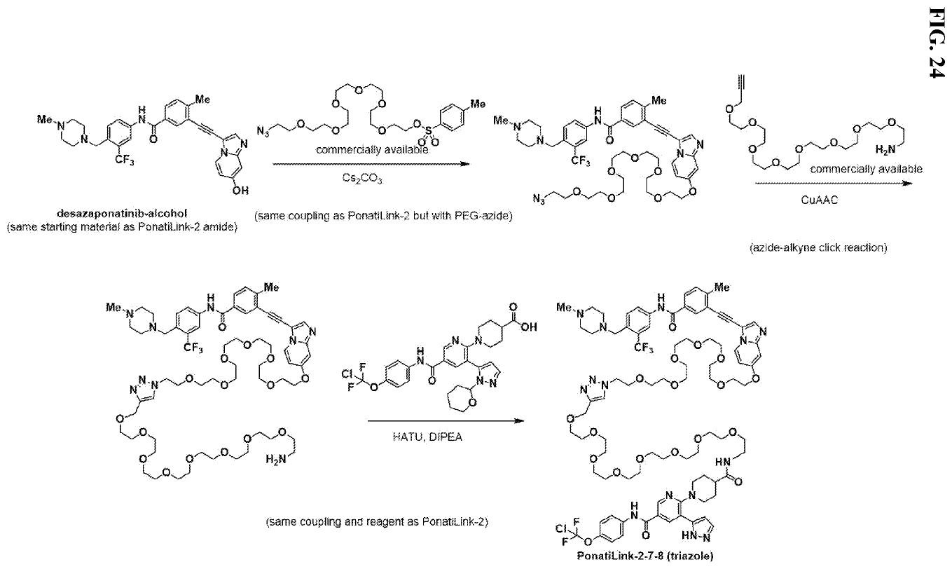

Dasatinib is a type-I kinase inhibitor that binds to an active conformation of the kinase and ponatinib is a type-II kinase inhibitor that binds to an inactive conformation of the kinase (https.//pubmed.ncbi.nlm.nih.gov/30612951/). Allosteric inhibitors may bind synergistically, additively, or antagonistically with various types of ATP-site binding inhibitors (48). Given that the bitopic inhibitors of BCR-ABL1 described herein are designed to simultaneously engage two binding pockets, it might be expected that the binding of one part of the inhibitor at one site may affect the binding of the other part of the inhibitor at the other site via allosteric interactions. We observed that PonatiLink compounds generally outperformed DasatiLink compounds in cell inhibition assays, particularly against T315I mutant cells (FIG. 17, FIG. 18, FIG. 19, FIG. 20, FIG. 21). This could be attributed to allosteric synergy in binding between a type II kinase inhibitor and and allosteric inhibitor, which by definition induces an inactive conformation of its target. Additionally, ponatinib is more potent than dasatinib against the T315I mutation in cells (FIG. 21), which could also contribute to the PonatiLink compounds' increased potency relative to DasatiLink compounds against the T315I mutant in cells.

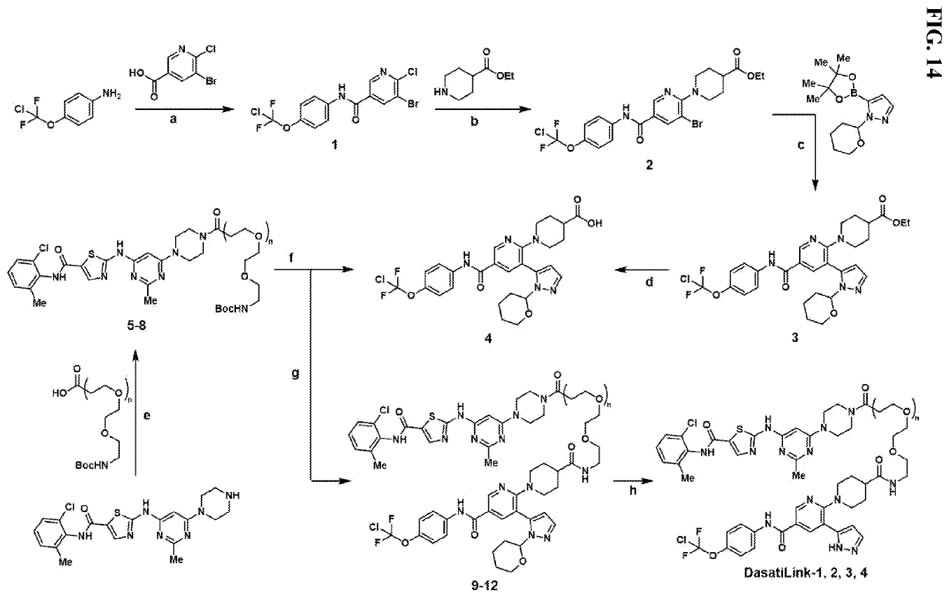

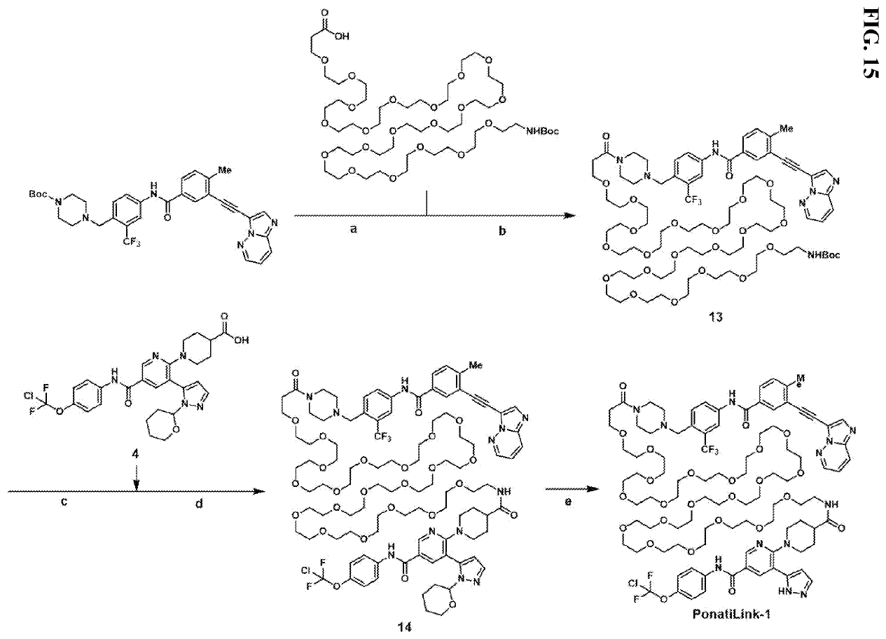

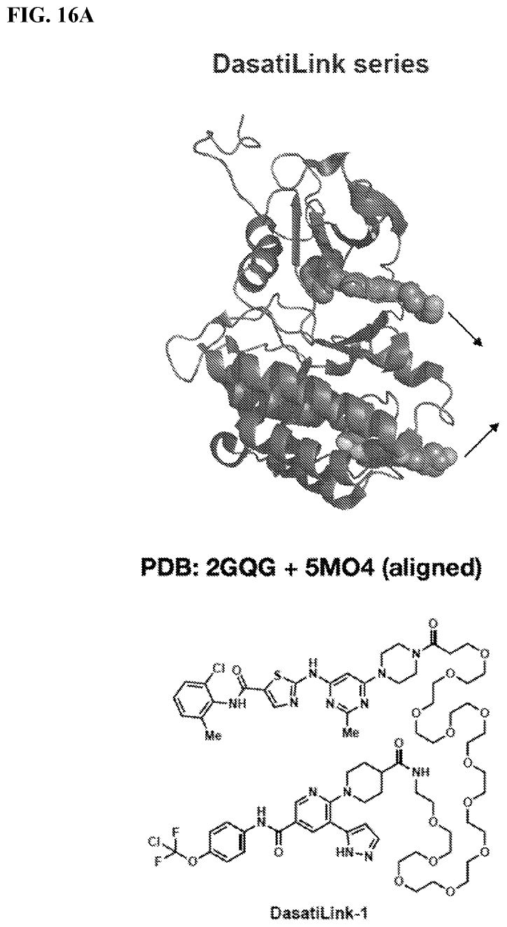

The flexible tether between the two binding elements of a bitopic BCR-ABL1 inhibitor serves as a restrictor of diffusion past a certain distance determined by the composition of the flexible tether. Therefore, the tether (i.e., linker) must be sufficiently long in order to allow the two binding elements to simultaneously engage the protein. Additionally, if the tether is too long, unfavorable entropic/steric forces and decreased membrane permeability (secondary to large molecular size) could limit the resultant molecule's potency. We synthesized chemical variants with differing linker lengths and determined patterns of biochemical and cellular inhibition for DasatiLink and PonatiLink compounds (FIG. 13B, FIG. 17, FIG. 18, FIG. 19, FIG. 20, FIG. 21). An ideal linker length for DasatiLink compounds is estimated to be approximately 40 atoms. An ideal linker length for PonatiLink-1 and PonatiLink-2 compounds is estimated to be approximately 75 and 27 atoms respectively, given that either decreasing linker length or increasing linker length from that distance was observed to hamper the potency of the inhibitor.

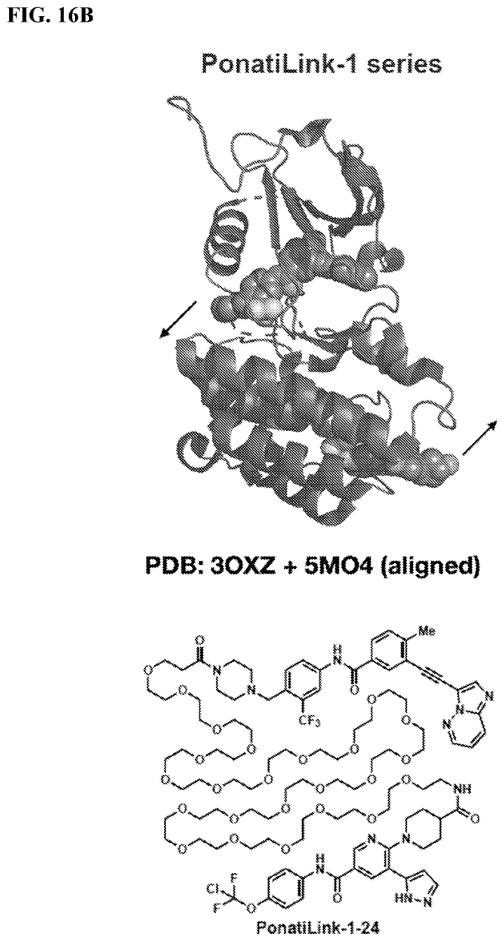

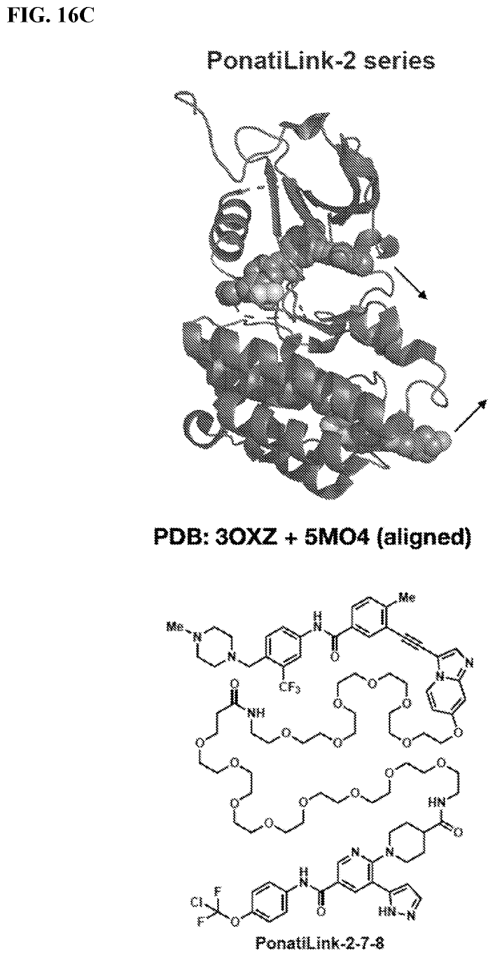

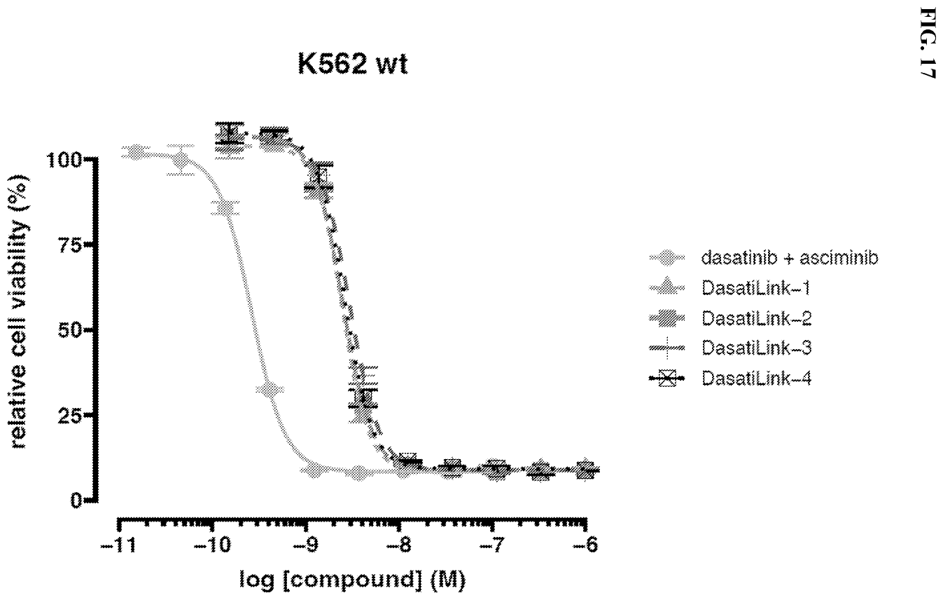

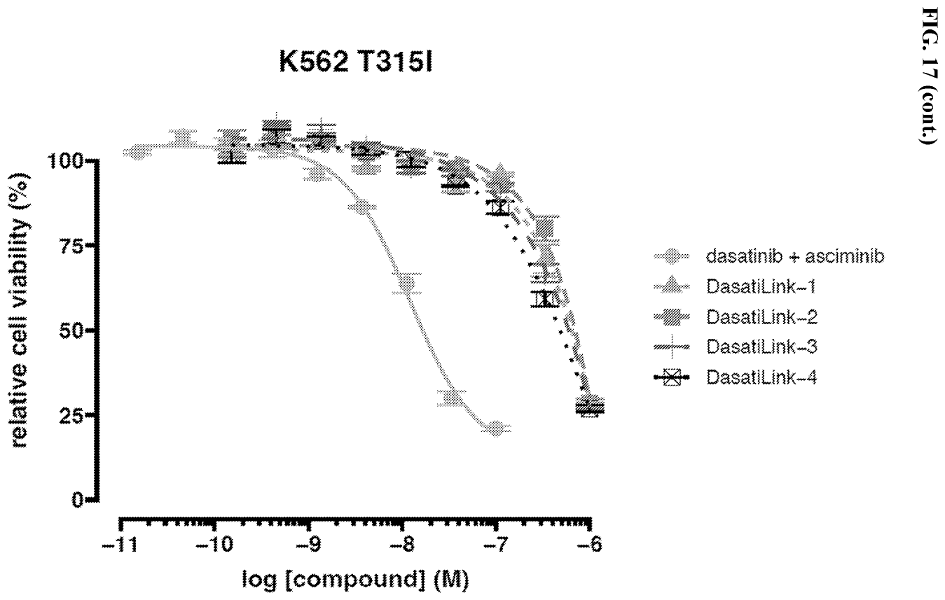

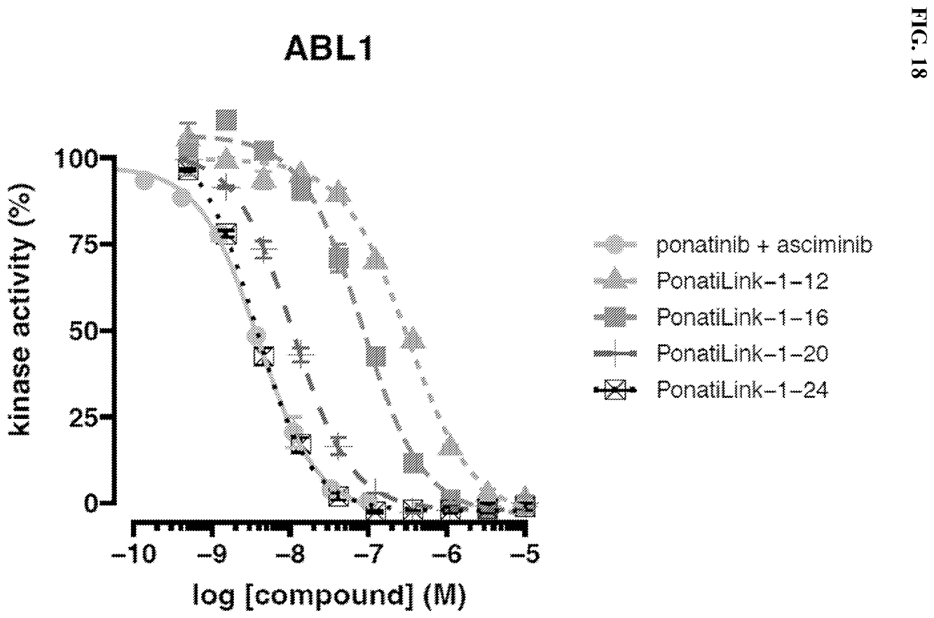

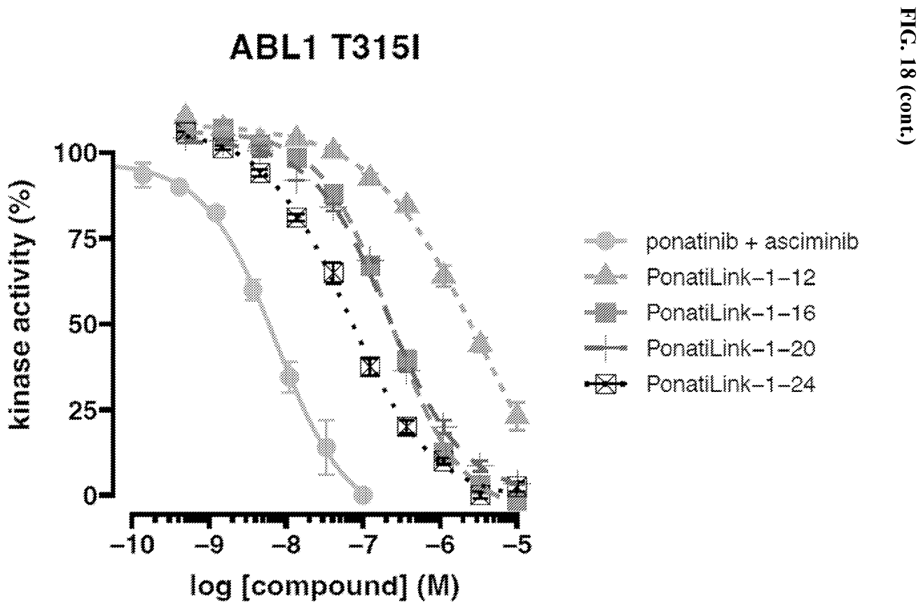

Furthermore, variation of the attachment vector at the ATP-site inhibitor was explored for PonatiLink compounds. PonatiLink-1 compounds were derivatized at the piperazine, which points in an opposite direction from the asciminib pocket on the target protein (FIGS. 16A-16C). PonatiLink-2 compounds were derivatized on the other end of the molecule (the heterocyclic hinge binding motif) which points more proximally to the asciminib pocket—notably, this is the same linkage orientation used by DasatiLink compounds based on binding poses (FIGS. 16A-16C). Optimal linker lengths differed within the two series. For PonatiLink-1 compounds, a linker length of 75 atoms was near-optimal in cell inhibition assays, while for PonatiLink-2 compounds a shorter linker length of 27 atoms was near-optimal (FIG. 19, FIG. 20). Our data are consistent with a conclusion that a linker vector long enough to traverse the path between the two binding sites without steric hindrance, but without unnecessary length, is advantageous for inhibitor potency and drug-like properties, represented by DasatiLink-1 and PonatiLink-2-7-8.

### REFERENCES FOR EXAMPLE 1

1. C. A. Lipinski, F. Lombardo, B. W. Dominy, P. J. Feeney, Adv Drug Deliver Rev. 23, 3-25 (1997). 2. D. F. Veber, S. R. Johnson, H.-Y. Cheng, B. R. Smith, K. W. Ward, K. D. Kopple, J Med Chem. 45, 2615-2623 (2002). 3. A. K. Ghose, V. N. Viswanadhan, J. J. Wendoloski, J Comb Chem. 1, 55-68 (1999). 4. V. S. Rodrik-Outmezguine, M. Okaniwa, Z. Yao, C. J. Novotny, C. McWhirter, A. Banaji, H. Won, W. Wong, M. Berger, E. de Stanchina, D. G. Barratt, S. Cosulich, T. Klinowska, N. Rosen, K. M. Shokat, Nature. 534, 272-6 (2016). 5. Q. Fan, O. Aksoy, R. A. Wong, S. Ilkhanizadeh, C. J. Novotny, W. C. Gustafson, A. Y.-Q. Truong, G. Cayanan, E. F. Simonds, D. Haas-Kogan, J. J. Phillips, T. Nicolaides, M. Okaniwa, K. M. Shokat, W. A. Weiss, Cancer Cell. 31, 424-435 (2017). 6. F. A. Meijer, G. J. M. Oerlemans, L. Brunsveld, Acs Chem Biol. 16, 510-519 (2021). 7. D. Spencer, T. Wandless, S. Schreiber, G. Crabtree, Science. 262, 1019-1024 (1993). 8. K. M. Sakamoto, K. B. Kim, A. Kumagai, F. Mercurio, C. M. Crews, R. J. Deshaies, Proc National Acad Sci. 98, 8554-8559 (2001). 9. G. S. Erwin, M. P. Grieshop, A. Ali, J. Qi, M. Lawlor, D. Kumar, I Ahmad, A. McNally, N. Teider, K. Worringer, R. Sivasankaran, D. N. Syed, A. Eguchi, Md. Ashraf, J. Jeffery, M. Xu, P. M. C. Park, H. Mukhtar, A. K. Srivastava, M. Faruq, J. E. Bradner, A. Z. Ansari, Science. 358, eaan6414 (2017). 10. M. G. Costales, Y. Matsumoto, S. P. Velagapudi, M. D. Disney, J Am Chem Soc. 140, 6741-6744 (2018). 11. M. Green, P. M. Loewenstein, Cell. 55, 1179-1188 (1988). 12. A. D. Frankel, C. O. Pabo, Cell. 55, 1189-1193 (1988). 13. E. L. Snyder, S. F. Dowdy, Pharmaceut Res. 21, 389-393 (2004). 14. A. C. Lai, C. M. Crews, Nat Rev Drug Discov. 16, 101-114 (2017). 15. C. A. Foley, F. Potjewyd, K. N. Lamb, L. I. James, S. V. Frye, Acs Chem Biol. 15, 290-295 (2019). 16. V. G. Klein, C. E. Townsend, A. Testa, M. Zengerle, C. Maniaci, S. J. Hughes, K.-H. Chan, A. Ciulli, R. S. Lokey, Acs Med Chem Lett. 11, 1732-1738 (2020). 17. D. E. Scott, T. P. C. Rooney, E. D. Bayle, T. Mirza, H. M. G. Willems, J. H. Clarke, S. P. Andrews, J. Skidmore, Acs Med Chem Lett (2020), doi:10.1021/acsmedchemlett.0c00194. 18. C. Cantrill, P. Chaturvedi, C. Rynn, J. P. Schaffland, I. Walter, M. B. Wittwer, Drug Discov Today. 25, 969-982 (2020). 19. G. Ermondi, M. Vallaro, G. Caron, Drug Discov Today. 25, 1585-1591 (2020). 20. Y. Atilaw, V. Poongavanam, C. S. Nilsson, D. Nguyen, A. Giese, D. Meibom, M. Erdelyi, J. Kihlberg, Acs Med Chem Lett. 12, 107-114 (2020). 21. J. D. Iuliucci, S. D. Oliver, S. Morley, C. Ward, J. Ward, D. Dalgarno, T. Clackson, H. J. Berger, Intravenous Safety and Pharmacokinetics of a Novel Dimerizer Drug, AP1903, in Healthy Volunteers (2001), pp. 870-879. 22. Z. Zhang, Q. Fan, X. Luo, K. J. Lou, W. A. Weiss, K. M. Shokat, Biorxiv, in press, doi:10.1101/2020.10.12.336677. 23. S. S. F. Leung, J. Mijalkovic, K. Borrelli, M. P. Jacobson, J Chem Inf Model. 52, 1621-1636 (2012). 24. B. J. Lee, J. A. Boyer, G. L. Burnett, A. P. Thottumkara, N. Tibrewal, S. L. Wilson, T. Hsieh, A. Marquez, E. G. Lorenzana, J. W. Evans, L. Hulea, G. Kiss, H. Liu, D. Lee, O. Larsson, S. McLaughlan, I. Topisirovic, Z. Wang, Z. Wang, Y. Zhao, D. Wildes, J. B. Aggen, M. Singh, A. L. Gill, J. A. M. Smith, N. Rosen, Nat Chem Biol, 1-10 (2021). 25. L. A. Gilbert, M. A. Horlbeck, B. Adamson, J. E. Villalta, Y. Chen, E. H. Whitehead, C. Guimaraes, B. Panning, H. L. Ploegh, M. C. Bassik, L. S. Qi, M. Kampmann, J. S. Weissman, Cell. 159, 647-661 (2014). 26. M. A. Horlbeck, L. A. Gilbert, J. E. Villalta, B. Adamson, R. A. Pak, Y. Chen, A. P. Fields, C. Park, J. E. Corn, M. Kampmann, J. S. Weissman, eLife. 5, e19760 (2016). 27. M. Jost, Y. Chen, L. A. Gilbert, M. A. Horlbeck, L. Krenning, G. Menchon, A. Rai, M. Y. Cho, J. J. Stern, A. E. Prota, M. Kampmann, A. Akhmanova, M. O. Steinmetz, M. E. Tanenbaum, J. S. Weissman, Molecular Cell. 68, 210-223.e6 (2017). 28. G. Y. Liu, D. M. Sabatini, Nat Rev Mol Cell Bio. 21, 183-203 (2020). 29. E. D. Zan, R. van Stiphout, B. V. Gapp, V. A. Blomen, T. R. Brummelkamp, S. M. B. Nijman, Sci Signal. 13, eaba5665 (2020). 30. K. J. Condon, J. M. Orozco, C. H. Adelmann, J. B. Spinelli, P. W. van der Helm, J. M. Roberts, T. Kunchok, D. M. Sabatini, Proc National Acad Sci. 118, e2022120118 (2021). 31. Z. Zhang, J. Liu, M. Li, H. Yang, C. Zhang, Plos One. 7, e49265 (2012). 32. P. Li, M.-L. Shi, W.-L. Shen, Z. Zhang, D.-J. Xie, X.-Y. Zhang, C. He, Y. Zhang, Z.-H. Zhao, Biochimica Et Biophysica Acta Bba—Gene Regul Mech. 1860, 885-893 (2017). 33. M. J. Garnett, E. J. Edelman, S. J. Heidorn, C. D. Greenman, A. Dastur, K. W. Lau, P. Greninger, I. R. Thompson, X. Luo, J. Soares, Q. Liu, F. Iorio, D. Surdez, L. Chen, R. J. Milano, G. R. Bignell, A. T. Tam, H. Davies, J. A. Stevenson, S. Barthorpe, S. R. Lutz, F. Kogera, K. Lawrence, A. McLaren-Douglas, X. Mitropoulos, T. Mironenko, H. Thi, L. Richardson, W. Zhou, F. Jewitt, T. Zhang, P. O'Brien, J. L. Boisvert, S. Price, W. Hur, W. Yang, X. Deng, A. Butler, H. G. Choi, J. W. Chang, J. Baselga, I. Stamenkovic, J. A. Engelman, S. V. Sharma, O. Delattre, J. Saez-Rodriguez, N. S. Gray, J. Settleman, P. A. Futreal, D. A. Haber, M. R. Stratton, S. Ramaswamy, U. McDermott, C. H. Benes, Nature. 483, 570-575 (2012). 34. M. G. Rees, B. Seashore-Ludlow, J. H. Cheah, D. J. Adams, E. V. Price, S. Gill, S. Javaid, M. E. Coletti, V. L. Jones, N. E. Bodycombe, C. K. Soule, B. Alexander, A. Li, P. Montgomery, J. D. Kotz, C. S.-Y. Hon, B. Munoz, T. Liefeld, V. Danc̆ik, D. A. Haber, C. B. Clish, J. A. Bittker, M. Palmer, B. K. Wagner, P. A. Clemons, A. F. Shamji, S. L. Schreiber, Nat Chem Biol. 12, 109-116 (2016). 35. F. Iorio, T. A. Knijnenburg, D. J. Vis, G. R. Bignell, M. P. Menden, M. Schubert, N. Aben, E. Gongalves, S. Barthorpe, H. Lightfoot, T. Cokelaer, P. Greninger, E. van Dyk, H. Chang, H. de Silva, H. Heyn, X. Deng, R. K. Egan, Q. Liu, T. Mironenko, X. Mitropoulos, L. Richardson, J. Wang, T. Zhang, S. Moran, S. Sayols, M. Soleimani, D. Tamborero, N. Lopez-Bigas, P. Ross-Macdonald, M. Esteller, N. S. Gray, D. A. Haber, M. R. Stratton, C. H. Benes, L. F. A. Wessels, J. Saez-Rodriguez, U. McDermott, M. J. Garnett, Cell. 166, 740-754 (2016). 36. K. Li, R. M. Markosyan, Y.-M. Zheng, O. Golfetto, B. Bungart, M. Li, S. Ding, Y. He, C. Liang, J. C. Lee, E. Gratton, F. S. Cohen, S.-L. Liu, Plos Pathog. 9, e1003124 (2013). 37. C. C. Bailey, G. Zhong, I.-C. Huang, M. Farzan, Annual Review of Virology. 1, 1-23 (2014). 38. J. S. Spence, R. He, H.-H. Hoffmann, T. Das, E. Thinon, C. M. Rice, T. Peng, K. Chandran, H. C. Hang, Nat Chem Biol. 15, 259-268 (2019). 39. F. Siegrist, M. Ebeling, U. Certa, J Interf Cytokine Res. 31, 183-197 (2011). 40. J. Lee, M. E. Robinson, N. Ma, D. Artadji, M. A. Ahmed, G. Xiao, T. Sadras, G. Deb, J. Winchester, K. N. Cosgun, H. Geng, L. N. Chan, K. Kume, T. P. Miettinen, Y. Zhang, M. A. Nix, L. Klemm, C. W. Chen, J. Chen, V. Khaimar, A. P. Wiita, A. Thomas-Tikhonenko, M. Farzan, J. U. Jung, D. M. Weinstock, S. R. Manalis, M. S. Diamond, N. Vaidehi, M. Mischen, Nature, 1-7 (2020). 41. J. Buchrieser, S. A. Degrelle, T. Couderc, Q. Nevers, O. Disson, C. Manet, D. A. Donahue, F. Porrot, K.-H. Hillion, E. Perthame, M. V. Arroyo, S. Souquere, K. Ruigrok, A. Dupressoir, T. Heidmann, X. Montagutelli, T. Fournier, M. Lecuit, O. Schwartz, Sci New York NY. 365, 176-180 (2019). 42. G. Shi, O. Schwartz, A. A. Compton, Retrovirology. 14, 53 (2017). 43. F. H. Epstein, R. Kurzrock, J. U. Gutterman, M. Talpaz, New Engl J Medicine. 319, 990-998 (1988). 44. T. Schindler, W. Bommann, P. Pellicena, W. T. Miller, B. Clarkson, J. Kuriyan, Science. 289, 1938-1942 (2000). 45. J. Zhang, F. J. Adriin, W. Jahnke, S. W. Cowan-Jacob, A. G. Li, R. E. Iacob, T. Sim, J. Powers, C. Dierks, F. Sun, G.-R. Guo, Q. Ding, B. Okram, Y. Choi, A. Wojciechowski, X. Deng, G. Liu, G. Fendrich, A. Strauss, N. Vajpai, S. Grzesiek, T. Tuntland, Y. Liu, B. Bursulaya, M. Azam, P. W. Manley, J. R. Engen, G. Q. Daley, M. Warmuth, N. S. Gray, Nature. 463, 501-506 (2010). 46. A. A. Wylie, J. Schoepfer, W. Jahnke, S. W. Cowan-Jacob, A. Loo, P. Furet, A. L. Marzinzik, X. Pelle, J. Donovan, W. Zhu, S. Buonamici, A. Q. Hassan, F. Lombardo, V. Iyer, M. Palmer, G. Berellini, S. Dodd, S. Thohan, H. Bitter, S. Branford, D. M. Ross, T. P. Hughes, L. Petruzzelli, K. G. Vanasse, M. Warmuth, F. Hofmann, N. J. Keen, W. R. Sellers, Nature. 543, 733-737 (2017). 47. T. P. Braun, C. A. Eide, B. J. Druker, Cancer Cell. 37, 530-542 (2020). 48. R. E. Iacob, J. Zhang, N. S. Gray, J. R. Engen, Plos One. 6, e15929 (2011). 49. N. Vajpai, A. Strauss, G. Fendrich, S. W. Cowan-Jacob, P. W. Manley, S. Grzesiek, W. Jahnke, J Biol Chem. 283, 18292-18302 (2008). 50. B. R. Stockwell, Nat Rev Genet. 1, 116-125 (2000). 51. M. A. Fabian, W. H. Biggs, D. K. Treiber, C. E. Atteridge, M. D. Azimioara, M. G. Benedetti, T. A. Carter, P. Ciceri, P. T. Edeen, M. Floyd, J. M. Ford, M. Galvin, J. L. Gerlach, R. M. Grotzfeld, S. Herrgard, D. E. Insko, M. A. Insko, A. G. Lai, J.-M. Lélias, S. A. Mehta, Z. V. Milanov, A. M. Velasco, L. M. Wodicka, H. K. Patel, P. P. Zarrinkar, D. J. Lockhart, Nat Biotechnol. 23, 329-336 (2005). 52. J. Das, P. Chen, D. Norris, R. Padmanabha, J. Lin, R. V. Moquin, Z. Shen, L. S. Cook, A. M. Doweyko, S. Pitt, S. Pang, D. R. Shen, Q. Fang, H. F. de Fex, K. W. McIntyre, D. J. Shuster, K. M. Gillooly, K. Behnia, G. L. Schieven, J. Wityak, J. C. Barrish, J Med Chem. 49, 6819-6832 (2006). 53. C. M. Gower, M. E. K. Chang, D. J. Maly, Crit Rev Biochem Mol. 49, 102-115 (2014). 54. Q. Zhao, X. Ouyang, X. Wan, K. S. Gajiwala, J. C. Kath, L. H. Jones, A. L. Burlingame, J. Taunton, Journal of the American Chemical Society. 139, 680-685 (2017). 55. B. F. Cravatt, A. T. Wright, J. W. Kozarich, Annu Rev Biochem. 77, 383-414 (2008). 56. A. C. Hsieh, Y. Liu, M. P. Edlind, N. T. Ingolia, M. R. Janes, A. Sher, E. Y. Shi, C. R. Stumpf, C. Christensen, M. J. Bonham, S. Wang, P. Ren, M. Martin, K. Jessen, M. E. Feldman, J. S. Weissman, K. M. Shokat, C. Rommel, D. Ruggero, Nature. 485, 55 (2012). 57. S. L. Schreiber, Science. 287, 1964-1969 (2000). 58. B. C. Doak, B. Over, F. Giordanetto, J. Kihlberg, Chem Biol. 21, 1115-1142 (2014).

### Example 2: Experimental Methods

### Cell Culture and Reagents

K562 CRISPRi and CRISPRa cell lines were generated as described previously (1). K562 cells were maintained in RPMI medium (Gibco) supplemented with 10% (v/v) fetal bovine serum (FBS) (Avantor Seradigm), penicillin (100 U/mL, Gibco), streptomycin (100 μg/mL, Gibco), and 0.1% (v/v) Pluronic F-68 (Gibco) unless otherwise specified. HEK293T cells were maintained in DMEM medium (Gibco) supplemented with 10% (v/v) FBS (Avantor Seradigm), penicillin (100 U/mL, Gibco), and streptomycin (100 g/mL, Gibco). All cells were grown in 37° C., 5% CO₂ stationary culture unless otherwise specified. Cell counting was performed on an Attune NxT (Thermo Fisher Scientific) or Countess II FL Automated Cell Counter (Thermo Fisher Scientific). Cells were periodically tested for mycoplasma contamination using the MycoAlert PLUS Mycoplasma Detection Kit (Lonza). Dasatinib, rapamycin, and sapanisertib were obtained from LC Laboratories. TAMRA-N₃ was obtained from BroadPharm. Asciminib, BETd-260, dBET6, GMB-475, GNF-2, HJB97, JQ1, and MZ1 were obtained from MedChemExpress. RapaLink-1, RapaTAMRA, and TAMRA-PEG8-N₃ were synthesized as described previously (2). DasatiLink-1 was synthesized as described herein. Compounds were stored at −20° C. as 10 mM stock solutions in dimethyl sulfoxide (DMSO) or dilutions thereof. The concentrations indicated for inhibitor combinations (e.g., sapanisertib+rapamycin) represent the stoichiometric abundance of each solute individually (i.e., a 1 nM sapanisertib+rapamycin treatment is equivalent to treating cells with 1 nM sapanisertib AND 1 nM rapamycin).

### DNA Transfections and Lentivirus Production

HEK293T cells were transfected with sgRNA expression vectors and standard packaging vectors (pCMV-dR8.91 and pMD2.G) using TransIT-LT1 Transfection Reagent (Mirus Bio). Lentiviral supernatant was collected 2-3 days following transfection, filtered through sterile 0.45 μm polyvinylidene difluoride filters (Millipore), and stored at −80° C.

### Genome-Scale CRISPRi/a Screening

Genome-scale CRISPRi/a screens were modeled after previous examples (1, 3). Over the course of the screens, cells were grown in 500 mL Optimum Growth Flasks (Thomson) in 37° C., 5% CO₂ shaking culture [1300 revolutions per minute in a Multitron Incubator (Infors HT)]. K562 CRISPRi or CRISPRa cells were transduced with the five-sgRNA/gene human CRISPRi v2 (hCRISPRi-v2) or five-sgRNA/gene human CRISPRa v2 (hCRISPRa-v2) library respectively in the presence of polybrene (8 μg/mL) (3). Viral transduction was tittered to maximize singly transduced cells, targeting a multiplicity of infection (MOI)≤1 (percentage of transduced cells 2 days after transduction=20-40%). Transduced (sgRNA+) cells were selected with 2 doses of puromycin (1 μg/mL) up to 80-95% sgRNA+ in the population over the course of 5 days. Before the initiation of compound treatment, TO samples were harvested with a minimum 1000-fold library coverage (approximately 100 million cells). The remaining cells were then divided into 5 treatment arms (DMSO, 1 nM sapanisertib, 1 nM rapamycin, 1 nM sapanisertib+rapamycin, and 1 nM RapaLink-1) with 2 biological replicates each. Cells were monitored for population doublings daily, and dilutions were made using complete media supplemented with the indicated compounds to maintain continuous selective pressure. Cells were cultured at a minimum 500-fold library coverage (approximately 50 million cells) over 10 days, after which T10 samples were harvested with a minimum 1000-fold library coverage (approximately 100 million cells). Genomic DNA (gDNA) was extracted from TO and T10 samples using NucleoSpin Blood XL (Macherey-Nagel). sgRNA protospacers were amplified directly from gDNA and processed for sequencing on an Illumina HiSeq 4000 as described previously (4).

### Screen Processing

Sequencing data from CRISPRi and CRISPRa screens were aligned to the hCRISPRi-v2 or hCRISPRa-v2 library respectively, counted, and quantified using the Python 2.7-based ScreenProcessing pipeline [https://github.com/mhorlbeck/ScreenProcessing (3)]. Phenotypes and Mann-Whitney P values were determined as described previously (1, 3), although data detailed herein are not normalized to total population doublings. Additional analysis and plotting were performed in Prism 9 (GraphPad Software).

### Large-Scale Chemogenomic Profiling

### High-Throughput Cell Viability Determination

High-throughput drug screening and sensitivity modeling (curve fitting and IC₅₀ estimation) was performed essentially as described previously (5). Cells were grown in RPMI or DMEM/F12 medium supplemented with 5% FBS and penicillin/streptomycin, and maintained at 37° C. in a humidified atmosphere at 5% CO₂. Cell lines were propagated in these two media in order to minimize the potential effect of varying the media on sensitivity to therapeutic compounds in our assay, and to facilitate high-throughput screening. To exclude cross-contaminated or synonymous lines, a panel of 92 SNPs was profiled for each cell line (Sequenom, San Diego, CA) and a pair-wise comparison score calculated. In addition, short tandem repeat (STR) analysis (AmpFlSTR Identifiler, Applied Biosystems, Carlsbad, CA) was performed and matched to an existing STR profile generated by the providing repository. Briefly, cells were seeded in 384 well plates at variable density to insure optimal proliferation during the assay. Drugs were added to the cells the day after seeding for adherent cell lines and the day of seeding for suspension cell lines. For tumor subtypes containing both adherent and suspension cells, all lines where drugged the same day (small cell lung cancer cell lines for example were all drugged the day after seeding). A series of nine doses was used with a 2-fold dilution factor for a total concentration range of 256 fold. Viability was determined using resazurin after 5 days of drug exposure, and data from treated wells were normalized to that of untreated wells.

### Correlation Analysis Between Drug Sensitivity and Basal Gene Expression

Dose-dependent growth inhibition of 935 cancer cell lines by RapaLink-1 and sapanisertib was determined as described above. Growth inhibition of 745 cell lines by rapamycin was obtained from the Genomics of Drug Sensitivity in Cancer database (GDSC2 release 8.3, accessed Oct. 4, 2020) (5, 6). Gene expression data from 1019 cell lines was obtained from the Cancer Cell Line Encyclopedia (CCLE 2019 release, accessed Sep. 28, 2020) (7). Spearman correlation coefficient between transcript level and area under the dose-response curve was calculated for each transcript using all cell lines present in both datasets (668 for RapaLink-1 and sapanisertib, 514 for rapamycin). Analysis and calculations were performed in R and plotted in Prism 9 (GraphPad Software).

Cloning of sgRNA Expression Vectors

sgRNA protospacers targeting FKBP12 (also known as FKBPIA), IFITM1, IFITM2, IFITM3, and a negative control (NegCtrl) sequence were individually cloned into pCRISPRia-v2 (Addgene 84832) as described previously (3). First, complementary synthetic oligonucleotide pairs (Integrated DNA Technologies) were designed containing protospacer sequences and flanking BstXI and BlpI restriction sites. Complementary oligonucleotides were mixed (2 μM each) in Nuclease-Free Duplex Buffer (Integrated DNA Technologies) and annealed by heating at 95° C. for 5 min, followed by gradual cooling to room temperature on the benchtop for 30 min. These duplexes were then ligated with BstXI, BlpI (New England Biolabs) doubly digested pCRISPRia-v2 (Addgene 84832) using T4 DNA Ligase (New England Biolabs). Standard transformation and preparation protocols were used to isolate individual vectors, which were sequence verified by Sanger sequencing (Quintara Biosciences).

### Stable Cell Line Generation

K562 CRISPRi or CRISPRa cells (200,000 cells in 1 mL per well) were seeded into 24-well plates and treated with lentivirus containing sgRNA expression vectors [marked with a puromycin resistance cassette and blue fluorescent protein (BFP)] in the presence of polybrene (8 μg/mL). 2 days after transduction, cells were selected for sgRNA+ populations with 3 doses of puromycin (2 μg/mL) over the course of 6 days. These cells could be stored under cryogenic conditions and were used for additional experiments described herein. The stability of cells were monitored by flow cytometry on an Attune NxT (Thermo Fisher Scientific), maintaining fluorescent marker expressing populations ≥90%.

Individual Evaluation of sgRNA Phenotypes

Cells were transduced as described herein. 5 days after transduction, cells were divided into 5 treatment conditions (DMSO, 1 nM sapanisertib, 1 nM rapamycin, 1 nM sapanisertib+rapamycin, and 1 nM RapaLink-1). Cells were monitored for the percentage of sgRNA+(BFP+) populations daily by flow cytometry, and dilutions were made using complete media supplemented with the indicated compounds to maintain continuous selective pressure. Increased relative sgRNA+ percentage over time corresponded to a resistance chemical-genetic interaction while decreased relative sgRNA+ percentage corresponded to a sensitizing chemical-genetic interaction.

### Immunoblotting

Cells (500,000 cells in 2 mL per well) were seeded into 6-well plates and incubated at 37° C. overnight. Following treatment with compounds at the concentrations and times indicated, cells were placed over ice, transferred to 2 mL microcentrifuge tubes, and pelleted at 500 g, 4° C. The pelleted cells were washed twice with ice-cold phosphate-buffered saline (PBS) and stored at −80° C. Pellets were disrupted using lysis buffer [100 mM Hepes (pH 7.5), 150 mM NaCl, and 0.1% NP-40] supplemented with cOmplete Protease Inhibitor Cocktail Tablets (Roche) and PhosSTOP (Roche), and protein concentrations of clarified lysates were determined by protein BCA assay (Thermo Fisher Scientific). Proteins were separated by polyacrylamide gel electrophoresis (PAGE), transferred to 0.2 μm pore size nitrocellulose membranes (Bio-Rad) and blocked using blocking buffer [5% bovine serum albumin (Millipore) in Tris-buffered saline, 0.1% Tween 20 (TBST) supplemented with 0.02% NaN₃]. Membranes were probed with primary antibodies against FKBP12 (ab58072) from Abcam and p-ABL1^{Y245} (2861), p-CRKL^{Y207} (3181), IFITM1 (13126), IFITM2 (13530), IFITM3 (59212), p-STAT5^{Y694} (4322), and Tubulin (3873) from Cell Signaling Technology diluted (1:1000) in blocking buffer. After primary antibody incubation, membranes were treated with IRDye secondary antibodies (LI-COR Biosciences) according to manufacturer's recommendations and scanned on an Odyssey Imaging System (LI-COR Biosciences). Immunoblot scans were processed using ImageStudioLite 5.2.5 (LI-COR).

### Internally Normalized Cellular Fluorescence Uptake Assay

K562 CRISPRi or CRISPRa cells stably expressing sgRNAs marked with BFP mixed at a 1:1 ratio with non-transduced (sgRNA−) cells (20,000 cells in 180 μL per well) were seeded into 96-well round bottom plates and incubated at 37° C. overnight. Cells were treated with fluorescent compounds at the concentrations indicated (200 μL final volume per well) and incubated at 37° C. for 24 h. Cells were pelleted at 500 g, washed twice with ice-cold PBS supplemented with 1% bovine serum albumin (Millipore) and 0.1% NaN₃, and resuspended in the same before assessment by flow cytometry on an Attune NxT (Thermo Fisher Scientific). TAMRA fluorescence (YL-H: 561 nm excitation laser, 585/16 emission filter) and BFP fluorescence (VL1-H: 405 nm excitation laser, 440/50 emission filter) was measured for cells within each well. Relative cellular uptake was determined by dividing the median TAMRA fluorescence intensity of BFP+ populations by that of BFP—populations (FIG. 2B). Relative cellular uptake <1 indicates decreased uptake resulting from the genetic perturbation and >1 indicates increased uptake.

### Protein Expression and Purification

Human ABL1 kinase domain (KD) encompassing residues 229-512 (isoform IA numbering) was cloned, expressed in Escherichia coli (E. coli), and purified as described previously (8). ABL1 KD containing a tobacco etch virus (TEV) protease-cleavable N-terminal hexahistidine tag (MKSSHHHHHHHHHHENLYFQSNA (SEQ ID NO:2)) was transformed into BL21(DE3) E. coli cells carrying a plasmid containing YopH phosphatase (pCDRF-Duet, streptomycin resistant) and a plasmid expressing GroEL and Trigger factor (pACYC-Duet, chloramphenicol resistant). ¹⁵N labeled ABL1 KD samples were produced in M9 minimal media containing 1 g/L ¹⁵NH₄Cl as the sole nitrogen source. Cells were grown at 37° C. to OD₆₀₀˜0.6-0.8. At OD₆₀₀˜0.6-0.8 cells were cooled to 16° C. for an hour, then expression was induced with 1 mM isopropyl-β-D-thiogalactoside (IPTG) and allowed to continue overnight (16-20 h). Proteins were purified with a 5 mL HisTrap HP (GE Healthcare) Ni affinity column (NiA buffer: 20 mM Tris pH 8.0, 500 mM NaCl, 5% glycerol; NiB buffer: 20 mM Tris pH 8.0, 500 mM NaCl, 5% glycerol, 500 mM imidazole), dialyzed overnight with TEV protease in 20 mM Tris pH 8.0, 100 mM NaCl, 1 mM DTT, 5% glycerol, and then purified with a 5 mL HiTrap anion exchange column (QA Buffer: 20 mM Tris pH 8.0, 1 mM TCEP, 5% glycerol; QB Buffer: 20 mM Tris pH 8.0, 1 M NaCl, 1 mM TCEP, 5% glycerol). Protein concentration was determined by absorbance measurement using a calculated extinction coefficient of 62590 M⁻¹ cm⁻¹ (ProtParam) (9). Purified samples were concentrated to 300 μM by ultrafiltration (molecular weight cut-off 10 kDa) and buffer exchanged into 50 mM sodium potassium phosphate pH 6.5, 50 mM NaCl, 5 mM DTT. Samples were snap frozen in liquid nitrogen and stored at −80° C.

### NMR Experiments

Dasatinib, asciminib, and their combination were added in five-fold molar excess to saturate binding sites during buffer exchange. For DasatiLink-1, the protein was diluted to ˜60 μM in 2500 μL and 25 μL of 5 mM bitopic ligand was added on ice to minimize solute precipitation. The process was repeated until the bitopic ligand reached 3-fold excess of the protein concentration. DMSO was maintained at 5% for all NMR samples. Samples were concentrated to a final protein concentration of 300 μM. 10% D₂O was added to NMR samples for signal locking. All ¹H-¹⁵N heteronuclear NMR experiments were acquired at 30° C. with 64 scans on a Bruker Avance III HD spectrometer operating at a ¹H frequency of 850 MHz equipped with a cryogenic probe. A standard Bruker pulse sequence for ¹H-¹⁵N TROSY-HSQC (trosyf3gpphsi19.2), was used. Backbone assignments for ABL1 KD used to interpret spectra described herein were obtained from the Biological Magnetic Resonance Data Bank (Entry ID: 15488) (10). Sample stability prior and post NMR experiments was assessed by acquiring 1-dimensional ¹H spectra to assess signal strength, sample concentration, and folding status.

### Cell Viability Assay

K562 CRISPRi or CRISPRa cells stably expressing sgRNAs (1,000 cells in 90 μL per well) were seeded into white opaque 96-well plates and incubated at 37° C. overnight. Cells were treated with the indicated concentrations of compound in 9-point 3-fold dilution series (100 μL final volume per well) and incubated at 37° C. for 72 h. Cell viability was assessed by CellTiter-Glo (CTG) Luminescent Cell Viability Assay (Promega). Cells were equilibrated to room temperature before the addition of diluted (1:4 CTG reagent:PBS) CTG reagent (100 μL per well). Plates were agitated on an orbital shaker and luminescence signal was measured on a SpectraMax M5 (Molecular Devices) or Spark (Tecan) plate reader. Repeated measurements of luminescence were performed as technical replicates to determine incubation times optimal for signal-to-noise. Luminescence measurements were normalized to DMSO-treated values to determine relative cell viability.

### ATP-Site Kinase Pulldown

ATP-site competition binding assay (KdELECT) was performed by Eurofins DiscoverX as described previously (11). Compounds were assessed in 11-point 3-fold dilution series and compound mixtures were analogously diluted from a DMSO stock containing the 2 compounds at the ratio indicated. Pulldown measurements of DNA-tagged kinase by quantitative polymerase chain reaction (qPCR) were normalized to DMSO-treated values to determine relative ATP-site pulldown. A 4-parameter nonlinear regression model was fit to the data using Prism 9 (GraphPad Software) with the top parameter constrained to 100%. An outlier point corresponding to 152% pulldown at 15.2 μM Dasa+Asc (1:100) was excluded from analysis.

### Live Cell Kinase Occupancy Profiling

### Compound Treatment and Preparation of Cell Lysates for Proteomics Analysis

K562 CRISPRi cells (1×10⁶/mL) were maintained in RPMI medium (Gibco) supplemented with 10% (v/v) fetal bovine serum (FBS) (Axenia BioLogix), penicillin (100 U/mL, Gibco), and streptomycin (100 μg/mL, Gibco). Cells were pretreated with DMSO, dasatinib+asciminib (100 nM), or DasatiLink-1 (100 nM) at 37° C. for 4 h, followed by treatment with XO44 (2 M) at 37° C. for another 30 min. Each sample was prepared in triplicate. Cell pellets were collected by centrifugation at 500 g, 4° C. and lysed in 100 mM HEPES pH 7.5, 150 mM NaCl, 0.1% NP-40, 1 mM PMSF, and 1× cOmplete EDTA-free protease inhibitor cocktail (Sigma-Aldrich #11873580001). Lysates were cleared by centrifugation (16,000 g, 4° C., 30 min). Protein concentration was determined by protein BCA assay (Thermo Fisher #23225). Cell lysates were normalized to 5 mg/mL with lysis buffer for subsequent pulldown-MS analysis.

### Pulldown of XO44-Modified Proteins and On-Bead Digestion

Cell lysates (5 mg/mL, 1.2 mL) were incubated with 40 μL of settled streptavidin agarose beads (Thermo Fisher Scientific #20353) at 4° C. overnight to remove endogenous biotinylated proteins. Beads were removed by filtration (Pall #4650). The filtrate (1 mL) was reacted with 191 μL of click chemistry cocktail, resulting in a final concentration of 1% SDS, 100 M DMTP biotin picolyl azide, 1 mM TCEP, 100 μM TBTA (from a 2 mM stock prepared in 1:4 DMSO:t-butyl alcohol), and 1 mM CuSO₄. After incubation at room temperature for 90 min, proteins were precipitated by adding 10 mL of prechilled acetone and incubating overnight at −20° C. The precipitated proteins were pelleted by centrifugation (3500 g, 4° C., 30 min), resuspended in cold MeOH and re-pelleted. The pellet was solubilized in 1% SDS in PBS, and then diluted to a final detergent concentration of 0.4% SDS, 0.6% NP40 in PBS before desalting on a NAP-10 column (Cytiva #17-0854-02). The column eluate was incubated with 40 μL of settled high-capacity neutravidin garose beads (Thermo Fisher Scientific #29204) at 4° C. overnight. The beads were then washed with 1% NP-40, 0.1% SDS in PBS (3×10 min, RT), freshly prepared 6 M urea in PBS (3×30 min, 4° C.) and PBS (3×10 min, RT). Disulfide reduction was performed with 5 mM DTT in 6M urea, PBS at 56° C. for 30 min, followed by alkylation with 20 mM iodoacetamide at room temperature for 15 min in the dark. On-bead digestion was performed in digestion buffer (2 M urea, 1 mM CaCl₂, PBS) by adding 1 g sequencing grade trypsin (Promega #V5113) to each sample, and incubating overnight at 37° C. Tryptic digests were collected by filtration. Peptide concentrations were determined by peptide BCA assay (Thermo Fisher Scientific #23275). An equal amount of peptides were removed from each sample and dried by Speedvac.

### TMT Labeling of Tryptic Peptides

TMT labeling was performed with the TMT10plex kit (Thermo Fisher Scientific #SK257743) according to manufacturer's recommendations with minor modifications. Briefly, peptides (25 μg) were reconstituted in 50 μL of 30% MeCN in 200 mM HEPES buffer pH 8.5. TMT reagents were reconstituted in 40 μL of MeCN per vial, and 6 μL of this solution was incubated with each sample for 1 h at RT. Reactions were quenched by adding 9 μL of 5% hydroxylamine and incubated at RT for 15 min, followed by adding 50 μL of 1% TFA to acidify the solution. TMT-labeled samples were pooled and concentrated by Speedvac to remove MeCN, and desalted using C18 OMIX Tips (Agilent #A57003100). Peptides were eluted with 50% MeCN, 0.1% TFA, and dried by Speedvac.

### LC-MS/MS Analysis

TMT labeled tryptic peptides were reconstituted in 5% MeCN, 0.1% TFA in water, and analyzed on a Orbitrap Eclipse Tribrid Mass Spectrometer (Thermo Fisher Scientific) connected to an UltiMate 3000 RSLCnano system with 0.1% FA as buffer A and 95% MeCN, 0.1% FA as buffer B. Peptides were separated on an EASY-Spray 3 μm, 75 μm×15 cm C18 column (Thermo Fisher Scientific #ES800) with the following LC settings: 0.3 mL/min flow rate, sample loading at 5% B for 20 min, then 5 to 7.4% B over 5 min, 7.4 to 50% B over 115 min, 50% to 95% B over 10 min and finally 95% B for 10 min. Data were acquired in a data-dependent mode. MS1 scans were acquired at a resolution of 120,000 with an AGC of 4e5, m/z scan range of 400-1600, a maximum ion injection time of 50 ms, a charge state of 2-6, and a 60 s dynamic exclusion time. MS2 spectra were acquired via collision-induced dissociation (CID) at a collision energy of 35%, in the ion trap with an automatic gain control (AGC) of 1e4, isolation width of 0.7 m/z and an auto maximum ion injection time. For real time search, MS2 spectra were searched against human reviewed Swiss-Prot database (accessed Sep. 16, 2020) with the digestion enzyme set to trypsin. Methionine oxidation was set as a variable modification, while carbamidomethylation of cysteine and TMT modification were set as constant modifications. For MS3 acquisition, a synchronous precursor selection (SPS) of 10 fragments was acquired in the orbitrap for a maximum ion injection time of 105 ms with an AGC of 2.5e5. MS3 spectra were collected at a resolution of 60,000 with higher-energy C-trap dissociation (HCD) collision energy of 55%.

### Protein Identification and TMT Quantification.

Raw files were analyzed with Thermo Scientific Proteome Discoverer (2.4) software against the human reviewed Swiss-Prot database (accessed Sep. 16, 2020). Trypsin was selected as the digestion enzyme with a maximum of 2 missed cleavages and a minimum peptide length of 6. Cysteine carbamidomethylation and TMT-6plex on K and peptide N-terminus were set as fixed modifications, while methionine oxidation and acetylation of protein N-terminus were set as variable modifications. Precursor tolerance was set to 10 ppm, and fragment tolerance was set to 0.6 Da. Peptide-spectrum match (PSM) and protein false discovery rate (FDR) were set to 1% and 5%, respectively. Reporter ion intensities were adjusted to correct for impurities during synthesis of different TMT reagents according to the manufacturer's recommendations. For quantification, PSMs with an average reporter signal-to-noise threshold (<9) and synchronous precursor selection (SPS) mass matches threshold (<75%) were removed from final dataset. Quantified PSMs were summarized to their matched proteins. Median TMT intensities at the protein level were normalized to the same across all TMT channels. Mean intensities from each group were log₂ transformed and used for calculation of the log₂ fold change between each condition.

### Chemical-Genetic Interaction Mapping

Cells treated with compounds were evaluated for viability as described herein. For K562 CRISPRi cells rapamycin (100 nM), asciminib (100 nM), HJB97 (10 μM), sapanisertib (1 μM), dasatinib (100 nM), JQ1 (10 μM), GNF-2 (10 μM), GMB-475 (10 μM), MZ1 (10 μM), BETd-260 (100 nM), dBET6 (1 μM), DasatiLink-1 (100 nM), and RapaLink-1 (100 nM) were evaluated using 9-point 3-fold dilution series starting from the highest concentrations indicated. The same top concentrations were used in K562 CRISPRa cells with the exception of HJB97 (1 μM), JQ1 (1 μM), MZ1 (1 μM), and dBET6 (100 nM). Using Prism 9 (GraphPad Software), a 4-parameter nonlinear regression model was fit to the viability data to determine IC₅₀ values. IC₅₀ values of sgRNA+ cells were normalized to that of non-sgRNA expressing cells to determine sensitivity/resistance chemical-genetic interactions mapped in FIG. 4B.

### Chemical Space Plotting

Data were drawn from the Protein Kinase Inhibitor Database (PKIDB) (12) and the Proteolysis-Targeting Chimera Database (PROTAC-DB) (13). 260 kinase inhibitors from the PKIDB (May 20, 2021 release, accessed Jun. 3, 2021) and 2258 PROTACs from the PROTAC-DB (May 27, 2021 release, accessed Jun. 16, 2021) were depicted in FIG. 4C. Molecular weight and topological surface area were plotted based on values associated with compounds in their respective databases. For bitopic inhibitors, physicochemical properties were computed as described herein.

### Physicochemical Property Determination

Unless otherwise specified, physicochemical properties of compounds were computed using SwissADME (14) and listed in FIG. 11. Simplified molecular-input line-entry system (SMILES) strings were inputted to http://www.swissadme.ch.

### Parallel Artificial Membrane Permeability Assay (PAMPA)

PAMPA was performed by Quintara Discovery using the BioCoat Pre-coated PAMPA Plate System (Corning) as described previously (15) and based on manufacturer's recommendations. All compounds were assessed at 100 μM in PBS. Compound concentrations in donor and acceptor compartments were quantified using liquid chromatography-mass spectrometry (LC-MS) 5 h following compound addition into the donor compartment.

### REFERENCES FOR EXAMPLE 2

1. L. A. Gilbert, M. A. Horlbeck, B. Adamson, J. E. Villalta, Y. Chen, E. H. Whitehead, C. Guimaraes, B. Panning, H. L. Ploegh, M. C. Bassik, L. S. Qi, M. Kampmann, J. S. Weissman, Cell. 159, 647-661 (2014). 2. Z. Zhang, Q. Fan, X. Luo, K. J. Lou, W. A. Weiss, K. M. Shokat, Biorxiv, in press, doi:10.1101/2020.10.12.336677. 3. M. A. Horlbeck, L. A. Gilbert, J. E. Villalta, B. Adamson, R. A. Pak, Y. Chen, A. P. Fields, C. Park, J. E. Com, M. Kampmann, J. S. Weissman, eLife. 5, e19760 (2016). 4. S. J. Liu, M. A. Horlbeck, J. S. Weissman, D. A. Lim, Methods Mol Biology. 2254, 323-338 (2020). 5. M. J. Garnett, E. J. Edelman, S. J. Heidorn, C. D. Greenman, A. Dastur, K. W. Lau, P. Greninger, I. R. Thompson, X. Luo, J. Soares, Q. Liu, F. Iorio, D. Surdez, L. Chen, R. J. Milano, G. R. Bignell, A. T. Tam, H. Davies, J. A. Stevenson, S. Barthorpe, S. R. Lutz, F. Kogera, K. Lawrence, A. McLaren-Douglas, X. Mitropoulos, T. Mironenko, H. Thi, L. Richardson, W. Zhou, F. Jewitt, T. Zhang, P. O'Brien, J. L. Boisvert, S. Price, W. Hur, W. Yang, X. Deng, A. Butler, H. G. Choi, J. W. Chang, J. Baselga, I. Stamenkovic, J. A. Engelman, S. V. Sharma, O. Delattre, J. Saez-Rodriguez, N. S. Gray, J. Settleman, P. A. Futreal, D. A. Haber, M. R. Stratton, S. Ramaswamy, U. McDermott, C. H. Benes, Nature. 483, 570-575 (2012). 6. F. Iorio, T. A. Knijnenburg, D. J. Vis, G. R. Bignell, M. P. Menden, M. Schubert, N. Aben, E. Gongalves, S. Barthorpe, H. Lightfoot, T. Cokelaer, P. Greninger, E. van Dyk, H. Chang, H. de Silva, H. Heyn, X. Deng, R. K. Egan, Q. Liu, T. Mironenko, X. Mitropoulos, L. Richardson, J. Wang, T. Zhang, S. Moran, S. Sayols, M. Soleimani, D. Tamborero, N. Lopez-Bigas, P. Ross-Macdonald, M. Esteller, N. S. Gray, D. A. Haber, M. R. Stratton, C. H. Benes, L. F. A. Wessels, J. Saez-Rodriguez, U. McDermott, M. J. Garnett, Cell. 166, 740-754 (2016). 7. M. Ghandi, F. W. Huang, J. Jane-Valbuena, G. V. Kryukov, C. C. Lo, E. R. McDonald, J. Barretina, E. T. Gelfand, C. M. Bielski, H. Li, K. Hu, A. Y. Andreev-Drakhlin, J. Kim, J. M. Hess, B. J. Haas, F. Aguet, B. A. Weir, M. V. Rothberg, B. R. Paolella, M. S. Lawrence, R. Akbani, Y. Lu, H. L. Tiv, P. C. Gokhale, A. de Weck, A. A. Mansour, C. Oh, J. Shih, K. Hadi, Y. Rosen, J. Bistline, K. Venkatesan, A. Reddy, D. Sonkin, M. Liu, J. Lehar, J. M. Kom, D. A. Porter, M. D. Jones, J. Golji, G. Caponigro, J. E. Taylor, C. M. Dunning, A. L. Creech, A. C. Warren, J. M. McFarland, M. Zamanighomi, A. Kauffmann, N. Stransky, M. Imielinski, Y. E. Maruvka, A. D. Cherniack, A. Tshemiak, F. Vazquez, J. D. Jaffe, A. A. Lane, D. M. Weinstock, C. M. Johannessen, M. P. Morrissey, F. Stegmeier, R. Schlegel, W. C. Hahn, G. Getz, G. B. Mills, J. S. Boehm, T. R. Golub, L. A. Garraway, W. R. Sellers, Nature. 569, 503-508 (2019). 8. M. A. Seeliger, M. Young, M. N. Henderson, P. Pellicena, D. S. King, A. M. Falick, J. Kuriyan, Protein Sci. 14, 3135-3139 (2005). 9. E. Gasteiger, C. Hoogland, A. Gattiker, S. Duvaud, M. R. Wilkins, R. D. Appel, A. Bairoch, The Proteomics Protocols Handbook, 571-607 (2005). 10. N. Vajpai, A. Strauss, G. Fendrich, S. W. Cowan-Jacob, P. W. Manley, W. Jahnke, S. Grzesiek, Biomol Nmr Assigm. 2, 41-42 (2008). 11. M. A. Fabian, W. H. Biggs, D. K. Treiber, C. E. Atteridge, M. D. Azimioara, M. G. Benedetti, T. A. Carter, P. Ciceri, P. T. Edeen, M. Floyd, J. M. Ford, M. Galvin, J. L. Gerlach, R. M. Grotzfeld, S. Herrgard, D. E. Insko, M. A. Insko, A. G. Lai, J.-M. Lelias, S. A. Mehta, Z. V. Milanov, A. M. Velasco, L. M. Wodicka, H. K. Patel, P. P. Zarrinkar, D. J. Lockhart, Nat Biotechnol. 23, 329-336 (2005). 12. F. Carles, S. Bourg, C. Meyer, P. Bonnet, Molecules. 23, 908 (2018). 13. G. Weng, C. Shen, D. Cao, J. Gao, X. Dong, Q. He, B. Yang, D. Li, J. Wu, T. Hou, Nucleic Acids Res. 49, gkaa807- (2020). 14. A. Daina, O. Michielin, V. Zoete, Sci Rep-uk. 7, 42717 (2017). 15. X. Chen, A. Murawski, K. Patel, C. L. Crespi, P. V. Balimane, Pharmaceut Res. 25, 1511-1520 (2008).

### Example 3: Chemical Synthesis Methods

Nuclear magnetic resonance (NMR) spectra were recorded on a Bruker spectrometer at 400 MHz or on a Bruker spectrometer at 600 MHz. Chemical shifts were reported as parts per million (ppm) from solvent references. Liquid chromatography-mass spectrometry (LC-MS) was performed on a Waters Xevo G2-XS QTof (0.6 mL/min) using an ACQUITY UPLC BEH C18 column (Waters) and a water/acetonitrile gradient (0.05% formic acid) using Optima LC-MS grade solvents (Fisher Scientific). All other solvents (Fisher Scientific, Millipore Sigma) and commercially available reagents were used without further purification. Analytical thin-layer chromatography was performed with silica gel 60 F254 glass plates (Millipore Sigma). Flash chromatography was performed with RediSep Rf normal-phase silica flash columns using a CombiFlash Rf+(Teledyne ISCO). Preparative high-performance liquid chromatography (HPLC) was performed on an AutoPurification System using an XBridge BEH C18 OBD Prep Column (Waters) or a CombiFlash EZ Prep using a RediSep C18 Prep HPLC Column (Teledyne ISCO) or a 50-g RediSep Gold C18 flash chromatography column with a water/acetonitrile gradient (0.1% formic acid or 0.1% trifluoroacetic acid). Microwave reactions were performed using a Discover SP (CEM).

Reagents and conditions (FIG. 14). (a) HATU, DIPEA, DMF, rt, 80%. (b) DIPEA, IPA, 140° C., 91%. (c) K₃PO₄, Pd(PPh₃)₄, toluene, 110° C., 40%. (d) LiOH·H₂O, H₂O, MeOH, 98%. (e) HATU, DIPEA, DMF, rt, 64-91%. (f) TFA, CH₂Cl₂, rt. (g) HATU, DIPEA, DMF, rt, 71-92% over two steps. (h) TFA, CH₂Cl₂, rt, 72-81%. Abbreviations: HATU, 1-[bis(dimethylamino)methylene]-1H-1,2,3-triazolo[4,5-b]pyridinium 3-oxide hexafluorophosphate; DIPEA, N,N-diisopropylethylamine; DMF, N,N-dimethylformamide; IPA, isopropyl alcohol; TFA, trifluoroacetic acid.

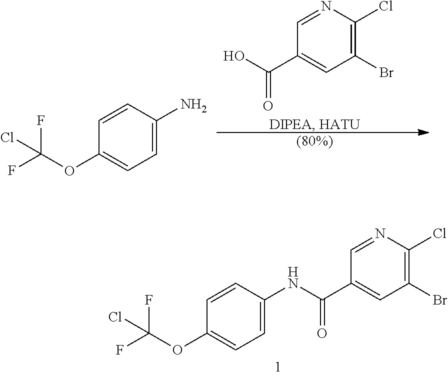 [SMILES: Nc1ccc(OC(F)(F)Cl)cc1 ; O=C(O)c1cnc(Cl)c(Br)c1 ; O=C(Nc1ccc(OC(F)(F)Cl)cc1)c1cnc(Cl)c(Br)c1]

Compound 1. To a mixture of 4-(chlorodifluoromethoxy)aniline (683 mg, 3.53 mmol) and 5-bromo-6-chloropicolinic acid (1001 mg, 4.23 mmol) in N,N-dimethylformamide (17.6 mL) was added N,N-diisopropylethylamine (614 μL, 3.53 mmol). The solution was cooled in an ice-water bath before the addition of 1-[bis(dimethylamino)methylene]-1H-1,2,3-triazolo[4,5-b]pyridinium 3-oxide hexafluorophosphate (1744 mg, 4.59 mmol) and stirred at room temperature overnight. The mixture was partitioned between ethyl acetate and water and the organic layer was washed with water (4×) and brine (2×), dried over sodium sulfate, filtered, and concentrated in vacuo. The crude was purified by flash chromatography over silica gel eluting with a gradient from 0% ethyl acetate-hexanes to 20% ethyl acetate-hexanes to afford compound 1 (1162 mg, 2.82 mmol, 80% yield) as a beige solid.

¹H NMR (400 MHz, DMSO-d₆) δ 10.69 (s, 1H), 8.92 (d, J=2.2 Hz, 1H), 8.73 (d, J=2.1 Hz, 1H), 7.87 (d, J=9.1 Hz, 2H), 7.39 (d, J=9.2 Hz, 2H). ¹³C NMR (100 MHz, DMSO-d₆) δ 161.8, 151.9, 147.8, 145.4, 141.8, 137.7, 130.9, 124.9 (t, J_{C-F}=287.1 Hz), 122.0 (2C), 121.7 (2C), 119.3. ¹⁹F NMR (376 MHz, DMSO-d₆) δ −24.8. HRMS (m/z): calculated for C₁₃H₈BrCl₂F₂N₂O₂⁺ [M+H]⁺ 410.9109, found 410.9123. TLC: R_{f}=0.4 (20% ethyl acetate-hexanes, UV).

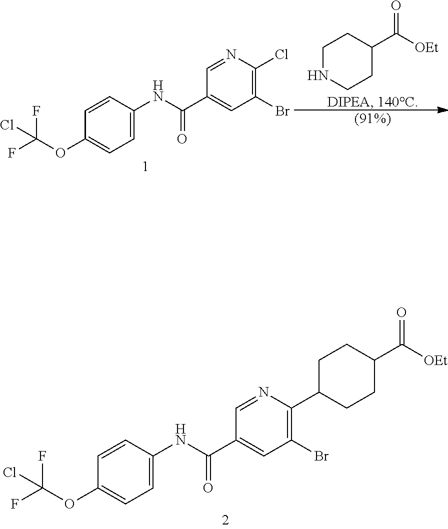 [SMILES: O=C(Nc1ccc(OC(F)(F)Cl)cc1)c1cnc(Cl)c(Br)c1 ; CCOC(=O)C1CCNCC1 ; CCOC(=O)C1CCC(c2ncc(C(=O)Nc3ccc(OC(F)(F)Cl)cc3)cc2Br)CC1]

Compound 2. To a mixture of compound 1 (1157 mg, 2.81 mmol) and ethyl 4-piperidinecarboxylate (0.563 mL, 3.65 mmol) in isopropanol (2.81 mL) was added N,N-diisopropylethylamine (2.45 mL, 14.0 mmol) in a microwave reaction vial. The reaction was heated in a microwave reactor at 140° C. for 1 h. The mixture was cooled to room temperature and diluted with ethyl acetate. The organic layer was washed with water (4×), 1 N HCl (2×), and brine (2×), dried over sodium sulfate, filtered, and concentrated in vacuo. The crude was purified by flash chromatography over silica gel eluting with a gradient from 0% ethyl acetate-hexanes to 30% ethyl acetate-hexanes to afford compound 2 (1354 mg, 2.54 mmol, 91% yield) as a white solid.

¹H NMR (400 MHz, DMSO-d₆) δ 10.40 (s, 1H), 8.78 (d, J=2.1 Hz, 1H), 8.44 (d, J=2.1 Hz, 1H), 7.86 (d, J=9.2 Hz, 2H), 7.35 (d, J=9.2 Hz, 2H), 4.09 (q, J=7.1 Hz, 2H), 3.98-3.75 (m, 2H), 3.08-2.87 (m, 2H), 2.66-2.53 (m, 1H), 2.04-1.88 (m, 2H), 1.82-1.62 (m, 2H), 1.20 (t, J=7.1 Hz, 3H). ¹³C NMR (100 MHz, DMSO-d₆) δ 174.1, 162.6, 160.5, 146.4, 145.1, 141.5, 138.2, 125.0 (t, J_{C-F}=287.0 Hz), 124.2, 121.9 (2C), 121.5 (2C), 109.5, 59.9, 48.3 (2C), 40.0, 27.7 (2C), 14.1. ¹⁹F NMR (376 MHz, DMSO-d₆) δ −24.7. HRMS (m/z): calculated for C₂₁H₂₂BrClF₂N₃O₄⁺ [M+H]⁺ 532.0445, found 532.0473. TLC: R_{f}=0.4 (30% ethyl acetate-hexanes, UV).

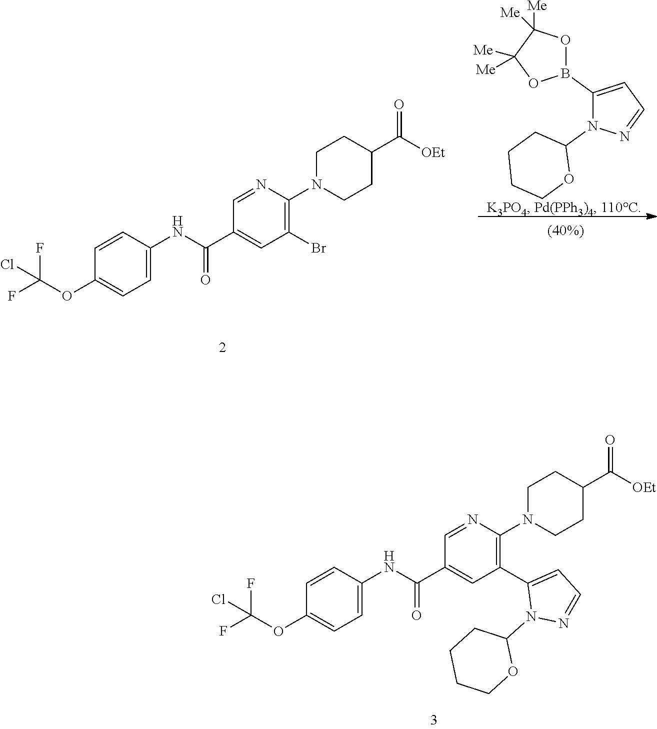 [SMILES: CCOC(=O)C1CCN(c2ncc(C(=O)Nc3ccc(OC(F)(F)Cl)cc3)cc2Br)CC1 ; CC1(C)OB(c2ccnn2C2CCCCO2)OC1(C)C ; CCOC(=O)C1CCN(c2ncc(C(=O)Nc3ccc(OC(F)(F)Cl)cc3)cc2-c2ccnn2C2CCCCO2)CC1]

Compound 3. To a mixture of compound 2 (600 mg, 1.13 mmol), 1-(tetrahydro-2H-pyran-2-yl)-1H-pyrazole-4-boronic acid pinacol ester (407 mg, 1.46 mmol), and K₃PO₄ (717 mg, 3.38 mmol) in toluene (1.13 mL) was added Pd(PPh₃)₄ (65 mg, 0.056 mmol). The reaction was sparged with argon for 5 min before stirring at 110° C. for 2 h. The mixture was diluted with ethyl acetate and the organic layer was washed with saturated sodium bicarbonate, water (2×), and brine (2×), dried over sodium sulfate, filtered, and concentrated in vacuo. The crude was purified by flash chromatography over silica gel eluting with a gradient from 0% ethyl acetate-hexanes to 50% ethyl acetate-hexanes to afford compound 3 (273 mg, 0.452 mmol, 40% yield) as a white solid.

¹H NMR (400 MHz, DMSO-d₆) δ 10.34 (s, 1H), 8.84 (d, J=2.4 Hz, 1H), 8.09 (d, J=2.5 Hz, 1H), 7.87 (d, J=9.2 Hz, 2H), 7.66 (d, J=1.7 Hz, 1H), 7.35 (d, J=8.7 Hz, 2H), 6.48 (d, J=1.8 Hz, 1H), 5.16 (dd, J=9.8, 2.4 Hz, 1H), 4.04 (q, J=7.1 Hz, 2H), 3.90-3.74 (m, 1H), 3.73-3.51 (m, 2H), 3.38-3.29 (m, 1H), 2.94-2.70 (m, 2H), 2.60-2.43 (m, 1H), 2.43-2.24 (m, 1H), 2.04-1.91 (m, 1H), 1.90-1.80 (m, 1H), 1.79-1.66 (m, 2H), 1.63-1.53 (m, 1H), 1.53-1.38 (m, 4H), 1.16 (t, J=7.1 Hz, 3H). ¹³C NMR (100 MHz, DMSO-d₆) δ 174.1, 163.6, 160.2, 148.3, 145.0, 140.7, 140.0, 139.5, 138.4, 125.0 (t, J_{C-F}=287.0 Hz), 121.8 (2C), 121.5 (2C), 120.9, 112.5, 106.6, 83.9, 66.7, 59.9, 47.0, 46.8, 40.0, 29.1, 27.5, 27.3, 24.5, 22.1, 14.1. ¹⁹F NMR (376 MHz, DMSO-d₆) δ −24.7. HRMS (m/z): calculated for C₂₉H₃₃ClF₂N₅O₅⁺ [M+H]⁺ 604.2133, found 604.2150. TLC: R_{f}=0.5 (50% ethyl acetate-hexanes, UV).

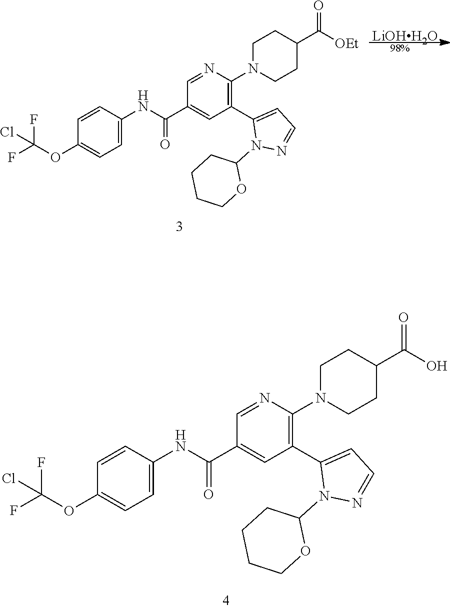

Compound 4. To a mixture of compound 3 (60 mg, 0.099 mmol) in methanol (2 mL) and water (0.2 mL) was added lithium hydroxide monohydrate (12.5 mg, 0.298 mmol). The reaction was stirred at room temperature overnight. The mixture was concentrated in vacuo and partitioned between ethyl acetate and 5% citric acid. The aqueous layer was extracted with ethyl acetate (3×), and the combined organics were washed with water (4×) and brine (2×), dried over sodium sulfate, filtered, and concentrated in vacuo. The crude was purified by flash chromatography over silica gel eluting with a gradient from 0% methanol-ethyl acetate to 5% methanol-ethyl acetate to afford compound 4 (56 mg, 0.097 mmol, 98% yield) as a white solid.

¹H NMR (400 MHz, DMSO-d₆) δ 12.19 (s, 1H), 10.33 (s, 1H), 8.84 (d, J=2.4 Hz, 1H), 8.09 (d, J=2.4 Hz, 1H), 7.86 (d, J=9.1 Hz, 2H), 7.66 (d, J=1.7 Hz, 1H), 7.35 (d, J=8.9 Hz, 2H), 6.48 (d, J=1.8 Hz, 1H), 5.16 (dd, J=9.7, 2.4 Hz, 1H), 3.88-3.77 (m, 1H), 3.70-3.53 (m, 2H), 3.38-3.33 (m, 1H), 2.90-2.72 (m, 2H), 2.46-2.38 (m, 1H), 2.38-2.27 (m, 1H), 2.02-1.91 (m, 1H), 1.91-1.80 (m, 1H), 1.78-1.65 (m, 2H), 1.64-1.52 (m, 1H), 1.52-1.39 (m, 4H). ¹³C NMR (100 MHz, DMSO-d₆) δ 175.8, 163.6, 160.3, 148.3, 145.0, 140.7, 140.0, 139.4, 138.4, 125.0 (t, J_{C-F}=286.9 Hz), 121.8 (2C), 121.5 (2C), 120.8, 112.4, 106.6, 83.9, 66.7, 47.1, 46.9, 40.0, 29.1, 27.6, 27.4, 24.5, 22.1. ¹⁹F NMR (376 MHz, DMSO-d₆) δ −24.7. HRMS (m/z): calculated for C₂₇H₂₉ClF₂N₅O₅⁺ [M+H]⁺ 576.1820, found 576.1816. TLC: R_{f}=0.5 (5% methanol-ethyl acetate, UV).

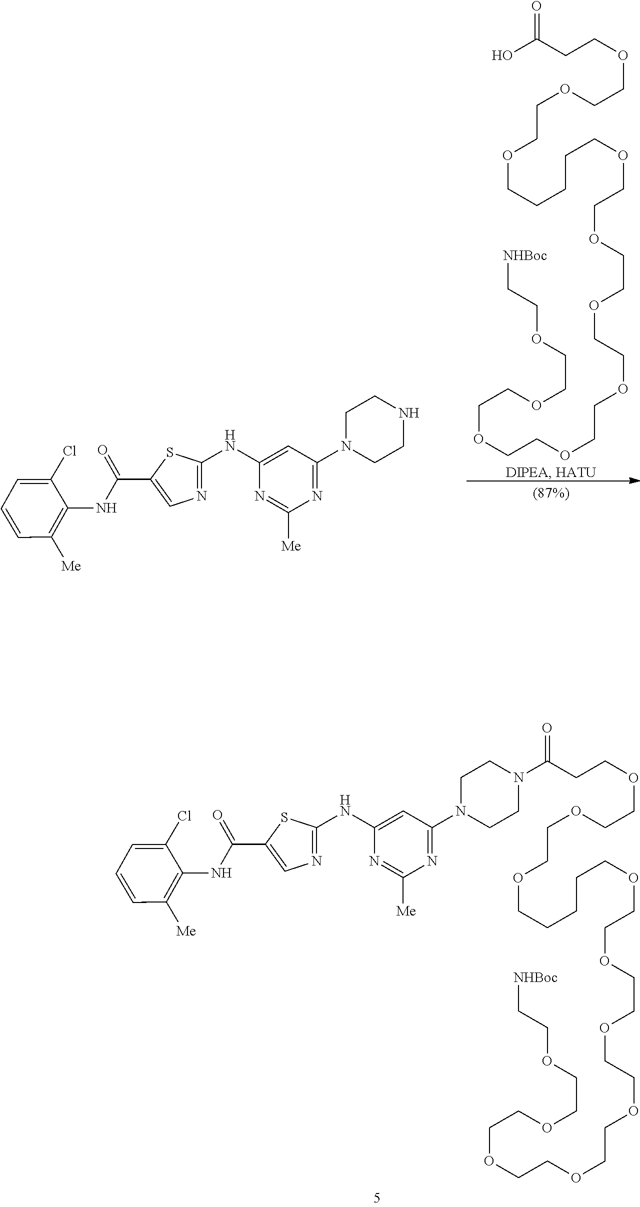 [SMILES: Cc1nc(Nc2ncc(C(=O)Nc3c(C)cccc3Cl)s2)cc(N2CCNCC2)n1 ; CC(C)(C)OC(=O)NCCOCCOCCOCCOCCOCCOCCOCCOCCCCCOCCOCCOCCC(=O)O ; Cc1nc(Nc2ncc(C(=O)Nc3c(C)cccc3Cl)s2)cc(N2CCN(C(=O)CCOCCOCCOCCCCCOCCOCCOCCOCCOCCOCCOCCOCCNC(=O)OC(C)(C)C)CC2)n1]

Compound 5. To a mixture of N-deshydroxyethyl dasatinib (100 mg, 0.225 mmol) and t-Boc-N-amido-PEG₁₂-acid (161.69 mg, 0.225 mmol) in N,N-dimethylformamide (1.13 mL) was added N,N-diisopropylethylamine (118 μL, 0.676 mmol). The solution was cooled in an ice-water bath before the addition of 1-[bis(dimethylamino)methylene]-1H-1,2,3-triazolo[4,5-b]pyridinium 3-oxide hexafluorophosphate (94 mg, 0.248 mmol) and stirred at room temperature overnight. The mixture was partitioned between ethyl acetate and water and the organic layer was washed with saturated sodium bicarbonate (3×), water (4×) and brine (2×), dried over sodium sulfate, filtered, and concentrated in vacuo. The crude was purified by flash chromatography over silica gel eluting with a gradient from 0% methanol-dichloromethane to 20% methanol-dichloromethane to afford compound 5 (223 mg, 0.195 mmol, 87% yield) as a pale yellow semisolid.

¹H NMR (600 MHz, DMSO-d₆) δ 11.50 (s, 1H), 9.88 (s, 1H), 8.22 (s, 1H), z7-7.34 (m, 1H), 7.34-7.19 (m, 2H), 6.81-6.63 (m, 1H), 6.06 (s, 1H), 3.65 (t, J=6.6 Hz, 2H), 3.63-3.52 (m, 8H), 3.52-3.42 (m, 44H), 3.37 (t, J=6.1 Hz, 2H), 3.12-2.97 (m, 2H), 2.62 (t, J=6.6 Hz, 2H), 2.42 (s, 3H), 2.24 (s, 3H), 1.37 (s, 9H). ¹³C NMR (151 MHz, DMSO-d₆) δ 169.1, 165.2, 162.5, 162.2, 159.9, 157.0, 155.6, 140.8, 138.8, 133.5, 132.4, 129.0, 128.2, 127.0, 125.8, 82.7, 77.6, 70.1-69.3 (m, 22C), 69.2, 66.8, 44.3, 43.5, 43.2, 40.5, 39.5, 32.9, 28.2 (3C), 25.6, 18.3. HRMS (m/z): calculated for C₅₂H₈₄ClN₈O₁₆S⁺ [M+H]⁺ 1143.5409, found 1143.5419. TLC: R_{f}=0.5 (20% methanol-dichloromethane).

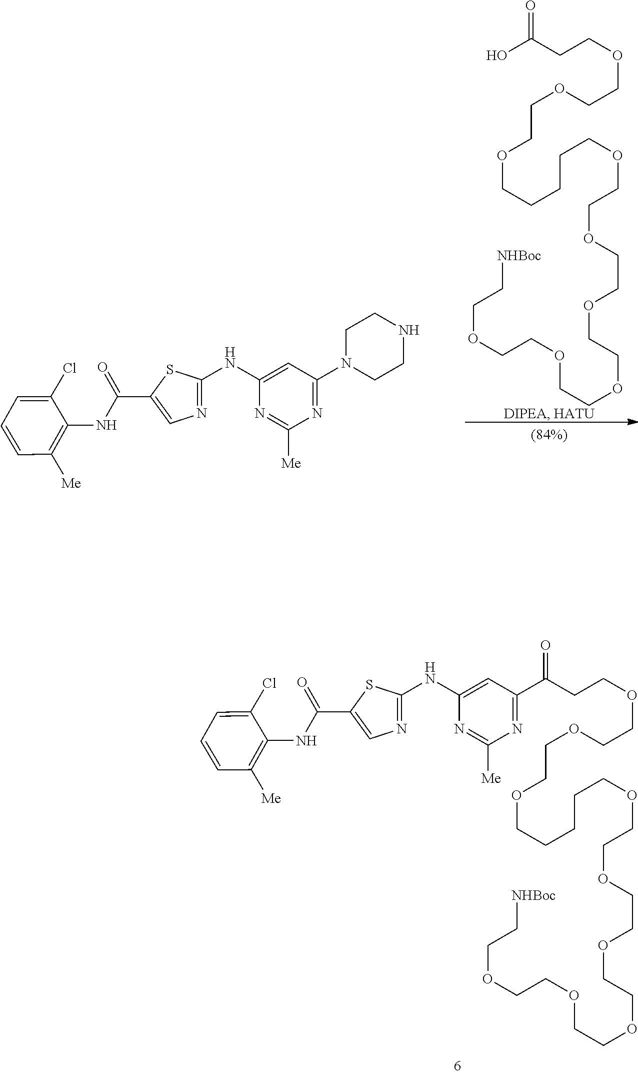 [SMILES: Cc1nc(Nc2ncc(C(=O)Nc3c(C)cccc3Cl)s2)cc(N2CCNCC2)n1 ; CC(C)(C)OC(=O)NCCOCCOCCOCCOCCOCCOCCCCCOCCOCCOCCC(=O)O ; Cc1nc(Nc2ncc(C(=O)Nc3c(C)cccc3Cl)s2)cc(C(=O)CCOCCOCCOCCCCCOCCOCCOCCOCCOCCOCCNC(=O)OC(C)(C)C)n1]

Compound 6. The same procedure as for compound 5, using t-Boc-N-amido-PEG₁₀-acid as starting material with scaled reagents, afforded compound 6 (200 mg, 0.189 mmol, 84% yield) as a pale yellow semisolid.

¹H NMR (600 MHz, DMSO-d₆) δ 11.51 (s, 1H), 9.88 (s, 1H), 8.22 (s, 1H), 7.45-7.35 (m, 1H), 7.33-7.21 (m, 2H), 6.80-6.68 (m, 1H), 6.06 (s, 1H), 3.65 (t, J=6.6 Hz, 2H), 3.62-3.52 (m, 8H), 3.52-3.43 (m, 36H), 3.36 (t, J=6.2 Hz, 2H), 3.10-3.00 (m, 2H), 2.62 (t, J=6.6 Hz, 2H), 2.42 (s, 3H), 2.24 (s, 3H), 1.36 (s, 9H). ¹³C NMR (151 MHz, DMSO-d₆) δ 169.1, 165.2, 162.5, 162.2, 159.9, 157.0, 155.6, 140.8, 138.8, 133.5, 132.4, 129.0, 128.2, 127.0, 125.8, 82.7, 77.6, 70.1-69.3 (m, 18C), 69.2, 66.8, 44.3, 43.5, 43.2, 40.5, 39.5, 32.9, 28.2 (3C), 25.6, 18.3. HRMS (m/z): calculated for C₄₈H₇₆ClN₈O₁₄S⁺ [M+H]⁺ 1055.4885, found 1055.4906. TLC: R_{f}=0.5 (20% methanol-dichloromethane).

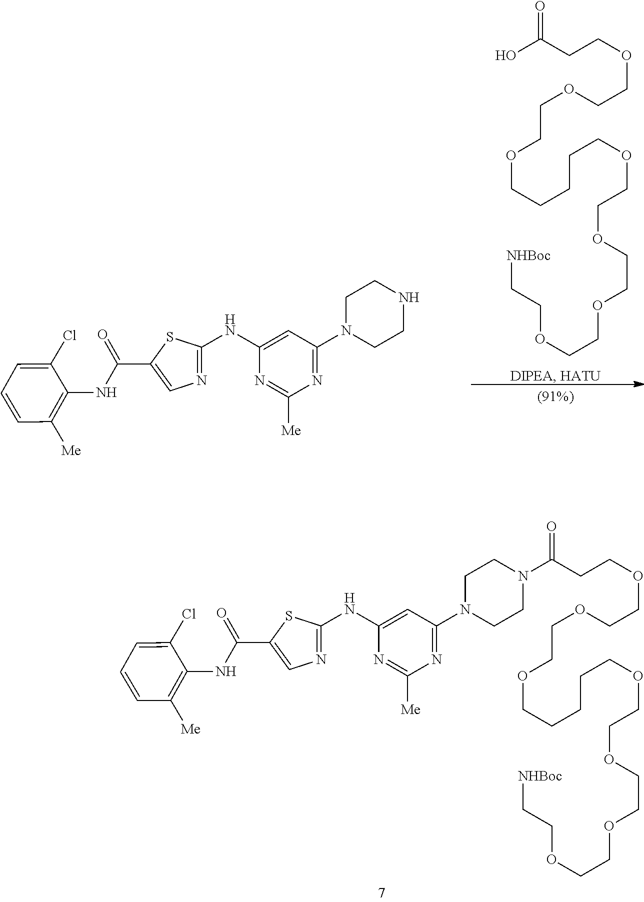 [SMILES: Cc1nc(Nc2ncc(C(=O)Nc3c(C)cccc3Cl)s2)cc(N2CCNCC2)n1 ; CC(C)(C)OC(=O)NCCOCCOCCOCCOCCCCCOCCOCCOCCC(=O)O ; Cc1nc(Nc2ncc(C(=O)Nc3c(C)cccc3Cl)s2)cc(N2CCN(C(=O)CCOCCOCCOCCCCCOCCOCCOCCOCCNC(=O)OC(C)(C)C)CC2)n1]

Compound 7. The same procedure as for compound 5, using t-Boc-N-amido-PEG₈-acid as starting material with scaled reagents, afforded compound 7 (159 mg, 0.164 mmol, 91% yield) as a white solid.

¹H NMR (600 MHz, DMSO-d₆) δ 11.52 (s, 1H), 9.88 (s, 1H), 8.22 (s, 1H), 7.48-7.34 (m, 1H), 7.34-7.17 (m, 2H), 6.83-6.64 (m, 1H), 6.06 (s, 1H), 3.65 (t, J=6.6 Hz, 2H), 3.62-3.52 (m, 8H), 3.52-3.43 (m, 28H), 3.36 (t, J=6.2 Hz, 2H), 3.10-2.97 (m, 2H), 2.62 (t, J=6.6 Hz, 2H), 2.42 (s, 3H), 2.24 (s, 3H), 1.36 (s, 9H). ¹³C NMR (151 MHz, DMSO-d₆) δ 169.1, 165.2, 162.5, 162.2, 159.9, 157.0, 155.6, 140.8, 138.8, 133.5, 132.4, 129.0, 128.2, 127.0, 125.7, 82.8, 77.6, 70.1-69.3 (m, 14C), 69.2, 66.8, 44.4, 43.6, 43.2, 40.5, 39.5, 32.9, 28.2 (3C), 25.6, 18.3. HRMS (m/z): calculated for C₄₄H₆₈ClN₈O₁₂S⁺ [M+H]⁺ 967.4360, found 967.4398. TLC: R_{f}=0.5 (20% methanol-dichloromethane).

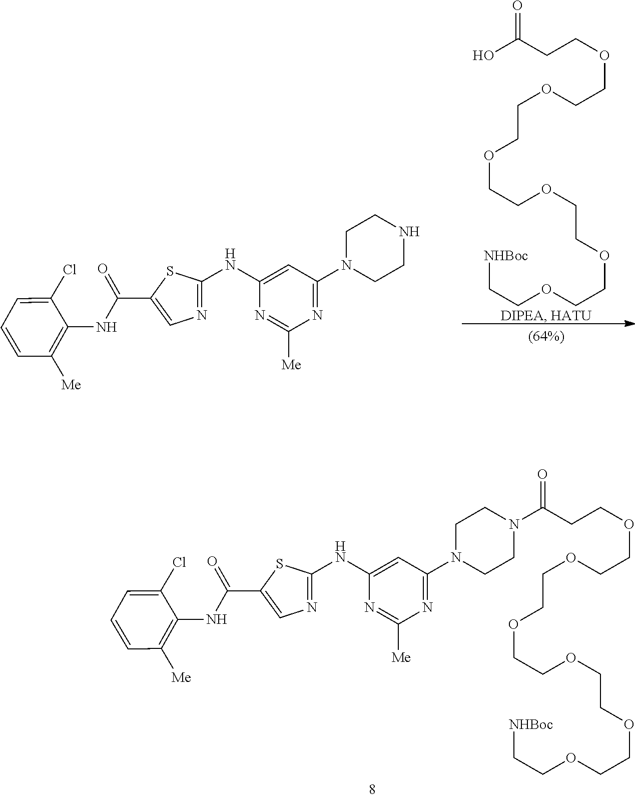 [SMILES: Cc1nc(Nc2ncc(C(=O)Nc3c(C)cccc3Cl)s2)cc(N2CCNCC2)n1 ; CC(C)(C)OC(=O)NCCOCCOCCOCCOCCOCCOCCC(=O)O ; Cc1nc(Nc2ncc(C(=O)Nc3c(C)cccc3Cl)s2)cc(N2CCN(C(=O)CCOCCOCCOCCOCCOCCOCCNC(=O)OC(C)(C)C)CC2)n1]

Compound 8. The same procedure as for compound 5, using t-Boc-N-amido-PEG6-acid as starting material with scaled reagents, with an additional purification by flash chromatography over silica gel eluting with a gradient from 10% methanol-ethyl acetate to 20% methanol-ethyl acetate afforded compound 8 (101 mg, 0.115 mmol, 64% yield) as a white solid.

¹H NMR (600 MHz, DMSO-d₆) δ 11.51 (s, 1H), 9.89 (s, 1H), 8.23 (s, 1H), 7.44-7.36 (m, 1H), 7.32-7.22 (m, 2H), 6.83-6.65 (m, 1H), 6.06 (s, 1H), 3.65 (t, J=6.6 Hz, 2H), 3.62-3.52 (m, 8H), 3.51-3.47 (m, 20H), 3.36 (t, J=6.1 Hz, 2H), 3.11-2.98 (m, 2H), 2.62 (t, J=6.6 Hz, 2H), 2.42 (s, 3H), 2.24 (s, 3H), 1.36 (s, 9H). ¹³C NMR (151 MHz, DMSO-d₆) δ 169.1, 165.2, 162.5, 162.2, 159.9, 157.0, 155.6, 140.8, 138.8, 133.5, 132.4, 129.0, 128.2, 127.0, 125.8, 82.8, 77.6, 70.1-69.3 (m, 10C), 69.2, 66.8, 44.3, 43.6, 43.2, 40.5, 39.5, 32.9, 28.2 (3C), 25.6, 18.3. HRMS (m/z): calculated for C₄₀H₆₀ClN₈O₁₀S⁺ [M+H]⁺ 879.3836, found 879.3857. TLC: R_{f}=0.6 (20% methanol-dichloromethane), R_{f}=0.5 (20% methanol-ethyl acetate).

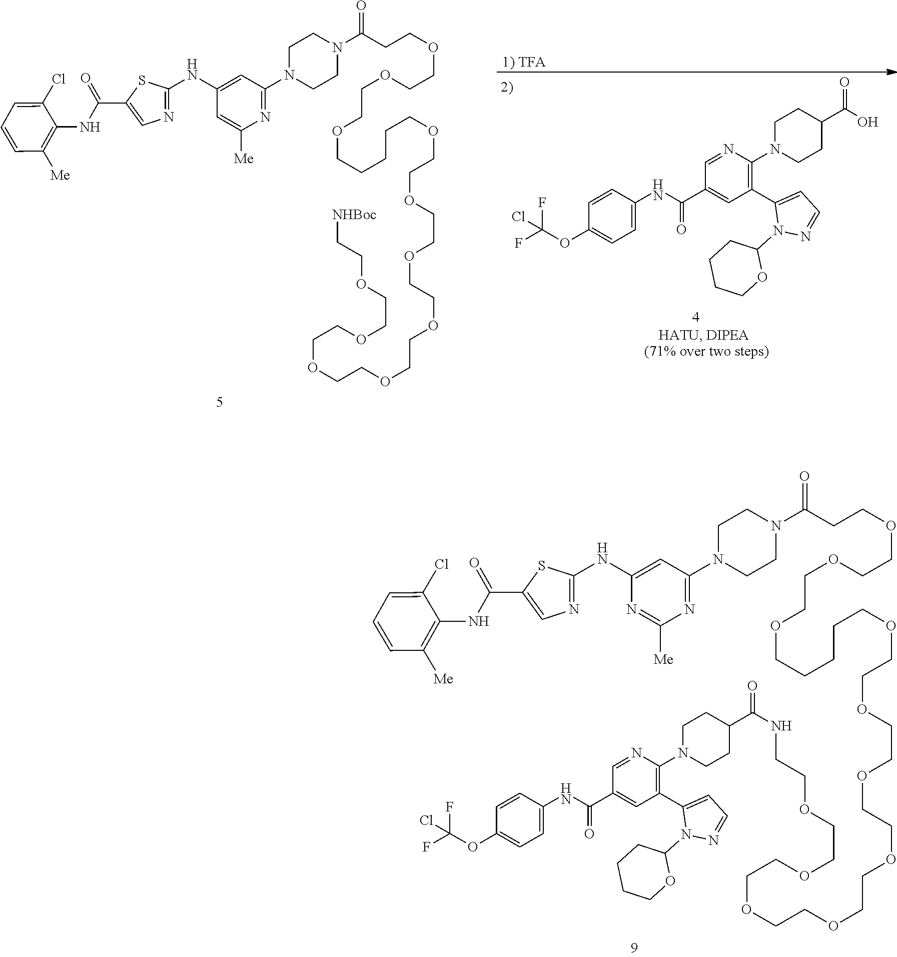 [SMILES: Cc1cc(Nc2ncc(C(=O)Nc3c(C)cccc3Cl)s2)cc(N2CCN(C(=O)CCOCCOCCOCCCCCOCCOCCOCCOCCOCCOCCOCCOCCNC(=O)OC(C)(C)C)CC2)n1 ; O=C(Nc1ccc(OC(F)(F)Cl)cc1)c1cnc(N2CCC(C(=O)O)CC2)c(-c2ccnn2C2CCCCO2)c1 ; Cc1nc(Nc2ncc(C(=O)Nc3c(C)cccc3Cl)s2)cc(N2CCN(C(=O)CCOCCOCCOCCCCCOCCOCCOCCOCCOCCOCCOCCOCCNC(=O)C3CCN(c4ncc(C(=O)Nc5ccc(OC(F)(F)Cl)cc5)cc4-c4ccnn4C4CCCCO4)CC3)CC2)n1]

Compound 9. To a mixture of compound 5 (100 mg, 0.0874 mmol) in dichloromethane (0.874 mL) was added trifluoroacetic acid (0.874 mL). The solution was stirred at room temperature for 1 h before concentrating in vacuo to afford a residue that was used directly in the next step. To a mixture of the crude amine, trifluoroacetic acid salt and compound 4 (55 mg, 0.0961 mmol) in N,N-dimethylformamide (0.874 mL) was added N,N-diisopropylethylamine (46 μL, 0.262 mmol). The solution was cooled in an ice-water bath before the addition of 1-[bis(dimethylamino)methylene]-1H-1,2,3-triazolo[4,5-b]pyridinium 3-oxide hexafluorophosphate (37 mg, 0.0961 mmol) and stirred at room temperature overnight. The mixture was partitioned between ethyl acetate and water and the organic layer was washed with water (4×) and brine (2×), dried over sodium sulfate, filtered, and concentrated in vacuo. The crude was purified by flash chromatography over silica gel as follows: the crude was dry loaded into silica gel and an initial elution with ethyl acetate was made to remove an impurity. Upon switching to a methanol-dichloromethane solvent system, the desired product immediately eluted with additional impurities from the column. Fractions containing the desired product were then re-subjected to flash chromatography over silica gel eluting with a gradient from 0% methanol-dichloromethane to 20% methanol-dichloromethane to afford compound 6 (99 mg, 0.0618 mmol, 71% yield over two steps) as a pale yellow solid.

¹H NMR (600 MHz, DMSO-d₆) δ 11.50 (s, 1H), 10.33 (s, 1H), 9.88 (s, 1H), 8.84 (d, J=2.4 Hz, 1H), 8.22 (s, 1H), 8.08 (d, J=2.4 Hz, 1H), 7.90-7.83 (m, 2H), 7.83-7.75 (m, 1H), 7.65 (d, J=1.7 Hz, 1H), 7.43-7.37 (m, 1H), 7.34 (d, J=9.2 Hz, 2H), 7.31-7.19 (m, 2H), 6.47 (d, J=1.7 Hz, 1H), 6.06 (s, 1H), 5.16 (dd, J=9.8, 2.5 Hz, 1H), 3.85-3.75 (m, 1H), 3.73-3.66 (m, 2H), 3.65 (t, J=6.6 Hz, 2H), 3.62-3.51 (m, 8H), 3.51-3.46 (m, 44H), 3.37 (t, J=6.0 Hz, 2H), 3.33-3.29 (m, 1H), 3.20-3.09 (m, 2H), 2.80-2.65 (m, 2H), 2.62 (t, J=6.6 Hz, 2H), 2.42 (s, 3H), 2.38-2.26 (m, 2H), 2.24 (s, 3H), 1.99-1.85 (m, 2H), 1.61-1.40 (m, 7H). ¹³C NMR (151 MHz, DMSO-d₆) δ 174.0, 169.1, 165.2, 163.6, 162.5, 162.2, 160.3, 159.9, 157.0, 148.3, 145.0, 140.8, 140.8, 140.0, 139.4, 138.8, 138.4, 133.5, 132.4, 129.0, 128.2, 127.0, 125.8, 125.0 (t, J_{C-F}=286.9 Hz), 121.9 (2C), 121.5 (2C), 120.8, 112.4, 106.6, 84.0, 82.7, 70.1-69.3 (m, 22C), 69.1, 66.8, 66.7, 47.4, 47.1, 44.3, 43.5, 43.2, 41.5, 40.5, 38.4, 32.9, 29.0, 28.1, 27.9, 25.6, 24.5, 22.1, 18.3. ¹⁹F NMR (564 MHz, DMSO-d₆) δ −24.7. HRMS (m/z): calculated for C₇₄H₁₀₂Cl₂F₂N₁₃O₁₈S⁺ [M+H]⁺ 1600.6526, found 1600.6644. TLC: R_{f}=0.0 (ethyl acetate), R_{f}=0.4 (20% methanol-dichloromethane).

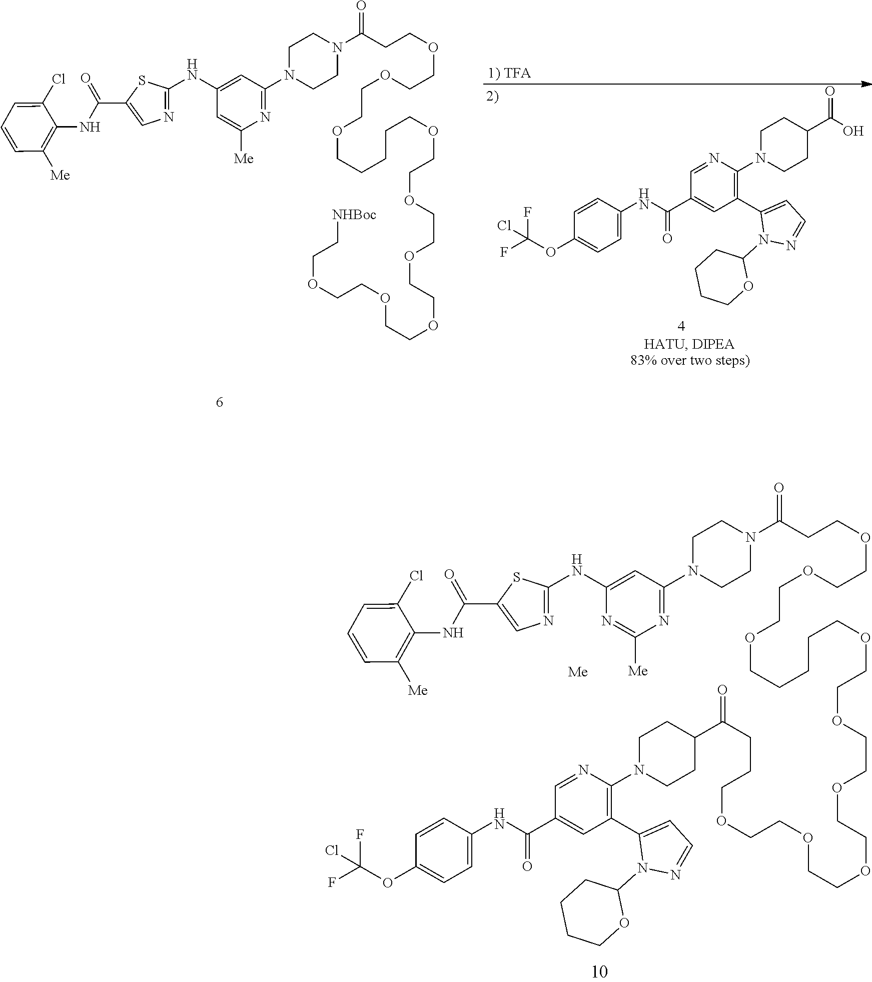 [SMILES: Cc1cc(Nc2ncc(C(=O)Nc3c(C)cccc3Cl)s2)cc(N2CCN(C(=O)CCOCCOCCOCCCCCOCCOCCOCCOCCOCCOCCNC(=O)OC(C)(C)C)CC2)n1 ; O=C(Nc1ccc(OC(F)(F)Cl)cc1)c1cnc(N2CCC(C(=O)O)CC2)c(-c2ccnn2C2CCCCO2)c1 ; Cc1nc(Nc2ncc(C(=O)Nc3c(C)cccc3Cl)s2)cc(N2CCN(C(=O)CCOCCOCCOCCCCCOCCOCCOCCOCCOCCOCCCC(=O)C3CCN(c4ncc(C(=O)Nc5ccc(OC(F)(F)Cl)cc5)cc4-c4ccnn4C4CCCCO4)CC3)CC2)n1 ; "Me"]

Compound 10. The same procedure as for compound 9, using compound 6 as starting material with scaled reagents, afforded compound 10 (213 mg, 0.141 mmol, 83% yield over two steps) as a pale yellow solid.

¹H NMR (600 MHz, DMSO-d₆) δ 11.51 (s, 1H), 10.33 (s, 1H), 9.88 (s, 1H), 8.84 (d, J=2.4 Hz, 1H), 8.22 (s, 1H), 8.08 (d, J=2.5 Hz, 1H), 7.90-7.84 (m, 2H), 7.84-7.77 (m, 1H), 7.65 (d, J=1.7 Hz, 1H), 7.43-7.37 (m, 1H), 7.34 (d, J=8.7 Hz, 2H), 7.31-7.22 (m, 2H), 6.47 (d, J=1.7 Hz, 1H), 6.06 (s, 1H), 5.16 (dd, J=9.7, 2.5 Hz, 1H), 3.84-3.75 (m, 1H), 3.72-3.66 (m, 2H), 3.64 (t, J=6.6 Hz, 2H), 3.62-3.51 (m, 8H), 3.51-3.44 (m, 36H), 3.37 (t, J=5.9 Hz, 2H), 3.33-3.29 (m, 1H), 3.19-3.13 (m, 2H), 2.81-2.65 (m, 2H), 2.62 (t, J=6.6 Hz, 2H), 2.42 (s, 3H), 2.39-2.26 (m, 2H), 2.24 (s, 3H), 1.99-1.85 (m, 2H), 1.62-1.40 (m, 7H). ¹³C NMR (151 MHz, DMSO-d₆) δ 174.0, 169.1, 165.2, 163.6, 162.5, 162.2, 160.3, 159.9, 157.0, 148.3, 145.0, 140.8, 140.8, 140.0, 139.4, 138.8, 138.4, 133.5, 132.4, 129.0, 128.2, 127.0, 125.8, 125.0 (t, J_{C-F}=286.9 Hz), 121.9 (2C), 121.5 (2C), 120.8, 112.4, 106.6, 84.0, 82.7, 70.1-69.3 (m, 18C), 69.1, 66.8, 66.8, 47.4, 47.1, 44.3, 43.5, 43.2, 41.5, 40.5, 38.4, 32.9, 29.0, 28.1, 27.9, 25.6, 24.6, 22.2, 18.3. ¹⁹F NMR (564 MHz, DMSO-d₆) δ −24.7. HRMS (m/z): calculated for C₇₀H₉₄Cl₂F₂N₁₃O₁₆S⁺ [M+H]⁺ 1512.6002, found 1512.6016. TLC: R_{f}=0.0 (ethyl acetate), R_{f}=0.4 (20% methanol-dichloromethane).

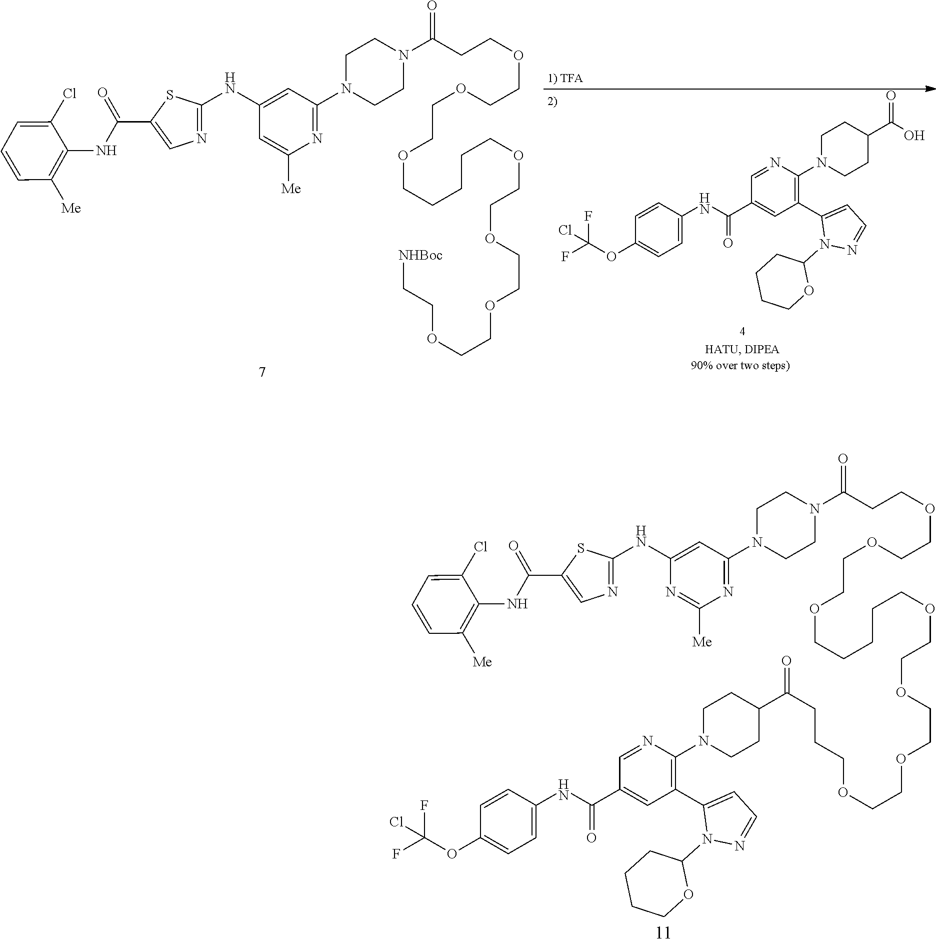

Compound 11. The same procedure as for compound 9, using compound 7 as starting material with scaled reagents, afforded compound 11 (185 mg, 0.130 mmol, 90% yield over two steps) as a pale yellow solid.

¹H NMR (600 MHz, DMSO-d₆) δ 11.51 (s, 1H), 10.34 (s, 1H), 9.88 (s, 1H), 8.84 (d, J=2.4 Hz, 1H), 8.22 (s, 1H), 8.08 (d, J=2.4 Hz, 1H), 7.89-7.84 (m, 2H), 7.84-7.78 (m, 1H), 7.65 (d, J=1.7 Hz, 1H), 7.42-7.37 (m, 1H), 7.34 (d, J=9.0 Hz, 2H), 7.31-7.22 (m, 2H), 6.47 (d, J=1.8 Hz, 1H), 6.06 (s, 1H), 5.16 (dd, J=9.7, 2.5 Hz, 1H), 3.85-3.75 (m, 1H), 3.72-3.66 (m, 2H), 3.64 (t, J=6.6 Hz, 2H), 3.62-3.51 (m, 8H), 3.51-3.46 (m, 28H), 3.37 (t, J=5.9 Hz, 2H), 3.32-3.29 (m, 1H), 3.19-3.12 (m, 2H), 2.78-2.65 (m, 2H), 2.62 (t, J=6.6 Hz, 2H), 2.42 (s, 3H), 2.38-2.26 (m, 2H), 2.24 (s, 3H), 2.00-1.84 (m, 2H), 1.62-1.39 (m, 7H). ¹³C NMR (151 MHz, DMSO-d₆) δ 174.0, 169.1, 165.2, 163.6, 162.5, 162.2, 160.3, 159.9, 157.0, 148.3, 145.0, 140.8, 140.8, 140.0, 139.4, 138.8, 138.4, 133.5, 132.4, 129.0, 128.2, 127.0, 125.8, 125.0 (t, J_{C-F}=286.9 Hz), 121.9 (2C), 121.5 (2C), 120.8, 112.4, 106.6, 84.0, 82.7, 70.1-69.3 (m, 14C), 69.1, 66.8, 66.8, 47.4, 47.1, 44.3, 43.5, 43.2, 41.5, 40.5, 38.4, 32.9, 29.0, 28.1, 27.9, 25.6, 24.6, 22.2, 18.3. ¹⁹F NMR (564 MHz, DMSO-d₆) δ −24.7. HRMS (m/z): calculated for C₆₆H₈₆Cl₂F₂N₁₃O₁₄S⁺ [M+H]⁺ 1424.5478, found 1424.5490. TLC: R_{f}=0.0 (ethyl acetate), R_{f}=0.6 (20% methanol-dichloromethane).

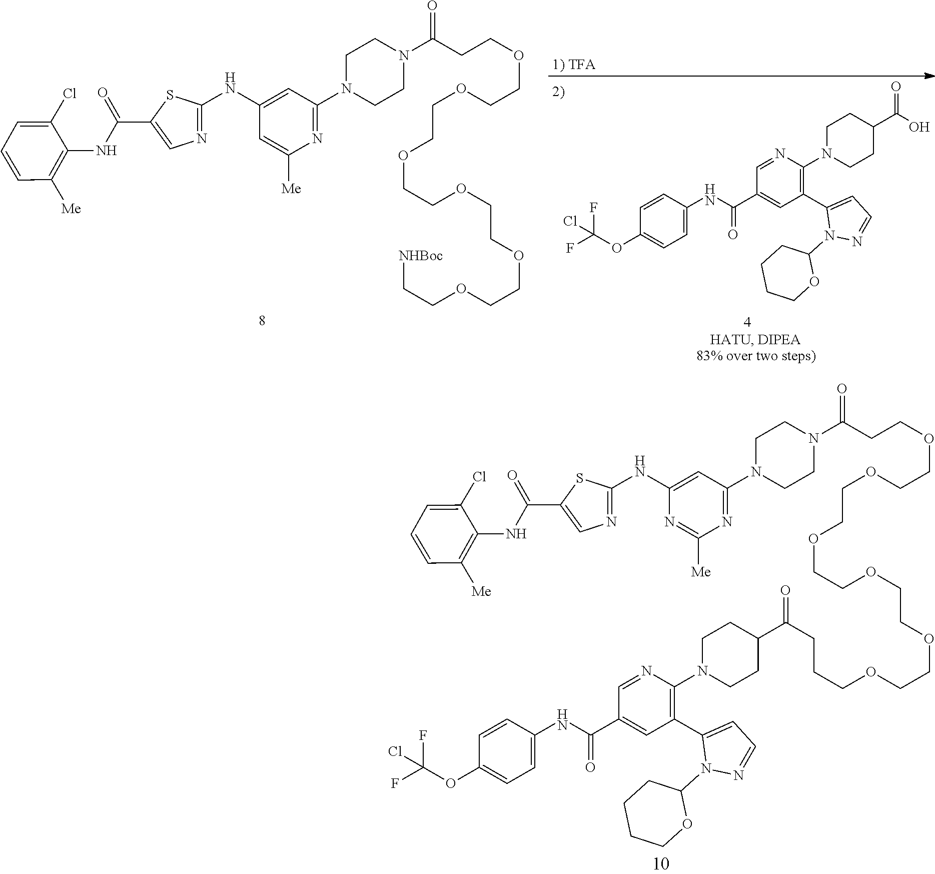 [SMILES: Cc1cc(Nc2ncc(C(=O)Nc3c(C)cccc3Cl)s2)cc(N2CCN(C(=O)CCOCCOCCOCCOCCOCCOCCNC(=O)OC(C)(C)C)CC2)n1 ; O=C(Nc1ccc(OC(F)(F)Cl)cc1)c1cnc(N2CCC(C(=O)O)CC2)c(-c2ccnn2C2CCCCO2)c1 ; Cc1nc(Nc2ncc(C(=O)Nc3c(C)cccc3Cl)s2)cc(N2CCN(C(=O)CCOCCOCCOCCOCCOCCOCCCC(=O)C3CCN(c4ncc(C(=O)Nc5ccc(OC(F)(F)Cl)cc5)cc4-c4ccnn4C4CCCCO4)CC3)CC2)n1]

Compound 12. The same procedure as for compound 9, using compound 8 as starting material with scaled reagents, afforded compound 12 (115 mg, 0.0860 mmol, 92% yield over two steps) as a pale yellow solid.

¹H NMR (600 MHz, DMSO-d₆) δ 11.51 (s, 1H), 10.33 (s, 1H), 9.88 (s, 1H), 8.84 (d, J=2.4 Hz, 1H), 8.22 (s, 1H), 8.08 (d, J=2.4 Hz, 1H), 7.90-7.84 (m, 2H), 7.84-7.78 (m, 1H), 7.65 (d, J=1.7 Hz, 1H), 7.43-7.37 (m, 1H), 7.34 (d, J=9.0 Hz, 2H), 7.31-7.22 (m, 2H), 6.47 (d, J=1.8 Hz, 1H), 6.06 (s, 1H), 5.16 (dd, J=9.8, 2.5 Hz, 1H), 3.85-3.75 (m, 1H), 3.72-3.66 (m, 2H), 3.64 (t, J=6.6 Hz, 2H), 3.62-3.51 (m, 8H), 3.51-3.46 (m, 20H), 3.37 (t, J=5.9 Hz, 2H), 3.32-3.29 (m, 1H), 3.19-3.13 (m, 2H), 2.79-2.65 (m, 2H), 2.62 (t, J=6.6 Hz, 2H), 2.42 (s, 3H), 2.39-2.26 (m, 2H), 2.23 (s, 3H), 2.00-1.84 (m, 2H), 1.65-1.36 (m, 7H). ¹³C NMR (151 MHz, DMSO-d₆) δ 174.1, 169.1, 165.2, 163.6, 162.5, 162.2, 160.3, 159.9, 157.0, 148.3, 145.0, 140.8, 140.8, 140.0, 139.4, 138.8, 138.4, 133.5, 132.4, 129.0, 128.2, 127.0, 125.8, 125.0 (t, J_{C-F}=286.9 Hz), 121.9 (2C), 121.5 (2C), 120.8, 112.4, 106.6, 84.0, 82.7, 70.1-69.3 (m, 10C), 69.1, 66.8, 66.8, 47.4, 47.1, 44.3, 43.5, 43.2, 41.5, 40.5, 38.4, 32.9, 29.0, 28.1, 27.9, 25.6, 24.6, 22.2, 18.3. ¹⁹F NMR (564 MHz, DMSO-d₆) δ −24.7. HRMS (m/z): calculated for C₆₂H₇₈Cl₂F₂N₁₃O₁₂S⁺ [M+H]⁺ 1336.4953, found 1336.4965. TLC: R_{f}=0.0 (ethyl acetate), R_{f}=0.6 (20% methanol-dichloromethane).

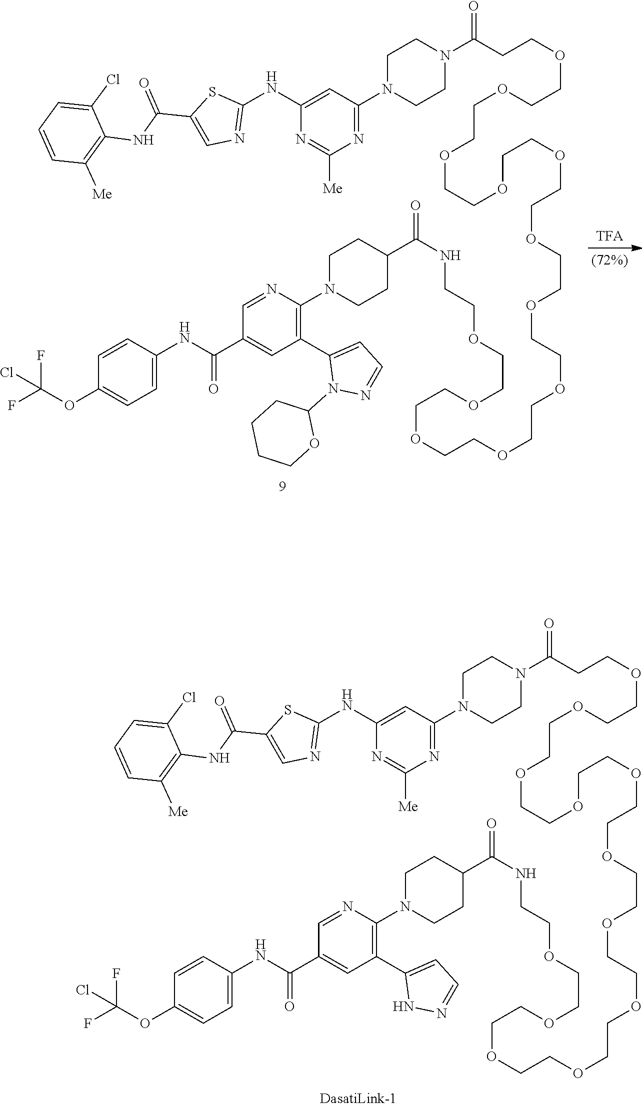 [SMILES: Cc1nc(Nc2ncc(C(=O)Nc3c(C)cccc3Cl)s2)cc(N2CCN(C(=O)CCOCCOCCOCCOCCOCCOCCOCCOCCOCCOCCOCCOCCNC(=O)C3CCN(c4ncc(C(=O)Nc5ccc(OC(F)(F)Cl)cc5)cc4-c4ccnn4C4CCCCO4)CC3)CC2)n1 ; Cc1nc(Nc2ncc(C(=O)Nc3c(C)cccc3Cl)s2)cc(N2CCN(C(=O)CCOCCOCCOCCOCCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

DasatiLink-1. To a mixture of compound 9 (47 mg, 0.0293 mmol) in dichloromethane (0.293 mL) was added trifluoroacetic acid (0.293 mL). The solution was stirred at room temperature for 6 h before concentrating in vacuo. The residue was partitioned between ethyl acetate and saturated sodium bicarbonate. The aqueous layer was extracted with ethyl acetate (3×) and the combined organics were washed with brine (2×), dried over sodium sulfate, filtered, and concentrated in vacuo. The crude was purified by flash chromatography over silica gel eluting with a gradient from 0% methanol-dichloromethane to 20% methanol-dichloromethane. Fractions containing the desired product were combined, concentrated in vacuo, and further purified by HPLC to afford DasatiLink-1 (32 mg, 0.0211 mmol, 72% yield) as a white solid.

¹H NMR (600 MHz, DMSO-d₆) δ 13.02 (s, 1H), 11.50 (s, 1H), 10.39 (s, 1H), 9.88 (s, 1H), 8.74 (d, J=2.4 Hz, 1H), 8.34 (s, 1H), 8.22 (s, 1H), 7.97-7.52 (m, 4H), 7.43-7.37 (m, 1H), 7.34 (d, J=8.7 Hz, 2H), 7.31-7.22 (m, 2H), 6.66 (s, 1H), 6.06 (s, 1H), 3.71-3.61 (m, 4H), 3.61-3.52 (m, 8H), 3.52-3.44 (m, 44H), 3.39 (t, J=6.0 Hz, 2H), 3.21-3.15 (m, 2H), 2.75-2.67 (m, 2H), 2.62 (t, J=6.6 Hz, 2H), 2.42 (s, 3H), 2.32-2.25 (m, 1H), 2.24 (s, 3H), 1.69-1.55 (m, 4H). ¹³C NMR (151 MHz, DMSO-d₆) δ 174.3, 169.1, 165.2, 164.1, 162.5, 162.2, 160.9, 159.9, 157.0, 148.4, 146.5, 144.9, 140.8, 138.8, 138.5, 137.6, 133.5, 132.4, 129.7, 129.0, 128.2, 127.0, 125.8, 125.0 (t, J_{C-F}=286.9 Hz), 122.1, 121.9 (2C), 121.5 (2C), 118.2, 103.5, 82.8, 70.3-69.3 (m, 22C), 69.1, 66.8, 48.4 (2C), 44.4, 43.5, 43.2, 41.7, 40.5, 38.5, 32.9, 28.1 (2C), 25.6, 18.3. ¹⁹F NMR (564 MHz, DMSO-d₆) δ −24.7. HRMS (m/z): calculated for C₆₉H₉₄Cl₂F₂N₁₃O₁₇S⁺ [M+H]⁺ 1516.5951, found 1516.6038. TLC: R_{f}=0.5 (20% methanol-dichloromethane).

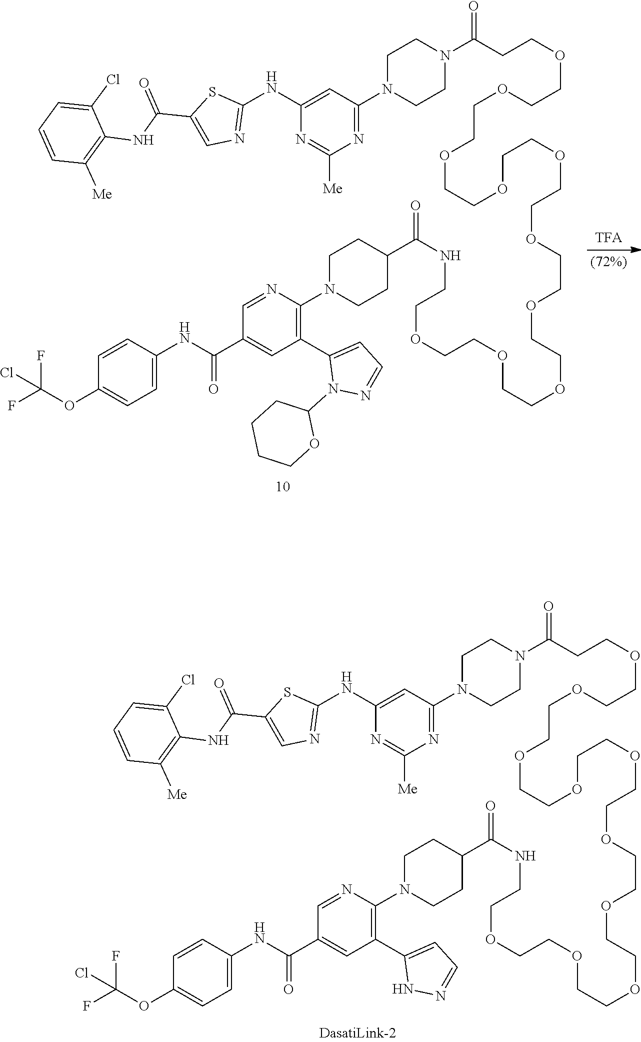 [SMILES: Cc1nc(Nc2ncc(C(=O)Nc3c(C)cccc3Cl)s2)cc(N2CCN(C(=O)CCOCCOCCOCCOCCOCCOCCOCCOCCOCCOCCNC(=O)C3CCN(c4ncc(C(=O)Nc5ccc(OC(F)(F)Cl)cc5)cc4-c4ccnn4C4CCCCO4)CC3)CC2)n1 ; Cc1nc(Nc2ncc(C(=O)Nc3c(C)cccc3Cl)s2)cc(N2CCN(C(=O)CCOCCOCCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

DasatiLink-2. The same procedure as for DasatiLink-1, using compound 10 as starting material with scaled reagents, afforded compound DasatiLink-2 (34 mg, 0.0238 mmol, 72% yield) as a white solid.

¹H NMR (600 MHz, DMSO-d₆) δ 13.04 (s, 1H), 11.52 (s, 1H), 10.41 (s, 1H), 9.89 (s, 1H), 8.74 (d, J=2.4 Hz, 1H), 8.34 (s, 1H), 8.22 (s, 1H), 7.97-7.51 (m, 4H), 7.42-7.38 (m, 1H), 7.34 (d, J=8.7 Hz, 2H), 7.31-7.22 (m, 2H), 6.66 (s, 1H), 6.06 (s, 1H), 3.71-3.62 (m, 4H), 3.62-3.52 (m, 8H), 3.52-3.46 (m, 36H), 3.39 (t, J=5.9 Hz, 2H), 3.22-3.15 (m, 2H), 2.76-2.66 (m, 2H), 2.62 (t, J=6.6 Hz, 2H), 2.42 (s, 3H), 2.32-2.25 (m, 1H), 2.24 (s, 3H), 1.74-1.52 (m, 4H). ¹³C NMR (151 MHz, DMSO-d₆) δ 174.3, 169.1, 165.2, 164.1, 162.5, 162.2, 161.0, 159.9, 157.0, 148.3, 146.4, 144.9, 140.8, 138.8, 138.5, 137.6, 133.5, 132.4, 129.6, 129.0, 128.2, 127.0, 125.8, 125.0 (t, J_{C-F}=286.9 Hz), 122.2, 121.9 (2C), 121.5 (2C), 118.3, 103.4, 82.8, 70.3-69.3 (m, 18C), 69.1, 66.8, 48.4 (2C), 44.3, 43.5, 43.2, 41.7, 40.5, 38.4, 32.9, 28.1 (2C), 25.6, 18.3. ¹⁹F NMR (564 MHz, DMSO-d₆) δ −24.7. HRMS (m/z): calculated for C₆₅H₈₆Cl₂F₂N₁₃O₁₅S⁺ [M+H]⁺ 1428.5427, found 1428.5439. TLC: R_{f}=0.6 (20% methanol-dichloromethane).

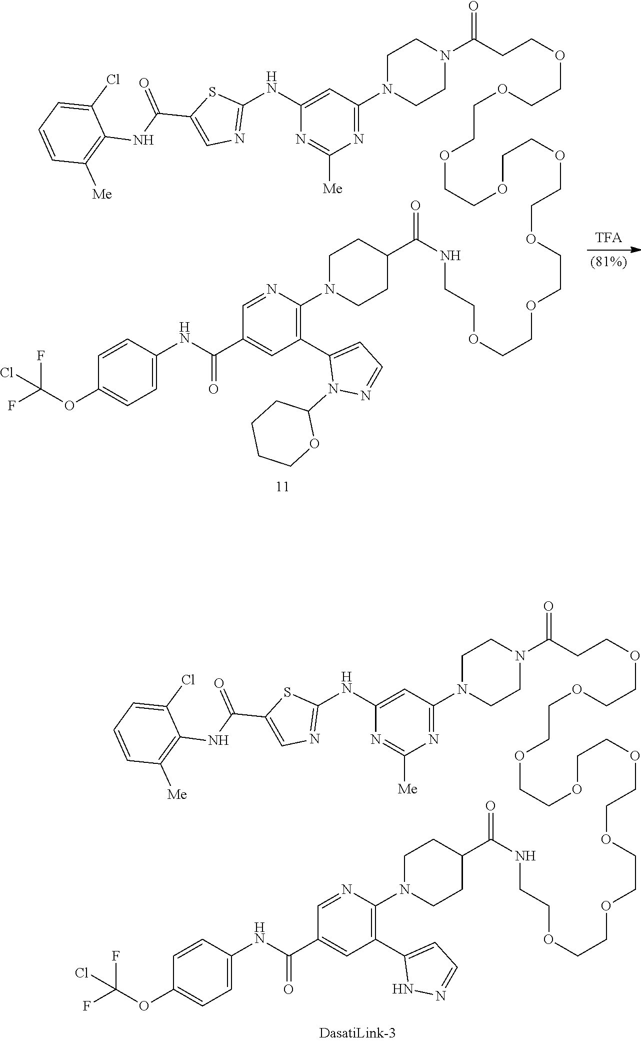 [SMILES: Cc1nc(Nc2ncc(C(=O)Nc3c(C)cccc3Cl)s2)cc(N2CCN(C(=O)CCOCCOCCOCCOCCOCCOCCOCCOCCNC(=O)C3CCN(c4ncc(C(=O)Nc5ccc(OC(F)(F)Cl)cc5)cc4-c4ccnn4C4CCCCO4)CC3)CC2)n1 ; Cc1nc(Nc2ncc(C(=O)Nc3c(C)cccc3Cl)s2)cc(N2CCN(C(=O)CCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

DasatiLink-3. The same procedure as for DasatiLink-1, using compound 11 as starting material with scaled reagents, afforded compound DasatiLink-3 (38 mg, 0.0283 mmol, 81% yield) as a white solid.

¹H NMR (600 MHz, DMSO-d₆) δ 13.05 (s, 1H), 11.53 (s, 1H), 10.42 (s, 1H), 9.90 (s, 1H), 8.74 (d, J=2.4 Hz, 1H), 8.33 (s, 1H), 8.23 (s, 1H), 7.99-7.52 (m, 4H), 7.44-7.37 (m, 1H), 7.34 (d, J=8.7 Hz, 2H), 7.31-7.22 (m, 2H), 6.66 (s, 1H), 6.07 (s, 1H), 3.69-3.62 (m, 4H), 3.62-3.51 (m, 8H), 3.51-3.46 (m, 28H), 3.39 (t, J=5.9 Hz, 2H), 3.21-3.16 (m, 2H), 2.77-2.66 (m, 2H), 2.61 (t, J=6.6 Hz, 2H), 2.42 (s, 3H), 2.32-2.25 (m, 1H), 2.24 (s, 3H), 1.70-1.55 (m, 4H). ¹³C NMR (151 MHz, DMSO-d₆) δ 174.3, 169.1, 165.2, 164.1, 162.5, 162.2, 160.9, 159.9, 157.0, 148.3, 146.4, 144.9, 140.8, 138.8, 138.5, 137.6, 133.5, 132.4, 129.6, 129.0, 128.2, 127.0, 125.8, 125.0 (t, J_{C-F}=286.8 Hz), 122.2, 121.9 (2C), 121.5 (2C), 118.3, 103.4, 82.8, 70.3-69.3 (m, 14C), 69.1, 66.8, 48.4 (2C), 44.4, 43.5, 43.2, 41.7, 40.5, 38.5, 32.9, 28.1 (2C), 25.6, 18.3. ¹⁹F NMR (564 MHz, DMSO-d₆) δ −24.7. HRMS (m/z): calculated for C₆₁H₇₈Cl₂F₂N₁₃O₁₃S⁺ [M+H]⁺ 1340.4902, found 1340.4911. TLC: R_{f}=0.6 (20% methanol-dichloromethane).

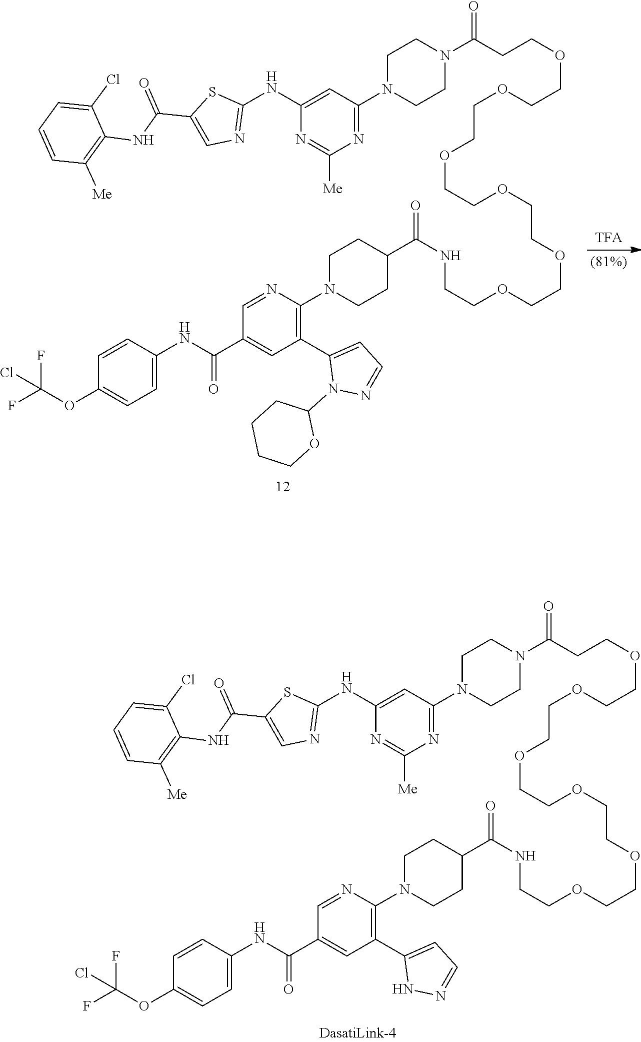 [SMILES: Cc1nc(Nc2ncc(C(=O)Nc3c(C)cccc3Cl)s2)cc(N2CCN(C(=O)CCOCCOCCOCCOCCOCCOCCNC(=O)C3CCN(c4ncc(C(=O)Nc5ccc(OC(F)(F)Cl)cc5)cc4-c4ccnn4C4CCCCO4)CC3)CC2)n1 ; Cc1nc(Nc2ncc(C(=O)Nc3c(C)cccc3Cl)s2)cc(N2CCN(C(=O)CCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

DasatiLink-4. The same procedure as for DasatiLink-1, using compound 12 as starting material with scaled reagents, afforded compound DasatiLink-4 (38 mg, 0.303 mmol, 81% yield) as a white solid.

¹H NMR (600 MHz, DMSO-d₆) δ 13.03 (s, 1H), 11.51 (s, 1H), 10.41 (s, 1H), 9.89 (s, 1H), 8.74 (d, J=2.4 Hz, 1H), 8.32 (s, 1H), 8.23 (s, 1H), 7.97-7.51 (m, 4H), 7.44-7.37 (m, 1H), 7.34 (d, J=8.7 Hz, 2H), 7.31-7.19 (m, 2H), 6.66 (s, 1H), 6.06 (s, 1H), 3.76-3.61 (m, 4H), 3.61-3.51 (m, 8H), 3.51-3.44 (m, 20H), 3.39 (t, J=6.0 Hz, 2H), 3.22-3.14 (m, 2H), 2.76-2.66 (m, 2H), 2.61 (t, J=6.6 Hz, 2H), 2.42 (s, 3H), 2.33-2.25 (m, 1H), 2.24 (s, 3H), 1.74-1.52 (m, 4H). ¹³C NMR (151 MHz, DMSO-d₆) δ 174.3, 169.1, 165.2, 164.1, 162.5, 162.2, 160.9, 159.9, 157.0, 148.3, 146.4, 144.9, 140.8, 138.8, 138.5, 137.6, 133.5, 132.4, 129.6, 129.0, 128.2, 127.0, 125.8, 125.0 (t, J_{C-F}=286.8 Hz), 122.2, 121.9 (2C), 121.5 (2C), 118.3, 103.5, 82.8, 70.3-69.3 (m, 10C), 69.1, 66.8, 48.4 (2C), 44.3, 43.5, 43.2, 41.7, 40.5, 38.4, 32.9, 28.1 (2C), 25.6, 18.3. ¹⁹F NMR (564 MHz, DMSO-d₆) δ −24.7. HRMS (m/z): calculated for C₅₇H₇₀Cl₂F₂N₁₃O₁₁S⁺ [M+H]⁺ 1252.4378, found 1252.4387. TLC: R_{f}=0.6 (20% methanol-dichloromethane).

Reagents and conditions (FIG. 15). (a) TFA, CH₂Cl₂, rt. (b) HATU, DIPEA, DMF, rt, 94% over two steps. (c) TFA, CH₂Cl₂, rt. (d) HATU, DIPEA, DMF, rt, 69% over two steps. (e) TFA, CH₂Cl₂, rt, 46%. Abbreviations: HATU, 1-[bis(dimethylamino)methylene]-1H-1,2,3-triazolo[4,5-b]pyridinium 3-oxide hexafluorophosphate; DIPEA, N,N-diisopropylethylamine; DMF, N,N-dimethylformamide; TFA, trifluoroacetic acid.

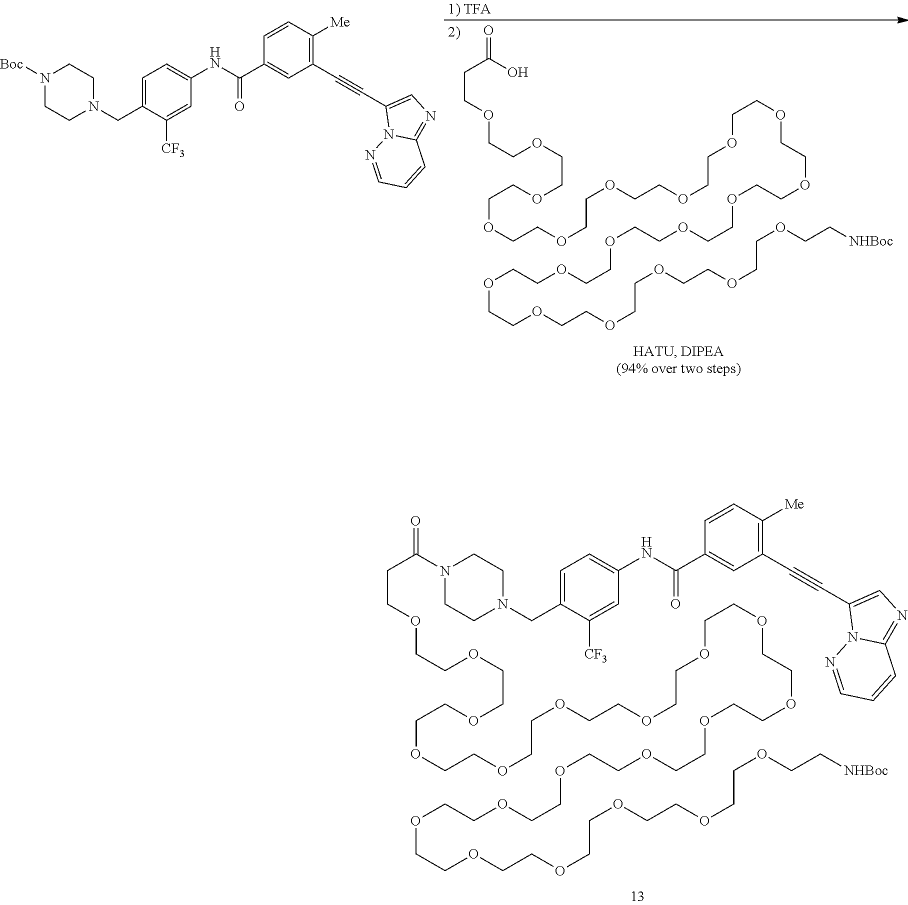

Compound 13. To a mixture of Boc-protected N-desmethyl ponatinib (131 mg, 0.212 mmol) in dichloromethane (2.12 mL) was added trifluoroacetic acid (2.12 mL). The solution was stirred at room temperature for 1 h before concentrating in vacuo. The residue was partitioned between ethyl acetate and saturated sodium bicarbonate. The aqueous layer was extracted with ethyl acetate (3×) and the combined organics were washed with brine (2×), dried over sodium sulfate, filtered, and concentrated in vacuo, and the residue was used directly in the next step without further purification. To a mixture of crude N-desmethyl ponatinib and t-Boc-N-amido-PEG₂₀-acid (225 mg, 0.210 mmol) in N,N-dimethylformamide (1.05 mL) was added N,N-diisopropylethylamine (110 μL, 0.631 mmol). The solution was cooled in an ice-water bath before the addition of 1-[bis(dimethylamino)methylene]-1H-1,2,3-triazolo[4,5-b]pyridinium 3-oxide hexafluorophosphate (88 mg, 0.231 mmol) and stirred at room temperature overnight. The mixture was partitioned between dichloromethane and brine. The aqueous layer was extracted with dichloromethane (6×), and the combined organics were dried over sodium sulfate, filtered, and concentrated in vacuo. The crude was purified by flash chromatography over silica gel as follows: the crude was dry loaded into silica gel and an initial gradient elution from 0% methanol-ethyl acetate to 20% methanol-ethyl acetate was made to remove impurities. Switching the gradient elution to 10% methanol-dichloromethane to 20% methanol-dichloromethane afforded compound 13 (310 mg, 0.197 mmol, 94% yield over two steps) as a pale yellow semisolid.

¹H NMR (400 MHz, DMSO-d₆) δ 10.57 (s, 1H), 8.73 (dd, J=4.5, 1.6 Hz, 1H), 8.26 (dd, J=9.3, 1.6 Hz, 1H), 8.25-8.20 (m, 3H), 8.10 (dd, J=8.4, 2.2 Hz, 1H), 7.95 (dd, J=7.9, 2.0 Hz, 1H), 7.75 (d, J=8.6 Hz, 1H), 7.55 (d, J=8.2 Hz, 1H), 7.40 (dd, J=9.2, 4.4 Hz, 1H), 6.84-6.62 (m, 1H), 3.70-3.42 (m, 84H), 3.37 (t, J=6.2 Hz, 2H), 3.10-3.01 (m, 2H), 2.61 (s, 3H), 2.56 (t, J=6.7 Hz, 2H), 2.44-2.29 (m, 4H), 1.36 (s, 9H). ¹⁹F NMR (376 MHz, DMSO-d₆) δ −58.0. HRMS (m/z): calculated for C₇₆H₁₁₉F₃N₇O₂₄⁺ [M+H]⁺ 1570.8253, found 1570.8188. TLC: R_{f}=0.6 (20% methanol-dichloromethane).

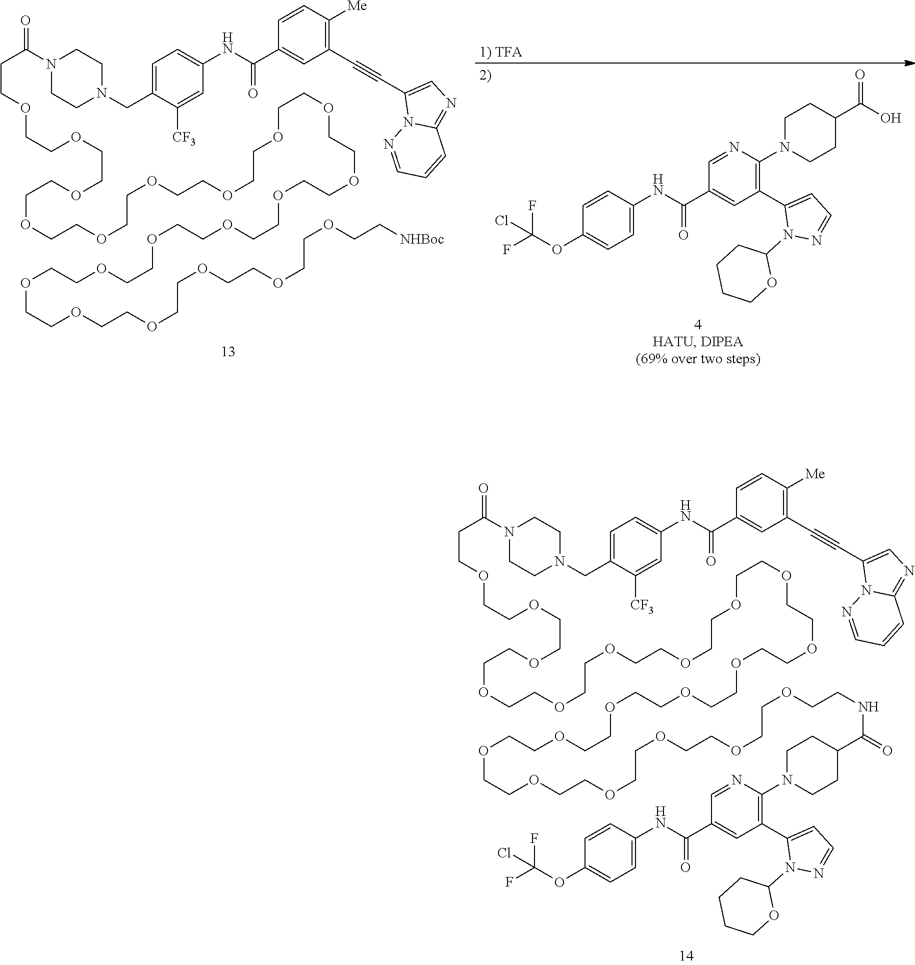 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCNC(=O)OC(C)(C)C)CC3)c(C(F)(F)F)c2)cc1C#Cc1cnc2cccnn12 ; O=C(Nc1ccc(OC(F)(F)Cl)cc1)c1cnc(N2CCC(C(=O)O)CC2)c(-c2ccnn2C2CCCCO2)c1 ; Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccnn5C5CCCCO5)CC4)CC3)c(C(F)(F)F)c2)cc1C#Cc1cnc2cccnn12]

Compound 14. To a mixture of compound 13 (281 mg, 0.179 mmol) in dichloromethane (1.79 mL) was added trifluoroacetic acid (1.79 mL). The solution was stirred at room temperature for 1 h before concentrating in vacuo to afford a residue that was used directly in the next step. To a mixture of the crude amine, trifluoroacetic acid salt and compound 4 (113 mg, 0.197 mmol) in N,N-dimethylformamide (1.79 mL) was added N,N-diisopropylethylamine (312 μL, 1.79 mmol). The solution was cooled in an ice-water bath before the addition of 1-[bis(dimethylamino)methylene]-1H-1,2,3-triazolo[4,5-b]pyridinium 3-oxide hexafluorophosphate (75 mg, 0.197 mmol) and stirred at room temperature overnight. The mixture was partitioned between dichloromethane and brine. The aqueous layer was extracted with dichloromethane (3×), and the combined organics were dried over sodium sulfate, filtered, and concentrated in vacuo. The crude was purified by flash chromatography over silica gel as follows: the crude was dry loaded into silica gel and an initial gradient elution from 0% methanol-ethyl acetate to 10% methanol-ethyl acetate was made to remove impurities. Switching the gradient elution to 0% methanol-dichloromethane to 20% methanol-dichloromethane afforded compound 13 (251 mg, 0.124 mmol, 69% yield over two steps) as a pale yellow semisolid.

¹H NMR (400 MHz, DMSO-d₆) δ 10.66 (s, 1H), 10.49 (s, 1H), 8.88 (d, J=2.4 Hz, 1H), 8.73 (dd, J=4.4, 1.6 Hz, 1H), 8.31-8.20 (m, 4H), 8.16-8.08 (m, 2H), 7.99 (dd, J=8.0, 2.0 Hz, 1H), 7.95-7.87 (m, 2H), 7.87-7.81 (m, 1H), 7.74 (d, J=8.6 Hz, 1H), 7.64 (d, J=1.4 Hz, 1H), 7.55 (d, J=8.1 Hz, 1H), 7.40 (dd, J=9.2, 4.5 Hz, 1H), 7.33 (d, J=8.6 Hz, 2H), 6.47 (d, J=1.6 Hz, 1H), 5.18 (dd, J=9.7, 2.4 Hz, 1H), 3.83-3.42 (m, 87H), 3.37 (t, J=6.3 Hz, 2H), 3.32-3.28 (m, 1H), 3.18-3.13 (m, 2H), 2.80-2.64 (m, 2H), 2.61 (s, 3H), 2.56 (t, J=6.7 Hz, 2H), 2.43-2.26 (m, 6H), 2.00-1.84 (m, 2H), 1.68-1.36 (m, 7H). ¹⁹F NMR (376 MHz, DMSO-d₆) δ −24.7, −58.0. HRMS (m/z): calculated for C₉₈H₁₃₇ClF₅N₁₂O₂₆⁺ [M+H]⁺ 2027.9370, found 2027.8849. TLC: R_{f}=0.7 (20% methanol-dichloromethane).

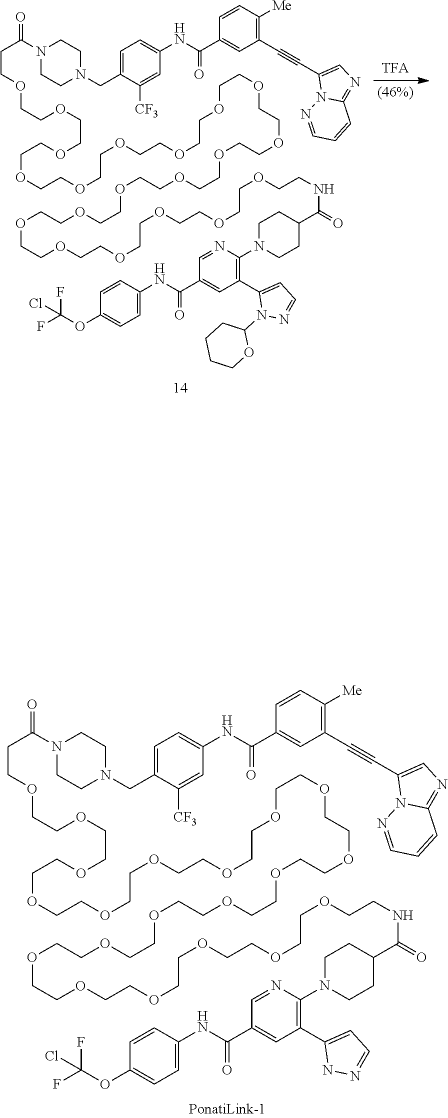 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccnn5C5CCCCO5)CC4)CC3)c(C(F)(F)F)c2)cc1C#Cc1cnc2cccnn12 ; Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

PonatiLink-1. The same procedure as for DasatiLink-1, using compound 14 as starting material with scaled reagents, afforded compound PonatiLink-1 (29 mg, 0.0238 mmol, 46% yield) as a pale yellow semisolid.

¹H NMR (400 MHz, DMSO-d₆) δ 13.01 (s, 1H), 10.57 (s, 1H), 10.39 (s, 1H), 8.80-8.68 (m, 2H), 8.33 (s, 1H), 8.26 (dd, J=9.2, 1.6 Hz, 1H), 8.25-8.20 (m, 3H), 8.10 (dd, J=8.6, 2.2 Hz, 1H), 7.95 (dd, J=8.0, 2.0 Hz, 1H), 7.92-7.79 (m, 4H), 7.75 (d, J=8.5 Hz, 1H), 7.55 (d, J=8.2 Hz, 1H), 7.40 (dd, J=9.2, 4.5 Hz, 1H), 7.34 (d, J=8.7 Hz, 2H), 6.65 (s, 1H), 3.72-3.42 (m, 86H), 3.39 (t, J=5.9 Hz, 2H), 3.24-3.14 (m, 2H), 2.78-2.65 (m, 2H), 2.61 (s, 3H), 2.56 (t, J=6.7 Hz, 2H), 2.42-2.24 (m, 5H), 1.74-1.52 (m, 4H). ¹⁹F NMR (376 MHz, DMSO-d₆) δ −24.7, −58.0. HRMS (m/z): calculated for C₉₃H₁₂₉ClF₅N₁₂O₂₅⁺ [M+H]⁺ 1943.8795, found 1943.8350. TLC: R_{f}=0.6 (20% methanol-dichloromethane).

### Example 4: Additional Chemical Synthesis Methods

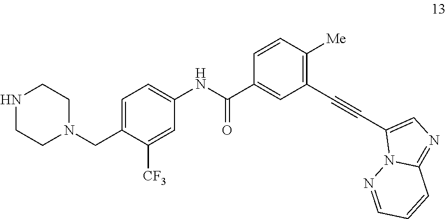 [SMILES: Cc1ccc(C(=O)Nc2ccc(CN3CCNCC3)c(C(F)(F)F)c2)cc1C#Cc1cnc2cccnn12]

Compound 13 has been previously described by Huang et al. J. Med. Chem. 2010 and was prepared according the synthetic routes provided in Huang et al. J. Med. Chem. 2010, Zhang et al. J. Med. Chem. 2015, and patent WO2019196812 by Pharmaron Inc. as the HCl salt as a white solid (3.3 g).

¹H NMR (400 MHz, Methanol-d4) δ 9.12-9.07 (m, 1H), 8.61 (s, 1H), 8.57-8.47 (m, 1H), 8.39-8.36 (m, 1H), 8.27-8.23 (m, 1H), 8.19-8.10 (m, 1H), 8.07-7.86 (m, 3H), 7.55 (d, J=8.1 Hz, 1H), 4.43 (s, 2H), 3.63-3.35 (m, 8H), 2.69 (s, 3H).

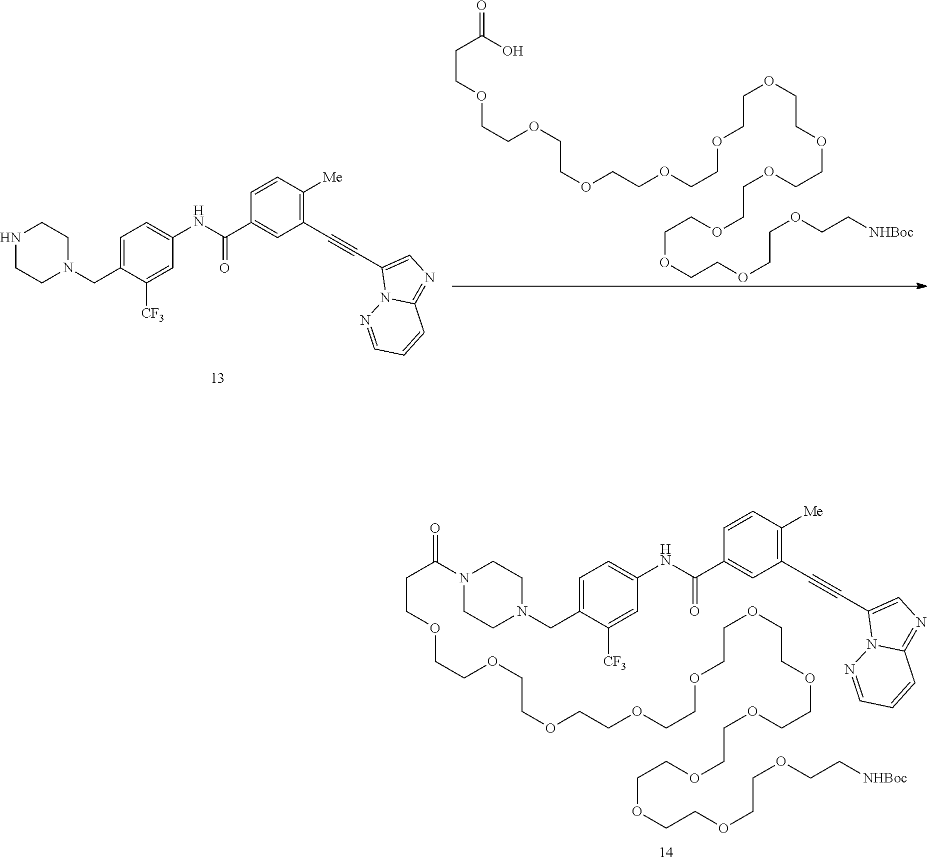 [SMILES: Cc1ccc(C(=O)Nc2ccc(CN3CCNCC3)c(C(F)(F)F)c2)cc1C#Cc1cnc2cccnn12 ; CC(C)(C)OC(=O)NCCOCCOCCOCCOCCOCCOCCOCCOCCOCCOCCOCCOCCC(=O)O ; Cc1ccc(C(=O)Nc2ccc(CN3CCN(C(=O)CCOCCOCCOCCOCCOCCOCCOCCOCCOCCOCCOCCOCCNC(=O)OC(C)(C)C)CC3)c(C(F)(F)F)c2)cc1C#Cc1cnc2cccnn12]

Compound 14. To a mixture of compound 13 (200 mg, 0.342 mmol) and t-Boc-N-amido-PEG12-acid (245 mg, 0.342 mmol) in N,N-dimethylformamide (1.71 mL) was added N,N-diisopropylethylamine (179 μL, 1.03 mmol). HATU (143 mg, 0.376 mmol) was added and the mixture was stirred at room temperature overnight. The mixture was partitioned between DCM and brine. The aqueous layer was extracted with DCM (3×), and the combined organics were dried over sodium sulfate and filtered, with ethyl acetate used to rinse the filter cake, and concentrated in vacuo. The crude was purified by flash chromatography over silica gel as follows: the crude was dry-loaded into silica gel and an initial gradient elution from 0% methanol-ethyl acetate to 10% methanol-ethyl acetate was made to remove impurities. Switching the gradient elution to 0% methanol-DCM to 10% methanol-DCM afforded compound 14 (328.1 mg, 0.269 mmol, 78% yield) as a pale yellow semisolid.

¹H NMR (400 MHz, DMSO-d₆) δ 10.57 (s, 1H), 8.72 (dd, J=4.5, 1.6 Hz, 1H), 8.25 (dd, J=9.3, 1.6 Hz, 1H), 8.24-8.20 (m, 3H), 8.09 (dd, J=8.5, 2.2 Hz, 1H), 7.95 (dd, J=8.0, 2.0 Hz, 1H), 7.74 (d, J=8.6 Hz, 1H), 7.55 (d, J=8.1 Hz, 1H), 7.39 (dd, J=9.2, 4.5 Hz, 1H), 6.74-6.68 (m, 1H), 3.66-3.57 (m, 4H), 3.49 (s, 43H), 3.05 (q, J=6.0 Hz, 2H), 2.61 (s, 3H), 2.56 (t, J=6.6 Hz, 2H), 2.36 (dt, J=18.4, 5.0 Hz, 4H), 1.36 (s, 9H). ¹⁹F NMR (376 MHz, DMSO-d₆) δ −58.00.

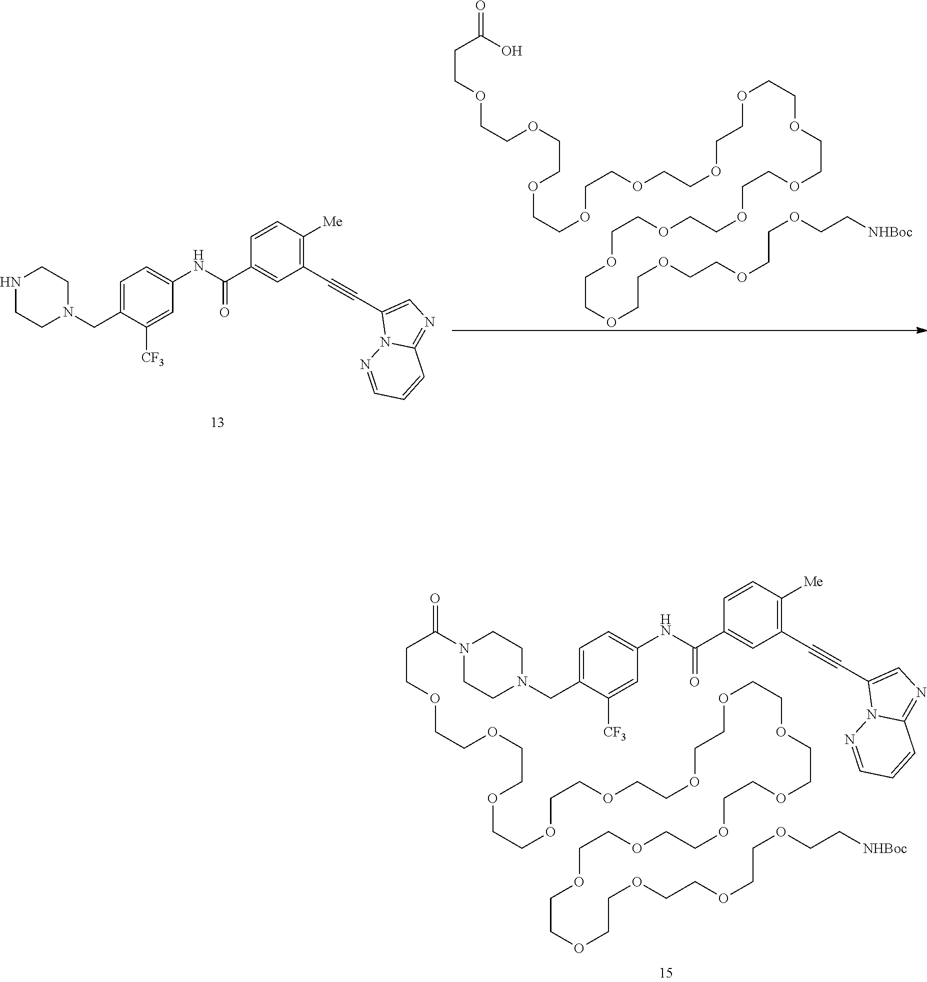 [SMILES: Cc1ccc(C(=O)Nc2ccc(CN3CCNCC3)c(C(F)(F)F)c2)cc1C#Cc1cnc2cccnn12 ; CC(C)(C)OC(=O)NCCOCCOCCOCCOCCOCCOCCOCCOCCOCCOCCOCCOCCOCCOCCOCCOCCC(=O)O ; Cc1ccc(C(=O)Nc2ccc(CN3CCN(C(=O)CCOCCOCCOCCOCCOCCOCCOCCOCCOCCOCCOCCOCCOCCOCCOCCOCCNC(=O)OC(C)(C)C)CC3)c(C(F)(F)F)c2)cc1C#Cc1cnc2cccnn12]

Compound 15. The same procedure as for compound 14, using t-Boc-N-amido-PEG₁₆-acid (274 mg, 0.256 mmol) as starting material with scaled reagents, afforded compound 15 (270.4 mg, 0.179 mmol, 70% yield) as a pale yellow semisolid.

¹H NMR (400 MHz, DMSO-d₆) δ 10.58 (s, 1H), 8.73 (dd, J=4.4, 1.5 Hz, 1H), 8.27 (dd, J=9.3, 1.6 Hz, 1H), 8.24-8.21 (m, 3H), 8.10 (d, J=8.5 Hz, 1H), 7.96 (dd, J=7.9, 1.9 Hz, 1H), 7.75 (d, J=8.6 Hz, 1H), 7.56 (d, J=8.2 Hz, 1H), 7.40 (dd, J=9.2, 4.4 Hz, 1H), 6.72 (s, 1H), 3.67-3.57 (m, 6H), 3.50 (d, J=0.9 Hz, 63H), 3.19-3.09 (m, 2H), 3.05 (q, J=6.0 Hz, 2H), 2.61 (s, 3H), 2.43-2.32 (m, 4H), 1.37 (s, 9H), 1.31-1.22 (m, 20H). ¹⁹F NMR (376 MHz, DMSO-d₆) δ −57.98. HRMS (m/z): calculated for C₆₈H₁₀₃F₃N₇O₂₀⁺ [M+H]⁺ 1394.7205, found 1394.7216.

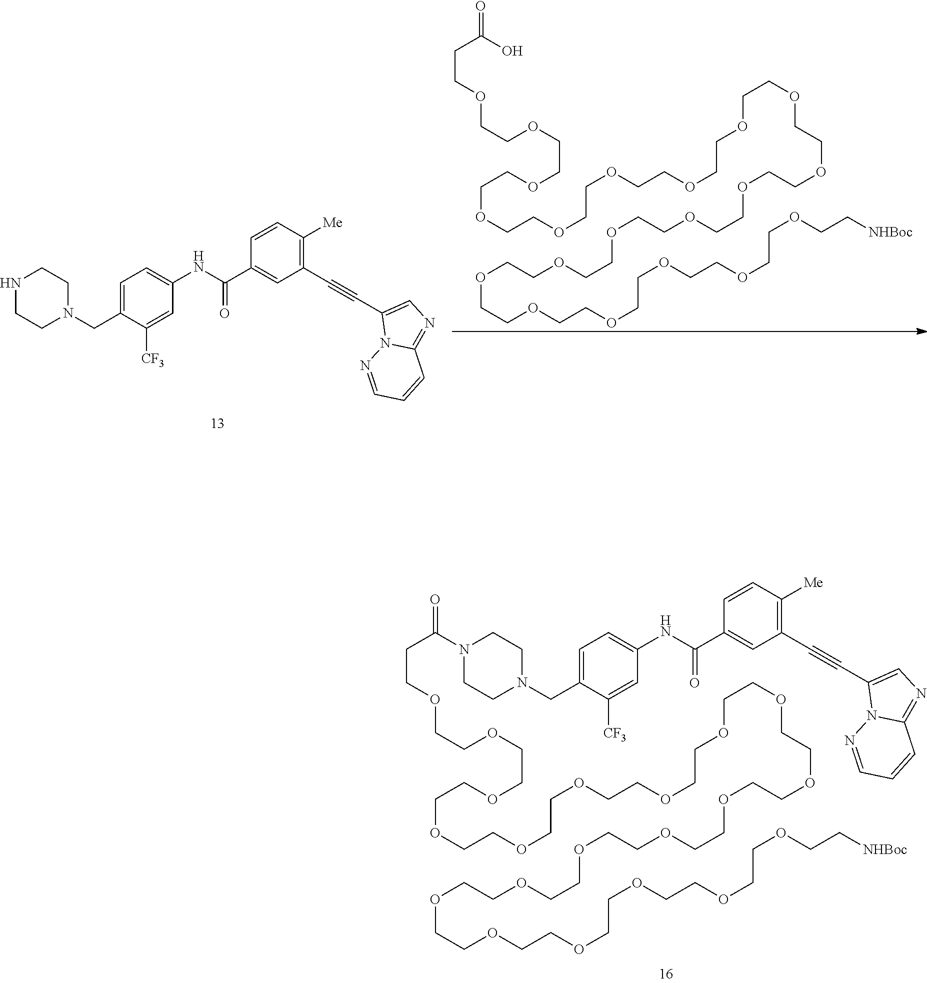 [SMILES: Cc1ccc(C(=O)Nc2ccc(CN3CCNCC3)c(C(F)(F)F)c2)cc1C#Cc1cnc2cccnn12 ; CC(C)(C)OC(=O)NCCOCCOCCOCCOCCOCCOCCOCCOCCOCCOCCOCCOCCOCCOCCOCCOCCOCCOCCOCCOCCC(=O)O ; Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCNC(=O)OC(C)(C)C)CC3)c(C(F)(F)F)c2)cc1C#Cc1cnc2cccnn12]

Compound 16. The same procedure as for compound 14, using t-Boc-N-amido-PEG₂₀-acid (125 mg, 0.117 mmol) as starting material with scaled reagents, afforded compound 16 (147 mg, 0.093 mmol, 80% yield) as a yellow semisolid.

¹H NMR (400 MHz, DMSO-d₆) δ 10.58 (s, 1H), 8.74 (dd, J=4.5, 1.6 Hz, 1H), 8.31-8.21 (m, 4H), 8.11 (d, J=8.7 Hz, 1H), 8.01-7.93 (m, 1H), 7.76 (d, J=8.5 Hz, 1H), 7.57 (d, J=8.1 Hz, 1H), 7.41 (dd, J=9.2, 4.5 Hz, 1H), 6.73 (s, 1H), 3.75-3.58 (m, 4H), 3.51 (t, J=1.8 Hz, 89H), 3.38 (t, J=6.1 Hz, 2H), 3.32 (s, 53H), 3.06 (q, J=6.1 Hz, 2H), 2.71-2.66 (m, 1H), 2.62 (s, 3H), 2.58 (d, J=6.7 Hz, 1H), 2.51 (p, J=1.8 Hz, 204H), 2.45-2.32 (m, 4H), 1.38 (s, 12H). HRMS (m/z): calculated for C₇₆H₁₁₉F₃N₇O₂₄⁺ [M+H]⁺ 1570.8253, found 1570.8065. TL C: R_{f}=0.7 (20% methanol-DCM).

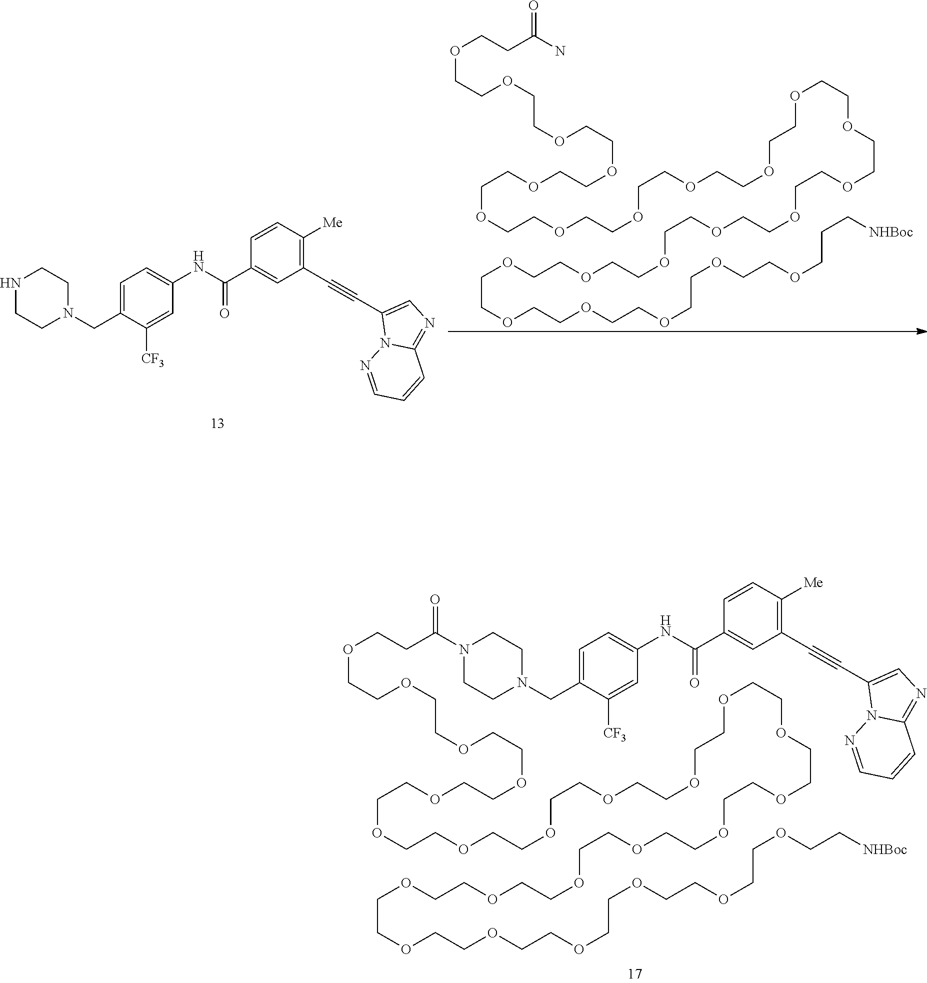 [SMILES: Cc1ccc(C(=O)Nc2ccc(CN3CCNCC3)c(C(F)(F)F)c2)cc1C#Cc1cnc2cccnn12 ; CC(C)(C)OC(=O)NCCCOCCOCCOCCOCCOCCOCCOCCOCCOCCOCCOCCOCCOCCOCCOCCOCCOCCOCCOCCOCCOCCOCCOCCC(N)=O ; Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCNC(=O)OC(C)(C)C)CC3)c(C(F)(F)F)c2)cc1C#Cc1cnc2cccnn12]

Compound 17. The same procedure as for compound 14, using t-Boc-N-amido-PEG24-acid (320 mg, 0.256 mmol) as starting material with scaled reagents, afforded compound 17 (325 mg, 0.175 mmol, 68% yield) as a pale yellow semisolid.

¹H NMR (400 MHz, DMSO-d₆) δ 10.72 (s, 1H), 8.73 (dd, J=4.5, 1.6 Hz, 1H), 7.97 (dd, J=8.0, 1.9 Hz, 1H), 7.84 (s, 1H), 7.58 (d, J=8.2 Hz, 1H), 7.41 (dd, J=9.2, 4.5 Hz, 1H), 6.72 (s, 1H), 3.70-3.60 (m, 2H), 3.50 (d, J=1.0 Hz, 88H), 3.37 (t, J=6.1 Hz, 2H), 3.05 (q, J=6.0 Hz, 2H), 2.62 (s, 3H), 2.50 (p, J=1.8 Hz, 57H), 1.37 (s, 9H). ¹⁹F NMR (376 MHz, DMSO-d₆) δ −58.00. HRMS (m/z): calculated for C₈₄H₁₃₅F₃N₇O₂₈⁺ [M+H]⁺ 1746.9302, found 1746.8936. TLC: R_{f}=0.7 (20% methanol-DCM).

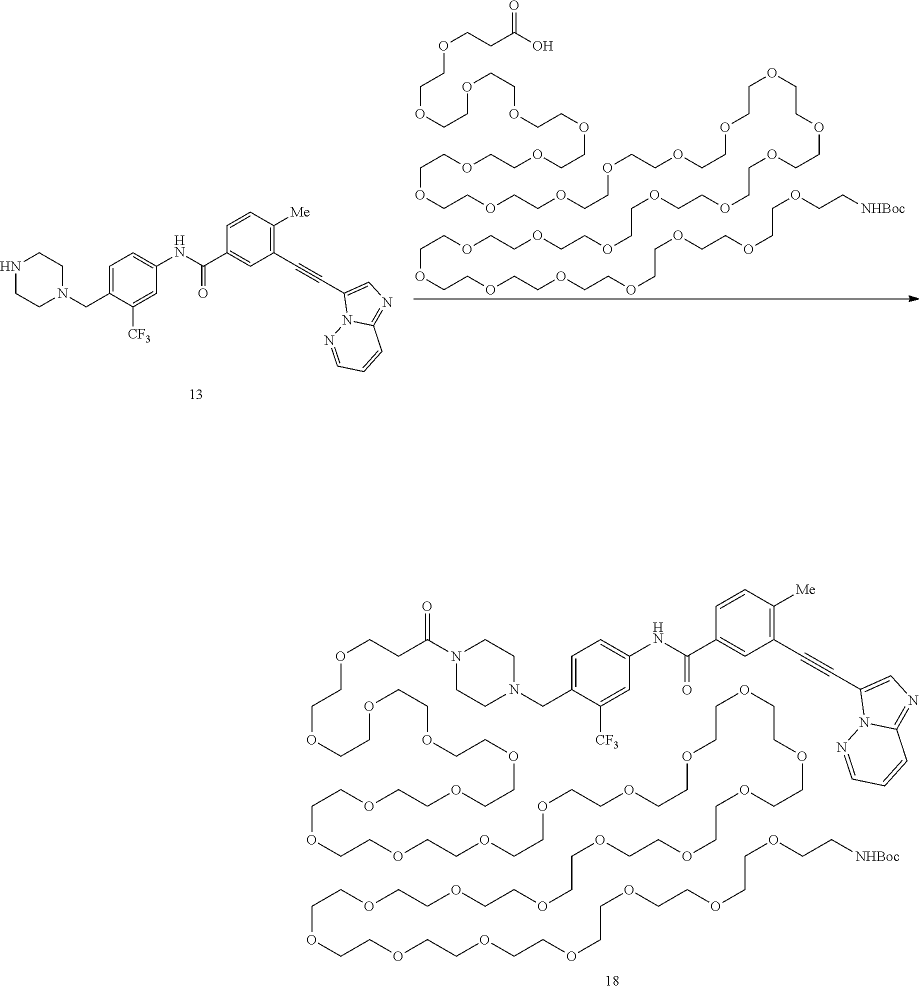 [SMILES: Cc1ccc(C(=O)Nc2ccc(CN3CCNCC3)c(C(F)(F)F)c2)cc1C#Cc1cnc2cccnn12 ; CC(C)(C)OC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCC(=O)O ; Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)OC(C)(C)C)CC3)c(C(F)(F)F)c2)cc1C#Cc1cnc2cccnn12]

Compound 18. The same procedure as for compound 14, using t-Boc-N-amido-PEG₂₈-acid (97 mg, 0.068 mmol) as starting material with scaled reagents, afforded compound 18 (98 mg, 0.051 mmol, 75% yield) as a clear semisolid.

¹H NMR (400 MHz, DMSO-d₆) δ 10.59 (s, 1H), 8.74 (dd, J=4.5, 1.6 Hz, 1H), 8.31-8.20 (m, 4H), 8.11 (d, J=8.6 Hz, 1H), 8.05-7.92 (m, 1H), 7.76 (d, J=8.6 Hz, 1H), 7.57 (d, J=8.1 Hz, 1H), 7.41 (dd, J=9.2, 4.5 Hz, 1H), 6.74 (s, 1H), 3.70-3.57 (m, 6H), 3.51 (d, J=1.7 Hz, 101H), 3.37 (t, J=6.1 Hz, 2H), 3.33 (d, J=0.7 Hz, 70H), 3.20-3.10 (m, 2H), 3.06 (q, J=6.0 Hz, 2H), 2.62 (s, 3H), 2.57 (t, J=6.6 Hz, 2H), 2.51 (p, J=1.9 Hz, 66H), 2.43-2.33 (m, 4H), 1.37 (s, 10H), 1.27 (dt, J=7.5, 5.4 Hz, 21H). ¹⁹F NMR (376 MHz, DMSO-d₆) δ −58.00. HRMS (m/z): calculated for C₉₂H₁₅₁F₃N₇O₃₂⁺ [M+H]⁺ 1923.0351, found 1922.9049.

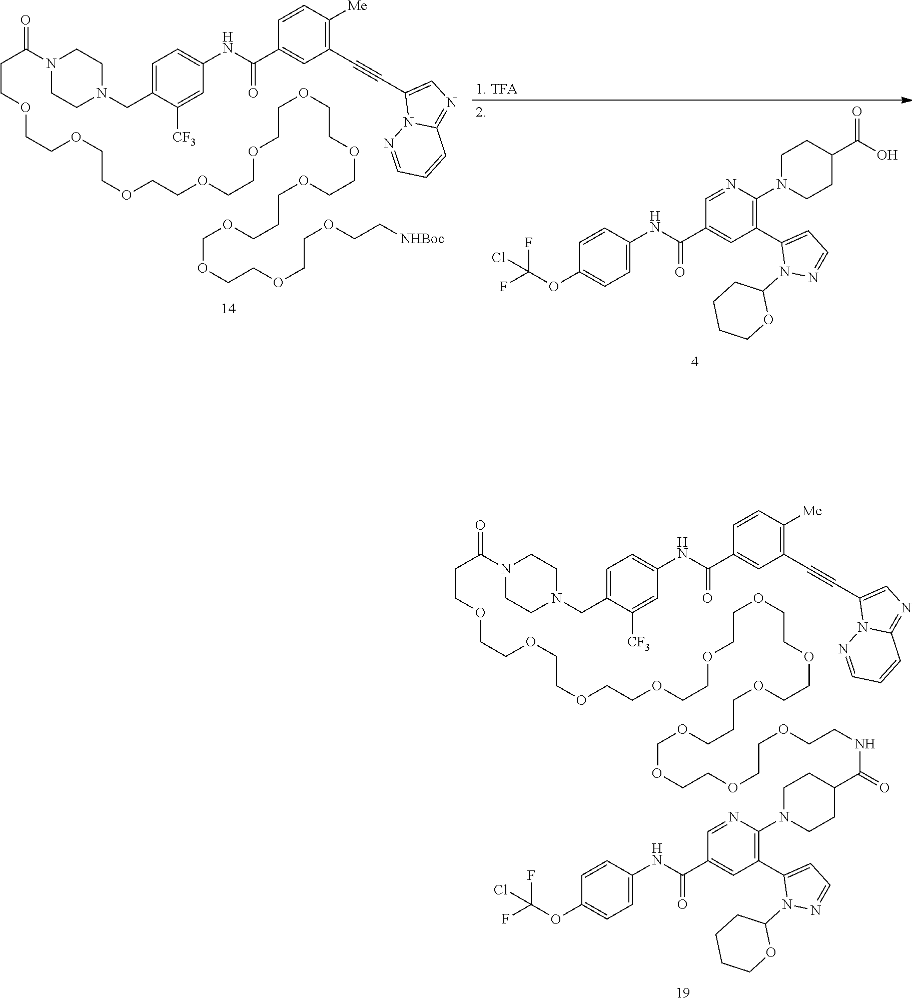 [SMILES: Cc1ccc(C(=O)Nc2ccc(CN3CCN(C(=O)CCOCCOCCOCCOCCOCCOCCOCCOCCCOCOCCOCCOCCNC(=O)OC(C)(C)C)CC3)c(C(F)(F)F)c2)cc1C#Cc1cnc2cccnn12 ; O=C(Nc1ccc(OC(F)(F)Cl)cc1)c1cnc(N2CCC(C(=O)O)CC2)c(-c2ccnn2C2CCCCO2)c1 ; Cc1ccc(C(=O)Nc2ccc(CN3CCN(C(=O)CCOCCOCCOCCOCCOCCOCCOCCOCCCOCOCCOCCOCCNC(=O)C4CCN(c5ncc(C(=O)Nc6ccc(OC(F)(F)Cl)cc6)cc5-c5ccnn5C5CCCCO5)CC4)CC3)c(C(F)(F)F)c2)cc1C#Cc1cnc2cccnn12]

Compound 19. To a solution of compound 14 (299 mg, 0.245 mmol) in DCM (2.45 mL) was added TFA (0.82 mL). The mixture was stirred at room temperature for 45 min, then concentrated in vacuo, and the residue was used directly in the next step without further purification. A mixture of the crude amine, trifluoroacetic acid salt, compound 4 (210 mg, 0.364 mmol), and DIPEA (641 μL, 3.68 mmol), in DCM (2.45 mL) was cooled in an ice-water bath before the addition of HATU (103 mg, 0.270 mmol), then warmed to room temperature, and the mixture was stirred at room temperature overnight. The mixture was partitioned between DCM and brine. The aqueous layer was extracted with DCM (3×), and the combined organics were dried over sodium sulfate and filtered, with ethyl acetate used to rinse the filter cake, and concentrated in vacuo. The residue was purified by reverse-phase C18 flash chromatography, 10-90% acetonitrile/water+0.1% TFA to afford compound 19 (183 mg, 1.09 mmol, 45% yield over two steps) as an off-white solid.

¹H NMR (400 MHz, DMSO-d₆) δ 10.74 (s, 1H), 10.41 (d, J=12.9 Hz, 1H), 8.73 (dt, J=4.2, 2.1 Hz, 2H), 8.36-8.21 (m, 6H), 8.03-7.93 (m, 1H), 7.92-7.77 (m, 4H), 7.58 (d, J=8.2 Hz, 1H), 7.41 (dd, J=9.2, 4.5 Hz, 1H), 7.34 (d, J=8.7 Hz, 2H), 6.72 (d, J=2.4 Hz, OH), 6.65 (d, J=2.2 Hz, 1H), 3.50 (d, J=1.5 Hz, 44H), 3.39 (t, J=5.9 Hz, 2H), 3.18 (q, J=5.8 Hz, 2H), 2.76-2.70 (m, 1H), 2.62 (s, 4H), 2.50 (p, J=1.9 Hz, 23H), 2.29 (s, 1H), 1.97 (d, J=11.9 Hz, 1H), 1.63 (s, 4H). ¹⁹F NMR (376 MHz, DMSO-d₆) δ −24.70, −56.74, −74.79 (d, J=39.5 Hz). HRMS (m/z): calculated for C₈₂H₁₀₄ClF₅N₂₁O₁₈⁺ [M+H]⁺ 1675.7273, found 1675.7035. TLC: R_{f}=0.7 (20% methanol-DCM).

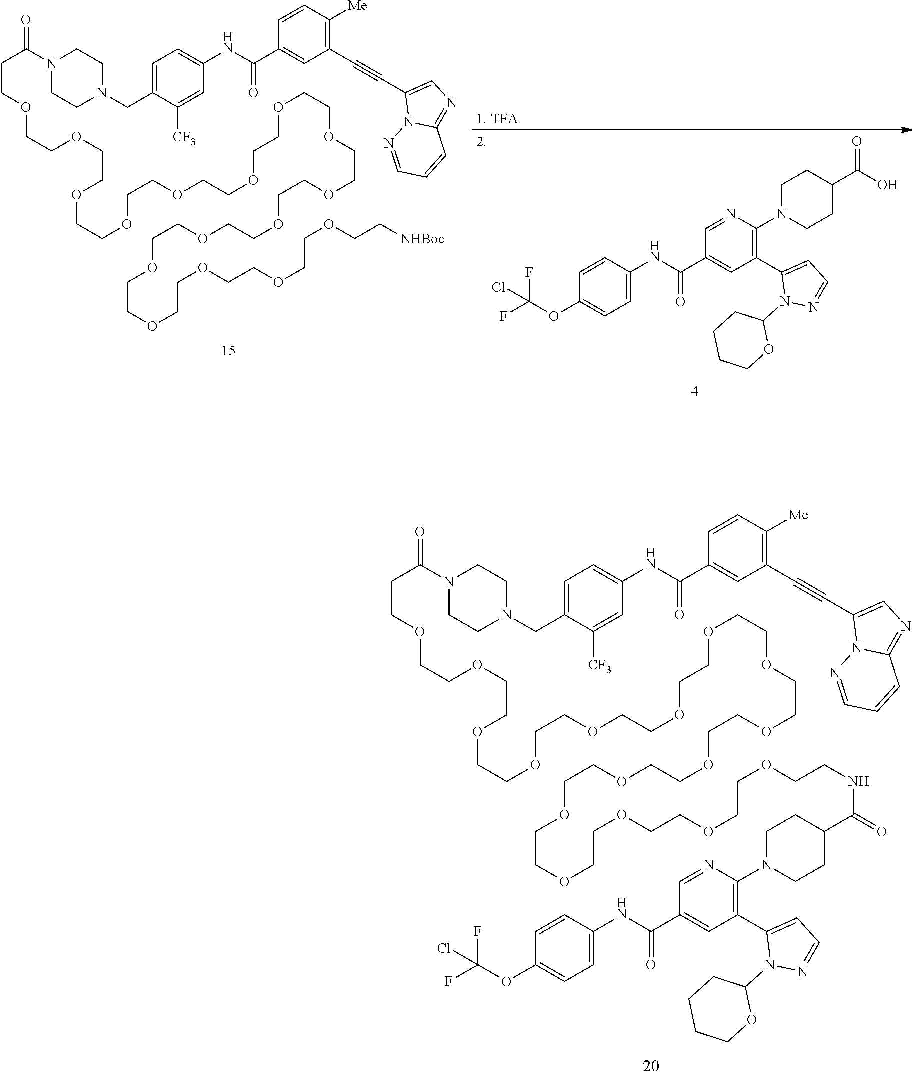 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCNC(=O)OC(C)(C)C)CC3)c(C(F)(F)F)c2)cc1C#Cc1cnc2cccnn12 ; O=C(Nc1ccc(OC(F)(F)Cl)cc1)c1cnc(N2CCC(C(=O)O)CC2)c(-c2ccnn2C2CCCCO2)c1 ; Cc1ccc(C(=O)Nc2ccc(CN3CCN(C(=O)CCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccnn5C5CCCCO5)CC4)CC3)c(C(F)(F)F)c2)cc1C#Cc1cnc2cccnn12]

Compound 20. To a solution of compound 15 (112 mg, 0.173 mmol) in DCM (1.73 mL) was added TFA (0.58 mL). The mixture was stirred at room temperature for 1 hr, then concentrated in vacuo, and the residue was used directly in the next step without further purification. To a mixture of the crude amine, trifluoroacetic acid salt, compound 4 (100 mg, 0.173 mmol), and DIPEA (90 μL, 0.519 mmol) in DCM (1.73 mL) was added HATU (72 mg, 0.190 mmol) and the mixture was stirred at room temperature overnight. The mixture was partitioned between DCM and brine. The aqueous layer was extracted with DCM (3×), and the combined organics were dried over sodium sulfate and filtered, with ethyl acetate used to rinse the filter cake, and concentrated in vacuo. The residue was purified as for compound 14 to afford compound 20 (206 mg, 0.111 mmol, 64% yield over two steps) as an off-white solid.

HRMS (m/z): calculated for C₉₀H₁₂₁ClF₅N₁₂O₂₂⁺ [M+H]⁺ 1851.8322, found 1851.9291. TLC: R_{f}=0.7 (20% methanol-DCM).

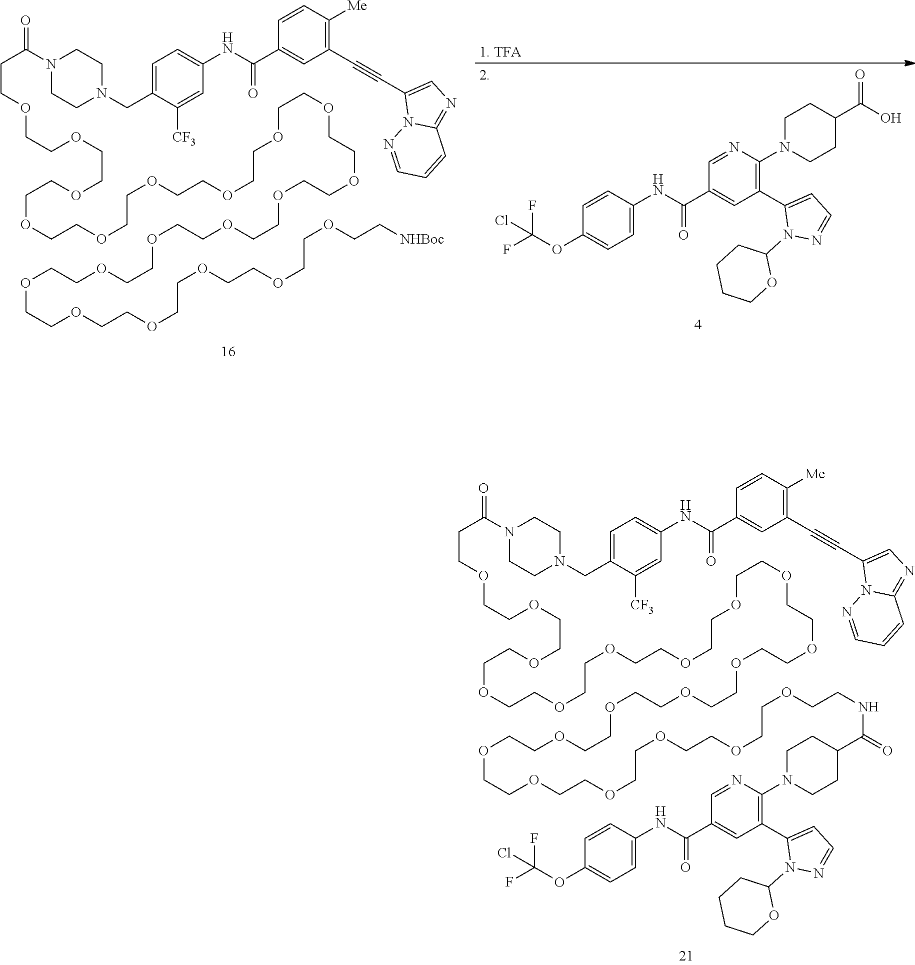 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCNC(=O)OC(C)(C)C)CC3)c(C(F)(F)F)c2)cc1C#Cc1cnc2cccnn12 ; O=C(Nc1ccc(OC(F)(F)Cl)cc1)c1cnc(N2CCC(C(=O)O)CC2)c(-c2ccnn2C2CCCCO2)c1 ; Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccnn5C5CCCCO5)CC4)CC3)c(C(F)(F)F)c2)cc1C#Cc1cnc2cccnn12]

Compound 21. The same procedure as for compound 20, using compound 16 (158 mg, 0.093 mmol) as starting material with scaled reagents, afforded compound 21 as a pale yellow oil (129 mg, 0.063 mmol, 68% yield over 2 steps).

¹H NMR (400 MHz, DMSO-d₆) δ 10.57 (s, 1H), 10.32 (s, 1H), 8.83 (d, J=2.4 Hz, 1H), 8.73 (dd, J=4.5, 1.6 Hz, 1H), 8.26 (dd, J=9.2, 1.6 Hz, 1H), 8.25-8.21 (m, 3H), 8.13-8.05 (m, 2H), 7.95 (dd, J=7.9, 1.9 Hz, 1H), 7.90-7.82 (m, 2H), 7.82-7.73 (m, 2H), 7.65 (d, J=1.8 Hz, 1H), 7.56 (d, J=8.2 Hz, 1H), 7.40 (dd, J=9.2, 4.5 Hz, 1H), 7.34 (d, J=8.8 Hz, 2H), 6.47 (d, J=1.7 Hz, 1H), 5.16 (dd, J=9.8, 2.4 Hz, 1H), 3.80 (d, J=11.3 Hz, 1H), 3.69 (d, J=13.1 Hz, 2H), 3.64-3.58 (m, 4H), 3.50 (d, J=1.8 Hz, 71H), 3.37 (t, J=6.0 Hz, 2H), 3.31 (s, 51H), 3.16 (q, J=5.8 Hz, 2H), 2.80-2.67 (m, 1H), 2.61 (s, 3H), 2.56 (t, J=6.7 Hz, 2H), 2.50 (p, J=1.8 Hz, 37H), 2.43-2.29 (m, 3H), 1.93 (q, J=14.2 Hz, 2H), 1.62-1.40 (m, 8H). ¹⁹F NMR (376 MHz, DMSO-d₆) δ −24.71, −57.98. HRMS (m/z): calculated for C₉₈H₁₃₇ClF₅N₁₂O₂₆⁺ [M+H]⁺ 2027.9370, found 2027.9443. TLC: R_{f}=0.7 (20% methanol-DCM).

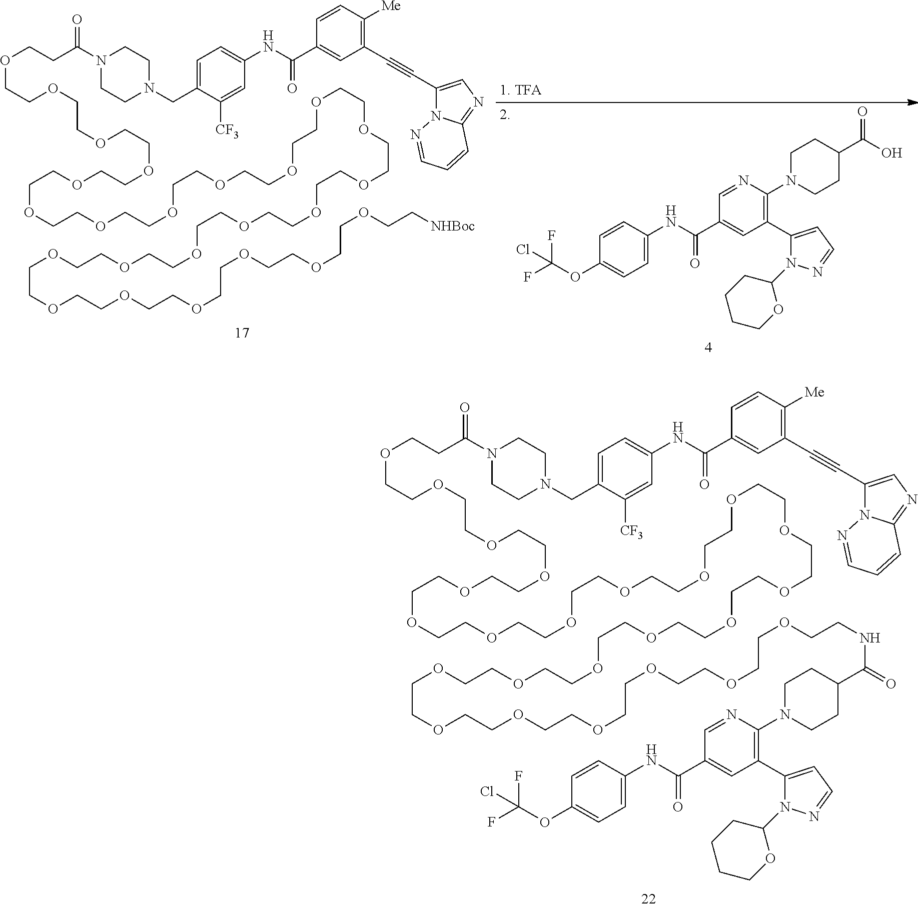 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCNC(=O)OC(C)(C)C)CC3)c(C(F)(F)F)c2)cc1C#Cc1cnc2cccnn12 ; O=C(Nc1ccc(OC(F)(F)Cl)cc1)c1cnc(N2CCC(C(=O)O)CC2)c(-c2ccnn2C2CCCCO2)c1 ; Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccnn5C5CCCCO5)CC4)CC3)c(C(F)(F)F)c2)cc1C#Cc1cnc2cccnn12]

Compound 22. The same procedure as for compound 20, using compound 17 (360 mg, 0.175 mmol) as starting material with scaled reagents, afforded compound 22 as a pale yellow oil (116 mg, 0.047 mmol, 27% yield over 2 steps).

HRMS (m/z): calculated for C₁₀₆H₁₅₃ClF₅N₁₂O₃₀⁺ [M+H]⁺ 2204.0419, found 2204.1145. and half 1102.5717. That's a little off, full would be 2204.1361. TLC: R_{f}=0.7 (20% methanol-DCM).

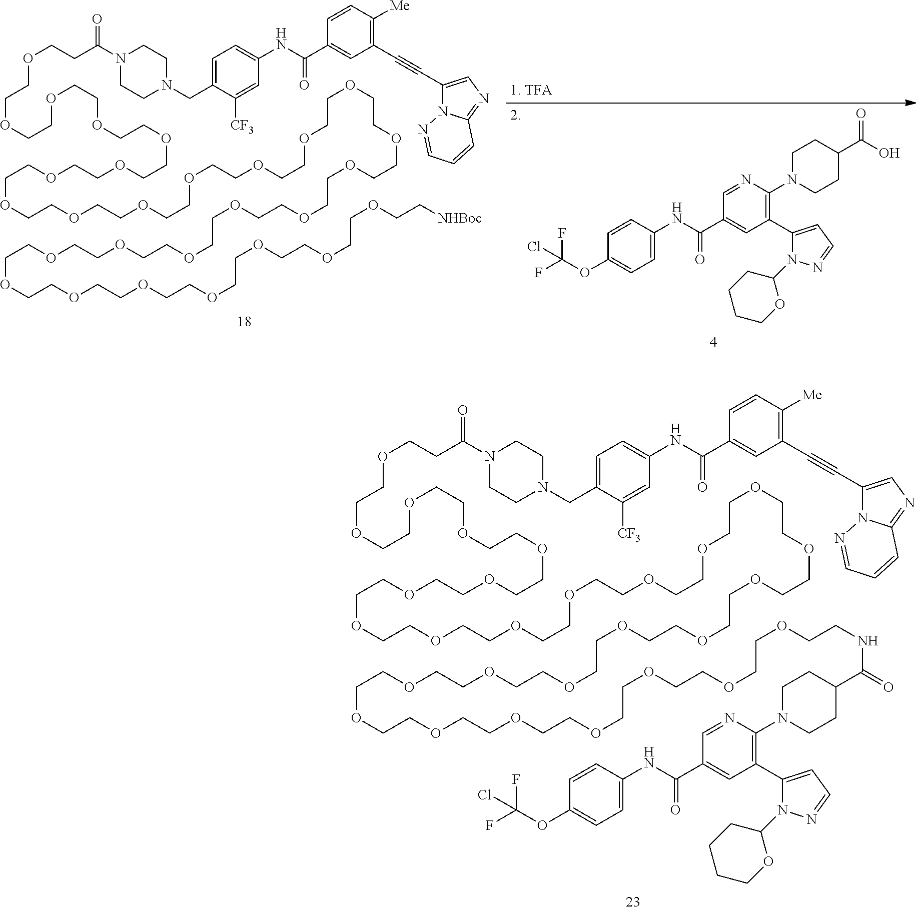 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)OC(C)(C)C)CC3)c(C(F)(F)F)c2)cc1C#Cc1cnc2cccnn12 ; O=C(Nc1ccc(OC(F)(F)Cl)cc1)c1cnc(N2CCC(C(=O)O)CC2)c(-c2ccnn2C2CCCCO2)c1 ; Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccnn5C5CCCCO5)CC4)CC3)c(C(F)(F)F)c2)cc1C#Cc1cnc2cccnn12]

Compound 23. The same procedure as for compound 20, using compound 18 (95 mg, 0.049 mmol) as starting material with scaled reagents, afforded compound 23 as a pale yellow oil (63.6 mg, 0.027 mmol, 54% yield over 2 steps).

¹H NMR (400 MHZ, DMSO-d₆) δ 13.00 (s, 1H), 11.21 (s, 0H), 9.52 (s, 1H), 8.95 (d, J=2.4 Hz, 1H), 8.68-8.58 (m, 2H), 8.52 (dd, J=4.4, 1.6 Hz, 1H), 8.26 (d, J=2.0 Hz, 1H), 8.13 (d, J=2.5 Hz, 1H), 8.10-8.03 (m, 2H), 7.99 (td, J=8.5, 2.2 Hz, 2H), 7.81 (d, J=9.1 Hz, 1H), 7.65 (d, J=1.7 Hz, 1H), 7.40 (d, J=8.1 Hz, 1H), 7.28 (s, 2H), 7.25-7.16 (m, 3H), 6.40 (d, J=1.8 Hz, 1H), 6.25 (d, J=5.6 Hz, 1H), 5.12 (dd, J=10.0, 2.4 Hz, 1H), 4.72 (s, 1H), 4.36 (s, 2H), 4.10 (s, 2H), 3.98 (d, J=11.3 Hz, 1H), 3.87 (d, J=13.0 Hz, 1H), 3.82-3.71 (m, OH), 3.71-3.54 (m, 113H), 3.42 (dd, J=10.0, 4.8 Hz, 3H), 3.09 (qd, J=7.5, 4.2 Hz, 1H), 2.82 (d, J=12.9 Hz, 5H), 2.65 (s, 3H), 2.48 (dd, J=17.4, 8.0 Hz, 1H), 2.29 (dt, J=11.0, 6.2 Hz, 1H), 2.06 (d, J=12.6 Hz, 1H), 1.91 (d, J=13.6 Hz, 1H), 1.69 (q, J=11.0 Hz, 5H), 1.61-1.50 (m, 3H), 1.45 (d, J=6.6 Hz, 2H), 1.26 (s, 1H). ¹⁹F NMR (376 MHz, DMSO-d₆) δ 25.61, −56.75. HRMS (m/z): calculated for C₁₁₄H₁₆₉ClF₅N₁₂O₃₄⁺ [M+H]⁺ 2380.1468, found 2380.2454. TLC: R_{f}=0.7 (20% methanol-DCM).

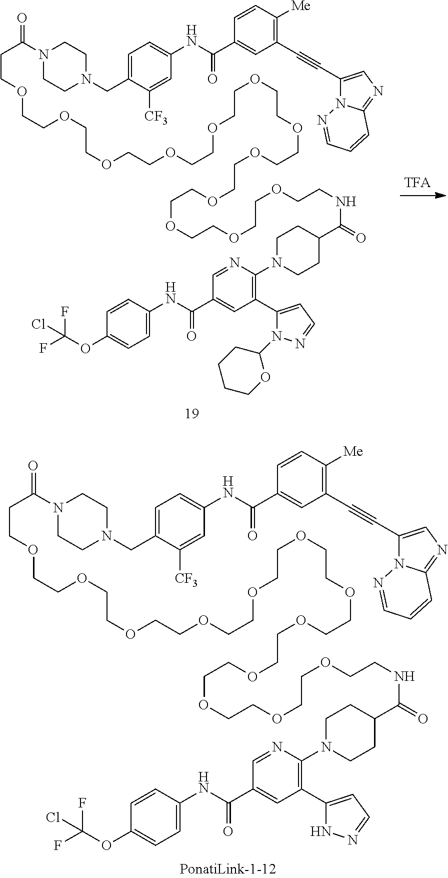 [SMILES: Cc1ccc(C(=O)Nc2ccc(CN3CCN(C(=O)CCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccnn5C5CCCCO5)CC4)CC3)c(C(F)(F)F)c2)cc1C#Cc1cnc2cccnn12 ; Cc1ccc(C(=O)Nc2ccc(CN3CCN(C(=O)CCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

PonatiLink-1-12. To a solution of compound 19 (174 mg, 0.097 mmol) in DCM (0.97 mL) was added TFA (0.32 mL). The mixture was stirred at room temperature for 6 hours, then concentrated under argon stream. The residue was dry-loaded on silica and purified by flash chromatography (0-20% methanol/DCM), then further purified by HPLC (30-90% acetonitrile/water+0.1% formic acid) to afford PonatiLink-1-12 (10 mg, 0.006 mmol, 6% yield) as a clear film.

¹H NMR (400 MHz, DMSO-d₆) δ 13.03 (s, 1H), 10.58 (s, 1H), 10.40 (s, 1H), 8.76-8.70 (m, 2H), 8.36-8.28 (m, 2H), 8.26 (dd, J=9.2, 1.6 Hz, 1H), 8.24-8.21 (m, 4H), 8.12-8.07 (m, 1H), 7.95 (dd, J=8.0, 2.0 Hz, 1H), 7.88 (d, J=9.0 Hz, 2H), 7.83 (t, J=5.7 Hz, 1H), 7.75 (d, J=8.6 Hz, 1H), 7.55 (d, J=8.2 Hz, 1H), 7.39 (dd, J=9.2, 4.5 Hz, 1H), 7.34 (d, J=8.8 Hz, 2H), 6.65 (s, 1H), 3.69-3.57 (m, 6H), 3.53-3.43 (m, 56H), 3.43-3.31 (m, 163H), 3.18 (q, J=5.9 Hz, 2H), 2.79-2.65 (m, 3H), 2.61 (s, 4H), 2.56 (t, J=6.7 Hz, 2H), 2.50 (p, J=1.9 Hz, 37H), 2.43-2.31 (m, 5H), 1.63 (s, 4H). ¹⁹F NMR (376 MHz, DMSO-d₆) δ −24.70, −56.82, −74.57. HRMS (m/z): calculated for C₇₇H₉₇ClF₅N₁₂O₁₇⁺ [M+H]⁺ 1591.6698, found 1591.6536. TLC: R_{f}=0.7 (20% methanol-DCM).

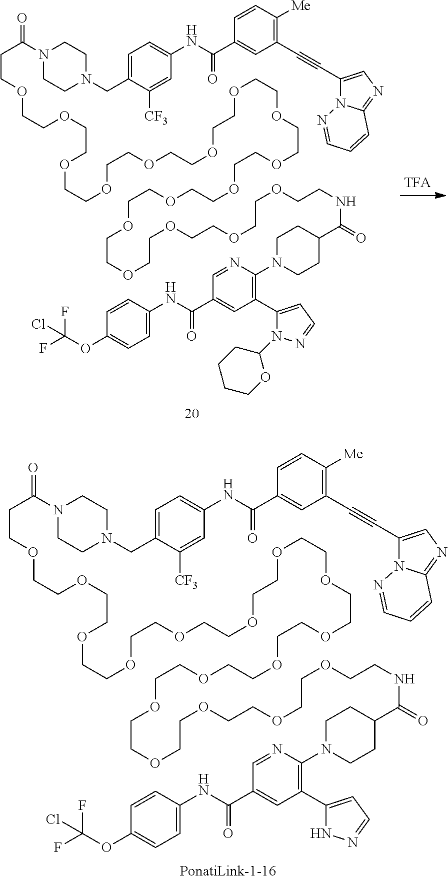 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccnn5C5CCCCO5)CC4)CC3)c(C(F)(F)F)c2)cc1C#Cc1cnc2cccnn12 ; Cc1ccc(C(=O)Nc2ccc(CN3CCN(C(=O)CCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

PonatiLink-1-6. The same procedure as for Ponati Link-1-12, using compound 20 (206 mg, 0.111 mmol) as starting material with scaled reagents and replacing formic acid with TFA in HPLC solvents, afforded PonatiLink-1-16 as the 1.5×TFA salt (106 mg, 0.0548 mmol, 49% yield) as a clear film.

¹H NMR (600 MHz, DMSO-d₆) δ 10.76 (s, 1H), 10.42 (s, 1H), 8.75 (d, J=2.4 Hz, 1H), 8.72 (dd, J=4.4, 1.5 Hz, 1H), 8.35 (s, 0H), 8.33 (d, J=2.4 Hz, 1H), 8.28-8.20 (m, 4H), 7.97 (dd, J=7.9, 2.0 Hz, 1H), 7.91-7.84 (m, 4H), 7.80 (d, J=2.1 Hz, 1H), 7.54 (d, J=8.1 Hz, 1H), 7.39 (dd, J=9.2, 4.4 Hz, 1H), 7.33 (d, J=8.5 Hz, 2H), 6.66 (d, J=2.2 Hz, 1H), 4.38 (s, 2H), 3.69-3.58 (m, 4H), 3.49 (s, 31H), 3.39 (t, J=6.0 Hz, 2H), 3.20 (q, J=5.9 Hz, 2H), 2.77-2.67 (m, 2H), 2.65-2.59 (m, 4H), 2.30 (hept, J=5.4 Hz, 1H), 1.68-1.60 (m, 4H). ¹⁹F NMR (376 MHz, DMSO-d₆) δ 24.75, −56.87, −74.44. ¹³C NMR (151 MHz, DMSO-d₆) δ 174.33, 169.17, 164.88, 164.11, 160.89, 158.58 (q, J=34.4 Hz), 146.51, 146.19, 145.12, 144.97, 143.83, 140.43, 139.68, 138.53, 138.15, 137.72, 133.59, 131.95, 130.25, 130.15, 129.02 (q, J=29.7 Hz), 128.58, 126.08, 125.05 (t, J=287.0 Hz), 123.80 (q, J=274.1 Hz), 123.47, 122.19, 121.83, 121.50, 119.19, 117.69-117.44 (m), 116.33 (q, J=294.2 Hz), 111.78, 103.50, 96.41, 81.23, 69.82, 69.64, 69.13, 66.69, 55.71, 51.80, 51.46, 48.44, 42.41, 41.72, 38.50, 32.63, 28.12, 20.44. HRMS (m/z): calculated for C₈₅H₁₁₃ClF₅N₁₂O₂₁⁺ [M+H]⁺ 1767.7747, found 1767.8757. TLC: R_{f}=0.7 (20% methanol-DCM).

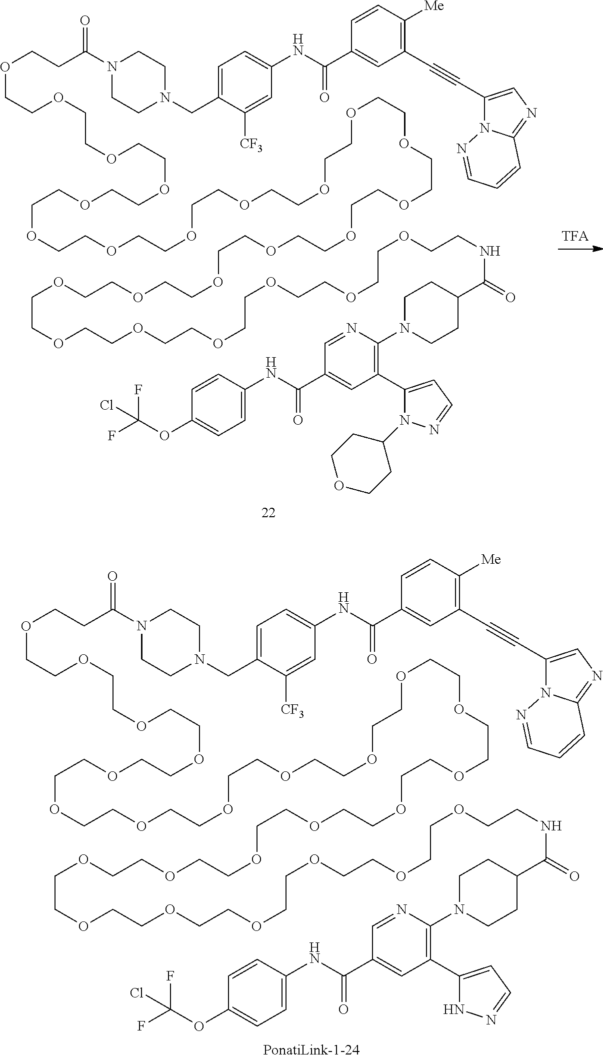 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccnn5C5CCOCC5)CC4)CC3)c(C(F)(F)F)c2)cc1C#Cc1cnc2cccnn12 ; Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

PonatiLink-1-24. The same procedure as for PonatiLink-1-16, using compound 22 (268 mg, 0.122 mmol) as starting material with scaled reagents, afforded PonatiLink-1-24 as the 3×TFA salt (116 mg, 0.047 mmol, 39% yield) as a clear film.

¹H NMR (400 MHz, DMSO-d₆) δ 10.78 (s, 1H), 10.43 (s, 1H), 8.78-8.73 (m, 2H), 8.35 (dd, J=14.8, 2.3 Hz, 2H), 8.31-8.23 (m, 4H), 7.98 (dd, J=8.0, 1.9 Hz, 1H), 7.93-7.84 (m, 4H), 7.81 (d, J=2.1 Hz, 1H), 7.57 (d, J=8.2 Hz, 1H), 7.42 (dd, J=9.2, 4.4 Hz, 1H), 7.34 (d, J=8.6 Hz, 2H), 6.67 (d, J=2.2 Hz, 1H), 4.46 (s, 2H), 3.70-3.59 (m, 5H), 3.50 (d, J=2.8 Hz, 110H), 3.40 (q, J=6.2 Hz, 3H), 3.20 (q, J=5.8 Hz, 2H), 2.74 (td, J=12.9, 4.5 Hz, 2H), 2.65 (d, J=9.5 Hz, 3H), 2.31 (tt, J=10.0, 5.7 Hz, 1H), 1.65 (td, J=10.1, 4.5 Hz, 4H). ¹⁹F NMR (376 MHz, DMSO-d₆) δ −24.75, −56.76, −74.84. HRMS (m/z): calculated for C₁₀₁H₁₄₅ClF₅N₁₂O₂₉⁺ [M+H]⁺ 2119.9844, found 2120.0686. TLC: R_{f}=0.7 (20% methanol-DCM).

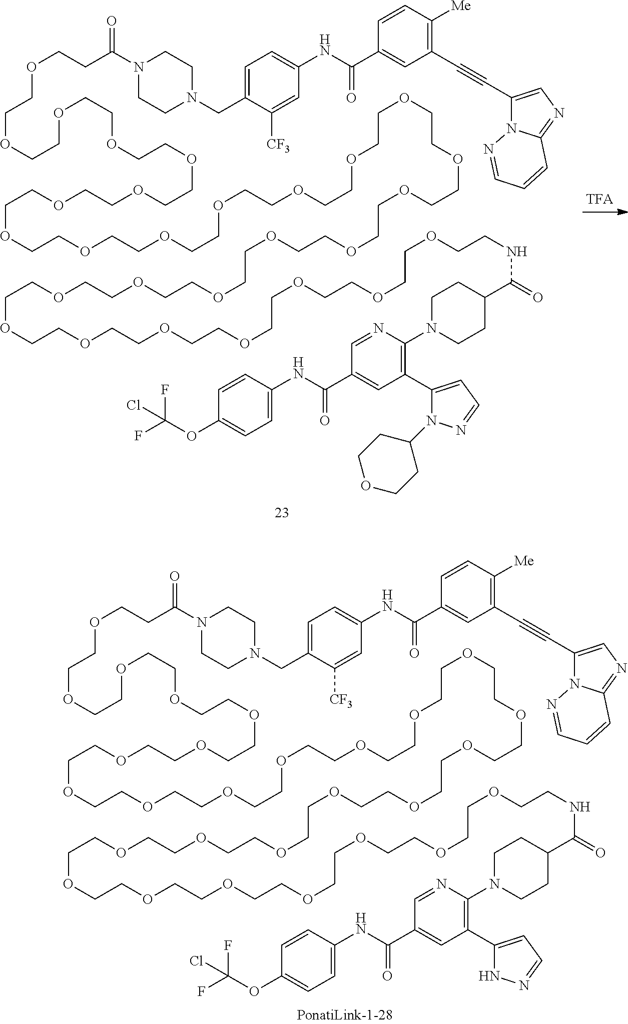

PonatiLink-1-28. The same procedure as for PonatiLink-1-16, using compound 23 (64 mg, 0.027 mmol) as starting material with scaled reagents, afforded PonatiLink-1-28 as the 2×TFA salt (27 mg, 0.011 mmol, 40% yield) as a clear film.

¹H NMR (400 MHZ, DMSO-d₆) δ 10.73 (s, 1H), 10.41 (s, 1H), 8.77-8.71 (m, 2H), 8.32 (d, J=2.5 Hz, 2H), 8.31-8.23 (m, 4H), 7.98 (dd, J=8.1, 1.9 Hz, 1H), 7.94-7.79 (m, 5H), 7.58 (d, J=8.2 Hz, 1H), 7.41 (dd, J=9.2, 4.5 Hz, 1H), 7.38-7.30 (m, 2H), 6.66 (d, J=2.1 Hz, 1H), 3.71-3.60 (m, 5H), 3.50 (s, 97H), 3.47-3.36 (m, 22H), 2.78-2.67 (m, 1H), 2.63 (s, 4H), 2.30 (p, J=8.0 Hz, 1H), 1.64 (s, 4H). ¹⁹F NMR (376 MHZ, DMSO-d₆) δ −24.71, −56.85, −74.19. HRMS (m/z): calculated for C₁₀₉H₁₆₁ClF₅N₁₂O₃₃⁺ [M+H]⁺ 2296.0892, found 2296.1729. TLC: R_{f}=0.7 (20% methanol-DCM).

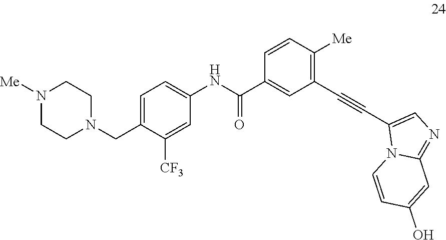 [SMILES: Cc1ccc(C(=O)Nc2ccc(CN3CCN(C)CC3)c(C(F)(F)F)c2)cc1C#Cc1cnc2cc(O)ccn12]

Compound 24 was prepared according to patent WO2015106292AI by Pharmaron Inc. as a pale brown solid (1.6 g).

¹H NMR (400 MHz, DMSO-d₆) δ 10.58 (s, 1H), 10.54 (s, 1H), 8.41 (d, J=7.3 Hz, 1H), 8.22 (s, 2H), 8.08 (dd, J=8.6, 2.2 Hz, 1H), 7.90 (dd, J=8.0, 2.0 Hz, 1H), 7.84 (s, 1H), 7.71 (d, J=8.5 Hz, 1H), 7.53 (d, J=8.1 Hz, 1H), 6.85 (d, J=2.3 Hz, 1H), 6.80 (dd, J=7.3, 2.4 Hz, 1H), 4.09 (s, 1H), 3.59 (s, 2H), 3.17 (s, 3H), 2.59 (s, 3H), 2.32 (s, 3H), 1.23 (s, 1H). ¹⁹F NMR (376 MHz, DMSO-d₆) δ −57.97. HRMS (m/z): calculated for C₃₀H₂₉F₃N₅O₂⁺ [M+H]⁺ 548.2268, found 548.2241.

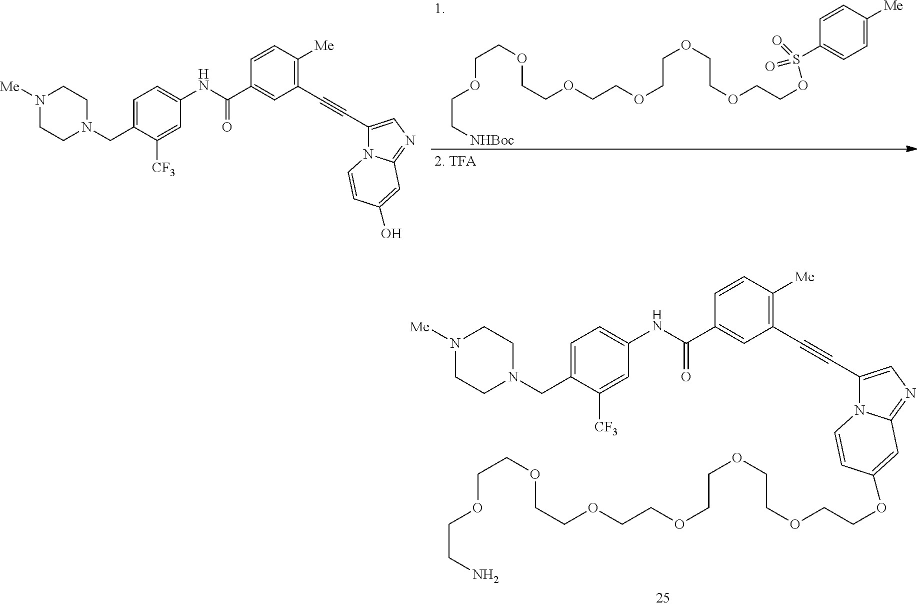 [SMILES: Cc1ccc(C(=O)Nc2ccc(CN3CCN(C)CC3)c(C(F)(F)F)c2)cc1C#Cc1cnc2cc(O)ccn12 ; Cc1ccc(S(=O)(=O)OCCOCCOCCOCCOCCOCCOCCNC(=O)OC(C)(C)C)cc1 ; Cc1ccc(C(=O)Nc2ccc(CN3CCN(C)CC3)c(C(F)(F)F)c2)cc1C#Cc1cnc2cc(OCCOCCOCCOCCOCCOCCOCCN)ccn12]

Compound 25. To a solution of compound 24 (595 mg, 1.087 mmol) and t-Boc-N-amido-PEG₇-Tos (630 mg, 1.087 mmol) in DMF (10.9 mL) was added cesium carbonate (708 mg, 2.175 mmol), and the mixture was stirred at 50° C. overnight. The mixture was partitioned between water and ethyl acetate. The aqueous layer was extracted with ethyl acetate (3×), and the combined organics were washed with water (1×) and brine (1×), dried over sodium sulfate, filtered and concentrated in vacuo, and the residue was used directly in the next step without further purification. To a solution of the crude residue in DCM (11.3 mL) was added TFA (3.65 mL, 47.7 mmol). The mixture was stirred at room temperature for 10 minutes, then concentrated under argon stream. The resulting residue was dry-loaded on celite and purified by reverse-phase C18 flash chromatography (10-60% acetonitrile/water+0.1% TFA) to afford compound 25 as a foamy orange solid (642 mg, 0.536 mmol, 49% yield over two steps).

¹H NMR (400 MHz, DMSO-d₆) δ 10.70-10.60 (m, 1H), 8.72 (s, 1H), 8.44-8.23 (m, 3H), 8.12 (dd, J=8.5, 2.2 Hz, 1H), 8.00 (d, J=8.0 Hz, 1H), 7.78 (d, J=33.1 Hz, 3H), 7.58 (d, J=8.2 Hz, 1H), 7.49-7.29 (m, 1H), 7.19 (s, 1H), 4.34 (s, 2H), 3.84 (s, 2H), 3.69 (s, 2H), 3.66-3.36 (m, 21H), 3.05 (s, 0H), 3.03-2.89 (m, 3H), 2.82 (s, 3H), 2.62 (s, 2H). ¹⁹F NMR (376 MHz, DMSO-d₆) δ 57.96, −74.10-−74.30 (m). HRMS (m/z): calculated for C₄₄H₅₈F₃N₆O₈⁺ [M+H]⁺ 855.4263, found 855.4293.

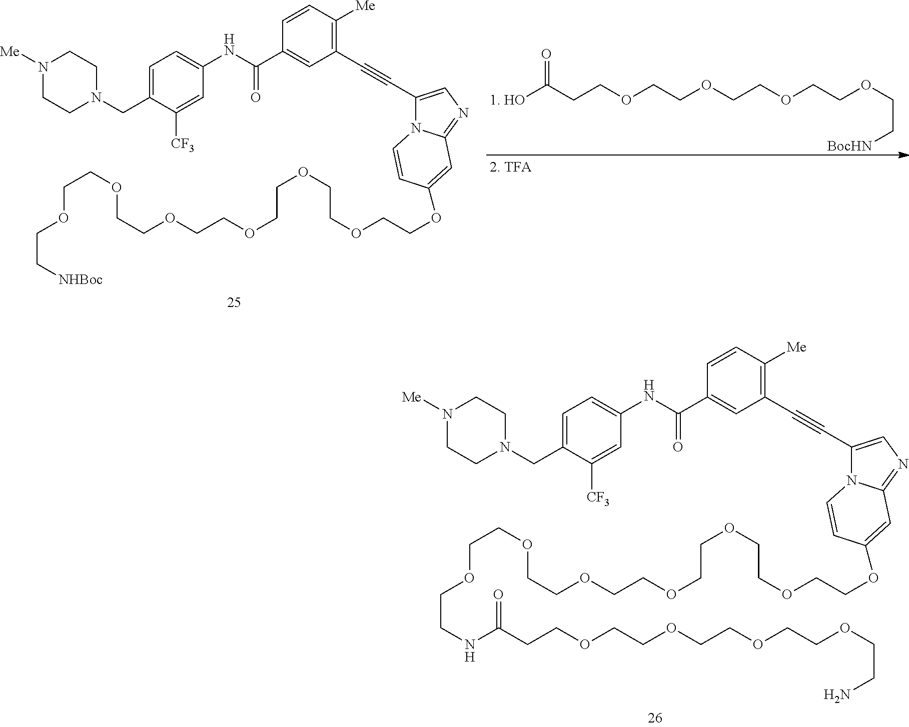

Compound 26. To a solution of compound 25 (154 mg, 0.129 mmol), t-Boc-N-amido-PEG4-acid (47 mg, 0.129 mmol) and DIPEA (224 μL, 1.286 mmol) in DMF (1.29 mL) was added HATU (98 mg, 0.257 mmol), and the mixture was stirred at room temperature for 30 minutes. The mixture was partitioned between ethyl acetate and water. The aqueous layer was extracted with ethyl acetate (3×), and the combined organics were washed with water (1×) and brine (1×), dried over sodium sulfate and concentrated in vacuo. The residue was used directly in the next step without further purification. To a solution of the crude residue in DCM (1.29 mL) was added TFA (0.41 mL). The mixture was stirred at room temperature for 10 minutes, then concentrated under argon stream. The resulting residue was dry-loaded on celite and purified by reverse-phase C18 flash chromatography (20-60% acetonitrile/water+0.1% TFA) to afford compound 26 as the 5×TFA salt (160 mg, 0.095 mmol, 74% yield over two steps) as an orange semisolid.

¹H NMR (400 MHz, DMSO-d₆) δ 10.63 (s, 1H), 8.70 (d, J=7.4 Hz, 1H), 8.32 (d, J=15.4 Hz, 2H), 8.24 (d, J=2.2 Hz, 1H), 8.12 (d, J=8.2 Hz, 1H), 7.99 (d, J=7.9 Hz, 1H), 7.92 (s, 1H), 7.73 (d, J=8.6 Hz, 1H), 7.59 (d, J=8.2 Hz, 1H), 7.36 (s, 1H), 7.18 (d, J=7.5 Hz, 1H), 4.35 (s, 2H), 3.97 (s, 1H), 3.84 (d, J=4.9 Hz, 2H), 3.69 (s, 2H), 3.66-3.54 (m, 12H), 3.54-3.47 (m, 26H), 3.45-3.32 (m, 4H), 3.17 (s, 12H), 2.82 (s, 3H), 2.70 (s, 2H), 2.62 (s, 3H), 2.32 (t, J=6.5 Hz, 2H), 1.33-1.18 (m, 2H), 1.13 (d, J=6.7 Hz, 1H). ¹⁹F NMR (376 MHz, DMSO-d₆) δ −57.96, −69.20, −71.09, −74.37 (d, J=5.7 Hz). HRMS (m/z): calculated for C₅₅H₇₉F₃N₇O₁₃⁺ [M+H]⁺ 1102.5683, found 1102.5707.

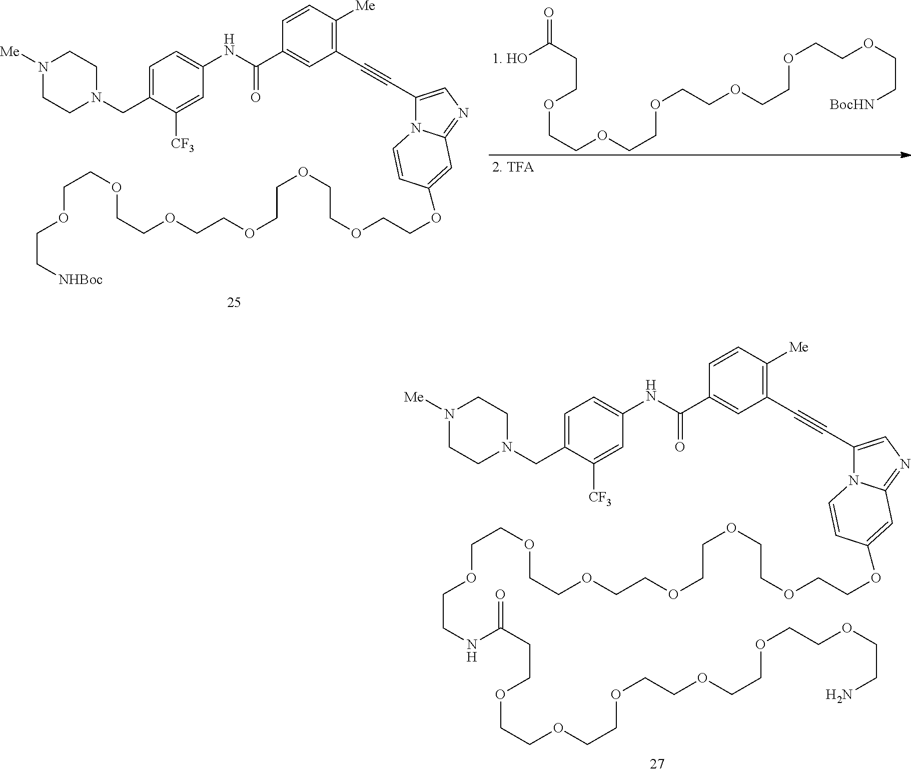

Compound 27. The same procedure as for compound 26, using t-Boc-N-amido-PEG₆-acid (62 mg, 0.136 mmol) as starting material with scaled reagents, afforded compound 27 as the 5×TFA salt (150 mg, 0.086 mmol, 63% yield over two steps) as an orange semisolid.

¹H NMR (400 MHz, DMSO-d₆) δ 10.63 (s, 1H), 8.71 (d, J=7.5 Hz, 1H), 8.35 (s, 1H), 8.30 (d, J=1.9 Hz, 1H), 8.24 (d, J=2.2 Hz, 1H), 8.16-8.09 (m, 1H), 7.99 (dd, J=8.0, 1.9 Hz, 1H), 7.90 (t, J=5.7 Hz, 1H), 7.73 (d, J=8.6 Hz, 1H), 7.59 (d, J=8.2 Hz, 1H), 7.36 (s, 1H), 7.19 (d, J=7.5 Hz, 1H), 4.35 (d, J=5.1 Hz, 2H), 3.97 (s, 2H), 3.86-3.82 (m, 2H), 3.69 (s, 2H), 3.66-3.53 (m, 12H), 3.53 (s, 2H), 3.39 (t, J=5.9 Hz, 2H), 3.19 (d, J=8.1 Hz, 3H), 3.07-2.89 (m, 6H), 2.82 (s, 3H), 2.62 (s, 3H), 2.44-2.28 (m, 4H). ¹⁹F NMR (376 MHz, DMSO-d₆) δ −57.96, −74.44. HRMS (m/z): calculated for C₅₉H₈₇F₃N₇O₁₅⁺ [M+H]⁺ 1190.6207, found 1190.6252.

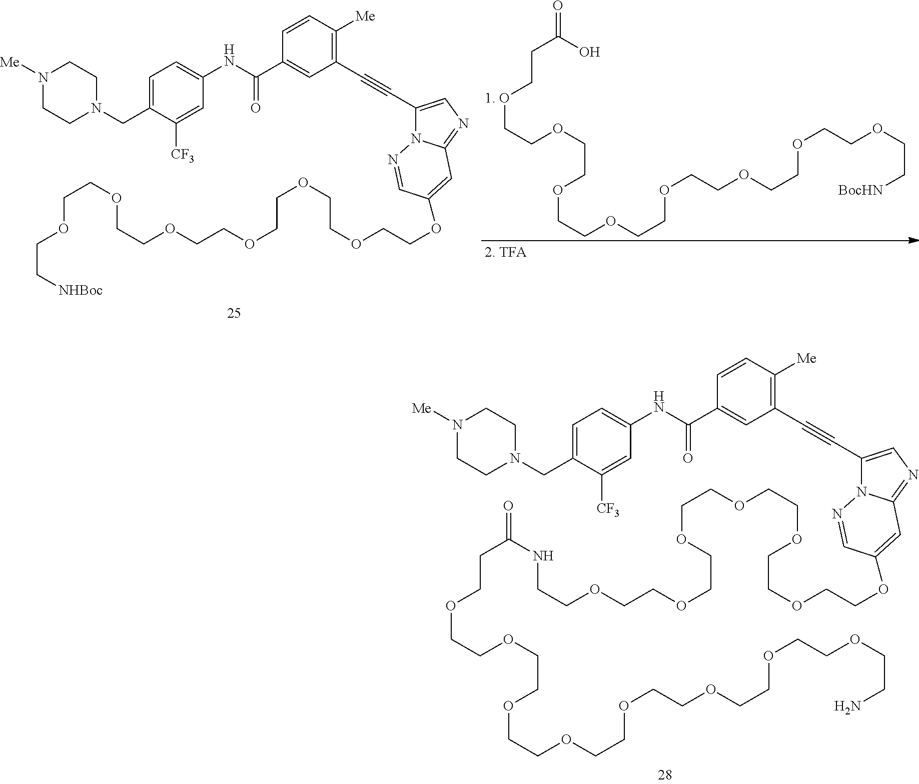

Compound 28. The same procedure as for compound 26, using t-Boc-N-amido-PEG₈-acid (60 mg, 0.112 mmol) as starting material with scaled reagents, afforded compound 17 as the 4×TFA salt (130 mg, 0.075 mmol, 68% yield over two steps) as an orange semisolid.

¹H NMR (400 MHz, DMSO-d₆) δ 10.64 (s, 1H), 8.73 (d, J=7.4 Hz, 1H), 8.38 (s, 1H), 8.30 (s, 1H), 8.24 (d, J=2.1 Hz, 1H), 8.16-8.09 (m, 1H), 8.00 (d, J=8.0 Hz, 1H), 7.90 (t, J=5.7 Hz, 1H), 7.73 (d, J=8.6 Hz, 1H), 7.59 (d, J=8.2 Hz, 1H), 7.38 (s, 1H), 7.21 (s, 1H), 4.36 (s, 2H), 3.97 (s, 0H), 3.84 (s, 2H), 3.69 (s, 2H), 3.66-3.55 (m, 12H), 3.54-3.47 (m, 45H), 3.39 (t, J=5.9 Hz, 3H), 3.23-3.14 (m, 3H), 3.07-2.89 (m, 5H), 2.82 (s, 3H), 2.62 (s, 3H), 2.44-2.37 (m, 7H), 2.31 (t, J=6.5 Hz, 2H). ¹⁹F NMR (376 MHz, DMSO-d₆) δ −57.96, −74.44 (d, J=5.1 Hz). HRMS (m/z): calculated for C₆₃H₉₅F₃N₇O₁₇⁺ [M+H]⁺ 1278.6731, found 1278.6783.

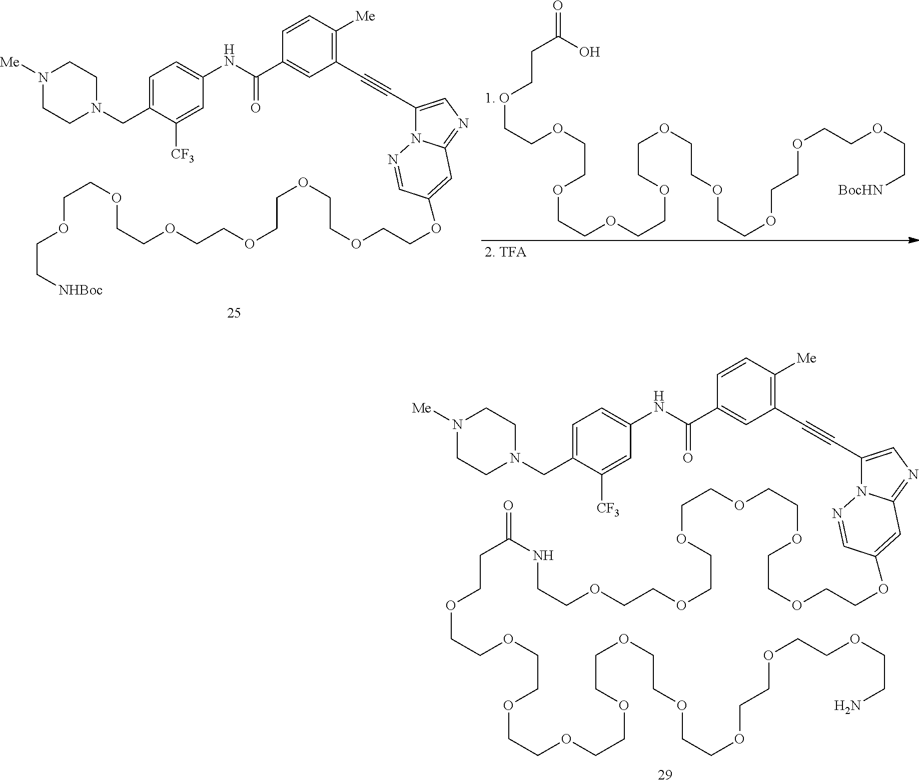

Compound 29. The same procedure as for compound 26, using t-Boc-N-amido-PEG10-acid (80 mg, 0.127 mmol) as starting material with scaled reagents, afforded compound 29 as the 4×TFA salt (163 mg, 0.089 mmol, 70% yield over two steps) as an orange semisolid.

¹H NMR (400 MHZ, DMSO-d₆) δ 10.63 (s, 1H), 8.70 (d, J=7.5 Hz, 1H), 8.36-8.27 (m, 2H), 8.24 (d, J=2.2 Hz, 1H), 8.12 (d, J=9.3 Hz, 1H), 7.99 (dd, J=8.1, 1.9 Hz, 1H), 7.89 (t, J=5.6 Hz, 1H), 7.73 (d, J=8.6 Hz, 2H), 7.59 (d, J=8.2 Hz, 1H), 7.35 (d, J=2.5 Hz, 1H), 7.18 (dd, J=7.5, 2.5 Hz, 1H), 4.34 (d, J=4.5 Hz, 2H), 3.97 (s, 1H), 3.83 (d, J=4.9 Hz, 2H), 3.69 (s, 2H), 3.66-3.51 (m, 23H), 3.51 (s, 29H), 3.39 (t, J=6.0 Hz, 2H), 3.18 (s, 10H), 3.07-2.96 (m, 2H), 2.93 (d, J=13.2 Hz, 3H), 2.82 (s, 3H), 2.62 (s, 3H), 2.40 (d, J=12.1 Hz, 1H), 2.31 (t, J=6.5 Hz, 2H). ¹⁹F NMR (376 MHz, DMSO-d₆) δ −57.96, −74.37. HRMS (m/z): calculated for C₆₇H₁₀₃F₃N₇O₁₉⁺ [M+H]⁺ 1366.7256, found 1366.7222.

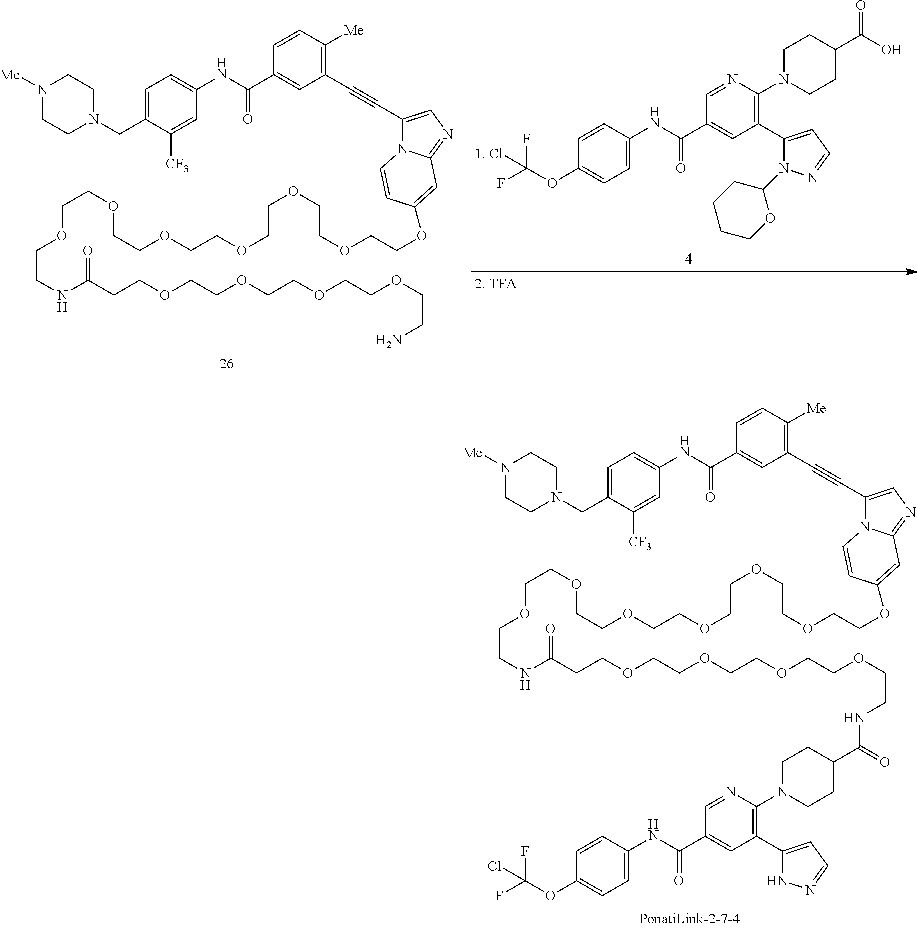

PonatiLink-2-7-4. To a solution of compound 26 (158 mg, 0.094 mmol), compound 4 (54 mg, 0.094 mmol), and HATU (72 mg, 0.189 mmol) in DCM (0.95 mL) was added DIPEA (165 μL, 0.945 mmol), and the mixture was stirred at room temperature for 5 hours. LCMS analysis indicated the formation of both desired product and the amine-methylguanidinium adduct as a side product. The mixture was partitioned between brine and DCM. The aqueous layer was extracted with DCM (3×), and the combined organics were washed with water (1×) and brine (1×), dried over sodium sulfate, filtered and concentrated in vacuo. The residue was used directly in the next step with further purification. To a solution of the crude residue in DCM (0.95 mL) was added TFA (0.58 mL, 7.57 mmol). The mixture was stirred at room temperature for 2 hours, then concentrated under argon stream. The resulting residue was dry-loaded on celite and purified by reverse-phase C18 flash chromatography (10-65% acetonitrile/water+0.1% TFA), which failed to fully remove the methylguanidinium impurity, then further purified by HPLC (10-65% acetonitrile/water+0.1% formic acid) to afford PonatiLink-2-7-4 as the 2×TFA salt (47 mg, 0.026 mmol, 47% yield over two steps) as a white solid.

¹H NMR (400 MHz, DMSO-d₆) δ 10.65 (s, 1H), 10.41 (s, 1H), 8.78-8.67 (m, 2H), 8.39-8.28 (m, 3H), 8.24 (d, J=2.2 Hz, 1H), 8.12 (dd, J=8.4, 2.2 Hz, 1H), 7.99 (dd, J=8.1, 1.9 Hz, 1H), 7.94-7.83 (m, 4H), 7.80 (d, J=2.1 Hz, 1H), 7.72 (d, J=8.6 Hz, 1H), 7.58 (d, J=8.2 Hz, 1H), 7.40-7.31 (m, 3H), 7.20 (d, J=7.5 Hz, 1H), 6.66 (d, J=2.2 Hz, 1H), 4.34 (dd, J=5.7, 3.1 Hz, 2H), 3.83 (dd, J=5.3, 3.5 Hz, 2H), 3.69 (s, 2H), 3.66-3.54 (m, 6H), 3.54-3.43 (m, 28H), 3.39 (td, J=5.9, 4.0 Hz, 4H), 3.19 (qd, J=5.8, 2.6 Hz, 4H), 3.05 (s, 1H), 2.93 (d, J=12.3 Hz, 2H), 2.82 (s, 3H), 2.78-2.66 (m, 2H), 2.61 (s, 3H), 2.40 (s, 2H), 2.30 (q, J=6.7 Hz, 3H), 1.63 (d, J=5.9 Hz, 4H). ¹⁹F NMR (376 MHz, DMSO-d₆) δ −24.72, −57.96, −74.21. HRMS (m/z): calculated for C₇₇H₉₇ClF₅N₁₂O₁₆ [M+H]⁺ 1575.6749, found 1575.6760.

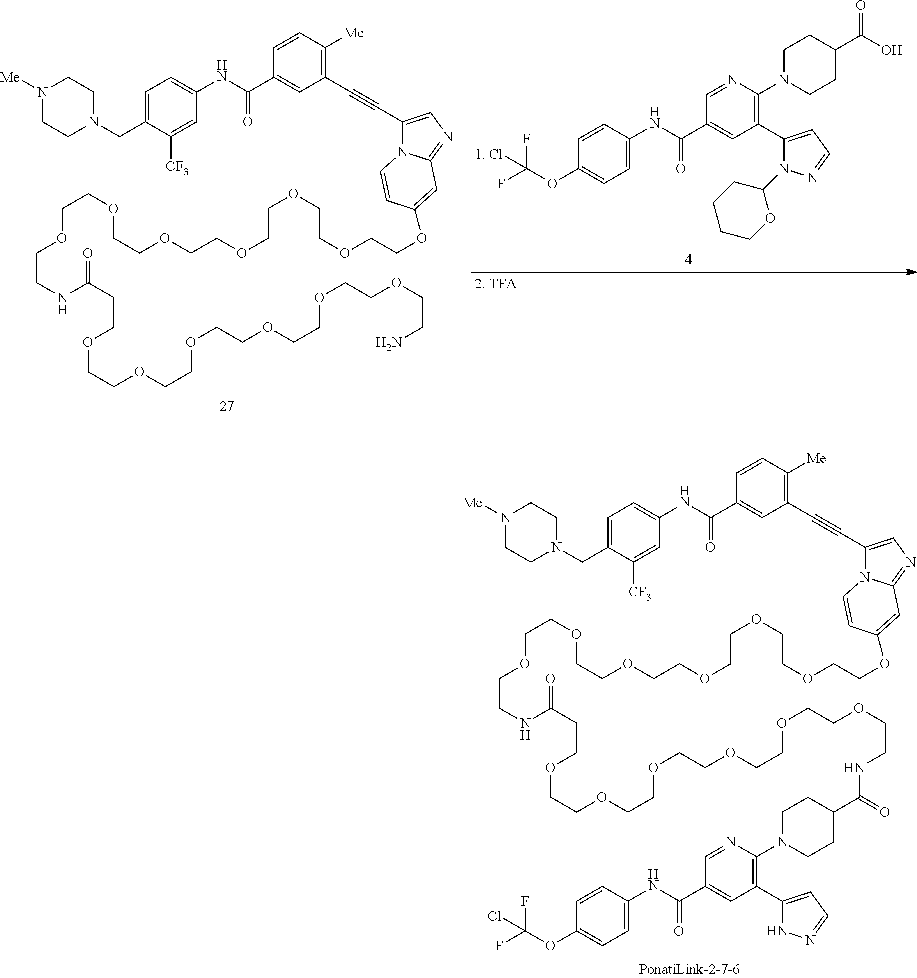

PonatiLink-2-7-6. To a solution of compound 4 (49 mg, 0.085 mmol) and DIPEA (147 μL, 0.846 mmol) in DCM (0.85 mL) was added HATU (64 mg, 0.169 mmol), and the mixture was stirred at room temperature for 15 minutes. The mixture was transferred to a vessel containing compound 27 (149 mg, 0.085 mmol) and stirred at room temperature for 2 hours. The mixture was partitioned between brine and DCM. The aqueous layer was extracted with DCM (3×), and the combined organics were washed with water (1×) and brine (1×), dried over sodium sulfate, filtered and concentrated in vacuo. The residue was used directly in the next step with further purification. To a solution of the crude residue in DCM (0.85 mL) was added TFA (0.52 mL, 6.77 mmol). The mixture was stirred at room temperature for 4 hours, then concentrated under argon stream. The resulting residue was dry-loaded on celite and purified by reverse-phase C18 flash chromatography (10-65% acetonitrile/water+0.1% TFA), then further purified by HPLC (10-65% acetonitrile/water+0.1% formic acid) to afford PonatiLink-2-7-6 as the 2×TFA salt (67 mg, 0.035 mmol, 41% yield over two steps) as a white solid.

¹H NMR (400 MHz, DMSO-d₆) δ 10.69 (d, J=4.1 Hz, 1H), 10.43 (d, J=2.2 Hz, 1H), 8.79-8.71 (m, 2H), 8.49-8.43 (m, 1H), 8.33 (d, J=2.4 Hz, 2H), 8.25 (d, J=2.5 Hz, 1H), 8.13 (dd, J=8.5, 2.2 Hz, 1H), 8.01 (dd, J=8.0, 1.9 Hz, 1H), 7.89 (dd, J=9.7, 2.6 Hz, 4H), 7.81 (d, J=2.1 Hz, 1H), 7.73 (d, J=8.5 Hz, 1H), 7.57 (dt, J=8.4, 4.5 Hz, 1H), 7.47 (dd, J=8.8, 2.5 Hz, 1H), 7.33 (dd, J=9.1, 3.0 Hz, 2H), 7.27 (d, J=7.5 Hz, 1H), 6.66 (d, J=2.1 Hz, 1H), 4.40-4.33 (m, 2H), 3.84 (dd, J=5.7, 3.1 Hz, 2H), 3.75-3.54 (m, 9H), 3.53-3.46 (m, 36H), 3.39 (q, J=5.7 Hz, 4H), 3.20 (tt, J=8.7, 4.7 Hz, 4H), 2.82 (d, J=1.6 Hz, 3H), 2.78-2.67 (m, 2H), 2.61 (d, J=2.4 Hz, 3H), 2.36-2.28 (m, 3H), 1.63 (d, J=9.2 Hz, 4H). ¹⁹F NMR (376 MHz, DMSO-d₆) δ −24.76 (d, J=9.7 Hz), −57.97, −74.45 (dd, J=25.9, 12.7 Hz). HRMS (m/z): calculated for C₈₁H₁₀₅ClF₅N₁₂O₁₈⁺ [M+H]⁺ 1663.7273, found 1663.7349.

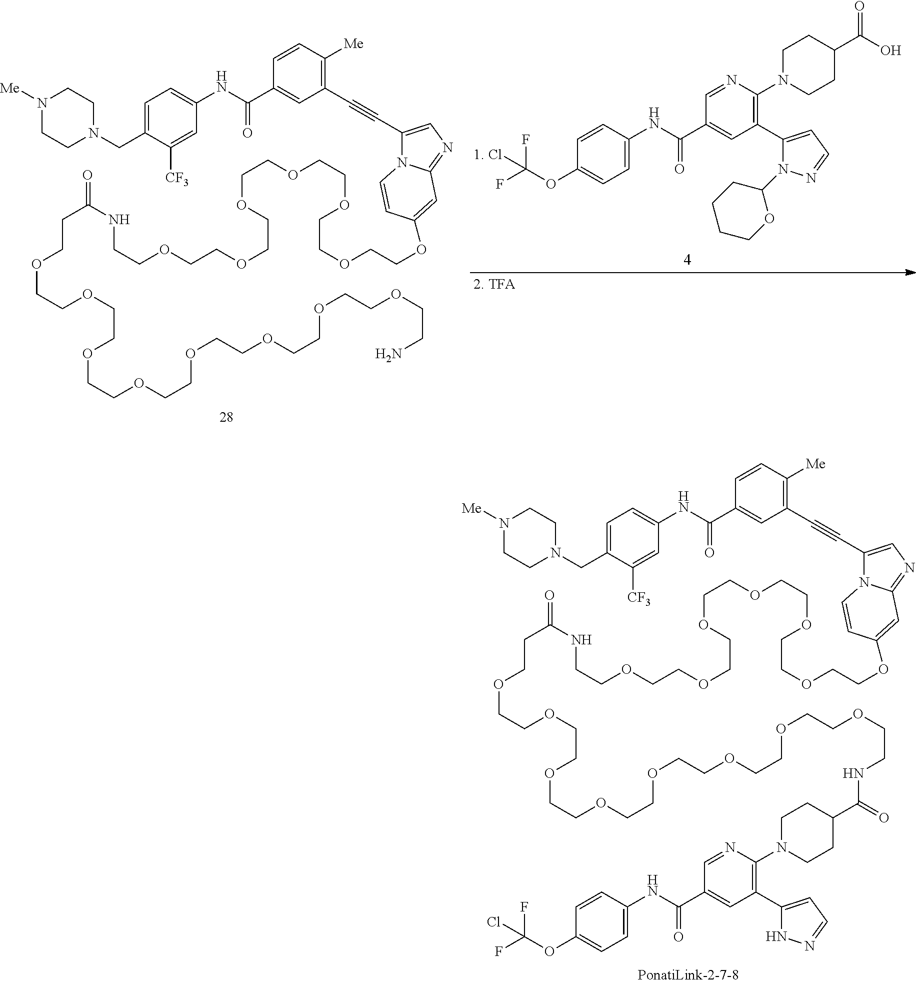

PonatiLink-2-7-8. The same procedure as for PonatiLink-2-7-6, using compound 28 (122 mg, 0.070 mmol) as starting material with scaled reagents and omitting the final HPLC purification, afforded PonatiLink-2-7-8 as the 3×TFA salt (70 mg, 0.033 mmol, 47% yield over two steps) as a white solid.

¹H NMR (400 MHz, DMSO-d₆) δ 10.66 (d, J=14.1 Hz, 1H), 10.41 (d, J=4.8 Hz, 1H), 8.77-8.68 (m, 2H), 8.42 (s, 1H), 8.32 (t, J=2.5 Hz, 2H), 8.24 (t, J=3.0 Hz, 1H), 8.12 (d, J=8.6 Hz, 1H), 8.00 (dd, J=7.9, 2.2 Hz, 1H), 7.87 (ddd, J=9.8, 7.5, 3.2 Hz, 4H), 7.80 (d, J=2.1 Hz, 1H), 7.72 (d, J=8.5 Hz, 1H), 7.56 (dd, J=8.3, 4.0 Hz, 1H), 7.44 (d, J=2.4 Hz, 1H), 7.33 (dd, J=9.1, 2.9 Hz, 3H), 7.23 (dd, J=7.5, 2.4 Hz, 1H), 6.65 (d, J=2.1 Hz, 1H), 4.35 (t, J=4.5 Hz, 2H), 3.83 (t, J=4.3 Hz, 2H), 3.72-3.53 (m, 8H), 3.53-3.43 (m, 46H), 3.23-3.14 (m, 5H), 3.05 (s, 1H), 2.96-2.87 (m, 1H), 2.81 (s, 3H), 2.77-2.66 (m, 2H), 2.61 (s, 3H), 2.43 (s, 3H), 2.30 (td, J=6.6, 1.9 Hz, 3H), 1.65 (d, J=12.2 Hz, 4H). ¹⁹F NMR (376 MHz, DMSO-d₆) δ −24.73 (d, J=8.5 Hz), −57.98, −74.30 (d, J=28.3 Hz). HRMS (m/z): calculated for C₈₅H₁₁₃ClF₅N₁₂O₂₀⁺ [M+H]⁺ 1751.7798, found 1751.7882.

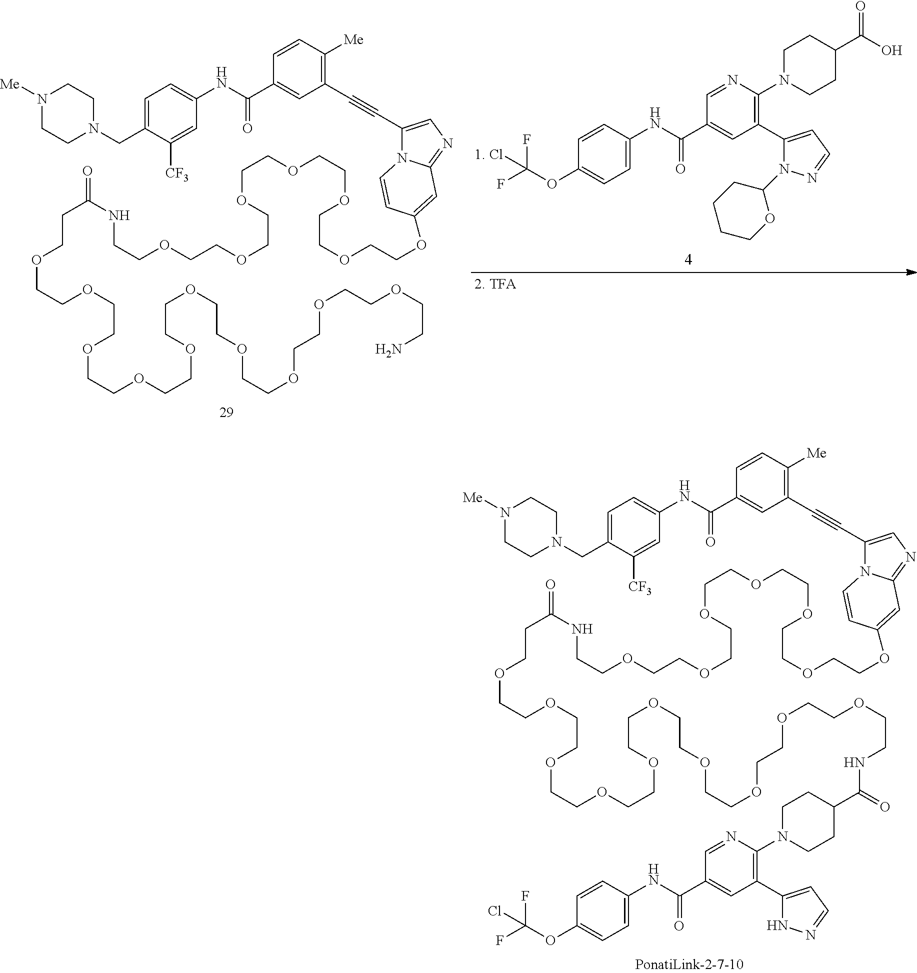

PonatiLink-2-7-10. The same procedure as for PonatiLink-2-7-4, using compound 29 (161 mg, 0.088 mmol) as starting material with scaled reagents, afforded PonatiLink-2-7-10 as the 2×TFA salt (40 mg, 0.019 mmol, 22% yield over two steps) as a white solid.

¹H NMR (400 MHz, DMSO-d₆) δ 10.64 (s, 1H), 10.41 (s, 1H), 8.74 (d, J=2.4 Hz, 1H), 8.67 (d, J=7.4 Hz, 1H), 8.30 (dd, J=7.9, 2.1 Hz, 3H), 8.23 (d, J=2.2 Hz, 1H), 8.11 (dd, J=8.6, 2.2 Hz, 1H), 7.98 (dd, J=8.0, 1.9 Hz, 1H), 7.87 (dq, J=7.9, 4.8 Hz, 4H), 7.80 (d, J=2.2 Hz, 1H), 7.72 (d, J=8.5 Hz, 1H), 7.56 (d, J=8.2 Hz, 1H), 7.34 (dd, J=10.4, 2.3 Hz, 4H), 7.16 (dd, J=7.4, 2.3 Hz, 1H), 6.65 (d, J=2.2 Hz, 1H), 4.36-4.29 (m, 2H), 3.86-3.79 (m, 2H), 3.67 (d, J=3.5 Hz, 3H), 3.65-3.52 (m, 7H), 3.52-3.44 (m, 54H), 3.38 (q, J=5.8 Hz, 5H), 3.18 (qd, J=5.8, 3.6 Hz, 4H), 3.04 (s, 2H), 2.92 (d, J=12.1 Hz, 2H), 2.81 (s, 3H), 2.77-2.66 (m, 2H), 2.60 (s, 3H), 2.41 (d, J=14.9 Hz, 1H), 2.30 (t, J=6.5 Hz, 3H), 1.68-1.58 (in, 4H). ¹⁹F NMR (376 MHz, DMSO-d₆) δ −24.72, −57.97, −74.07 (d, J=3.2 Hz). HRMS (m/z): calculated for C₈₉H₁₂₁ClF₅N₁₂O₂₂⁺ [M+H]⁺ 1839.8322, found 1839.8459.

### Example 5: Biological Data

TABLE 1
EC₅₀ values determined in CellTiter-Glo
and SelectScreen Z'LYTE assays.

| | | | EC₅₀ | EC₅₀ |
|---|---|---|---|---|
| | EC₅₀ | EC₅₀ | (nM) | (nM) |
| | (nM) | (nM) | Se- | Se- |
| | CTG | CTG K562 | lectScreen | lectScreen |
| Compound | K562 wt | T315I | Abl wt | Abl_T315I |
| asciminib | 7.59 | 88.07 | 28.19 | 6.45 |
| dasatinib | 0.28 | n/a | 0.13 | n/a |
| dasatinib + | 0.26 | 11.99 | 0.13 | n/a |
| asciminib | | | | |
| DasatiLink-1 | 2.47 | >300 nM | 0.44 | 7.23 |
| DasatiLink-2 | 2.49 | >300 nM | 0.8 | 23.8 |
| DasatiLink-3 | 2.94 | >300 nM | 1.04 | n/a |
| DasatiLink-4 | 2.62 | >300 nM | 1.81 | n/a |
| ponatinib | 0.4 | 1.77 | 2.8 | 4.18 |
| ponatinib + | 0.39 | 1.49 | 3.89 | 7.39 |
| asciminib | | | | |
| (conc for each) | | | | |
| PonatiLink-1-12 | 2.76 | 43.68 | 318.59 | 3583.02 |
| PonatiLink-1-16 | 1.4 | 28.72 | 81.99 | 228.8 |
| PonatiLink-1-20 | 1.02 | 26.2 | 10.77 | 214.69 |
| (PonatiLink-1) | | | | |
| PonatiLink-1-24 | 0.87 | 26.29 | 3.57 | 64.03 |
| PonatiLink-1-28 | 1.82 | 49.14 | n/a | n/a |
| PonatiLink-2-7-4 | 0.39 | 1.3 | n/a | n/a |
| PonatiLink-2-7-6 | 0.38 | 1.06 | n/a | n/a |
| PonatiLink-2-7-8 | 0.36 | 0.94 | n/a | n/a |
| PonatiLink-2-7-10 | 0.43 | 0.94 | n/a | n/a |

1. A compound comprising a monovalent ABL ATP binding site inhibitor covalently bound to a monovalent ABL myristoyl binding site inhibitor.

2. The compound of claim 1, having the formula:
A-L¹-B; or a pharmaceutically salt thereof, wherein
A is said monovalent ABL ATP binding site inhibitor;
B is said monovalent ABL myristoyl binding site inhibitor; and
L¹ is a divalent linker.

3. (canceled)

4. (canceled)

5. The compound of claim 2, wherein
L¹ is -L¹⁰¹-L¹⁰²-L¹⁰³-L¹⁰⁴-L¹⁰⁵-;
L¹⁰¹ is connected directly to said monovalent ABL ATP binding site inhibitor;
L¹⁰¹ is a bond, —C(O)—, —C(O)O—, —OC(O)—, —O—, —S—, —NR¹⁰¹, —C(O)NR¹⁰¹—, —NR¹⁰¹C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰² is a bond, —C(O)—, —C(O)O—, —OC(O)—, —O—, —S—, —NR¹⁰²—, —C(O)NR¹⁰²—, —NR¹⁰²C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰³ is a bond, —C(O)—, —C(O)O—, —OC(O)—, —O—, —S—, —NR¹⁰³—, —C(O)NR¹⁰³—, —NR¹⁰³C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁴ is a bond, —C(O)—, —C(O)O—, —OC(O)—, —O—, —S—, —NR¹⁰⁴—, —C(O)NR¹⁰⁴—, —NR¹⁰⁴C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁰⁵ is a bond, —C(O)—, —C(O)O—, —OC(O)—, —O—, —S—, —NR¹⁰⁵—, —C(O)NR¹⁰⁵—, —NR¹⁰⁵C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁰¹, R¹⁰², R¹⁰³, R¹⁰⁴, and R¹⁰⁵ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

6.-8. (canceled)

9. The compound of claim 5, wherein L¹ is -L¹⁰¹-(OCH₂CH₂)ₙ-L¹⁰⁴-L¹⁰⁵-;
L¹⁰¹ is substituted oxo-substituted C₁-C₆ alkylene;
L¹⁰⁴ is —NHC(O)—;
L¹⁰⁵ is unsubstituted piperidinylene; and
n is an integer from 3 to 50.

10. (canceled)

11. The compound of claim 2, wherein A is a monovalent form of dasatinib, a monovalent form of ponatinib, a monovalent form of imatinib, a monovalent form of nilotinib, a monovalent form of bosutinib, a monovalent form of bafetinib, a monovalent form of olverembatinib, a monovalent form of tozasertib, a monovalent form of PF-114, a monovalent form of rebastinib, a monovalent form of danusertib, or a monovalent form of HG-7-85-01.

12. The compound of claim 2, wherein A is a monovalent form of

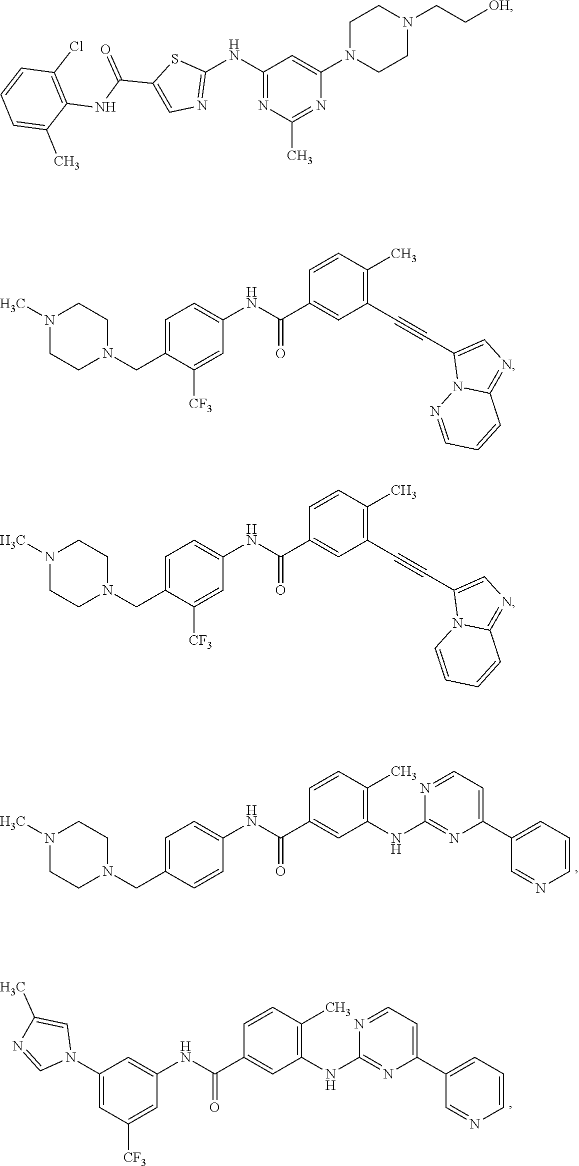 [SMILES: Cc1nc(Nc2ncc(C(=O)Nc3c(C)cccc3Cl)s2)cc(N2CCN(CCO)CC2)n1 ; Cc1ccc(C(=O)Nc2ccc(CN3CCN(C)CC3)c(C(F)(F)F)c2)cc1C#Cc1cnc2cccnn12 ; Cc1ccc(C(=O)Nc2ccc(CN3CCN(C)CC3)c(C(F)(F)F)c2)cc1C#Cc1cnc2ccccn12 ; Cc1ccc(C(=O)Nc2ccc(CN3CCN(C)CC3)cc2)cc1Nc1nccc(-c2cccnc2)n1 ; Cc1cn(-c2cc(NC(=O)c3ccc(C)c(Nc4nccc(-c5cccnc5)n4)c3)cc(C(F)(F)F)c2)cn1]

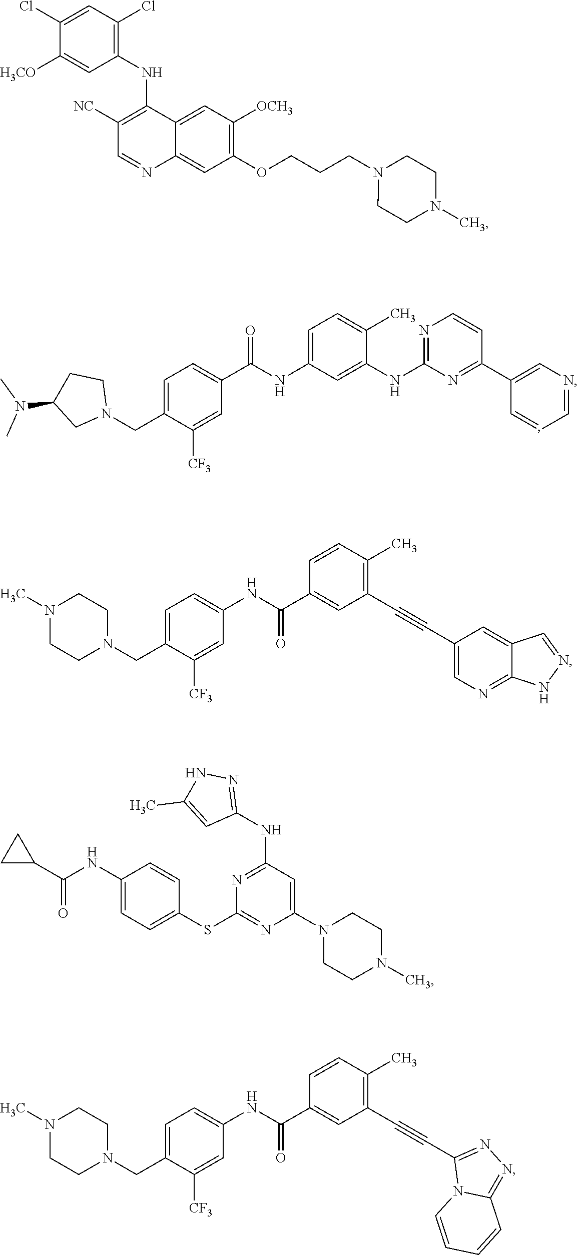

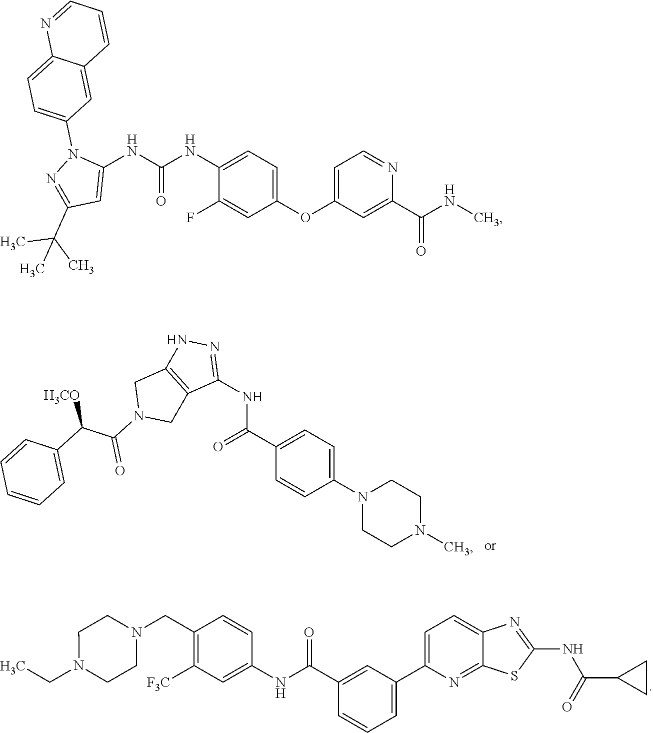 [SMILES: CNC(=O)c1cc(Oc2ccc(NC(=O)Nc3cc(C(C)(C)C)nn3-c3ccc4ncccc4c3)c(F)c2)ccn1 ; CO[C@@H](C(=O)N1Cc2[nH]nc(NC(=O)c3ccc(N4CCN(C)CC4)cc3)c2C1)c1ccccc1 ; "or" ; CCN1CCN(Cc2ccc(NC(=O)c3cccc(-c4ccc5nc(NC(=O)C6CC6)sc5n4)c3)cc2C(F)(F)F)CC1]

13. The compound of claim 12, wherein A is

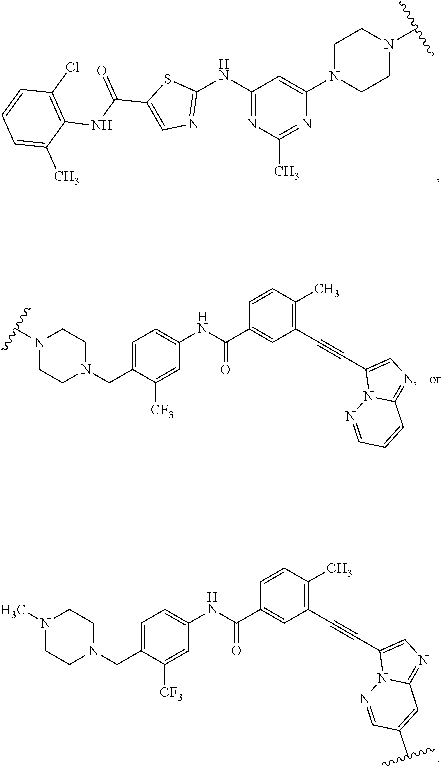 [SMILES: *N1CCN(c2cc(Nc3ncc(C(=O)Nc4c(C)cccc4Cl)s3)nc(C)n2)CC1 ; *N1CCN(Cc2ccc(NC(=O)c3ccc(C)c(C#Cc4cnc5cccnn45)c3)cc2C(F)(F)F)CC1 ; *c1cnn2c(C#Cc3cc(C(=O)Nc4ccc(CN5CCN(C)CC5)c(C(F)(F)F)c4)ccc3C)cnc2c1]

14. (canceled)

15. The compound of claim 13, wherein L¹ is 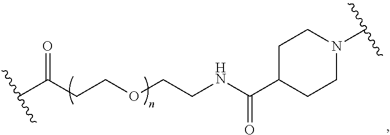
wherein n is an integer from 3 to 50.

16.-24. (canceled)

25. The compound of claim 12, wherein A is

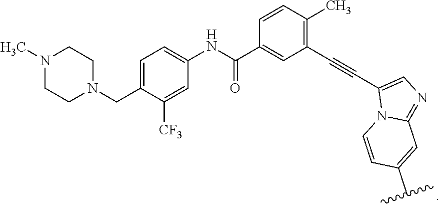 [SMILES: *c1ccn2c(C#Cc3cc(C(=O)Nc4ccc(CN5CCN(C)CC5)c(C(F)(F)F)c4)ccc3C)cnc2c1]

26. (canceled)

27. The compound of claim 25, wherein L¹ is 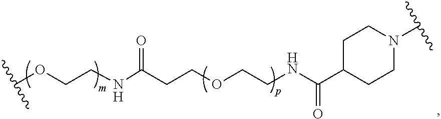
wherein m and p are independently an integer from 3 to 50.

28.-31. (canceled)

32. The compound of claim 12, wherein A is

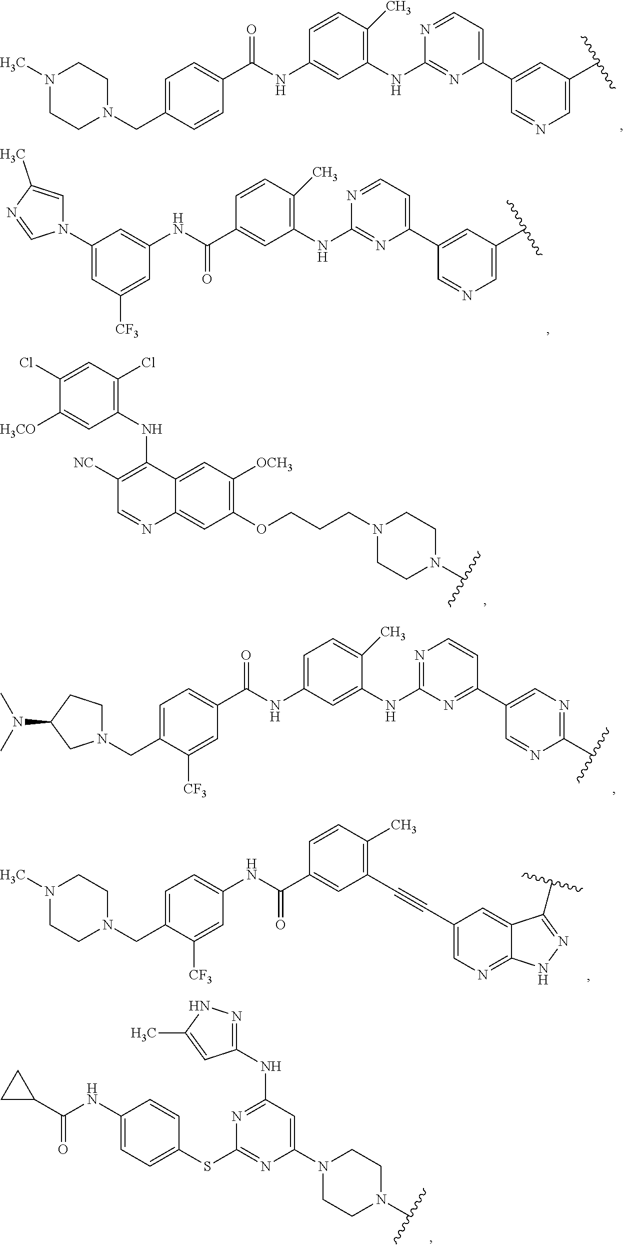

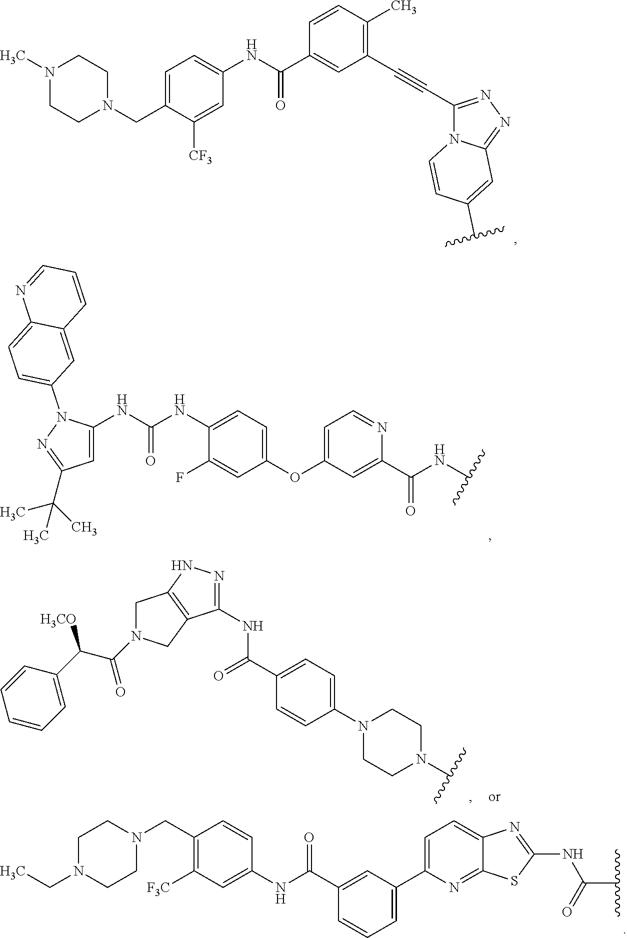

33.-50. (canceled)

51. The compound of claim 2, wherein B is a monovalent form of asciminib or a monovalent form of GNF-2.

52. The compound of claim 2, wherein B is a monovalent form of

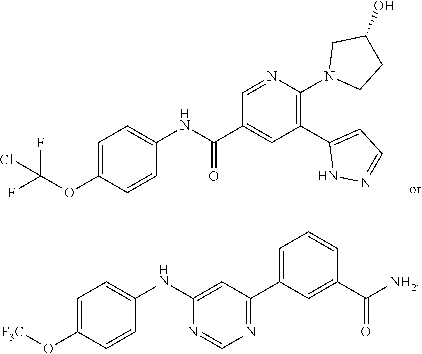 [SMILES: O=C(Nc1ccc(OC(F)(F)Cl)cc1)c1cnc(N2CC[C@@H](O)C2)c(-c2ccn[nH]2)c1 ; NC(=O)c1cccc(-c2cc(Nc3ccc(OC(F)(F)F)cc3)ncn2)c1]

53. The compound of claim 2, wherein B is

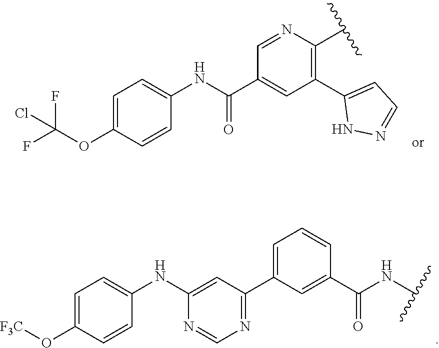 [SMILES: *c1ncc(C(=O)Nc2ccc(OC(F)(F)Cl)cc2)cc1-c1ccn[nH]1 ; *NC(=O)c1cccc(-c2cc(Nc3ccc(OC(F)(F)F)cc3)ncn2)c1]

54. (canceled)

55. (canceled)

56. The compound of claim 1, having the formula:

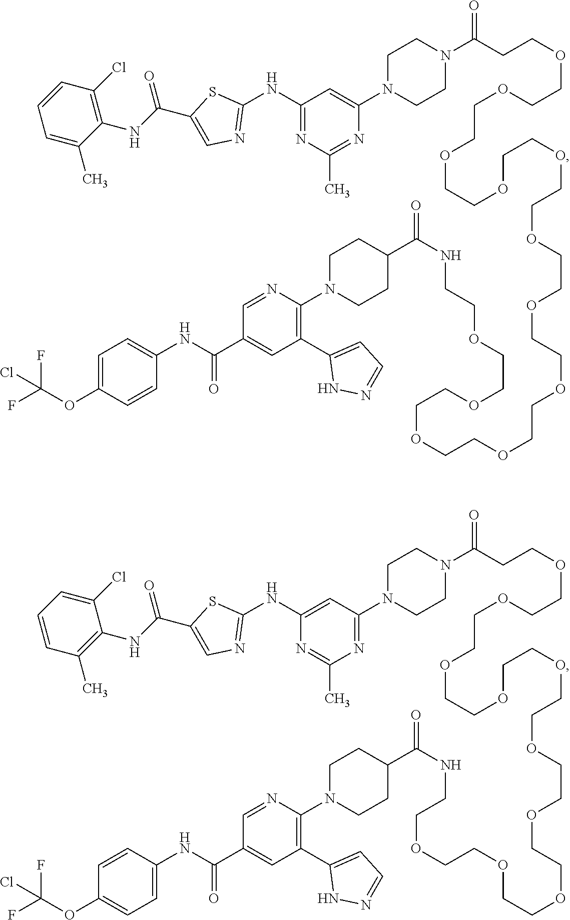 [SMILES: Cc1nc(Nc2ncc(C(=O)Nc3c(C)cccc3Cl)s2)cc(N2CCN(C(=O)CCOCCOCCOCCOCCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1 ; Cc1nc(Nc2ncc(C(=O)Nc3c(C)cccc3Cl)s2)cc(N2CCN(C(=O)CCOCCOCCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

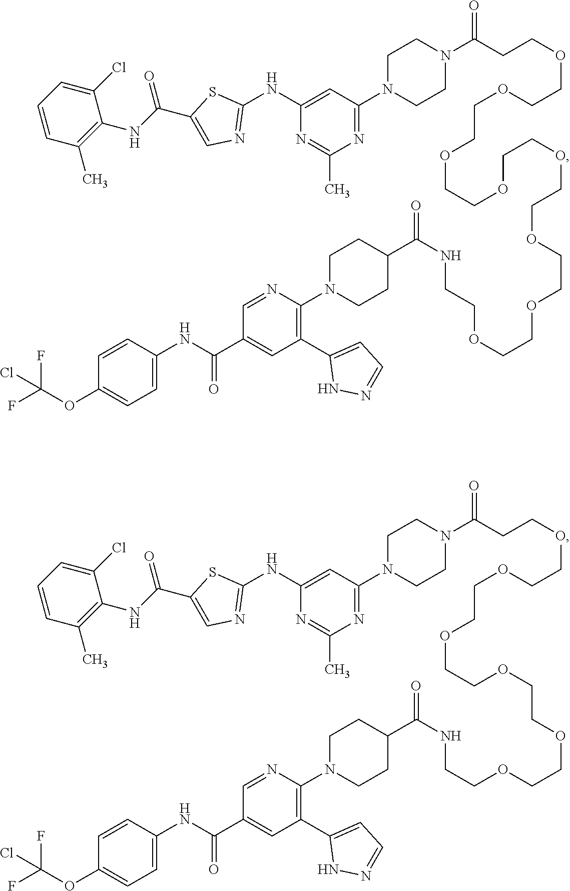 [SMILES: Cc1nc(Nc2ncc(C(=O)Nc3c(C)cccc3Cl)s2)cc(N2CCN(C(=O)CCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1 ; Cc1nc(Nc2ncc(C(=O)Nc3c(C)cccc3Cl)s2)cc(N2CCN(C(=O)CCOCCOCCOCCOCCOCCOCCNC(=O)C3CCN(c4ncc(C(=O)Nc5ccc(OC(F)(F)Cl)cc5)cc4-c4ccn[nH]4)CC3)CC2)n1]

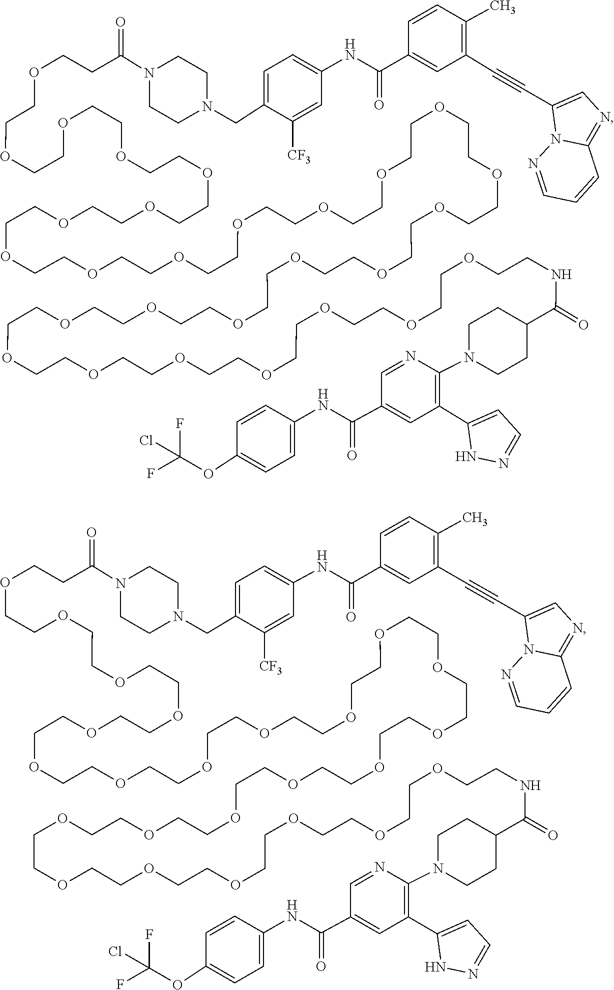 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12 ; Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

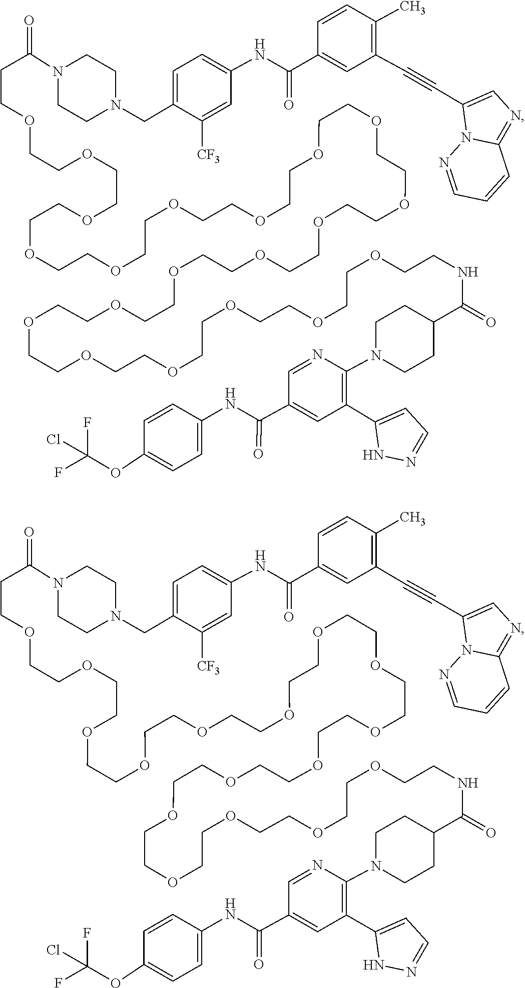 [SMILES: Cc1ccc(C(=O)Nc2ccc(CN3CCN(C(=O)CCOCCOCCOCCOCCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12 ; Cc1ccc(C(=O)Nc2ccc(CN3CCN(C(=O)CCOCCOCCOCCOCCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12]

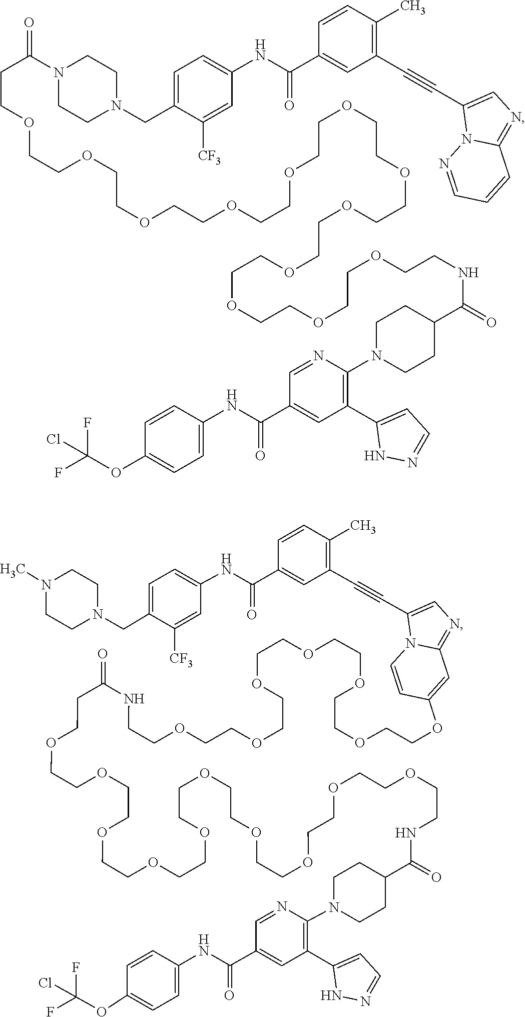 [SMILES: Cc1ccc(C(=O)Nc2ccc(CN3CCN(C(=O)CCOCCOCCOCCOCCOCCOCCOCCOCCOCCOCCOCCOCCNC(=O)C4CCN(c5ncc(C(=O)Nc6ccc(OC(F)(F)Cl)cc6)cc5-c5ccn[nH]5)CC4)CC3)c(C(F)(F)F)c2)cc1C#Cc1cnc2cccnn12 ; Cc1ccc(C(=O)Nc2ccc(CN3CCN(C)CC3)c(C(F)(F)F)c2)cc1C#Cc1cnc2cc(OCCOCCOCCOCCOCCOCCOCCNC(=O)CCOCCOCCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)ccn12]

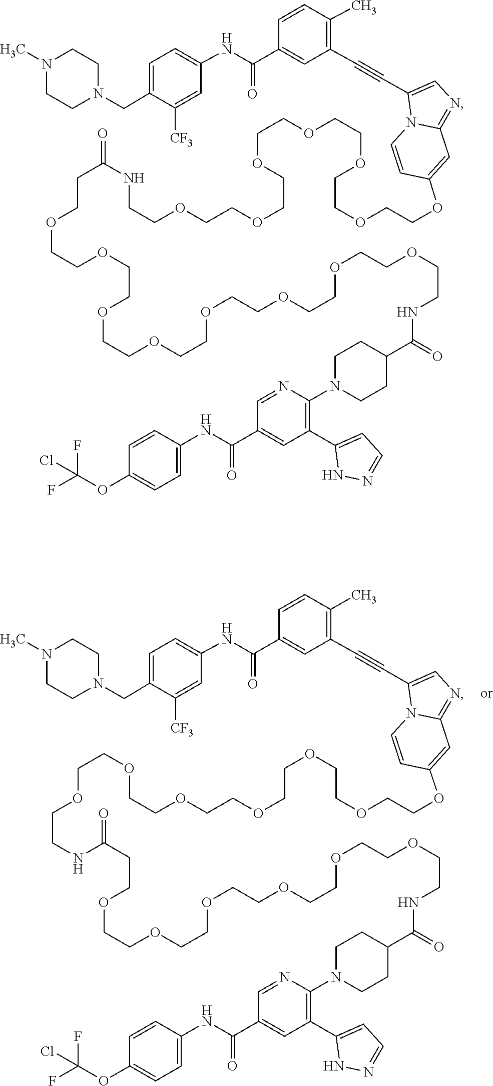 [SMILES: Cc1ccc(C(=O)Nc2ccc(CN3CCN(C)CC3)c(C(F)(F)F)c2)cc1C#Cc1cnc2cc(OCCOCCOCCOCCOCCOCCOCCNC(=O)CCOCCOCCOCCOCCOCCOCCOCCOCCNC(=O)C3CCN(c4ncc(C(=O)Nc5ccc(OC(F)(F)Cl)cc5)cc4-c4ccn[nH]4)CC3)ccn12 ; Cc1ccc(C(=O)Nc2ccc(CN3CCN(C)CC3)c(C(F)(F)F)c2)cc1C#Cc1cnc2cc(OCCOCCOCCOCCOCCOCCOCCNC(=O)CCOCCOCCOCCOCCOCCOCCNC(=O)C3CCN(c4ncc(C(=O)Nc5ccc(OC(F)(F)Cl)cc5)cc4-c4ccn[nH]4)CC3)ccn12]

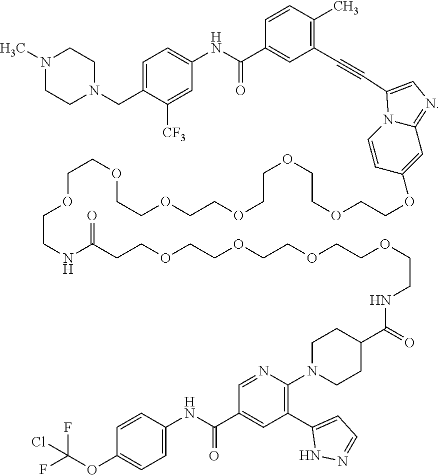 [SMILES: Cc1ccc(C(=O)Nc2ccc(CN3CCN(C)CC3)c(C(F)(F)F)c2)cc1C#Cc1cnc2cc(OCCOCCOCCOCCOCCOCCOCCNC(=O)CCOCCOCCOCCOCCNC(=O)C3CCN(c4ncc(C(=O)Nc5ccc(OC(F)(F)Cl)cc5)cc4-c4ccn[nH]4)CC3)ccn12]

57.-68. (canceled)

69. A pharmaceutical composition comprising a pharmaceutically acceptable excipient and the compound of claim 1, or a pharmaceutically acceptable salt thereof.

70. A method of treating cancer in a subject in need thereof, said method comprising administering to the subject in need thereof a therapeutically effective amount of the compound of claim 1, or a pharmaceutically acceptable salt thereof.

71. (canceled)

72. (canceled)

73. A method of treating a neurodegenerative disease in a subject in need thereof, said method comprising administering to the subject in need thereof a therapeutically effective amount of the compound of claim 1, or a pharmaceutically acceptable salt thereof.

74. (canceled)

75. A method of treating an ABL-associated disease in a subject in need thereof, said method comprising administering to the subject in need thereof a therapeutically effective amount of the compound of claim 1, or a pharmaceutically acceptable salt thereof.

76.-80. (canceled)

81. A method of reducing the level of activity of ABL in a cell, said method comprising contacting the cell with an effective amount of the compound of claim 1, or a pharmaceutically acceptable salt thereof.

82.-85. (canceled)
